(19) United States
(12) Patent Application Publication
(10) Pub. No.: US 2022/0033446 A1
(43) Pub. Date: Feb. 3, 2022

(54) SYSTEMS AND METHODS FOR DISCOVERING AND OPTIMIZING LASSO PEPTIDES

(71) Applicant: Lassogen, Inc., San Diego, CA (US)

(72) Inventors: Mark J. Burk, San Diego, CA (US); I-Hsiung Brandon Chen, La Mesa, CA (US)

(21) Appl. No.: 17/296,372

(22) PCT Filed: Dec. 9, 2019
(86) PCT No.: PCT/US2019/065244

§ 371 Date: May 24, 2021

Related U.S. Application Data
(60) Provisional application No. 62/777,702, filed on Dec. 10, 2018.

(51) Int. Cl. C07K 14/245; C07K 14/36; C07K 7/56; C40B 30/00; C40B 40/10

(52) U.S. Cl. CPC C07K 14/245; C07K 14/36; C07K 7/56; C07K 2319/70; C40B 40/10; C07K 2319/21; C07K 2319/50; C40B 30/00

(57) ABSTRACT

Provided herein are lasso peptides libraries, and particularly molecular display libraries of lasso peptides. Also provided herein are related methods and systems for producing the libraries and for screening the libraries to identify candidate lasso peptides having desirable properties.

### CROSS REFERENCE TO RELATED APPLICATIONS

This application claims the benefit of U.S. Provisional Application No. 62/777,702, filed Dec. 10, 2018; the disclosure of which is incorporated herein by reference in its entirety.

### REFERENCE TO A SEQUENCE LISTING

This application is being filed with a computer readable form (CRF) copy of a Sequence Listing named 14619-003-228_ST25.txt, created on Dec. 9, 2019, and being 103,638 bytes in size; which is incorporated herein by reference in its entirety.

### 1. FIELD

Provided herein are systems and related methods for discovering and optimizing lasso peptides.

### 2. BACKGROUND

Peptides serve as useful tools and leads for drug development since they often combine high affinity and specificity for their target receptor with low toxicity. However, their clinical use as efficacious drugs has been limited due to undesirable physicochemical and pharmacokinetic properties, including poor solubility and cell permeability, low bioavailability, and instability due to rapid proteolytic degradation under physiological conditions.

Ribosomally assembled natural peptides having a knotted topology may be used as molecular scaffold for drug design. For example, ribosomally assembled natural peptides sharing the cyclic cystine knot (CCK) motif, as exemplified by the cyclotides and conotoxins, recently have been introduced as stable molecular frameworks for potential therapeutic applications (Weidmann, J.; Craik, D. J., J. Experimental Bot., 2016, 67, 4801-4812; Burman, R., et al., J. Nat. Prod. 2014, 77, 724-736; Reinwarth, M., et al., Molecules, 2012, 17, 12533-12552; Lewis, R. J., et al., Pharmacol. Rev., 2012, 64, 259-298). But these knotted peptides require the formation of three disulfide bonds to hold them into a defined conformation. As the biosynthetic machinery of plant-derived cyclotides and animal-derived conotoxins is not well understood, these knotted peptide scaffolds are not readily accessible by genetic manipulation and heterologous production in cells and discovery relies on traditional extraction and fractionation methods that are slow and costly. Moreover, their production relies either on solid phase peptide synthesis (SPPS) or on expressed protein ligation (EPL) methods to generate the circular peptide backbone, followed by oxidative folding to form the correct three disulfide bonds required for the knotted structure (Craik, D. J., et al., Cell Mol. Life Sci. 2010, 67, 9-16; Berrade, L. & Camarero, J. A. Cell Mol. Life Sci., 2009, 66, 3909-22).

There exists a need for new classes of peptide-based therapeutic compounds with readily available methods for their discovery, genetic manipulation and evolution, cost-effective production, and high-throughput screening. The present disclosure provided herein meet these needs.

### 3. SUMMARY

Provided herein are lasso peptides and related molecules, libraries and compositions. Also provided herein are methods for optimizing and screening lasso peptide libraries for candidates having desirable properties.

In one aspect, provided herein are lasso peptide display libraries. In some embodiments, provided is a lasso peptide display library comprising a plurality of members, wherein each member comprises a lasso peptide or a functional fragment of lasso peptide; and wherein each member is associated with a unique identification mechanism for distinguishing the plurality of members from one another, wherein the unique identification mechanism is a unique nucleic acid molecule or a unique location.

In some embodiments, the library further comprises a solid support. In some embodiments, each member is associated with the unique identification mechanism through the solid support. In some embodiments, the solid support comprises a plurality of unique locations, and each member is associated with one of the plurality of unique locations.

In some embodiments of the lasso peptide display library, at least one of the lasso peptide and/or functional fragment of lasso peptide forms part of a fusion protein. In some embodiments, at least one of the lasso peptide and/or functional fragment of lasso peptide forms part of a protein complex. In some embodiments, at least one of the lasso peptide and/or functional fragment of lasso peptide forms part of a conjugate. In some embodiments, the unique identification mechanism is a unique nucleic acid molecule.

In some embodiments of the lasso peptide display library, the lasso peptide or functional fragment of lasso peptide is fused to a first binding partner; and wherein the unique nucleic acid molecule is conjugated with a second binding partner. In some embodiments, the first binding partner and the second binding partner are capable of directly or indirectly associating with one another. In some embodiments, the first binding partner and the second binding partner are both configured to associate with the solid support. In some embodiments, the solid support is coated with or comprises a third binding partner capable of associating with the first binding partner and the second binding partner.

In some embodiments of the lasso peptide display library, the first binding partner is streptavidin; and wherein the second binding partner is biotin moiety conjugated with the unique nucleic acid molecule. In some embodiments, the first binding partner is a nucleic acid binding protein and the second binding partner is target nucleic acid sequence that is a fragment of the unique nucleic acid molecule. In some embodiments, the nucleic acid binding protein is replication protein RepA and the unique nucleic acid molecule comprises replication origin R (oriR) and cis-acting element (CIS) of RepA.

In some embodiments of the lasso peptide display library, the first binding partner is a streptavidin binding protein; wherein the second binding partner is biotin moiety conjugated with the unique nucleic acid molecule; and wherein the third binding partner is streptavidin. In some embodiments, the solid support is a magnetic bead. In some embodiments, the lasso peptide or functional fragment thereof is associated with the unique nucleic acid molecule through a cleavable linker.

In some embodiments of the lasso peptide display library, the unique nucleic acid molecule is a nucleic acid barcode. In some embodiments, the unique nucleic acid molecule encodes at least a portion of the lasso peptide or functional fragment thereof associated with the unique nucleic acid.

In some embodiments, the lasso peptide display library further comprises a cell-free biosynthesis system configured for providing the plurality of members. In some embodiments, the cell-free biosynthesis system comprises a minimal set of lasso peptide biosynthesis components.

In some embodiments of the lasso peptide display library, the minimal set of lasso peptide biosynthesis components comprises (i) at least one lasso precursor peptide or (ii) a first nucleic acid sequence encoding the at least one lasso precursor peptide and cell-free transcription-translation machinery. In some embodiments, the minimal set of lasso peptide biosynthesis components comprises (i) at least one lasso core peptide or (ii) a second nucleic acid sequence encoding the at least one lasso core peptide and cell-free transcription-translation machinery. In some embodiments, the minimal set of lasso peptide biosynthesis components comprises (i) at least one lasso peptidase or (ii) a third nucleic acid sequence encoding the at least one lasso peptidase and cell-free transcription-translation machinery. In some embodiments, the minimal set of lasso peptide biosynthesis components comprises (i) at least one lasso cyclase or (ii) a fourth nucleic acid sequence encoding the at least one lasso cyclase and cell-free transcription-translation machinery. In some embodiments, the minimal set of lasso peptide biosynthesis components comprises (i) at least one RiPP recognition element (RRE) or (ii) a fifth nucleic acid sequence encoding the at least one RRE and cell-free transcription-translation machinery.

In some embodiments of the lasso peptide display library, the minimal set of lasso peptide biosynthesis components comprises (i) a plurality of a first nucleic acid sequences each encoding a unique lasso precursor peptide; (ii) at least one lasso peptidase or a third nucleic acid sequence encoding the lasso peptidase; (iii) at least one lasso cyclase or a fourth nucleic acid sequence encoding the lasso cyclase; and (iv) cell-free transcription-translation machinery.

In some embodiments of the lasso peptide display library, the plurality of the first nucleic acid sequences are derived from a same lasso peptide biosynthesis gene cluster. In some embodiments, the plurality of the first nucleic acid sequences are obtained by randomly mutating Gene A of the same lasso peptide biosynthesis gene cluster. In some embodiment, the random mutation is introduced to all codons of Gene A except for the ring-forming residue. In some embodiments, the ring-forming residue is Glu at position 6, 7, 8, 9, 10, 11, 12, 13, 14, 15, 16, 17, 18, 19 or 20, or Asp at position 6, 7, 8, 9, 10, 11, 12, 13, 14, 15, 16, 17, 18, 19 or 20.

In some embodiments of the lasso peptide display library, the plurality of the first nucleic acid sequences are obtained by changing the position of the codon coding for the ring-forming residue in Gene A of the same lasso peptide biosynthesis gene cluster. In some embodiments, the plurality of the first nucleic acid sequences are derived from a plurality of lasso peptide biosynthesis gene cluster. In some embodiments, the minimal set of lasso peptide biosynthesis components further comprises at least one RiPP recognition element (RRE) or a fifth nucleic acid sequence encoding the RRE.

In some embodiments of the lasso peptide display library, at least one of the first, second, third, fourth and fifth nucleic acid sequences are operably linked to an expression control fragment. In some embodiments, at least two of the first, second, third, fourth and fifth nucleic acid sequences form part of a same nucleic acid molecule. In some embodiments, at least two of the third, fourth and fifth nucleic acid sequences are fused in frame with each other in the same nucleic acid molecule. In some embodiments, at least two of the first, second, third, fourth and fifth nucleic acids sequences comprise sequences derived from the same lasso peptide biosynthesis gene cluster. In some embodiments, at least two of the first, second, third, fourth and fifth nucleic acid sequences comprise sequences derived from different lasso peptide biosynthesis gene clusters. In some embodiments, the third, fourth and fifth nucleic acid sequences comprise sequences derived from the same lasso peptide biosynthesis gene cluster of a host organism; and wherein the transcription-translation machinery is a cell lysate of the same host organism. In some embodiments, at least one of the first, second, third, fourth and fifth nucleic acid sequences is DNA, mRNA or cDNA sequence.

In some embodiments of the lasso peptide display library, at least one of the first, second, third, fourth and fifth nucleic acid sequences further comprises a sequence encoding for a peptidic tag. In some embodiments, the peptidic tag is a purification tag. In some embodiments, the peptidic tag comprises a cleavable linker. In some embodiments, the peptidic tag forms part of a binding partner. In some embodiments, the peptidic tag produces a detectable signal.

In some embodiments of the lasso peptide display library, the cell-free biosynthesis system comprises cell lysate or supplemented cell lysate. In some embodiments, the cell-free biosynthesis system comprises components of cellular transcription-translation machinery purified from a cell. In some embodiments, the cell-free biosynthesis system comprises synthetic or recombinantly produced components of cellular transcription-translation machinery. In some embodiments, the lasso peptide or a functional fragment of lasso peptide comprises at least one unnatural or unusual amino acid.

In some embodiments of the lasso peptide display library, the lasso peptide display library is not a bacteriophage display library that comprises lasso peptides or related molecules fused to a phage coat protein. In some embodiments, the lasso peptide display library is a molecular display library as provided herein.

In another aspect, provided herein are fusion proteins comprising a lasso peptide component fused to a binding partner. In some embodiments, the lasso peptide component is (i) a lasso peptide, (ii) a functional fragment of lasso peptide; (iii) a lasso precursor peptide; or (iv) a lasso core peptide. In some embodiments, the lasso peptide component is fused to the binding partner via a cleavable linker. In some embodiments, the binding partner is a streptavidin binding peptide (SBP), a streptavidin protein, or a nucleic acid binding protein. In some embodiments, the nucleic acid binding protein is replication protein RepA. In some embodiments, the fusion protein further comprises a purification tag. In some embodiments, the purification tag is a His Tag.

In another aspect, provided herein are nucleic acid molecules encoding a fusion protein containing a lasso peptide component. In some embodiments, the encoded lasso peptide component is (i) a lasso peptide, (ii) a functional fragment of lasso peptide; (iii) a lasso precursor peptide; or (iv) a lasso core peptide. In some embodiments, the nucleic acid molecule is biotinylated. In some embodiments, the nucleic acid molecule further comprises the replication origin R (oriR) and cis-acting element (CIS) of RepA.

In another aspect, provided herein is a molecular complex comprising a fusion protein containing a lasso peptide fragment and a nucleic acid molecule. In some embodiments, the lasso peptide component is (i) a lasso peptide, (ii) a functional fragment of lasso peptide; (iii) a lasso precursor peptide; or (iv) a lasso core peptide. In some embodiments, the nucleic acid molecule encodes at least a portion of the lasso peptide fragment. In some embodiments, the nucleic acid molecule is a unique member of a set of nucleic acid barcodes.

In some embodiments of the molecular complex, the nucleic acid molecule is biotinylated. In some embodiments, the binding partner in the fusion protein is the streptavidin protein. In some embodiments, the binding partner is the streptavidin binding peptide (SBP), and wherein the molecular complex further comprises a streptavidin protein.

In some embodiments of the molecular complex, the nucleic acid molecule comprises the replication origin R (oriR) and cis-acting element (CIS) of RepA, and wherein the first binding partner is RepA. In some embodiments of the molecular complex, the nucleic acid molecule is a nucleic acid molecule as provided herein.

In another aspect, provided herein is a composition comprising a plurality of the molecular complexes as provided herein. In some embodiments, each of the plurality of the molecular complexes comprises a unique lasso peptide or functional fragment of lasso peptide.

In another aspect, provided herein are methods for optimizing a lasso peptide of interest. In some embodiments, provided herein is a method for evolving a lasso peptide of interest for a target property, the method comprising a) providing a first lasso peptide display library comprising members derived from the lasso peptide of interest, wherein each member of the first lasso peptide display library comprises at least one mutation to the lasso peptide of interest; b) subjecting the library to a first assay under a first condition to identify members having the target property; c) identifying the mutations of the identified members as beneficial mutations; and d) introducing the beneficial mutations into the lasso peptide of interest to provide an evolved lasso peptide.

In some embodiments of the method for evolving a lasso peptide of interest, the method further comprises: f) providing an evolved lasso peptide display library comprising members derived from the evolved lasso peptide, wherein the members of the second library retain at least one beneficial mutation; and g) repeating steps b) through d). In some embodiments, the method further comprises repeating steps f) and g) for at least one more round.

In some embodiments of the method for evolving a lasso peptide of interest, the evolved lasso peptide display library is subjected to the first assay under a second condition more stringent for the target property than the first condition. In some embodiments, the evolved lasso peptide display library is subjected to a second assay to identify members having the target property. In some embodiments, the method further comprises validating the evolved lasso peptide using at least one additional assay different from the first or second assay.

In some embodiments of the method for evolving a lasso peptide of interest, the target property is binding affinity for a target molecule. In some embodiments, the target property is binding specificity for a target molecule. In some embodiments, the target property is capability of modulating a cellular activity or cell phenotype. In some embodiments, the modulation is antagonist modulation or agonist modulation.

In some embodiments of the method for evolving a lasso peptide of interest, the mutation comprises substituting at least one amino acid with an unusual or unnatural amino acid. In some embodiments, the target property is at least two target properties screened simultaneously.

In another aspect, provided herein is a method for identifying a lasso peptide that specifically binds to a target molecule, the method comprising: providing a lasso peptide display library comprising a plurality of members, each member comprising a lasso peptide or a functional fragment of lasso peptide; contacting the library with the target molecule under a suitable condition that allows at least one member of the library to form a complex with the target molecule; and identifying the member of in the complex.

In some embodiments of the method for identifying a lasso peptide that specifically binds to a target molecule, the contacting is performed by contacting the library with the target molecule in the presence of a reference binding partner of the target molecule under a suitable condition that allows at least one member of the library to compete with the reference binding partner for binding to the target molecule; and wherein the identifying step is performed by detecting reduced binding of the reference binding partner to the target molecule; and identifying the member responsible for the reduced binding.

In some embodiments of the method for identifying a lasso peptide that specifically binds to a target molecule, the reference binding partner is a ligand for the target molecule. In some embodiments, the target molecule comprises one or more target sites, and the reference binding partner specifically binds to a target site of the target molecule. In some embodiments, the reference binding partner is a natural ligand or synthetic ligand for the target molecule.

In some embodiments of the method for identifying a lasso peptide that specifically binds to a target molecule, the target molecule is at least two target molecules.

In another aspect, provided herein is a method for identifying a lasso peptide that modulates a cellular activity, the method comprising: a) providing a lasso peptide display library comprising a plurality of members, each member comprising a lasso peptide or a functional fragment of lasso peptide; b) subjecting the library to a suitable biological assay configured for measuring the cellular activity; c) detecting a change in the cellular activity; and d) identifying the members responsible for the detected change. In some embodiments, step b) is performed by subjecting the library to multiple biological assays configured for measuring the cellular activity; and the method further comprises selecting the members that have a high probability of being identified as responsible for the detected change in the cellular activity.

In another aspect, provided herein is a method for identifying an agonist or antagonist lasso peptide for a target molecule, the method comprising providing a lasso peptide display library comprising a plurality of members, each member comprising a lasso peptide or a functional fragment of lasso peptide; contacting the library with a cell expressing the target molecule under a suitable condition that allows at least one member of the library to bind to the target molecule; measuring a cellular activity mediated by the target molecule; and identifying the member as an agonist ligand for the target molecule if said cellular activity is increased; or identifying the member as an antagonist ligand if said cellular activity is decreased.

### 4. BRIEF DESCRIPTION OF THE FIGURES

The details of one or more embodiments of the present disclosure are set forth in the accompanying drawings and the description below. Other features, objects, and benefits of the present disclosure will be apparent from the description and drawings, and from the claims. All publications, patents and patent applications cited herein are hereby expressly incorporated by reference for all purposes.

The embodiments of the description described herein are not intended to be exhaustive or to limit the disclosure to the precise forms disclosed in the following drawings or detailed description. Rather, the embodiments are chosen and described so that others skilled in the art can appreciate and understand the principles and practices of the description.

FIG. 1 is a schematic illustration of the conversion of a lasso precursor peptide into a lasso peptide having the general structure 1 with the lariat-like topology.

FIG. 2 is a schematic illustration of a 26-mer linear core peptide corresponding to a lasso peptide.

FIG. 3 shows the results for detecting MccJ25 by LC/MS analysis.

FIG. 4 shows the results for detecting ukn22 by LC/MS analysis.

FIG. 5A is a schematic illustration of several exemplary embodiments of the construction of a display library for lasso peptides, including the use of DNA barcodes as a library member identification mechanism.

FIG. 5B is a schematic illustration of several exemplary embodiments of the construction of a display library for lasso peptides, including the use of linear encoding nucleic acid molecules.

FIG. 6A is a schematic illustration of several exemplary embodiments of the construction of a molecular display library for lasso peptides using lasso-encoding DNA as a library member identification mechanism, where in certain embodiments, the library utilizes beads as a solid support.

FIG. 6B is a schematic illustration of several exemplary embodiments of the construction of a molecular display library for lasso peptides using lasso-encoding DNA as a library member identification mechanism, where in certain embodiments, the library does not have a solid support.

FIG. 6C is a schematic illustration of several exemplary embodiments of the construction of a molecular display library for lasso peptides using lasso-encoding DNA as a library member identification mechanism, where in certain embodiments, the library does not have a solid support.

FIG. 7A is a schematic illustration of an exemplary embodiment of the screening of a molecular library for candidate library member(s) having a desirable property, including assaying in vitro binding of an isolated target molecule to immobilized lasso peptides of a library.

FIG. 7B is a schematic illustration of an exemplary embodiment of the screening of a molecular library for candidate library member(s) having a desirable property, including assaying in vitro binding of lasso peptides to isolated and immobilized target molecules.

FIG. 7C is a schematic illustration of an exemplary embodiment of the screening of a molecular library for candidate library member(s) having a desirable property, including assaying in vitro binding of lasso peptides to target molecules expressed on adherent cells.

FIG. 7D is a schematic illustration of an exemplary embodiment of the screening of a molecular library for candidate library member(s) having a desirable property, including assaying in vitro binding of lasso peptides to target molecules expressed on suspended cells.

FIG. 8 is a schematic illustration of several exemplary embodiments of methods for identifying candidate lasso peptides using flow cytometry.

FIG. 9 is a schematic illustration of an exemplary embodiment of methods for identifying candidate lasso peptides using single cell binding assay.

FIG. 10 is a schematic illustration showing conversion of biotinylated DNA into MBP-FusA-TEV-SAV (SEQ ID NO:62), the binding of MBP-FusA-TEV-SAV to its cognate biotin-DNA, or conversion of MBP-FusA-TEV-SAV into Fusilassin-TEV-SAV (SEQ ID NO:63) and subsequent TEV cleavage to release the matured lasso peptide (SEQ ID NO:59), as demonstrated by the mass spectrum analysis.

### 5. DETAILED DESCRIPTION

The features of the present disclosure are set forth specifically in the appended claims. A better understanding of the features and benefits of the present disclosure will be obtained by reference to the following detailed description that sets forth illustrative embodiments, in which the principles of the disclosure are utilized. To facilitate a full understanding of the disclosure set forth herein, a number of terms are defined below.

### 5.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (4th ed. 2012); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2nd ed. 2010). Molecular Biology of the Cell (6th Ed., 2014). Organic Chemistry, (Thomas Sorrell, 1999). March's Advanced Organic Chemistry (6^{th} ed. 2007). Lasso Peptides, (Li, Y.; Zirah, S.; Rebuffet, S., Springer; New York, 2015).

### 5.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

Generally, the nomenclature used herein and the laboratory procedures in organic chemistry, medicinal chemistry, molecular biology, microbiology, biochemistry, enzymology, computational biology, computational chemistry, and pharmacology described herein are those well-known and commonly employed in the art. Unless defined otherwise, all technical and scientific terms used herein generally have the same meaning as commonly understood by one of ordinary skill in the art to which this disclosure belongs. Methods and compounds of the present disclosure include those described generally above, and are further illustrated by the classes, subclasses, and species disclosed herein. As used herein, the following definitions shall apply unless otherwise indicated. For purposes of the present disclosure, the chemical elements are identified in accordance with the Periodic Table of the Elements, CAS version, Handbook of Chemistry and Physics, 75th Ed. General methods and principles of molecular biology and cloning are described in “Molecular Cloning: A Laboratory Manual”, 4^{th} edition, Michael R. Green and Joseph Sambrook, Cold Spring Harbor Laboratory Press, 2012 and “Molecular Biology of the Cell”, 6^{th} Ed., Bruce Alberts, Alexander Johnson, Julian Lewis, David Morgan, Martin Raff, Keith Roberts, Peter Walter, Garland Science Press, 2014, the entire contents of which are hereby incorporated by reference. Additionally, general principles of organic chemistry are described in “Organic Chemistry”, Thomas Sorrell, University Science Books, Sausalito: 1999, and “March's Advanced Organic Chemistry”, 6^{th}Ed. Ed.: Smith, M. B. and March, J., John Wiley & Sons, New York: 2007, the entire contents of which are hereby incorporated by reference.

As used herein, the singular terms “a,” “an,” and “the” include the plural reference unless the context clearly indicates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

As used herein, the term “naturally occurring” or “naturally existing” or “natural” or “native” when used in connection with biological materials such as nucleic acid molecules, polypeptides, host cells, oligonucleotides, amino acids, polypeptides, peptides, metabolites, small molecule natural products, host cells, and the like, refers to those that are found in or isolated directly from Nature and are not changed or manipulated by humans.

The term “natural” or “naturally occurring” refers to organisms, cells, genes, biosynthetic gene clusters, enzymes, proteins, oligonucleotides, and the like that are found in Nature and are unchanged relative to these components found in Nature. The term “wild-type” refers to organisms, cells, genes, biosynthetic gene clusters, enzymes, proteins, oligonucleotides, and the like that are found in Nature and are unchanged relative to these components found in Nature (in the wild).

As defined herein, the term “natural product” refers to any product, a small molecule, organic compound, or peptide produced by living organisms, e.g., prokaryotes or eukaryotes, found in Nature, and which are produced through natural biosynthetic processes. As defined herein, “natural products” are produced through an organism's secondary metabolism or through biosynthetic pathways that are not essential for survival and not directly involved in cell growth and proliferation.

As used herein, the terms “non-naturally occurring” or “non-natural” or “unnatural” or “non-native” refer to a material, substance, molecule, cell, enzyme, protein or peptide that is not known to exist or is not found in Nature or that has been structurally modified and/or synthesized by humans. The terms “non-natural” or “unnatural” or “non-naturally occurring” when used in reference to a microbial organism or microorganism or cell extract or gene or biosynthetic gene cluster of the present disclosure is intended to mean that the microbial organism or derived cell extract or gene or biosynthetic gene cluster has at least one genetic alteration not normally found in a naturally occurring strain or a naturally occurring gene or biosynthetic gene cluster of the referenced species, including wild-type strains of the referenced species. Genetic alterations include, for example, introduction of expressible oligonucleotides or nucleic acids encoding polypeptides, other nucleic acid additions, nucleic acid deletions and/or other functional disruption of the microbial organism's genetic material. Such modifications include, for example, nucleotide changes, additions, or deletions in the genomic coding regions and functional fragments thereof, used for heterologous, homologous or both heterologous and homologous expression of polypeptides. Additional modifications include, for example, nucleotide changes, additions, or deletions in the genomic non-coding and/or regulatory regions in which the modifications alter expression of a gene or operon. Exemplary polypeptides include enzymes, proteins, or peptides within a lasso peptide biosynthetic pathway.

The terms “oligonucleotide” and “nucleic acid” refer to oligomers of deoxyribonucleotides (e.g., DNA) or ribonucleotides (e.g., RNA) and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer, M. A., et al., Nucleic Acid Res., 1991, 19, 5081-1585; Ohtsuka, E. et al., J. Biol. Chem., 1985, 260, 2605-2608; and Rossolini, G. M., et al., Mol. Cell. Probes, 1994, 8, 91-98). “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell or CFB system that produces a lasso peptide of the present disclosure may include a bacterial and eukaryotic host cells or cell lysates into which nucleic acids encoding the lasso peptide have been introduced. Suitable host cells and CFB systems are disclosed below.

Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “encoding nucleic acid” or grammatical equivalents thereof as it is used in reference to nucleic acid molecule refers to a nucleic acid molecule in its native state or when manipulated by methods well known to those skilled in the art that can be transcribed to produce mRNA, which is then translated into a polypeptide and/or a fragment thereof. The antisense strand is the complement of such a nucleic acid molecule, and the encoding sequence can be deduced therefrom.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

As used herein, the term “biosynthetic gene cluster” refers to one or more nucleic acid molecule(s) independently or jointly comprising one or more coding sequences for a precursor and processing machinery capable of maturing the precursor into a biosynthetic end product. The coding sequences can comprise multiple open reading frames (ORFs) each independently coding for one component of the precursor and processing machinery. Alternatively, the coding sequences can comprise an ORF coding for two or more components of the precursor and processing machinery fused together, as further described herein. A biosynthetic gene cluster can be identified and isolated from the genome of an organism. Computer-based analytical tools can be used to mine genomic information and identify biosynthetic gene clusters encoding lasso peptides. For example, the genome-mining tool known as Rapid ORF Description and Evaluation Online (RODEO) has been used to identify more than a thousand of lasso biosynthetic gene clusters based on available genomic information (Tietz et al. Nat Chem Biol. 2017 May; 13(5): 470-478). Alternatively, a biosynthetic gene cluster can be assembled by artificially producing and combining the nucleic acid components of the gene cluster, using genetic manipulating methods and technology known in the art.

The term “amino acid” refers to naturally occurring and non-naturally occurring alpha-amino acids, as well as alpha-amino acid analogs and amino acid mimetics that function in a manner similar to the naturally occurring alpha-amino acids. Naturally encoded amino acids are the 22 common amino acids (alanine, arginine, asparagine, aspartic acid, cysteine, glutamine, glutamic acid. glycine, histidine, isoleucine, leucine, lysine, methionine, phenylalanine, proline, serine, threonine, tryptophan, tyrosine, valine, pyrrolysine and selenocysteine). Amino acid analogs or derivatives refers to compounds that have the same basic chemical structure as a naturally occurring amino acid, i.e., a carbon that is bound to a hydrogen, a carboxyl group, an amino group, and a side chain R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “non-natural amino acid” or “non-proteinogenic amino acid” or “unnatural amino acid” refer to alpha-amino acids that contain different side chains (different R groups) relative to those that appear in the twenty-two common or naturally occurring amino acids listed above. In addition, these terms also can refer to amino acids that are described as having D-stereochemistry, rather than L-stereochemistry of natural amino acids, despite the fact that some amino acids do occur in the D-stereochemical form in Nature (e.g., D-alanine and D-serine). Additional examples of non-natural amino acids are known in the art, such as those found in Hartman et al. PLoS One. 2007 Oct. 3; 2(10):e972; Hartman et al., Proc Natl Acad Sci USA. 2006 Mar. 21; 103(12):4356-61; and Fiacco et al. Chembiochem. 2016 Sep. 2; 17(17):1643-51.

The terms “polypeptide” and “protein” are used interchangeably herein to refer to a polymer of greater than about fifty (50) amino acid residues. That is, a description directed to a polypeptide applies equally to a description of a protein, and vice versa. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins (i.e., antigens), wherein the amino acid residues are linked by covalent peptide bonds.

The term “peptide” as used herein refers to a polymer chain containing between two and fifty (2-50) amino acid residues. The terms apply to naturally occurring amino acid polymers as well as amino acid polymers in which one or more amino acid residues is a non-naturally occurring amino acid, e.g., an amino acid analog or non-natural amino acid.

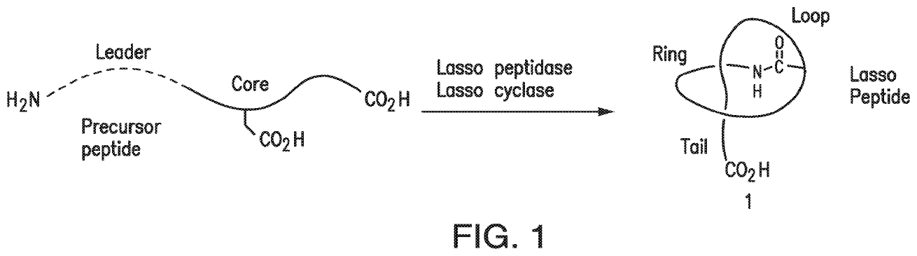

The terms “lasso peptide” and “lasso” are used interchangeably herein, and is used to refer to a class of peptide or polypeptide having the general lariat-like topology as exemplified in FIG. 1. As shown in the figure, the lariat-like topology can be generally divided into a terminal ring portion, a middle loop portion, and a terminal tail portion. Particularly, a region on one end of the peptide forms the ring around the tail on the other end of the peptide, the tail is threaded through the ring, and a middle loop portion connects the ring and the tail, together forming the lariat-like topology. Particularly, the amino acid residues that are joined together to form the ring are herein referred to as the “ring-forming amino acid.” A ring-forming amino acid can located at the N- or C-terminus of the lasso peptide (“terminal ring-forming amino acid”), or in the middle (but not necessarily the center) of a lasso peptide (“internal ring-forming amino acid”). The fragment of a lasso peptide between and including the two ring-forming amino acid residues is the ring portion; the fragment of a lasso peptide between the internal ring-forming amino acid and where the peptide threaded through the plane of the ring is the loop portion; and the remaining fragment of a lasso peptide starting from where the peptide threaded through the plane of the ring is the tail portion. In addition to the lariat-like topology, additional topological features of a lasso peptide may further include intra-peptide disulfide bonding, such as disulfide bond(s) between the tail and the ring, between the ring and the loop, and/or between different locations within the tail. As used herein, “lasso peptide” or “lasso” refers to both naturally-existing peptides and artificially produced peptides that have the lariat-like topology as described herein. Similarly, “lasso peptide” or “lasso” also refers to analogs, derivatives, or variants of a lasso peptide, which analogs, derivatives or variants are also lasso peptides themselves.

The term “lasso precursor peptide” or “precursor peptide” as used herein refers to a precursor that is processed into or otherwise form a lasso core peptide. In some embodiments, a lasso precursor peptide comprises at least one a lasso core peptide portion. In some embodiments, a lasso precursor peptide comprises one or more amino acid residues or amino acid fragments that do not belong to a lasso core peptide, such as a leader sequence that facilitates recognition of the lasso precursor peptide by one or more lasso processing enzymes. In some embodiments, the lasso precursor peptide is enzymatically processed into a lasso core peptide by removing the amino acid residues or fragments that do not belong to a lasso core peptide. In some embodiments, a lasso precursor peptide is the substrate of an enzyme that cleaves off the additional amino acid residues or fragments from a lasso precursor peptide to produce the lasso core peptide. As used herein, the enzyme capable of catalyzing this reaction is referred to as the “lasso peptidase”.

The term “lasso core peptide” or “core peptide” refers to the peptide that is processed into or otherwise forms a lasso peptide having the lariat-like topology. In some embodiments, a core peptide has the same amino acid sequence as a lasso peptide, but has not matured to have the lariat-like topology of a lasso peptide. In various embodiments, core peptides can have different amino acid sequences of lengths. In some embodiments, the core peptide is at least about 5 amino acid long. In some embodiments, the core peptide is at least about 10 amino acid long. In some embodiments, the core peptide is at least about 11 amino acid long. In some embodiments, the core peptide is at least about 12 amino acid long. In some embodiments, the core peptide is at least about 13 amino acid long. In some embodiments, the core peptide is at least about 14 amino acid long. In some embodiments, the core peptide is at least about 15 amino acid long. In some embodiments, the core peptide is at least about 16 amino acid long. In some embodiments, the core peptide is at least about 17 amino acid long. In some embodiments, the core peptide is at least about 18 amino acid long. In some embodiments, the core peptide is at least about 19 amino acid long. In some embodiments, the core peptide is at least about 20 amino acid long. In some embodiments, the core peptide is at least about 25 amino acid long. In some embodiments, the core peptide is at least about 30 amino acid long. In some embodiments, the core peptide is at least about 35 amino acid long. In some embodiments, the core peptide is at least about 40 amino acid long. In some embodiments, the core peptide is at least about 45 amino acid long. In some embodiments, the core peptide is at least about 50 amino acid long. In some embodiments, the core peptide is at least about 55 amino acid long. In some embodiments, the core peptide is at least about 60 amino acid long. In some embodiments, the core peptide is at least about 65 amino acid long.

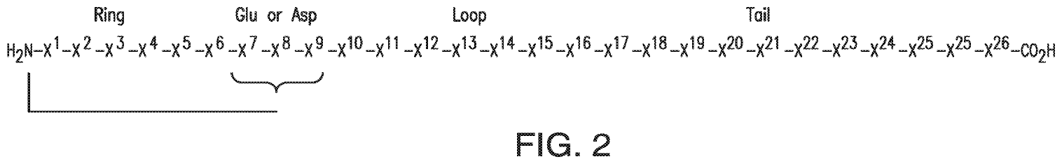

FIG. 2 shows an exemplary 26-mer linear lasso core peptide. Mutational analysis of the lasso precursor peptides McjA of microcin J25 and CapA of capistruin has revealed the high promiscuity of the biosynthetic machineries and the high plasticity of the lasso peptide structure, including the introduction of non-natural amino acids (See: Knappe, T. A., et al., Chem. Biol., 2009, 16, 1290-1298; Pavlova, O., et al. J. Biol. Chem., 2008, 283, 25589-25595; A1 Toma, R. S., et al., ChemBioChem, 2015, 16, 503-509). In addition, the feasible heterologous production of various variants in bacterial strains such as Escherichia coli and Streptomyces lividans indicates the relative ease of lasso peptide production. (See: Hegemann, J. D., et al., Biopolymers, 2013, 100, 527-542). The C-terminus of some lasso peptides has been shown to provide a source for diversification, for example through the formation of fusion peptides and proteins (See: Zong, C., et al., ACS Chem. Biol., 2016, 11, 61-68). Finally, the unique three-dimensional lariat-like topology of lasso peptides are difficult to achieve during chemical synthesis processes, but can be produced using a biosynthetically processes either in a host organism, or in a CFB system, having lasso precursors and lasso peptide biosynthetic enzymes.

Some naturally existing lasso peptides are encoded by a lasso peptide biosynthetic gene cluster, which typically comprises three main genes: one encodes for a lasso precursor peptide (referred to as Gene A), and two encode for processing enzymes including a lasso peptidase (referred to as Gene B) and a lasso cyclase (referred to as Gene C). The lasso precursor peptide comprises a lasso core peptide and additional peptidic fragments known as the “leader sequence” that facilitates recognition and processing by the processing enzymes. The leader sequence may determine substrate specificity of the processing enzymes. The processing enzymes encoded by the lasso peptide gene cluster convert the lasso precursor peptide into a matured lasso peptide having the lariat-like topology. Particularly, the lasso peptidase remove additional sequences from the precursor peptide to generate a lasso core peptide, and the lasso cyclase cyclize a terminal portion of the core peptide around a terminal tail portion to form the lariat-like topology.

Some lasso gene clusters further encodes for additional protein elements that facilitates the post-translational modification, including a facilitator protein known as the post-translationally modified peptide (RiPP) recognition element (RRE). A lasso peptide biosynthetic gene clusters may encode two or more of lasso peptidase, lasso cyclase and RRE as different domains in the same protein. Some lasso gene clusters further encodes for lasso peptide transporters, kinases, or proteins that play a role in immunity, such as isopeptidase. (Burkhart, B. J., et al., Nat. Chem. Biol., 2015, 11, 564-570; Knappe, T. A. et al., J. Am. Chem. Soc., 2008, 130, 11446-11454; Solbiati, J. O. et al. J. Bacteriol., 1999, 181, 2659-2662; Fage, C. D., et al., Angew. Chem. Int. Ed., 2016, 55, 12717-12721; Zhu, S., et al., J. Biol. Chem. 2016, 291, 13662-13678).

Artificially produced lasso peptides may or may not be the same as a naturally-existing lasso peptide. For example, some artificially produced lasso peptides are non-naturally occurring lasso peptides. Some artificially produced lasso peptides can have a unique amino acid sequence and/or structure (e.g. lariat-like topology) that is different from those of any naturally-existing lasso peptide. Some artificially produced lasso peptides are analogs or derivatives of naturally-existing lasso peptides.

The terms “analog” and “derivative” are used interchangeably to refer to a molecule such as a lasso peptide, that have been modified in some fashion, through chemical or biological means, to produce a new molecule that is similar but not identical to the original molecule. For example, analogs or derivatives of a naturally-existing lasso peptide include a peptide or polypeptide that comprises an amino acid sequence of the naturally-existing lasso peptide, which has been altered by the introduction of amino acid residue substitutions, deletions, or additions. Analogs or derivatives of a naturally-existing lasso peptide also include a lasso peptide which has been chemically modified, e.g., by the covalent attachment of any type of molecule to the polypeptide. For example, but not by way of limitation, a lasso peptide may be chemically modified, e.g., by increase or decrease of glycosylation, acetylation, pegylation, phosphorylation, amidation, derivatization by known protecting/blocking groups, proteolytic cleavage, chemical cleavage, linkage to a cellular ligand or other protein, etc. The derivatives are modified in a manner that is different from naturally occurring or starting peptide or polypeptides, either in the type or location of the molecules attached. Derivatives further include deletion of one or more chemical groups which are naturally present on the peptide or polypeptide. Further, a derivative of a lasso peptide, or a fragment of a lasso peptide may contain one or more non-classical or non-natural amino acids. A peptide or polypeptide derivative possesses a similar or identical function as a lasso peptide or a fragment of a lasso peptide. As used herein, an analog or derivative of a lasso peptide may but not necessary have a similar amino acid sequence as the original lasso peptide. A peptide or polypeptide that has a similar amino acid sequence refers to a peptide or polypeptide that satisfies at least one of the followings: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a lasso peptide or a fragment of a lasso peptide; (b) a peptide of polypeptide encoded by a nucleotide sequence that hybridizes under stringent conditions to a nucleotide sequence encoding a lasso peptide or a fragment of a GPR132 polypeptide described herein of at least 5 amino acid residues, at least 10 amino acid residues, at least 15 amino acid residues, at least 20 amino acid residues, at least 25 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues (see, e.g., Sambrook et al., Molecular Cloning: A Laboratory Manual (2001); and Maniatis et al., Molecular Cloning: A Laboratory Manual (1982)); or (c) a peptide or polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the nucleotide sequence encoding a lasso peptide or a fragment of a lasso peptide. A peptide or polypeptide with similar structure to a lasso peptide or a fragment of a lasso peptide refers to a peptide or polypeptide that has a similar secondary, tertiary, or quaternary structure of a lasso peptide or a fragment of a lasso peptide. The structure of a peptide or polypeptide can be determined by methods known to those skilled in the art, including but not limited to, X-ray crystallography, nuclear magnetic resonance, and crystallographic electron microscopy.

The term “variant” as used herein refers to a peptide or polypeptide comprising one or more (such as, for example, about 1 to about 25, about 1 to about 20, about 1 to about 15, about 1 to about 10, about 1 to about 5, or about 1 to about 3) amino acid sequence substitution, deletions, and/or additions as compared to a native or unmodified sequence. For example, a lasso peptide variant may result from one or more (such as, for example, about 1 to about 25, about 1 to about 20, about 1 to about 15, about 1 to about 10, about 1 to about 5, or about 1 to about 3) changes to an amino acid sequence of the native counterpart. Variants may be naturally occurring, such as allelic or splice variants, or may be artificially constructed. Polypeptide variants may be prepared from the corresponding nucleic acid molecules encoding the variants. In specific embodiments, the lasso peptide variant at least retains functionality of the native lasso peptide. For example, a variant of an antagonist lasso peptide. In specific embodiments, a lasso peptide variant binds to a target molecule and/or is antagonistic to the target molecule activity. In specific embodiments, a lasso peptide variant binds a target molecule and/or is agonistic to the target molecule activity. In certain embodiments, the variant is encoded by a single nucleotide polymorphism (SNP) variant of a nucleic acid molecule that encodes a lasso peptide, regions or sub-regions thereof, such as the ring, loop and/or tail portions of the lasso core peptide. In certain embodiments, variants of lasso peptides can be generated by modifying a lasso peptide, for example, by (i) introducing an amino acid sequence substitution or mutation, including the introduction of an unnatural or unusual amino acid, (ii) creating fragment of a lasso peptide; (iii) creating a fusion protein comprising one or more lasso peptides or fragment(s) of lasso peptides, and/or other non-lasso proteins or peptides, (iv) introducing chemical or biological transformation of the chemical functionality present in naturally-existing lasso peptides (e.g., inducing acylation, biotinylation, O-methylation, N-methylation, amidation, etc.), (v) making isotopic variants of naturally-existing lasso peptides, or any combinations of (i) to (v). For example, in one embodiment, one or more target-binding motif is introduced into a lasso peptide to provide a lasso peptide that specifically binds to a target molecule. For example, in some embodiments, a tripeptide Arg-Gly-Asp consists of Arginine, Glycine and Aspartate residues is introduced into a lasso peptide to create a lasso peptide variant that binds to a target integrin receptor.

Artificially produced lasso peptides can be recombinantly produced using, for example, in vitro or in vivo recombinant expression systems, or synthetically produced.

The term “isotopic variant” when used in relation to a lasso peptide, refers to lasso peptides that contains an unnatural proportion of an isotope at one or more of the atoms that constitute such a peptide. In certain embodiments, an “isotopic variant” of a lasso peptide contains unnatural proportions of one or more isotopes, including, but not limited to, hydrogen (¹H), deuterium CH), tritium (³H), carbon-11 (¹¹C), carbon-12 (¹²C) carbon-13 (¹³C), carbon-14 (¹⁴C), nitrogen-13 (¹³N), nitrogen-14 (¹⁴N), nitrogen-15 (¹⁵N), oxygen-14 (¹⁴0), oxygen-15 (¹⁵0), oxygen-16 (¹⁶O), oxygen-17 (¹⁷0), oxygen-18 (¹⁸O) fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I) iodine-125 (¹²⁵I) iodine-127 (¹²⁷I) iodine-129 (¹²⁹I) and iodine-131 (¹³¹I). In certain embodiments, an “isotopic variant” of a lasso peptide is in a stable form, that is, non-radioactive. In certain embodiments, an “isotopic variant” of a lasso peptide contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O) oxygen-17 (¹⁷0), oxygen-18 (¹⁸O) fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a lasso peptide is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18(¹⁸F), phosphorus-32 (³²P), phosphorus-33 (³³P), sulfur-35 (³⁵S), chlorine-36 (³⁶Cl), iodine-123 (¹²³I) iodine-125 (¹²⁵I), iodine-129 (¹²⁹I) and iodine-131 (¹³¹I). It will be understood that, in a lasso peptide as provided herein, any hydrogen can be ²H, as example, or any carbon can be ¹³C, as example, or any nitrogen can be ¹⁵N, as example, and any oxygen can be ¹⁸O, as example, where feasible according to the judgment of one of skill in the art. In certain embodiments, an “isotopic variant” of a lasso peptide contains an unnatural proportion of deuterium. Unless otherwise stated, structures depicted herein are also meant to include lasso peptides that differ only in the presence of one or more isotopically enriched atoms from their naturally-existing counterparts. For example, lasso peptides having the present structures including the replacement of hydrogen by deuterium or tritium, or the replacement of a carbon by a ¹³C- or ¹⁴C-enriched carbon are within the scope of the present disclosure. Such lasso peptides are useful, for example, as analytical tools, as probes in biological assays, or as therapeutic agents in accordance with the present disclosure.

An “isolated” peptide or polypeptide (e.g., lasso peptide or a lasso processing enzyme) is substantially free of cellular material or other contaminating proteins from the cell or tissue source and/or other contaminant components from which the peptide or polypeptide is derived (such as culture medium of the host organism or the CFB reaction mixture), or substantially free of chemical precursors or other chemicals when chemically synthesized. The language “substantially free” of cellular material or other contaminant components includes preparations of a peptide or polypeptide in which the peptide or polypeptide is separated from components of the cells or CFB system from which it is isolated, recombinantly produced or biosynthesized. Thus, a peptide or polypeptide that is substantially free of cellular material includes preparations of lasso peptide having less than about 30%, 25%, 20%, 15%, 10%, 5%, or 1% (by dry weight) of heterologous protein (also referred to herein as a “contaminating protein”). In certain embodiments, when the peptide or polypeptide is recombinantly produced, it is substantially free of culture medium, e.g., culture medium represents less than about 20%, 15%, 10%, 5%, or 1% of the volume of the protein preparation. In certain embodiments, when the peptide or polypeptide is produced by chemical synthesis, it is substantially free of chemical precursors or other chemicals, for example, it is separated from chemical precursors or other chemicals that are involved in the synthesis of the protein. In specific embodiments, where a lasso peptide is produced by cell-free biosynthesis, it is substantially free of lasso precursors, lasso processing enzymes, and/or in vitro TX-TL machinery in the CFB system. Accordingly such preparations of the lasso peptide have less than about 30%, 25%, 20%, 15%, 10%, 5%, or 1% (by dry weight) of chemical precursors or compounds other than the lasso peptide of interest. Contaminant components can also include, but are not limited to, materials that would interfere with therapeutic uses for the lasso peptide, and may include enzymes, hormones, and other proteinaceous or nonproteinaceous solutes. In certain embodiments, a peptide or polypeptide will be purified (1) to greater than 95% by weight of lasso peptide as determined by the Lowry method (Lowry et al., 1951, J. Bio. Chem. 193: 265-75), such as 96%, 97%, 98%, or 99%, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. In specific embodiments, an isolated lasso peptide includes the lasso peptide in situ within recombinant cells since at least one component of the lasso peptide's natural environment will not be present. Ordinarily, however, isolated peptide and polypeptide will be prepared by at least one purification step. In specific embodiments, lasso peptides, or lasso precursors, one or more of lasso processing enzymes and co-factors provided herein are isolated.

The term “minimal set of lasso peptide biosynthesis components” as used herein refers to the minimum combination of components that is able to biosynthesize a lasso peptide without the help of any additional substance or functionality. The make-up of the minimal set of lasso peptide biosynthesis components may vary depending on the content and functionality of its component. Furthermore, the components forming the minimal set may present in varied forms, such as peptide, proteins, and nucleic acids. For the sole purpose of illustration and by way of non-exhaustive and non-limiting examples, in some embodiments, a minimal set of lasso peptide biosynthesis components comprises a lasso precursor, a lasso peptidase and a lasso cyclase in a condition suitable for lasso formation. In alternative embodiments, a minimal set of lasso peptide biosynthesis components comprises a lasso core peptide and a lasso cyclase in a condition suitable for lasso formation. In yet alternative embodiments, a minimal set of lasso peptide biosynthesis components comprises a lasso peptide biosynthesis gene cluster and in vitro transcription and translation (TX-TL) machinery in a condition suitable for lasso formation. In particular embodiments as described further below, certain components of a minimal set of lasso peptide biosynthesis components can be recombinantly produced, while other components synthesized; the differentially produced components can be combined into a minimal set of lasso peptide biosynthesis components to produce a lasso peptide.

As used herein, the terms “in vitro transcription and translation” and “in vitro TX-TL” are used interchangeably and refer to a biosynthetic process outside an intact cell, where genes or oligonucleotides are transcribed into messenger ribonucleic acids (mRNAs), and mRNAs are translated into proteins or peptides. As used herein, the term “in vitro TX-TL machinery” refers to the components that act in concert to carry out the in vitro TX-TL. For the sole purpose of illustration, and by way of non-exhaustive and non-limiting examples, in some embodiments, an in vitro TX-TL machinery comprises enzyme(s) and co-factor(s) that carry out DNA transcription and/or mRNA translation. In some embodiments, an in vitro TX-TL machinery further comprises other small organic or inorganic molecules, such as amino acids, tRNAs or ATP, that facilitate the DNA transcription and/or mRNA translation. Various cellular components known to participate in in vivo transcription and translation can form part of the in vitro TX-TL machinery, see for example, Matsubayashi et al, “Purified cell-free systems as standard parts for synthetic biology.”; Curr Opin Chem Biol. 2014 October; 22:158-62; Li, et al. “Improved cell-free RNA and protein synthesis system.” PLoS One. 2014 Sep. 2; 9 (9):e106232. In some embodiments, different components can be provided individually and combined to assemble the in vitro TX-TL machinery. Exemplary ways of providing the in vitro TX-TL machinery components include recombinantly production, synthesis, and isolation from a cell. In some embodiments, the in vitro TX-TL machinery is provided in the form of one or more cell extract, or one or more supplemented cell extract that comprises the in vitro TX-TL machinery.

The terms “cell-free biosynthesis” and “CFB” are used interchangeably herein and refer to an in vitro (outside the cell) biosynthetic process for the production of one or more peptides or proteins. In some embodiments, cell-free biosynthesis occurs in a “cell-free biosynthesis reaction mixture” or “CFB reaction mixture” which provides various components, such as RNA, proteins, enzymes, co-factors, natural products, small molecules, organic molecules, to carry out protein synthesis outside a living cell. In some embodiments, the CFB reaction mixture can comprise one or more cell extracts or supplemented cell extracts, or commercially available cell-free reaction media (e.g. PURExpress®). In some embodiments, the CFB reaction mixture supports and facilitates the formation of a lasso peptide through the activity of one or more lasso peptide biosynthetic enzymes and proteins, including lasso peptidase, lasso cyclase and RRE. Exemplary CFB methods and systems, including those involving the use of in vitro TX-TL, are described in Culler, S. et al., PCT Application WO2017/031399 A1, and is incorporated herein by reference.

The terms “cell-free biosynthesis system” and “CFB system” are used interchangeably and refer to a system configured to produce one or more lasso peptide in vitro. For example, the CFB system can be an experimental design or set-up, apparatus or equipment, compositions of materials, or combinations of the foregoing, configured to produce one or more lasso peptide outside an intact cell. In some embodiments, the CFB system comprises a minimal set of lasso peptide biosynthesis components in a condition suitable for lasso formation.

Depending on the context, the term “condition suitable for lasso formation” may refer to, for example, a condition suitable for the expression of one or more protein products in the CFB system (e.g., a lasso precursor peptide, or a processing enzyme), including for example conditions suitable for the components of an in vitro TX-TL machinery to perform the intended function. Exemplary suitable conditions included are not limited to a suitable pH or the presence of a suitable concentration of co-factor for an enzymatic component of the in vitro TX-TL machinery to catalyze the TX-TL reaction. Additionally or alternatively, depending on the context, the term “condition suitable for lasso formation” may refer to, for example, a condition suitable for post-translational modification of a lasso precursor peptide. Exemplary suitable conditions include but are not limited to a suitable temperature and/or incubation time for a lasso cyclase and/or lasso peptidase to process the lasso precursor in to a matured lasso peptide.

The term “lasso peptide library” refers to a collection comprising (i) intact lasso peptides, (ii) functional fragments of lasso peptides, (iii) fusion proteins each comprising a lasso peptide or a functional fragment of lasso peptide, (iv) protein complexes each comprising a lasso peptide or a functional fragment of lasso peptide, (v) conjugates each comprising a lasso peptide or a functional fragment of lasso peptide, or (vi) any combinations of (i) to (v). Particularly, the alternative forms of molecules or complexes as provided in (ii), (iii), (iv) and (v) are herein collectively referred to as the “related molecules” of lasso peptides.

The term “display” and its grammatical variants, as used herein with respect to a chemical entity (e.g. a lasso peptide or functional fragment of lasso peptide), means to present or the presentation of the chemical entity (the “displayed entity”) in a manner so that it is chemically accessible in its environment and can be identified and/or distinguished from other chemical entities also present in the same environment. For example, a displayed entity can interact (e.g., bind to) or react (e.g. form covalent bonds) with other chemical entities (e.g., a target molecule) when the displayed entity is in contact with the other chemical entities. A displayed entity may be free-floating or affixed on an insoluble substrate. The insoluble substrate may assume various forms, as long as it does not interfere with the chemical accessibility, activity, or reactivity intended for the displayed entity. For example, in certain embodiments, where the displayed entity is a lasso peptide for binding with a target protein (e.g., a cell surface protein), and/or modulating a biological activity of the target protein, then the insoluble substrate can be made of a material that is chemically inert with respect to the intended target binding or modulating activity of the lasso peptide, such as a solid support made of a polymer or metal, or a microbial particle or cell (e.g., phage).

The term “display library” as used herein refers to the collection of a plurality of displayed entities, and each of the plurality of displayed entities in a library is a “member” of the library. To be clear, a “member” of the library refers to a unique displayed entity that is distinct from any other displayed entity(ies) that are present in the library. A library may comprise multiple identical copies of the same displayed entity, and the identical copies are collectively referred to as one member of the library. As used herein, two lasso peptides are considered “different” or “distinct” if they have different amino acid sequences or different structures (e.g., secondary, tertiary, or quaternary structure), or both different amino acid sequences and structures with respect to each other. For example, lasso cyclases having different selectivity for ring-forming amino acid residues can produce different lasso peptides from the same lasso core peptide by forming different ring structures. Distinct lasso peptides or functional fragments of a library are collectively referred to as “lasso species.” In some embodiments, a member of a lasso peptide display library can comprise one or more than one lasso species. For example, a member of a lasso peptide display library can be a fusion protein comprising two different species of lasso peptides.

In certain embodiments, a display library comprises a mechanism for identifying a member or distinguishing one member from another. A display library comprises a mechanism for identifying a member or distinguishing a member from other members of the library. Particularly, a “molecular display library” is a display library that utilizes sequence information of a nucleic acid molecule, e.g., DNA or RNA, to identify a displayed member or distinguishing one displayed member from another. In certain embodiments, each member of the library is associated with a unique nucleic acid sequence, and by obtaining and analyzing the sequence information, a user of the library can identify the particular member associated with the nucleic acid or distinguishing the particular member from other members of the library. In specific embodiment, the displayed entity is a peptide or polypeptide (e.g., lasso peptide), and is associated with a unique nucleic acid molecule that encodes at least a portion of the peptide or polypeptide (e.g., lasso peptide). As used herein, a “unique” nucleic acid is one having a sequence different from any other co-present nucleic acids. For example, in some embodiments, a set of DNA barcodes are synthetic nucleic acid molecules having unique sequences with respect to each other, which can be used as the member identifying/distinguishing mechanism of a molecular display library. As used herein, a molecular display library is not a bacteriophage display library, and does not utilize components of a bacteriophage as the identification mechanism for identifying or distinguishing members of the library. As used herein, in a molecular display library, the nucleic acid sequence that is used to identify a displayed member or distinguish one displayed member from another is not part of a phagemid or a bacteriophage.

In certain embodiments, a display library comprises a solid support, and each member of a display library is located at a particular location on the solid support. In some embodiments, the location of a member by itself can be used to identify the member or distinguish the member from other members of the library. In certain embodiments, the location together with other member identifying mechanism can identify a member or distinguish a member from other members of the library. For example, in specific embodiments, multiple locations each house one or more members of the library, and a set of DNA barcodes can be used at each location for identifying and/or distinguishing the members. In some embodiments, identical nucleic acid sequences used at different locations can still be unique nucleic acid sequences because when used at different locations, they are not considered co-present.

The term “solid support” or “solid surface” means, without limitation, any column (or column material), plate (including multi-well plates), bead, test tube, microtiter dish, solid particle (for example, agarose or sepharose), microchip (for example, silicon, silicon-glass, or gold chip), or membrane (for example, the membrane of a liposome or vesicle) to which a sample may be placed or affixed, either directly or indirectly (for example, through other binding partner intermediates such as antibodies).

In certain embodiments, each member of the library is associated with a detectable probe purported to produce a unique detectable signal, and the detectable signal is sufficiently unique to distinguish the associated member from another member of the library, exemplary detectable signals that can be used in connection with the present disclosure include but are not limited to a chemiluminescent signal, a radiological signal, a fluorescent signal, a digital signal, a color signal, etc.

The term “attached” or “associated” as used herein describes the interaction between or among two or more groups, moieties, compounds, monomers etc., e.g., a lasso peptide and a nucleic acid molecule. When two or more entities are “attached” to or “associated” with one another as described herein, they are linked by a direct or indirect covalent or non-covalent interaction. In some embodiments, the attachment is covalent. The covalent attachment may be, for example, but without limitation, through an amide, ester, carbon-carbon, disulfide, carbamate, ether, thioether, urea, amine, or carbonate linkage. The covalent attachment may also include a linker moiety, for example, a cleavable linker. Exemplary non-covalent interactions include hydrogen bonding, van der Waals interactions, dipole-dipole interactions, pi stacking interactions, hydrophobic interactions, magnetic interactions, electrostatic interactions, etc. Exemplary non-covalent binding pairs that can be used in connection with the present disclosure includes but are not limited to binding interaction between a ligand and its receptor, such as avidin or streptavidin and its binding moieties, including biotin or other streptavidin binding proteins.

The term “intact” as used herein with respect to a lasso peptide refers to the status of topologically intact. Thus, an “intact” lasso peptide is one comprising the complete lariat-like topology as described herein, including the terminal ring, middle loop and terminal tail. A sequence variant or a fragment of a lasso peptide may still be an intact lasso peptide, as long as the sequence variant or fragment of the lasso peptide still forms the lariat-like topology. For example, a lasso peptide having an amino acid residue truncated from its tail portion and another amino acid residue deleted from its ring portion may still form the lariat-like topology, even though the tail is shortened, and the ring is tightened. Such a variant is still considered an intact lasso peptide. In some embodiments, an intact lasso peptide has one or more effector functions.

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In various embodiments, protein fragments include polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 30 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, at least 250, at least 300, at least 350, at least 400, at least 450, at least 500, at least 550, at least 600, at least 650, at least 700, at least 750, at least 800, at least 850, at least 900, or at least 950 contiguous amino acid residues of the protein. In a specific embodiment, a fragment of a protein retains at least 1, at least 2, at least 3, or more functions of the protein.

A “functional fragment,” “binding fragment,” or “target-binding fragment” of a lasso peptide retains some but not all of the topological features of an intact lasso peptide, while retaining at least one if not some or all of the biological functions attributed to the intact lasso peptide. The function comprises at least binding to or associating with a target molecule, directly or indirectly. For example, a functional fragment of a lasso peptide may retain only the ring structure without the loop and the tail (i.e., a head-to-tail cyclic peptide) or with an unthreaded tail loosely extended from the ring (i.e., a branched-cyclic peptide). In some embodiments, the loose tail may have the complete or partial amino acid sequence of the loop and tail portions of an intact lasso peptide. For example, lassomycin as described in Garvish et al. (Chem Biol. 2014 Apr. 24; 21(4): 509-518) is a functional fragment of lasso peptide that has the same amino acid sequence as lassomycin and the lariat-like topology. A functional fragment of a lasso peptide may only retain the ring and the loop structures without a tail portion. The various topologies assumed by functional fragments of lasso peptides are herein collectively referred to as the “lasso-related topologies.” Functional fragments of lasso peptides can be recombinantly produced or produced via cell-free biosynthesis as described further below.

The term “fusion protein” when used with respect to a lasso peptide refers to a peptide or polypeptide that comprises an amino acid sequence of the lasso peptide joined with an amino acid sequence that is not normally a part of the same lasso peptide. The fusion protein may comprise the entire amino acid sequence of a lasso peptide, or only a portion thereof. The lasso portion of the fusion protein retains at least one, if not some or all, of the topological features of an intact lasso peptide, and is fused to the other peptide or polypeptide in a manner that does not interfere with its lasso-related topologies. For example, in certain embodiments, a fusion protein comprises an intact lasso peptide fused at the end of the tail portion to another peptide or polypeptide. In certain embodiments, a fusion protein comprises two intact lasso peptides fused together by joining the ends of the two lasso tails. In various embodiments, fusion proteins may comprise lasso functional fragments having various lasso-related topologies. Fusion proteins comprising lasso peptides can be recombinantly produced or produced via cell-free biosynthesis as described further below.

The term “protein complex” when used with respect to a lasso peptide refers to a protein complex comprising at least two subunits, where at least one subunit comprises a lasso peptide, or a functional fragment of a lasso peptide. In certain embodiments, the subunit comprising the lasso peptide or functional fragment thereof is a fusion protein. In certain embodiments, multiple subunits of a protein complex may each contains a lasso peptide or a functional fragment thereof, where the multiple lasso peptides or functional fragments thereof may be the same or different.

The term “conjugate” when used with respect to a lasso peptide refers to an entity formed as a result of covalent or non-covalent attachment or linkage of a lasso peptide or functional fragment thereof to at least one non-peptidic entity, such as a nucleic acid or a small molecule compound.

As used herein, the term “contacting” and its grammatical variations, when used in reference to two or more components, refers to any process whereby the approach, proximity, mixture or commingling of the referenced components is promoted or achieved without necessarily requiring physical contact of such components, and includes mixing of solutions containing any one or more of the referenced components with each other. The referenced components may be contacted in any particular order or combination and the particular order of recitation of components is not limiting. For example, “contacting A with B and C” encompasses embodiments where A is first contacted with B then C, as well as embodiments where C is contacted with A then B, as well as embodiments where a mixture of A and C is contacted with B, and the like. Furthermore, such contacting does not necessarily require that the end result of the contacting process be a mixture including all of the referenced components, as long as at some point during the contacting process all of the referenced components are simultaneously present or simultaneously included in the same mixture or solution. Where one or more of the referenced components to be contacted includes a plurality (e.g., “contacting a library of candidate lasso peptides with the target molecule”), then each member of the plurality can be viewed as an individual component of the contacting process, such that the contacting can include contacting of any one or more members of the plurality with any other member of the plurality and/or with any other referenced component (e.g., some or all of the plurality of candidate lasso peptides can be contacted with a target molecule) in any order or combination.

The terms “target molecule” and “target protein” are used interchangeably herein and refer to a protein with which a lasso peptide binds under a physiological condition that mimics the native environment where the protein is isolated or derived from. As used herein, the target molecule is a cell surface protein or an extracellularly secreted protein. “Cell surface protein” is a term of art, and is used herein to refer to any protein that is known by the skilled person as a cell surface protein, and including those with any form of post-translational modifications, such as glycosylation, phosphorylation, lipidation, etc. In various embodiments, a cell surface protein can be a peptide or protein that has at least one part exposed to the extracellular environment, while embedded in or span the lipid layer of the cell membrane, or associated with a molecule integrated in the lipid layer. Exemplary types of cell surface proteins that can be used in connection with the present application include but are not limited to cell surface receptors, biomarkers, transporters, ion channels, and enzymes, where one particular protein may fit into one or more of these categories. In specific embodiments, cell surface protein is a cell surface receptor, such as a glucagon receptor, an endothelin receptor, an atrial natriuretic factor receptor, a G protein-coupled receptor (GPCR). In certain embodiments, a target molecule mediates one or more cellular activities (e.g., through a cellular signaling pathway), and as a result of the binding of a lasso peptide to the target molecule, the cellular activities is modulated. In some embodiments, a target molecule can be a protein secreted by a cell to the extracellular environment, such as growth factors, cytokines, etc.

The term “target site” as used herein refers to the amino acid residue or the group of amino acid residues with which a particular lasso peptide interacts to form the binding with the target molecule. According to the present disclosure, different lasso peptides may bind to different target sites or compete for binding with the same target site of a target molecule. In some embodiments, a lasso peptide specifically binds to a target molecule or a target site thereof.

The term “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single target-binding site of a binding protein and a single target site of a target molecule is the affinity of the binding protein or functional fragment for that target site. The ratio of dissociation rate (k_{off}) to association rate (kₒₙ) of a binding protein to a monovalent target site (k_{off}/kₒₙ) is the dissociation constant K_{D}, which is inversely related to affinity. The lower the K_{D} value, the higher the affinity of the antibody. The value of K_{D} varies for different complexes of lasso peptides or target proteins depends on both kₒₙ and k_{off}. The dissociation constant K_{D} for a binding protein (e.g., a lasso peptide) provided herein can be determined using any method provided herein or any other method well known to those skilled in the art. The affinity at one binding site does not always reflect the true strength of the interaction between a binding protein and the target molecule. When complex target molecule containing multiple, repeating target sites, such as a polyvalent target protein, come in contact with lasso peptides containing multiple target binding sites, the interaction of the lasso peptide with the target protein at one site will increase the probability of a reaction at a second site.

The terms “lasso peptides that specifically bind to a target molecule,” “lasso peptides that specifically bind to a target site,” and analogous terms are also used interchangeably herein and refer to lasso peptides that specifically bind to a target molecule, such as a polypeptide, or fragment, or ligand-binding domain. A lasso peptide that specifically binds to a target protein may bind to the extracellular domain or a peptide derived from the extracellular domain of the target protein. A lasso peptide that specifically binds to a target protein of a specific species origin (e.g., a human protein) may be cross-reactive with the target protein of a different species origin (e.g., a cynomolgus protein). In certain embodiments, a lasso peptide that specifically binds to a target protein of a specific species origin does not cross-react with the target protein from another species of origin.

A lasso peptide that specifically binds to a target protein can be identified, for example, by immunoassays (e.g., ELISA, fluorescent immunosorbent assay, chemiluminescence immune assay, radioimmunoassay (MA), enzyme multiplied immunoassay, solid phase radioimmunoassay (SPRIA), a surface plasmon resonance (SPR) assay (e.g., Biacore®), a fluorescence polarization assay, a fluorescence resonance energy transfer (FRET) assay, Dot-blot assay, fluorescence activated cell sorting (FACS) assay, or other techniques known to those of skill in the art. A lasso peptide binds specifically to a target protein when it binds to the target protein with higher affinity than to any cross-reactive target molecule as determined using experimental techniques, such as radioimmunoassays (MA) and enzyme linked immunosorbent assays (ELISAs). Typically a specific or selective reaction will be at least twice background signal or noise and may be more than 10 times background.

A lasso peptide which “binds a target molecule of interest” is one that binds the target molecule with sufficient affinity such that the lasso peptide is useful as a therapeutic agent in targeting a cell or tissue expressing the target molecule, and does not significantly cross-react with other molecules. In such embodiments, the extent of binding of the lasso peptide to a “non-target” molecule will be less than about 10% of the binding of the lasso peptide to its particular target molecule, for example, as determined by fluorescence activated cell sorting (FACS) analysis or MA.

With regard to the binding of a lasso peptide to a target molecule, the term “specific binding,” “specifically binds to,” or “is specific for” a particular polypeptide or an fragment on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding,” “specifically binds to,” or “is specific for” a particular polypeptide or a fragment on a particular polypeptide target as used herein refers to binding where a molecule binds to a particular polypeptide or fragment on a particular polypeptide without substantially binding to any other polypeptide or polypeptide fragment. In certain embodiments, a lasso peptide that binds to a target molecule has a dissociation constant (K_{D}) of less than or equal to 100 μM, 80 μM, 50 μM, 25 μM, 10 μM, 5 μM, 1 μM, 900 nM, 800 nM, 700 nM, 600 nM, 500 nM, 400 nM, 300 nM, 200 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM.

In the context of the present disclosure, a target protein is said to specifically bind or selectively bind to a lasso peptide, for example, when the dissociation constant (K_{D}) is ≤10⁻⁷M. In some embodiments, the lasso peptides specifically bind to a target protein with a K_{D} of from about 10^{31 7}M to about 10⁻¹²M. In certain embodiments, the lasso peptides specifically bind to a target protein with high affinity when the K_{D} is ≤10⁻⁸M or K_{D} is ≤10⁻⁹M. In one embodiment, the lasso peptides may specifically bind to a purified human target protein with a K_{D} of from 1×10⁻⁹M to 10×10⁻⁹M as measured by^{Biacore}®. In another embodiment, the lasso peptides may specifically bind to a purified human target protein with a K_{D} of from 0.1×10⁻⁹M to 1×10⁻⁹M as measured by KinExA™ (Sapidyne, Boise, Id.). In yet another embodiment, the lasso peptides specifically bind to a target protein expressed on cells with a K_{D} of from 0.1×10⁻⁹M to 10×10⁻⁹M. In certain embodiments, the lasso peptides specifically bind to a human target protein expressed on cells with a K_{D} of from 0.1×10⁻⁹M to 1×10⁻⁹M. In some embodiments, the lasso peptides specifically bind to a human target protein expressed on cells with a K_{D} of 1×10⁻⁹M to 10×10⁻⁹M. In certain embodiments, the lasso peptides specifically bind to a human target protein expressed on cells with a K_{D} of about 0.1×10⁻⁹M, about 0.5×10⁻⁹M, about 1×10⁻⁹M, about 5×10⁻⁹M, about 10×10⁻⁹M, or any range or interval thereof. In still another embodiment, the lasso peptides specifically bind to a non-human target protein expressed on cells with a K_{D} of 0.1×10⁻⁹M to 10×10⁻⁹M. In certain embodiments, the lasso peptides specifically bind to a non-human target protein expressed on cells with a K_{D} of from 0.1×10⁻⁹M to 1×10⁻⁹M. In some embodiments, the lasso peptides specifically bind to a non-human target protein expressed on cells with a K_{D} of 1×10⁻⁹M to 10×10⁻⁹M. In certain embodiments, the lasso peptides specifically bind to a non-human target protein expressed on cells with a K_{D} of about 0.1×10⁻⁹M, about 0.5×10⁻⁹M, about 1×10⁻⁹M, about 5×10⁻⁹M, about 10×10⁻⁹M, or any range or interval thereof.

“Binding affinity” generally refers to the strength of the sum total of noncovalent interactions between a single binding site of a molecule (e.g., a binding protein such as a lasso peptide) and its binding partner (e.g., a target protein). Unless indicated otherwise, as used herein, “binding affinity” refers to intrinsic binding affinity which reflects a 1:1 interaction between members of a binding pair (e.g., lasso peptide and target protein). The affinity of a binding molecule X for its binding partner Y can generally be represented by the dissociation constant (K_{D}). Affinity can be measured by common methods known in the art, including those described herein. Low-affinity lasso peptides generally bind target proteins slowly and tend to dissociate readily, whereas high-affinity lasso peptides generally bind target proteins faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the lasso peptide of interest and its target protein. The K_{D} or K_{D} value may also be measured by using surface plasmon resonance assays by Biacore®, using, for example, a Biacore®-2000 or a Biacore®-3000, or by biolayer interferometry using, for example, the Octet®QK384 system. An “on-rate” or “rate of association” or “association rate” or “kₒₙ,” may also be determined with the same surface plasmon resonance or biolayer interferometry techniques described above using, for example, a Biacore®-2000 or a Biacore®-3000, or the Octet®QK384 system.

The term “compete” when used in the context of lasso peptides (e.g., a lasso peptide and other binding proteins that bind to and compete for the same target molecule or target site on the target molecule) means competition as determined by an assay in which the lasso peptide (or binding fragment) thereof under study prevents or inhibits the specific binding of a reference molecule (e.g., a reference ligand of the target molecule) to a common target molecule. Numerous types of competitive binding assays can be used to determine if a test lasso peptide competes with a reference ligand for binding to a target molecule. Examples of assays that can be employed include solid phase direct or indirect RIA, solid phase direct or indirect enzyme immunoassay (EIA), sandwich competition assay (see, e.g., Stahli et al., 1983, Methods in Enzymology 9:242-53), solid phase direct biotin-avidin EIA (see, e.g., Kirkland et al., 1986, J. Immunol. 137:3614-19), solid phase direct labeled assay, solid phase direct labeled sandwich assay (see, e.g., Harlow and Lane, Antibodies, A Laboratory Manual (1988)), solid phase direct label RIA using I-125 label (see, e.g., Morel et al., 1988, Mol. Immunol. 25:7-15), and direct labeled RIA (Moldenhauer et al., 1990, Scand. J. Immunol. 32:77-82). Typically, such an assay involves the use of a purified target molecule bound to a solid surface, or cells bearing either of an unlabeled test target-binding lasso peptide or a labeled reference target-binding protein (e.g., reference target-binding ligand). Competitive inhibition may be measured by determining the amount of label bound to the solid surface in the presence of the test target-binding lasso peptide. Usually the test target-binding protein is present in excess. Target-binding lasso peptides identified by competition assay (e.g., competing lasso peptides) include lasso peptides binding to the same target site as the reference and lasso peptides binding to an adjacent target site sufficiently proximal to the target site bound by the reference for steric hindrance to occur. Additional details regarding methods for determining competitive binding are described herein. Usually, when a competing lasso peptide is present in excess, it will inhibit specific binding of a reference to a common target molecule by at least 30%, for example 40%, 45%, 50%, 55%, 60%, 65%, 70%, or 75%. In some instance, binding is inhibited by at least 80%, 85%, 90%, 95%, 96%, 97%, 98%, 99%, or more.

A “blocking” lasso peptide or an “antagonist” lasso peptide is one which inhibits or reduces biological activity of the target molecule it binds. For example, blocking lasso peptide or antagonist lasso peptide may substantially or completely inhibit the biological activity of the target molecule.

The term “inhibition” or “inhibit,” when used herein, refers to partial (such as, 1%, 2%, 5%, 10%, 20%, 25%, 50%, 75%, 90%, 95%, 99%) or complete (i.e., 100%) inhibition.

The term “attenuate,” “attenuation,” or “attenuated,” when used herein, refers to partial (such as, 1%, 2%, 5%, 10%, 20%, 25%, 50%, 75%, 90%, 95%, 99%) or complete (i.e., 100%) reduction in a property, activity, effect, or value.

An “agonist” lasso peptide is a lasso peptide that triggers a response, e.g., one that mimics at least one of the functional activities of a polypeptide of interest (e.g., an agonist lasso peptide for glucagon-like peptide-1 receptor (GLP-1R) wherein the agonist lasso peptide mimics the functional activities of glucagon-like peptide-1). An agonist lasso peptide includes a lasso peptide that is a ligand mimetic, for example, wherein a ligand binds to a cell surface receptor and the binding induces cell signaling or activities via an intercellular cell signaling pathway and wherein the lasso peptide induces a similar cell signaling or activation. For the sole purpose of illustration, an “agonist” of glucagon-like peptide-1 receptor refers to a molecule that is capable of activating or otherwise increasing one or more of the biological activities of glucagon-like peptide-1 receptor, such as in a cell expressing glucagon-like peptide-1 receptor. In some embodiments, an agonist of glucagon-like peptide-1 receptor (e.g., an agonistic lasso peptide as described herein) may, for example, act by activating or otherwise increasing the activation and/or cell signaling pathways of a cell expressing a glucagon receptor protein, thereby increasing a glucagon-like peptide-1 receptor—mediated biological activity of the cell relative to the glucagon-like peptide-1 receptor—mediated biological activity in the absence of agonist.

The phrase “substantially similar” or “substantially the same” denotes a sufficiently high degree of similarity between two numeric values (e.g., one associated with a lasso peptide of the present disclosure and the other associated with a reference ligand) such that one of skill in the art would consider the difference between the two values to be of little or no biological and/or statistical significance within the context of the biological characteristic measured by the values (e.g., K_{D} values). For example, the difference between the two values may be less than about 50%, less than about 40%, less than about 30%, less than about 20%, less than about 10%, or less than about 5%, as a function of the value for the reference ligand.

The phrase “substantially increased,” “substantially reduced,” or “substantially different,” as used herein, denotes a sufficiently high degree of difference between two numeric values (e.g., one associated with a lasso peptide of the present disclosure and the other associated with a reference ligand) such that one of skill in the art would consider the difference between the two values to be of statistical significance within the context of the biological characteristic measured by the values. For example, the difference between said two values can be greater than about 10%, greater than about 20%, greater than about 30%, greater than about 40%, or greater than about 50%, as a function of the value for the reference ligand.

As used herein, the term “modulating” or “modulate” refers to an effect of altering a biological activity (i.e. increasing or decreasing the activity), especially a biological activity associated with a particular biomolecule such as a cell surface receptor. For example, an inhibitor of a particular biomolecule modulates the activity of that biomolecule, e.g., an enzyme, by decreasing the activity of the biomolecule, such as an enzyme. Such activity is typically indicated in terms of an inhibitory concentration (IC₅₀) of the compound for an inhibitor with respect to, for example, an enzyme.

By “assaying” is meant the creation of experimental conditions and the gathering of data regarding a particular result of the exposure to specific experimental conditions. For example, enzymes can be assayed based on their ability to act upon a detectable substrate. A compound can be assayed based on its ability to bind to a particular target molecule or molecules.

The term “IC₅₀” refers to an amount, concentration, or dosage of a substance that is required for 50% inhibition of a maximal response in an assay that measures such response. The term “EC₅₀” refers to an amount, concentration, or dosage of a substance that is required for 50% of a maximal response in an assay that measures such response. The term “CC₅₀” refers an amount, concentration, or dosage of a substance that results in 50% reduction of the viability of a host. In certain embodiments, the CC₅₀ of a substance is the amount, concentration, or dosage of the substance that is required to reduce the viability of cells treated with the compound by 50%, in comparison with cells untreated with the compound. The term “K_{d}” refers to the equilibrium dissociation constant for a ligand and a protein, which is measured to assess the binding strength that a small molecule ligand (such as a small molecule drug) has for a protein or receptor, such as a cell surface receptor. The dissociation constant, K_{d}, is commonly used to describe the affinity between a ligand and a protein or receptor; i.e., how tightly a ligand binds to a particular protein or receptor, and is the inverse of the association constant. Ligand-protein affinities are influenced by non-covalent intermolecular interactions between the two molecules such as hydrogen bonding, electrostatic interactions, hydrophobic and van der Waals forces. The analogous term “Kᵢ” is the inhibitor constant or inhibition constant, which is the equilibrium dissociation constant for an enzyme inhibitor, and provides an indication of the potency of an inhibitor.

The term “identity” refers to a relationship between the sequences of two or more polypeptide molecules or two or more nucleic acid molecules, as determined by aligning and comparing the sequences. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared. Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution), insertion of one or more (e.g., generally fewer than 5, 4, or 3) amino acids adjacent to said residue/position, and/or deletion of said residue/position.

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

The term “vector” refers to a substance that is used to carry or include a nucleic acid sequence, including for example, a nucleic acid sequence encoding a lasso precursor peptide, or lasso processing enzymes as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both a lasso core peptide and a lasso cyclase), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., a lasso precursor peptide as described herein), and it is further understood that expression levels can be optimized to obtain sufficient expression using methods well known in the art.

The term “detectable probe” refers to a composition that provides a detectable signal. The term includes, without limitation, any fluorophore, chromophore, radiolabel, enzyme, antibody or antibody fragment, and the like, that provide a detectable signal via its activity.

The term “detectable agent” refers to a substance that can be used to ascertain the existence or presence of a desired molecule, such as a complex between a lasso peptide and a target molecule as described herein, in a sample or subject. A detectable agent can be a substance that is capable of being visualized or a substance that is otherwise able to be determined and/or measured (e.g., by quantitation).

5.3 Library of Lasso Peptides and Methods of Making the Same.

Provided herein are libraries that comprise diversified species of lasso peptides or functional fragments of lasso peptides. The lasso peptides or functional fragments of lasso peptides of the library may be isolated natural products (e.g., products of naturally-occurring lasso peptide biosynthesis gene clusters) or artificially produced (e.g., biosynthesized using an engineered producer organism or a CFB system). The lasso peptides of the library may be naturally-existing (e.g., having the same amino acid sequence and structure as a lasso peptide found in nature) or non-naturally occurring (e.g., having an amino acid sequence or structure that is different from any known natural lasso peptide).

The lasso peptides and functional fragments of lasso peptides provided herein can find uses in various aspects, including but are not limited to, diagnostic uses, prognostic uses, therapeutic uses, or as nutraceuticals or food supplements, for humans and animals. In some embodiments, the lasso peptide library provided herein can be screened for members having one or more desirable properties, for example, by subjecting the lasso peptide library to various biological assays. In some embodiments, the lasso peptide library can be screened using assays known in the art.

### 5.3.1 Lasso Peptides

As provided herein, an intact lasso peptide comprises the complete lariat-like topology as exemplified in FIG. 1. In some embodiments, the ring structure of a lasso peptide is formed through, for example, covalent bonding between a terminal amino acid residue and an internal amino acid residue. In some embodiments, the ring is formed via disulfide bonding between two or more amino acid residues of the lasso peptide. In alternative embodiments, the ring is formed via non-covalent interaction between two or more amino acid residues of the lasso peptide. In yet alternative embodiments, the ring is formed via both covalent and non-covalent interactions between at least two amino acid residues of the lasso peptide. In some embodiments, the ring is located at the C-terminus of the lasso peptide. In other embodiments, the ring is located at the N-terminus of the lasso peptide.

In specific embodiments, an N-terminal ring structure is formed by the formation of a bond between the N-terminal amino acid residue of the lasso peptide and an internal amino acid residue of the lasso peptide. In specific embodiment, an N-terminal ring structure is formed by formation of an isopeptide bond between the N-terminal amino group and the carboxyl group in the side chain of an internal amino acid residue, such as glutamate or aspartate residue, of the lasso peptide. In specific embodiments, an N-terminal ring structure is formed by the formation of an isopeptide bond between the N-terminal amino group and the carboxyl group in the side chain of an internal amino acid residue, such as glutamate or aspartate residue, located at the 6^{th} to 20^{th} position in the lasso peptide amino acid sequence, counting from its N terminus.

In specific embodiments, an N-terminal ring structure is formed by the formation of an isopeptide bond between the N-terminal amino group and the carboxyl group in the side chain of a glutamate located at the 6^{th} position in the lasso peptide amino acid sequence, counting from its N terminus, such that the lasso peptide has an N-terminal 6-member ring. In specific embodiments, an N-terminal ring structure is formed by the formation of an isopeptide bond between the N-terminal amino group and the carboxyl group in the side chain of a glutamate located at the 7^{th} position in the lasso peptide amino acid sequence, counting from its N terminus, such that the lasso peptide has an N-terminal 7-member ring. In specific embodiments, an N-terminal ring structure is formed by the formation of an isopeptide bond between the N-terminal amino group and the carboxyl group in the side chain of a glutamate located at the 8^{th} position in the lasso peptide amino acid sequence, counting from its N terminus, such that the lasso peptide has an N-terminal 8-member ring. In specific embodiments, an N-terminal ring structure is formed by the formation of an isopeptide bond between the N-terminal amino group and the carboxyl group in the side chain of a glutamate located at the 9^{th} position in the lasso peptide amino acid sequence, counting from its N terminus, such that the lasso peptide has an N-terminal 9-member ring. In specific embodiments, an N-terminal ring structure is formed by the formation of an isopeptide bond between the N-terminal amino group and the carboxyl group in the side chain of a glutamate located at the 10^{th} position in the lasso peptide amino acid sequence, counting from its N terminus, such that the lasso peptide has an N-terminal 10-member ring. In specific embodiments, an N-terminal ring structure is formed by the formation of an isopeptide bond between the N-terminal amino group and the carboxyl group in the side chain of a glutamate located at the 11^{th} position in the lasso peptide amino acid sequence, counting from its N terminus, such that the lasso peptide has an N-terminal 11-member ring. In specific embodiments, an N-terminal ring structure is formed by the formation of an isopeptide bond between the N-terminal amino group and the carboxyl group in the side chain of a glutamate located at the 12^{th} position in the lasso peptide amino acid sequence, counting from its N terminus, such that the lasso peptide has an N-terminal 12-member ring. In specific embodiments, an N-terminal ring structure is formed by the formation of an isopeptide bond between the N-terminal amino group and the carboxyl group in the side chain of a glutamate located at the 13^{th} position in the lasso peptide amino acid sequence, counting from its N terminus, such that the lasso peptide has an N-terminal 13-member ring. In specific embodiments, an N-terminal ring structure is formed by the formation of an isopeptide bond between the N-terminal amino group and the carboxyl group in the side chain of a glutamate located at the 14^{th} position in the lasso peptide amino acid sequence, counting from its N terminus, such that the lasso peptide has an N-terminal 14-member ring. In specific embodiments, an N-terminal ring structure is formed by the formation of an isopeptide bond between the N-terminal amino group and the carboxyl group in the side chain of a glutamate located at the 15^{th} position in the lasso peptide amino acid sequence, counting from its N terminus, such that the lasso peptide has an N-terminal 15-member ring. In specific embodiments, an N-terminal ring structure is formed by the formation of an isopeptide bond between the N-terminal amino group and the carboxyl group in the side chain of a glutamate located at the 16^{th} position in the lasso peptide amino acid sequence, counting from its N terminus, such that the lasso peptide has an N-terminal 16-member ring. In specific embodiments, an N-terminal ring structure is formed by the formation of an isopeptide bond between the N-terminal amino group and the carboxyl group in the side chain of a glutamate located at the 17^{th} position in the lasso peptide amino acid sequence, counting from its N terminus, such that the lasso peptide has an N-terminal 17-member ring. In specific embodiments, an N-terminal ring structure is formed by the formation of an isopeptide bond between the N-terminal amino group and the carboxyl group in the side chain of a glutamate located at the 18^{th} position in the lasso peptide amino acid sequence, counting from its N terminus, such that the lasso peptide has an N-terminal 18-member ring. In specific embodiments, an N-terminal ring structure is formed by the formation of an isopeptide bond between the N-terminal amino group and the carboxyl group in the side chain of a glutamate located at the 19^{th} position in the lasso peptide amino acid sequence, counting from its N terminus, such that the lasso peptide has an N-terminal 19-member ring. In specific embodiments, an N-terminal ring structure is formed by the formation of an isopeptide bond between the N-terminal amino group and the carboxyl group in the side chain of a glutamate located at the 20^{th} position in the lasso peptide amino acid sequence, counting from its N terminus, such that the lasso peptide has an N-terminal 20-member ring.

In specific embodiments, an N-terminal ring structure is formed by the formation of an isopeptide bond between the N-terminal amino group and the carboxyl group in the side chain of an aspartate located at the 6^{th} position in the lasso peptide amino acid sequence, counting from its N terminus, such that the lasso peptide has an N-terminal 6-member ring. In specific embodiments, an N-terminal ring structure is formed by the formation of an isopeptide bond between the N-terminal amino group and the carboxyl group in the side chain of an aspartate located at the 7^{th} position in the lasso peptide amino acid sequence, counting from its N terminus, such that the lasso peptide has an N-terminal 7-member ring. In specific embodiments, an N-terminal ring structure is formed by the formation of an isopeptide bond between the N-terminal amino group and the carboxyl group in the side chain of an aspartate located at the 8^{th} position in the lasso peptide amino acid sequence, counting from its N terminus, such that the lasso peptide has an N-terminal 8-member ring. In specific embodiments, an N-terminal ring structure is formed by the formation of an isopeptide bond between the N-terminal amino group and the carboxyl group in the side chain of an aspartate located at the 9^{th} position in the lasso peptide amino acid sequence, counting from its N terminus, such that the lasso peptide has an N-terminal 9-member ring. In specific embodiments, an N-terminal ring structure is formed by the formation of an isopeptide bond between the N-terminal amino group and the carboxyl group in the side chain of an aspartate located at the 10^{th} position in the lasso peptide amino acid sequence, counting from its N terminus, such that the lasso peptide has an N-terminal 10-member ring. In specific embodiments, an N-terminal ring structure is formed by the formation of an isopeptide bond between the N-terminal amino group and the carboxyl group in the side chain of an aspartate located at the 11^{th} position in the lasso peptide amino acid sequence, counting from its N terminus, such that the lasso peptide has an N-terminal 11-member ring. In specific embodiments, an N-terminal ring structure is formed by the formation of an isopeptide bond between the N-terminal amino group and the carboxyl group in the side chain of an aspartate located at the 12^{th} position in the lasso peptide amino acid sequence, counting from its N terminus, such that the lasso peptide has an N-terminal 12-member ring. In specific embodiments, an N-terminal ring structure is formed by the formation of an isopeptide bond between the N-terminal amino group and the carboxyl group in the side chain of an aspartate located at the 13^{th} position in the lasso peptide amino acid sequence, counting from its N terminus, such that the lasso peptide has an N-terminal 13-member ring. In specific embodiments, an N-terminal ring structure is formed by the formation of an isopeptide bond between the N-terminal amino group and the carboxyl group in the side chain of an aspartate located at the 14^{th} position in the lasso peptide amino acid sequence, counting from its N terminus, such that the lasso peptide has an N-terminal 14-member ring. In specific embodiments, an N-terminal ring structure is formed by the formation of an isopeptide bond between the N-terminal amino group and the carboxyl group in the side chain of an aspartate located at the 15^{th} position in the lasso peptide amino acid sequence, counting from its N terminus, such that the lasso peptide has an N-terminal 15-member ring. In specific embodiments, an N-terminal ring structure is formed by the formation of an isopeptide bond between the N-terminal amino group and the carboxyl group in the side chain of an aspartate located at the 16^{th} position in the lasso peptide amino acid sequence, counting from its N terminus, such that the lasso peptide has an N-terminal 16-member ring. In specific embodiments, an N-terminal ring structure is formed by the formation of an isopeptide bond between the N-terminal amino group and the carboxyl group in the side chain of an aspartate located at the 17^{th} position in the lasso peptide amino acid sequence, counting from its N terminus, such that the lasso peptide has an N-terminal 17-member ring. In specific embodiments, an N-terminal ring structure is formed by the formation of an isopeptide bond between the N-terminal amino group and the carboxyl group in the side chain of an aspartate located at the 18^{th} position in the lasso peptide amino acid sequence, counting from its N terminus, such that the lasso peptide has an N-terminal 18-member ring. In specific embodiments, an N-terminal ring structure is formed by the formation of an isopeptide bond between the N-terminal amino group and the carboxyl group in the side chain of an aspartate located at the 19^{th} position in the lasso peptide amino acid sequence, counting from its N terminus, such that the lasso peptide has an N-terminal 19-member ring. In specific embodiments, an N-terminal ring structure is formed by the formation of an isopeptide bond between the N-terminal amino group and the carboxyl group in the side chain of an aspartate located at the 20^{th} position in the lasso peptide amino acid sequence, counting from its N terminus, such that the lasso peptide has an N-terminal 20-member ring.

In specific embodiments, a C-terminal ring structure is formed by the formation of a bond between the C-terminal amino acid residue of the lasso peptide and an internal amino acid residue of the lasso peptide. In specific embodiment, a C-terminal ring structure is formed by formation of an isopeptide bond between the C-terminal carboxyl group and the amino group in the side chain of an internal amino acid residue, such as Asparagine or Glutamine residue, of the lasso peptide. In specific embodiments, a C-terminal ring structure is formed by the formation of an isopeptide bond between the C-terminal carboxyl group and the amino group in the side chain of an internal amino acid residue, such as Asparagine or Glutamine residue, located at the 6^{th} to 20^{th} position in the lasso peptide amino acid sequence, counting from its C terminus.

As described herein, a lasso peptide can have one or more structural features that contribute to the stability of the lariat-like topology of the lasso peptide. In some embodiments, the ring is formed around the tail, which is threaded through the ring, and a middle loop portion connects the ring and the tail portions of the lasso peptide. In some embodiments, one or more disulfide bond(s) are formed (i) between the ring and tail portions, (ii) between the ring and loop portions, (iii) between the loop and tail portions; (iv) between different amino acid residues of the tail portion, or (v) any combination of (i) through (iv), which contribute to hold the lariat-like topology in place and increase the stability of the lasso peptide. In particular embodiments, one or more disulfide bonds are formed between the loop and the ring. In particular embodiments, one or more disulfide bonds are formed between the ring and the tail. In particular embodiments, one or more disulfide bonds are formed between the tail and the loop. In particular embodiments, one or more disulfide bonds are formed between different amino acid residues of the tail.

In particular embodiments, at least one disulfide bond is formed between the loop and ring portions of a lasso peptide, and at least one disulfide bond is formed between the tail and ring portions of a lasso peptide. In particular embodiments, at least one disulfide bond is formed between the loop and ring portions of a lasso peptide, and at least one disulfide bond is formed between the loop and tail portions of a lasso peptide. In particular embodiments, at least one disulfide bond is formed between the loop and ring portions of a lasso peptide, and at least one disulfide bond is formed between the different amino acid residues of the tail portion of a lasso peptide. In particular embodiments, at least one disulfide bond is formed between the tail and ring portions of a lasso peptide, and at least one disulfide bond is formed between the loop and tail portions of a lasso peptide. In particular embodiments, at least one disulfide bond is formed between the tail and ring portions of a lasso peptide, and at least one disulfide bond is formed between the different amino acid residues of the tail portion of a lasso peptide. In particular embodiments, at least one disulfide bond is formed between the loop and tail portions of a lasso peptide, and at least one disulfide bond is formed between the different amino acid residues of the tail portion of a lasso peptide. In particular embodiments, at least one disulfide bond is formed between the loop and ring portions of a lasso peptide, and at least one disulfide bond is formed between the tail and ring portions of a lasso peptide, and at least one disulfide bond is formed between the loop and tail portions of a lasso peptide. In particular embodiments, at least one disulfide bond is formed between the loop and ring portions of a lasso peptide, and at least one disulfide bond is formed between the tail and ring portions of a lasso peptide, an and at least one disulfide bond is formed between the different amino acid residues of the tail portion of a lasso peptide. In particular embodiments, at least one disulfide bond is formed between the loop and ring portions of a lasso peptide, and at least one disulfide bond is formed between the loop and tail portions of a lasso peptide, an and at least one disulfide bond is formed between the different amino acid residues of the tail portion of a lasso peptide. In particular embodiments, at least one disulfide bond is formed between the tail and ring portions of a lasso peptide, and at least one disulfide bond is formed between the loop and tail portions of a lasso peptide, an and at least one disulfide bond is formed between the different amino acid residues of the tail portion of a lasso peptide. In particular embodiments, at least one disulfide bond is formed between the loop and ring portions of a lasso peptide, and at least one disulfide bond is formed between the tail and ring portions of a lasso peptide, and at least one disulfide bond is formed between the loop and tail portions of a lasso peptide, and at least one disulfide bond is formed between the different amino acid residues of the tail portion of a lasso peptide.

In some embodiments, structural features of a lasso peptide that contribute to its topological stability comprise bulky side chains of amino acid residues located on the ring, the tail and/or the loop portion(s) of the lasso peptide, and these bulky side chains create an steric effect that holds the lariat-like topology in place. In some embodiments, the tail portion comprises at least one amino acid residue having a sterically bulky side chain. In some embodiments, the tail portion comprises at least one amino acid residue having a sterically bulky side chain that is located approximate to where the tail threads through the ring. In some embodiments, the amino acid residue having the sterically bulky side chain is located on the tail portion and is about 1, 2 or 3 amino acid residue(s) away from where the tail threads through the plane of the ring.

In some embodiments, the loop portion comprises at least one amino acid residue having a sterically bulky side chain that is located approximate to where the tail threads through the plane of the ring. In some embodiments, the amino acid residue having the sterically bulky side chain is located on the loop portion and is about 1, 2 or 3 amino acid residue(s) away from where the tail threads through the plane of the ring.

In some embodiments, the loop portion and the tail portion each comprises at least one amino acid residue having a sterically bulky side chain, and the bulky side chains from the tail and the loop portions flank the plane of the ring to hold the tail in position with respect to the ring. In some embodiments, the loop portion and the tail portion each comprises at least one amino acid residues having a sterically bulky side chain that is about 1, 2, 3 amino acid residue(s) away from where the tail threads through the plane of the ring.

In some embodiments, structural features of a lasso peptide that contribute to its topological stability comprise the size of the ring and the number of amino acid residues in the ring that have a sterically bulky side chain. Without being bound by the theory, it is contemplated that the larger the size of the ring is, the greater number of amino acid residues having sterically bulky side chains are needed to maintain topological stability of a lasso peptide. In some embodiments, a lasso peptide has a 6-member ring, and about 0 to about 3 amino acid residues in the ring that has a bulky side chain. In some embodiments, a lasso peptide has a 7-member ring, and about 0 to about 3 amino acid residues in the ring that has a bulky side chain. In some embodiments, a lasso peptide has an 8-member ring, and about 0 to about 4 amino acid residues in the ring that has a bulky side chain. In some embodiments, a lasso peptide has a 9-member ring, and about 0 to about 4 amino acid residues in the ring that has a bulky side chain.

In various embodiments, the amino acid residues having a sterically bulky side chain are natural amino acids, such as one or more selected from Proline (Pro), Phenylalanine (Phe), Tryptophan (Trp), Methionine (Met), Tyrosine (Tyr), Lysine (Lys), Arginine (Arg), and Histidine (His) residues. In some embodiments, the amino acid residues having a sterically bulky side chain can be unusual amino acids, such as citrulline (Cit), hydroxyproline (Hyp), norleucine (Nle), 3-nitrotyrosine, nitroarginine, ornithine (Orn), naphtylalanine (Nal), Abu, DAB, methionine sulfoxide or methionine sulfone, and those commercially available or known to one of ordinary skill in the art.

According to the present disclosure, the size of ring, loop and/or tail portions of a lasso peptide can be variable. In certain embodiments, the ring portion has about 6 to about 20 amino acid residues including the two ring-forming amino acid residues. In certain embodiments, the loop portion has more than 4 amino acid residues. In certain embodiments, the tail portion has more than 1 amino acid residue.

### 5.3.2 Members of Lasso Peptide Libraries

Provided herein are libraries comprising a plurality of distinct lasso peptides or functional fragments of lasso peptides. In some embodiments, the library comprising the plurality of distinct lasso peptides or functional fragments of lasso peptides is a lasso peptide display library. In some embodiments, the display library comprises a mechanism for distinguishing one member from another. In certain embodiments, each member of the library is associated with a spatial location within the library, such that the members can be identified and/or distinguished from one another based on the spatial information. In certain embodiments, association of the members of the library with a unique location is achieved by individually producing each member of the library at a unique location on a solid support. In certain embodiments, each member of the library is associated with a unique nucleic acid molecule (e.g., a nucleic acid barcode or a nucleic acid encoding a peptidic portion of the displayed entity), and the sequence information of the nucleic acid molecule is sufficient to identify the associated member and/or distinguish the associated member from another member of the library. In certain embodiments, each member of the library is associated with a detectable probe purported to produce a unique detectable signal, and the detectable signal is sufficiently unique to identify the associated member and/or distinguish the associated member from another member of the library, exemplary detectable signals that can be used in connection with the present disclosure include but are not limited to a chemiluminescent signal, a radiological signal, a fluorescent signal, a digital signal, a color signal, etc.

In various embodiments, the lasso peptide display library comprises a plurality members that are (i) intact lasso peptides, (ii) functional fragments of lasso peptides, (iii) fusion proteins each comprising a lasso peptide or a functional fragment of lasso peptide, (iv) protein complexes each comprising a lasso peptide or a functional fragment of lasso peptide, (v) conjugates each comprising a lasso peptide or a functional fragment of lasso peptide, or (vi) any combinations of (i) to (v). The lasso peptide display library as provided herein can be screened for members having one or more desirable properties or functions, such as a desirable activity in binding and/or modulating a cell surface protein to illicit a beneficial cellular response. The lasso peptide display library can be screened for members comprising lasso peptides or functional fragments of lasso peptides suitable for various uses, such as diagnostic uses, prognostic uses, therapeutic uses, or uses as nutraceuticals or food supplements, for human and animals.

### 5.3.2.1 Fusion Proteins

In some embodiments, the lasso peptide display library as provided herein comprises lasso peptides and functional fragments of lasso peptides that form part of a fusion protein, and the fusion protein retains one or more desirable properties or functions (e.g., specifically binds to a target molecule) of the lasso peptide or functional fragment of lasso peptide. In some embodiments, the lasso peptide or functional fragment of lasso peptide is fused at the end of the lasso tail portion to an amino acid sequence that is not a lasso peptide or functional fragment of lasso peptide.

In various embodiments, the fusion proteins are further configured to perform a function different from the desired properties or functions of the lasso peptide or functional fragment of lasso peptide. In specific embodiments, the fusion protein is configured to associate with an identification mechanism that carries sufficient information for identifying the lasso peptide or functional fragment of lasso peptide forming part of the fusion protein. In specific embodiments, the fusion protein is configured to associate with an identification mechanism of a lasso peptide display library that carries sufficient information for distinguishing the lasso peptide or functional fragment of lasso peptide forming part of the fusion protein from other members of the library. In some embodiments, the association between the fusion protein and the identification mechanism is reversible. In some embodiments, the association between the fusion protein and the identification mechanism is via interaction between non-covalent binding pairs. Various types of non-covalent binding pairs are known in the art and can be used in connection with the present application, such as, antibody/antigen, receptor/ligand, streptavidin/biotin, streptavidin/streptavidin binding protein, avidin/biotin, nucleic acid/nucleic acid binding protein, ion/ion-chelating agent, ion/ion-binding protein, and others known in the art. In some embodiments, the fusion comprises a cleavable peptidic linker between the portion comprising the lasso peptide or functional fragment of lasso peptide and the portion configured to associate with the identification mechanism, and upon cleavage of the peptidic linker, the lasso peptide or functional fragment of lasso peptide can be released from the fusion protein.

In specific embodiments, the fusion protein is configured to associate with a unique nucleic acid molecule, where the unique sequence information is sufficient to identify the lasso peptide or functional fragment of lasso peptide forming part of the fusion protein. In specific embodiments, the fusion protein is configured to associate with a unique nucleic acid molecule, where the unique sequence information is sufficient to distinguish the lasso peptide or functional fragment of lasso peptide forming part of the fusion protein from other members of a lasso peptide display library. In specific embodiments, the unique nucleic acid molecule is synthetic DNA barcode. In specific embodiments, the unique nucleic acid molecule comprises a sequence encoding at least a portion of the lasso peptide or functional fragment of lasso peptide forming part of the fusion protein. In specific embodiments, the sequence information carried by the unique nucleic acid molecule can be obtained by amplifying and sequencing the nucleic acid molecule via methods known in the art.

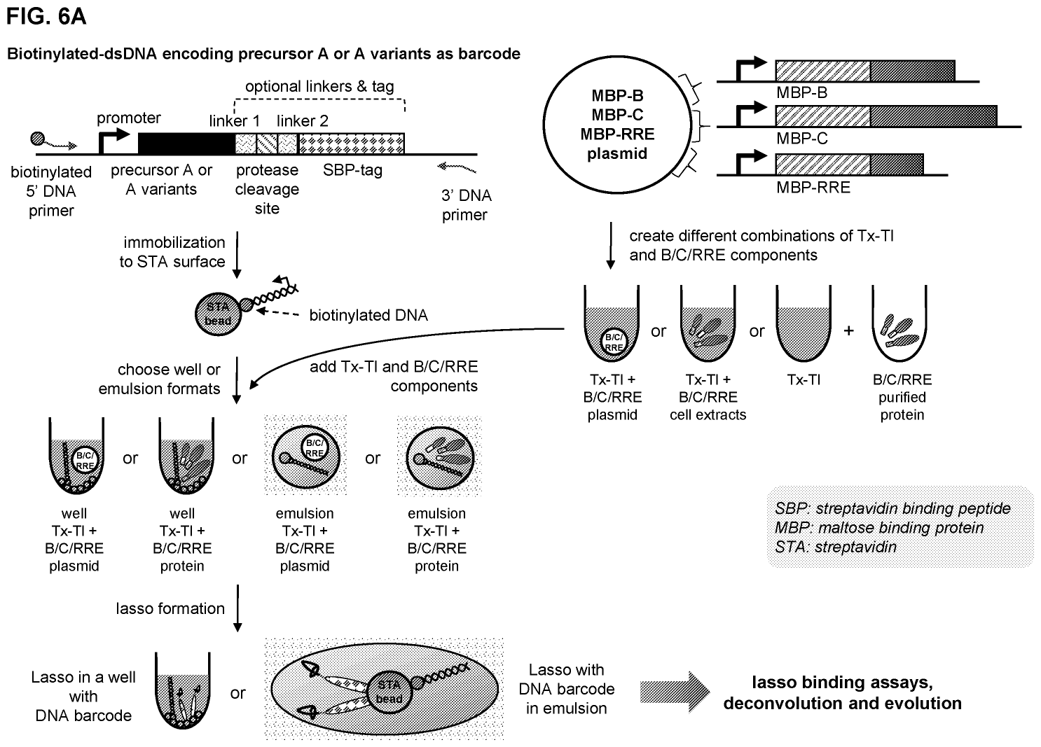

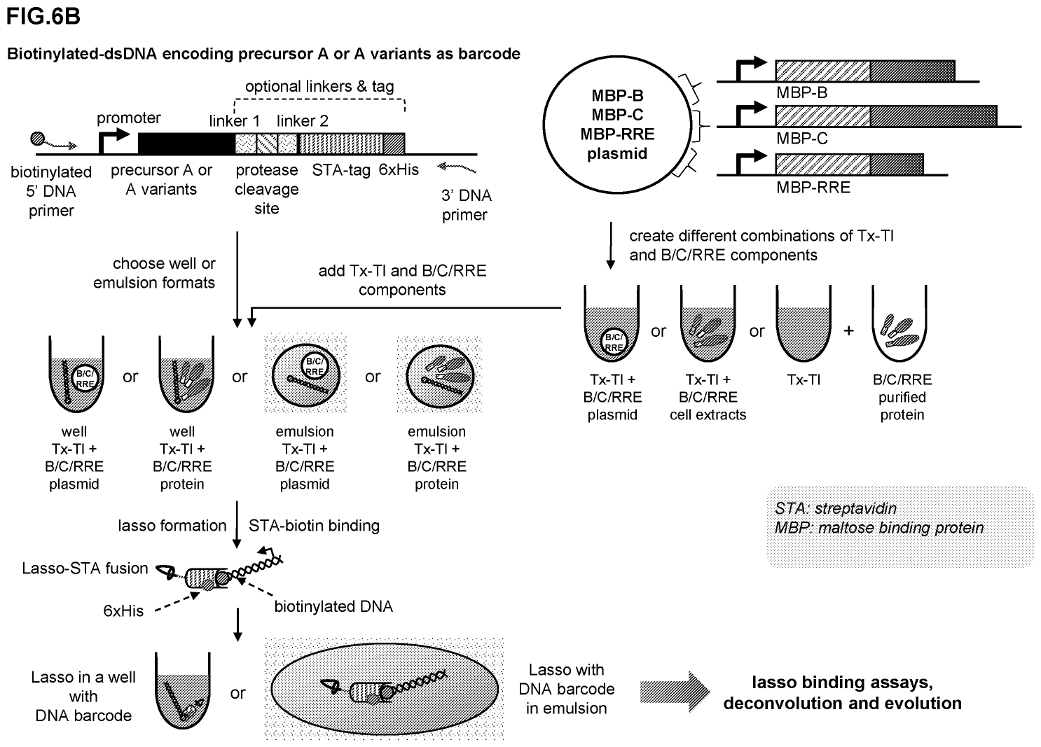

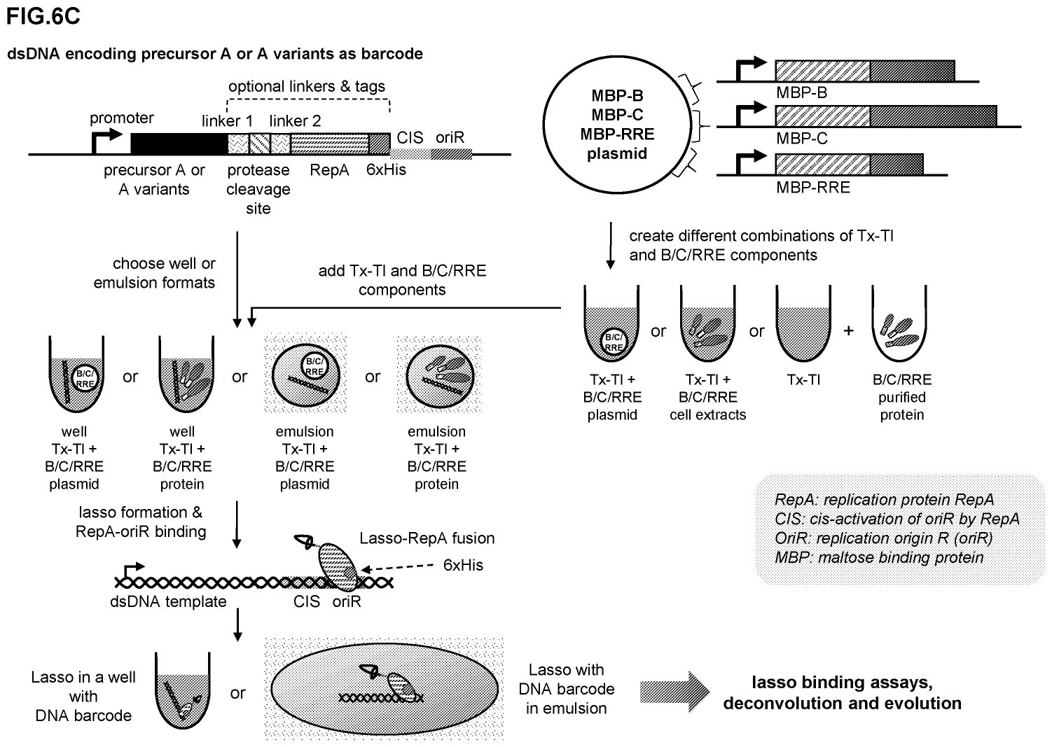

In some embodiments, the fusion protein and the unique nucleic acid molecule directly associate with each other. For example, in specific embodiments, the unique nucleic acid molecule is biotinylated, and the fusion protein comprises a domain capable of associating with the biotin moiety on the unique nucleic acid molecule. For example, in specific embodiments, unique nucleic acid molecule is biotinylated, and the fusion protein comprises a streptavidin (STA) domain, and the fusion protein is associated with the unique nucleic acid via the binding between the streptavidin domain of the fusion protein and the biotin moiety on the unique nucleic acid molecule. See FIG. 6B. In specific embodiments, the fusion protein comprises a nucleic acid binding domain capable of binding to the unique nucleic acid molecule directly. For example, in specific embodiments, the fusion protein comprises a lasso peptide fused to replication protein RepA, and the unique nucleic acid molecule comprises the replication origin R (oriR) sequence and the cis-acting element (CIS) of RepA, and the fusion protein directly associates with the unique nucleic acid molecule via the binding between the RepA domain and the oriR sequence. See FIG. 6C.

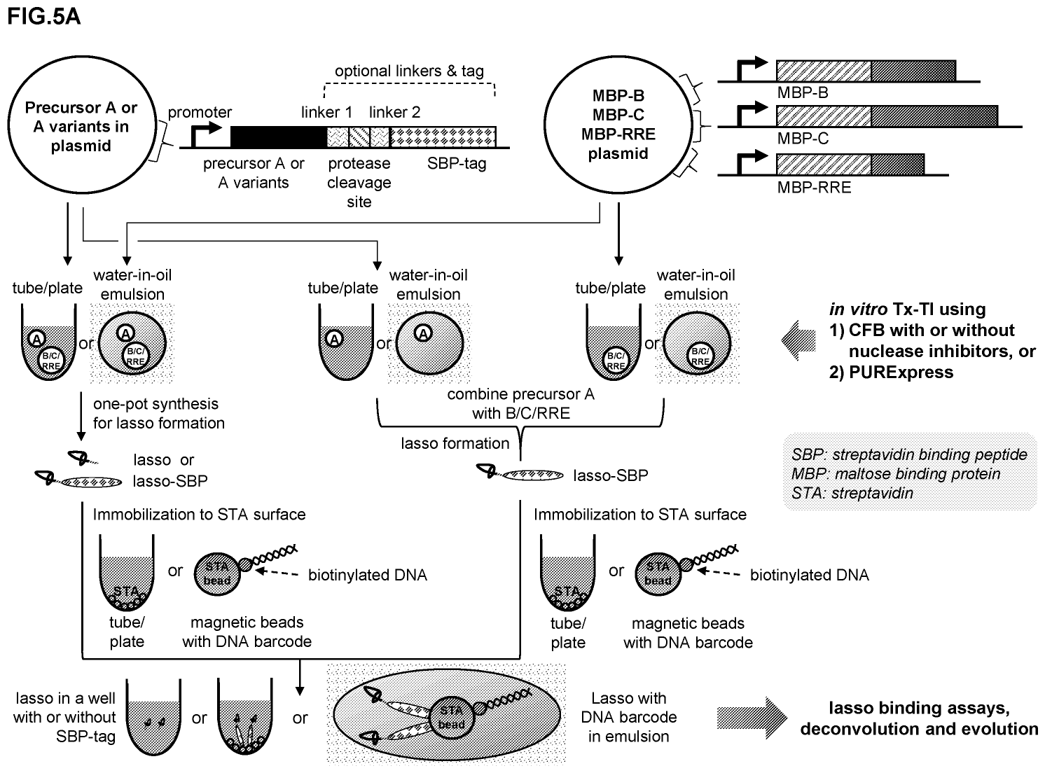

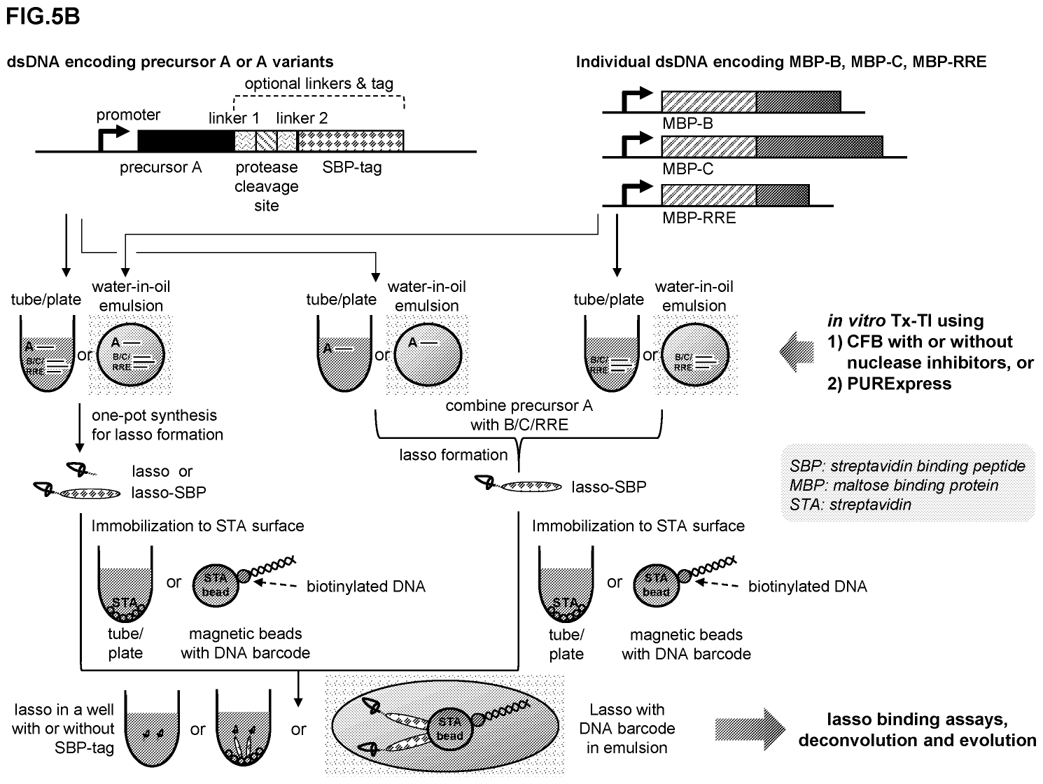

In other embodiments, the fusion protein and the unique nucleic acid molecule associate with each other indirectly, e.g. through another protein or another chemical moiety. For example, in specific embodiments, the fusion protein comprises a streptavidin binding domain, and the unique nucleic acid molecule is biotinylated, and both the fusion protein and the unique nucleic acid molecule associate with a solid support coated with streptavidin. See FIGS. 5A and 6A.

In some embodiments, the fusion protein is configured to associate with a unique location, where the spatial information of the unique location is sufficient to identify the lasso peptide or functional fragment of lasso peptide forming part of the fusion protein. In some embodiments, the fusion protein is configured to associate with a unique location in a lasso peptide display library, where the spatial information of the unique location is sufficient to distinguish the lasso peptide or functional fragment of lasso peptide forming part of the fusion protein from other members of the library. In specific embodiments, the unique location is on a solid support, e.g. a particular well on a multi-well plate, or a particular reaction tube. In some embodiments, the fusion protein comprises a domain capable of binding to a molecule affixed at the unique location. In specific embodiments, the molecule affixed at the unique location and the binding domain in the fusion protein bind with each other via non-covalent interaction. Various types of non-covalent binding pairs are known in the art and can be used in connection with the present application, such as, antibody/antigen, receptor/ligand, streptavidin/biotin, streptavidin/streptavidin binding protein, avidin/biotin, and others known in the art. In specific embodiments, the spatial information of the unique location is obtained by placing fusion proteins comprising lasso peptides or functional fragments of lasso peptide of known identity to a unique location, and associating the identity of the lasso peptide or functional fragment of the lasso peptide with the unique location. In some embodiments, the fusion proteins comprising lasso peptides or functional fragments of lasso peptides are associated with the unique location by producing such fusion protein at the unique location. In specific embodiments, each unique location houses a system for recombinantly producing a fusion protein comprising a distinct lasso peptide or functional fragment of lasso peptide. In specific embodiments, each unique location houses a system for cell-free biosynthesis of a fusion protein comprising a distinct lasso peptide or functional fragment of lasso peptide. In specific embodiments, each unique location houses a system for chemically synthesis of a fusion protein comprising a distinct lasso peptide or functional fragment of lasso peptide.

In some embodiments, the fusion protein comprises a domain that serves as a purification tag. In some embodiments, the fusion protein comprises a domain that produces a detectable signal. In some embodiments, the fusion protein comprises a domain capable of modulating a biological activity. In some embodiments, the fusion protein comprises a domain having therapeutic effect. In some embodiments, the fusion protein comprises a domain that serves as a delivery agent for moving the lasso peptide or functional fragment of lasso peptide to a target location. In various embodiments, the production of fusion proteins can be performed with systems and methods known in the art.

In some embodiments, the lasso precursor peptide genes are fused at the 5′-terminus of the DNA template strand of the gene to oligonucleotide sequences that encode peptides or proteins, such as sequences encoding maltose-binding protein (MBP) or small ubiquitin-like modifier protein (SUMO), which enhance the stability, solubility, and production of the desired TX-TL products (See: Marblestone, J. G., et al., Protein Sci, 2006, 15, 182-189). In some embodiments, the lasso precursor peptides are fused at the N-terminus of the leader sequences with peptides or proteins, such as maltose-binding protein or small ubiquitin-like modifier protein, which enhance the stability, solubility, and production of the fused MBP-lasso or SUMO-lasso precursor peptide. In alternative embodiments, the lasso precursor peptide genes or lasso core peptide genes are fused at the 5′-terminus of the DNA template strand of the gene to oligonucleotide sequences that encode a peptide or protein, with or without a linker, such as sequences encoding amino acid linkers connected to antibodies or antibody fragments, which provide bivalent lasso-antibody products that have enhanced activity against a single target cell or receptor or enhanced activity against two different target cells or receptors. In yet other embodiments, the lasso precursor peptides, lasso core peptides, or lasso peptides are fused at the C-terminus, with or without a linker, to peptides or proteins, such as amino acid linkers connected to antibodies or antibody fragments, which provide bivalent lasso-antibody products that have enhanced activity against a single target cell or receptor or enhanced activity against two different target cells or receptors.

In some embodiments, the MBP-lasso precursor peptide is further fused with a lasso peptidase via a cleavable linker configured to release the lasso peptidase upon cleavage. In some embodiments, the MBP-lasso precursor peptide is further fused with a lasso cyclase via a cleavable linker configured to release the lasso cyclase upon cleavage. In some embodiments, the MBP-lasso precursor peptide is further fused with both of a lasso peptidase and a lasso cyclase via cleavable linkers that are configured to release the two enzymes sequentially or simultaneously.

In certain embodiments, the lasso precursor peptide genes or lasso core peptide genes are fused at the 5′-terminus of the DNA template strand of the gene to oligonucleotide sequences that encode peptides or proteins, with or without a linker, such as sequences encoding peptide tags for affinity purification or immobilization, including his-tags, Strep-tags, or a FLAG-tag. In some embodiments, the lasso precursor peptides, lasso core peptides, or lasso peptides are fused at the C-terminus of the core peptides with other peptides or proteins, with or without a linker, such as peptide tags for affinity purification or immobilization, including his-tags, Strep-tags, or a FLAG-tag.

In some embodiments, the lasso precursor peptide genes or lasso core peptide genes are fused at the 5′-terminus of the DNA template strand of the gene to oligonucleotide sequences that encode peptides or proteins, with or without a linker, such as sequences encoding peptide epitopes that are known to bind with high affinity to antibodies, cell surface proteins, or cell surface receptors, including cytokine binding epitopes, integrin ligand binding epitopes, and the like. In some embodiments, the lasso precursor peptides, lasso core peptides, or lasso peptides are fused at the C-terminus to peptides or proteins, with or without a linker, such as peptide epitopes that are known to bind with high affinity to antibodies, cell surface proteins, or cell surface receptors, including cytokine binding epitopes, integrin ligand binding epitopes, and the like.

In some embodiments, lasso precursor peptides, lasso core peptides, lasso peptides, lasso peptide analogs, lasso peptidases, and/or lasso cyclases are fused to other peptides or proteins, with or without linkers between the partners, to enhance expression, to enhance solubility, to provide stability, to facilitate isolation and purification, and/or to add a distinct functionality. A variety of protein scaffolds may be used as fusion partners for lasso peptides, functional fragments of lasso peptides, lasso core peptides, lasso precursor peptides, lasso peptidases, and/or lasso cyclases, including but not limited to maltose-binding protein (MBP), glutathione S-transferase (GST), thioredoxin (TRX), Nus A protein, ubiquitin (UB), and the small ubiquitin-like modifier protein SUMO (See: De Marco, V., et al., Biochem. Biophys. Res. Commun., 2004, 322, 766-771; Wang, C., et al., Biochem. 1, 1999, 338, 77-81). In other embodiments, peptide fusion partners are used for rapid isolation and purification of lasso precursor peptides, lasso core peptides, lasso peptides, functional fragments of lasso peptides, lasso peptidases, and/or lasso cyclases, including His6-tags, Strep-tags, and FLAG-tags (See: Pryor, K. D., Leiting, B., Protein Expr. Purif., 1997, 10, 309-319; Einhauer A., Jungbauer A., J. Biochem. Biophys. Methods, 2001, 49, 455-465; Schmidt, T. G., Skerra, A., Nature Protocols, 2007, 2, 1528-1535).

In other embodiments, peptide or protein fusion partners are used to introduce new functionality into lasso core peptides, lasso peptides or functional fragments of lasso peptides, such as the ability to bind to a separate biological target, e.g., to form a bispecific molecule for multitarget engagement. In such cases, a variety of peptide or protein partners may be fused with lasso core peptides, lasso peptides or functional fragments of lasso peptides, with or without linkers between the partners, including but not limited to peptide binding epitopes, cytokines, antibodies, monoclonal antibodies, single domain antibodies, antibody fragments, nanobodies, monobodies, affibodies, nanofitins, fluorescent proteins (e.g., GFP), avimers, fibronectins, designed ankyrins, lipocallans, cyclotides, conotoxins, or a second lasso peptide with the same or different binding specificity, e.g., to form bivalent or bispecific lasso peptides (See: Huet, S., et al., PLoS One, 2015, 10 (11): e0142304., doi:10.1371/journal.pone.0142304; Steeland, S., et al., Drug Discov. Today, 2016, 21, 1076-1113; Lipovsek, D., Prot. Eng., Des. Sel., 2011, 24, 3-9; Sha, F., et al., Prot. Sci., 2017, 26, 910-924; Silverman, J., et al., Nat. Biotech., 2005, 23, 1556-1561; Pluckthun, A., Diagnostics, and Therapy, Annu. Rev. Pharmacol. Toxicol., 2015, 55, 489-511; Nelson, A. L., mAbs, 2010, 2, 77-83; Boldicke, T., Prot. Sci, 2017, 26, 925-945; Liu, Y., et al., ACS Chem Biol., 2016, 11, 2991-2995; Liu, T., et al., Proc. Nat. Acad. Sci. U.S.A., 2015, 112, 1356-1361; Mûller D., Pharmacol Ther., 2015, 154, 57-66; Weidmann, J.; Craik, D. J., J. Experimental Bot., 2016, 67, 4801-4812; Burman, R., et al., J. Nat. Prod. 2014, 77, 724-736; Reinwarth, M., et al., Molecules, 2012, 17, 12533-12552; Uray, K., Hudecz, F., Amino Acids, Pept. Prot., 2014, 39, 68-113).

In other embodiments, a lasso precursor peptide gene is fused at the 3′-terminus of the leader sequence, or at the 5′-terminus of the core peptide sequence of the DNA template strand of the gene, to oligonucleotide sequences that encode peptides or proteins, including sequences that encode maltose-binding protein (MBP) or small ubiquitin-like modifier protein (SUMO), which enhance the stability and/or production of the desired products formed using a TX-TL-based CFB method or process (See: Marblestone, J. G., et al., Protein Sci, 2006, 15, 182-189). In some embodiments, the lasso precursor peptides are fused at the N-terminus of the leader sequence or at the C-terminus of the core sequence to form fusion proteins with peptides or proteins, including maltose-binding protein or small ubiquitin-like modifier protein, which enhance the stability and/or production of the lasso peptide precursor fusion product, e.g., MBP-lasso precursor peptide or SUMO-lasso precursor peptide. In yet other embodiments, a lasso core peptide gene is fused at the 5′-terminus of the core peptide sequence of the DNA template strand of the gene to oligonucleotide sequences that encode peptides or proteins, including sequences that encode maltose-binding protein (MBP) or small ubiquitin-like modifier protein (SUMO), which enhance the stability and/or production of the desired products formed using a TX-TL-based CFB method or process. In alternative embodiments, a lasso core peptide is fused at the C-terminus of the core sequence to form fusion proteins with peptides or proteins, including maltose-binding protein or small ubiquitin-like modifier protein, which enhance the stability and/or production of the lasso peptide precursor fusion product, e.g., MBP-lasso core peptide or SUMO-lasso core peptide. In alternative embodiments, a lasso peptide is fused at the N-terminus or at the C-terminus of the lasso peptide to form fusion proteins with peptides or proteins, including maltose-binding protein or small ubiquitin-like modifier protein, which enhance the stability and/or production of the lasso peptide precursor fusion product, e.g., MBP-lasso peptide or SUMO-lasso peptide.

In other embodiments, lasso peptidase or lasso cyclase genes are fused at the 5′- or 3′-terminus with oligonucleotide sequences that encode peptides or proteins, including sequences that encode maltose-binding protein (MBP) or small ubiquitin-like modifier protein (SUMO). In alternative embodiments, lasso peptidases or lasso cyclases are fused at the N-terminus or the C-terminus to peptides or proteins, such as maltose-binding protein (MBP) or small ubiquitin-like modifier protein (SUMO), which enhance the stability and/or production of the desired TX-TL products.

In alternative embodiments, the lasso precursor peptide genes or lasso core peptide genes are fused at the 5′-terminus of the DNA template strand of the gene to oligonucleotide sequences that encode a peptide or protein, with or without a linker, such as sequences encoding amino acid linkers connected to antibodies or antibody fragments, which provide bivalent lasso-antibody products that exhibit enhanced activity against an individual biological target, receptor, or cell type, or enhanced activity against two different biological targets, receptors, or cell types. In some embodiments, the lasso precursor peptides or lasso core peptides or lasso peptides are fused at the C-terminus to form fusion proteins with peptides or proteins, such as amino acid linkers connected to antibodies or antibody fragments, which provide bivalent lasso-antibody products that exhibit enhanced activity against an individual biological target, receptor, or cell type, or enhanced activity against two different biological targets, receptors, or cell types.

### 5.3.2.2 Protein Complexes

In certain embodiments, the lasso peptides and functional fragments of lasso peptides provided herein form part of a protein complex, and the protein complex retains one or more desirable properties or functions (e.g., specifically bind to a target molecule) of the lasso peptide or functional fragment of lasso peptide.

In specific embodiments, the protein complex is configured to associate with an identification mechanism that carries sufficient information for identifying the lasso peptide or functional fragment of lasso peptide forming part of the protein complex. In specific embodiments, the protein complex is configured to associate with an identification mechanism of a lasso peptide display library, in which identification mechanism carries sufficient information for distinguishing the lasso peptide or functional fragment of lasso peptide forming part of the protein complex from other members of the library. In some embodiments, the association between the protein complex and the identification mechanism is reversible. In some embodiments, the association between the protein complex and the identification mechanism is via interaction between non-covalent binding pairs. Various types of non-covalent binding pairs are known in the art and can be used in connection with the present application, such as, antibody/antigen, receptor/ligand, streptavidin/biotin, streptavidin/streptavidin binding protein, avidin/biotin, and others known in the art.

In specific embodiments, the protein complex is configured to associate with a unique nucleic acid molecule, where the unique sequence information is sufficient to identify the lasso peptide or functional fragment of lasso peptide forming part of the protein complex. In specific embodiments, the protein complex is configured to associate with a unique nucleic acid molecule, where the unique sequence information is sufficient to distinguish the lasso peptide or functional fragment of lasso peptide forming part of the protein complex from other members of a lasso peptide display library. In specific embodiments, the unique nucleic acid molecule is synthetic DNA barcode. In specific embodiments, the unique nucleic acid molecule comprises a sequence encoding at least a portion of the lasso peptide or functional fragment of lasso peptide forming part of the protein complex. In specific embodiments, the sequence information carried by the unique nucleic acid molecule can be obtained by amplifying and sequencing the nucleic acid molecule via methods known in the art.

In some embodiments, the protein complex and the unique nucleic acid molecule directly associate with each other. For example, in specific embodiments, the protein complex comprises a nucleic acid binding domain or subunit capable of binding to the unique nucleic acid molecule directly. For example, in specific embodiments, the protein complex comprises a domain or a subunit that comprises the replication protein RepA, and the unique nucleic acid molecule comprises the replication origin R (oriR) sequence and the cis-acting element (CIS) of RepA, and the protein complex directly associates with the unique nucleic acid molecule via the binding between the RepA domain and the oriR sequence.

In other embodiments, the protein complex and the unique nucleic acid molecule associate with each other indirectly, e.g. through another protein or another chemical moiety. For example, in specific embodiments, the unique nucleic acid molecule is biotinylated, and the protein complex comprises a domain or subunit capable of associating with the biotin moiety on the unique nucleic acid molecule. For example, in specific embodiments, unique nucleic acid molecule is biotinylated, and the protein complex comprises a domain or subunit that comprises streptavidin, and the protein complex associates with the unique nucleic acid via the binding between the streptavidin in the protein complex and the biotin moiety on the unique nucleic acid molecule. In specific embodiments, the protein complex comprises a streptavidin binding domain or subunit, and the unique nucleic acid molecule is biotinylated, and both the protein complex and the unique nucleic acid molecule associate with a solid support coated with streptavidin.

In some embodiments, the protein complex is configured to associate with a unique location, where the spatial information of the unique location is sufficient to identify the lasso peptide or functional fragment of lasso peptide forming part of the protein complex. In some embodiments, the protein complex is configured to associate with a unique location in a lasso peptide display library, where the spatial information of the unique location is sufficient to distinguish the lasso peptide or functional fragment of lasso peptide forming part of the protein complex from other members of the library. In specific embodiments, the unique location is on a solid support, e.g. a particular well on a multi-well plate, or a particular reaction tube. In some embodiments, the protein complex comprises a domain or subunit capable of binding to a molecule affixed at the unique location. In specific embodiments, the molecule affixed at the unique location and the binding domain or subunit of the protein complex bind with each other via non-covalent interaction. Various types of non-covalent binding pairs are known in the art and can be used in connection with the present application, such as, antibody/antigen, receptor/ligand, streptavidin/biotin, streptavidin/streptavidin binding protein, avidin/biotin, and others known in the art. In specific embodiments, the spatial information of the unique location is obtained by placing protein complexes comprising lasso peptides or functional fragments of lasso peptide of known identity to a unique location, and associating the identity of the lasso peptide or functional fragment of the lasso peptide with the unique location. In some embodiments, the protein complexes comprising lasso peptides or functional fragments of lasso peptides are associated with the unique location by individually producing each protein complex at a unique location. In specific embodiments, each unique location houses a system for recombinantly producing a protein complex comprising a distinct lasso peptide or functional fragment of lasso peptide. In specific embodiments, each unique location houses a system for cell-free biosynthesis of a protein complex comprising a distinct lasso peptide or functional fragment of lasso peptide. In specific embodiments, each unique location houses a system for chemically synthesis of a protein complex comprising a distinct lasso peptide or functional fragment of lasso peptide.

In some embodiments, the protein complex comprises a domain or subunit that serves as a purification tag. In some embodiments, the protein complex comprises a domain or subunit that produces a detectable signal. In some embodiments, the protein complex comprises a domain or subunit capable of modulating a biological activity. In some embodiments, the protein complex comprises a domain or subunit capable of producing a therapeutic effect. In some embodiments, the fusion protein comprises a domain or subunit that serves as a delivery agent for moving the lasso peptide or functional fragment of lasso peptide to a target location. In various embodiments, the production of fusion proteins can be performed with systems and methods known in the art.

### 5.3.2.3 Conjugates

In certain embodiments, the lasso peptides and functional fragments of lasso peptides provided herein is conjugated to an identification mechanism that carries sufficient information for identifying the lasso peptide or functional fragment of lasso peptide forming part of the conjugate. In certain embodiments, the lasso peptides and functional fragments of lasso peptides provided herein is conjugated to a unique nucleic acid molecule, and the lasso peptide conjugate retains one or more desirable properties or functions (e.g., specifically binds to a target molecule) of the lasso peptide or functional fragment of lasso peptide.

In specific embodiments, the lasso peptide or functional fragment of lasso peptide is conjugated with a non-peptidic entity that carries sufficient information for identifying the lasso peptide or functional fragment of lasso peptide forming part of the conjugate. In specific embodiments, the lasso peptide or functional fragment of lasso peptide is conjugated with an identification mechanism of a lasso peptide display library, which identification mechanism carries sufficient information for distinguishing the lasso peptide or functional fragment of lasso peptide forming part of the conjugate from other members of the library. In some embodiments, the conjugation between the protein complex and the identification mechanism is reversible. Conjugation of the unique nucleic acid molecule to a lasso peptide or functional fragment of lasso peptide can occur at one or more amino acid residues, including amino acid residues located in the ring portion, loop portion and/or tail portion of the lasso peptide or functional fragment of lasso peptide.

In specific embodiments, the lasso peptide or functional fragment of lasso peptide is conjugated to a unique nucleic acid molecule, where the unique sequence information is sufficient to identify the lasso peptide or functional fragment of lasso peptide forming part of the conjugate. In specific embodiments, the lasso peptide or functional fragment of lasso peptide is conjugated with a unique nucleic acid molecule, where the unique sequence information is sufficient to distinguish the lasso peptide or functional fragment of lasso peptide forming part of the conjugate from other members of a lasso peptide display library. In specific embodiments, the unique nucleic acid molecule is synthetic DNA barcode. In specific embodiments, the unique nucleic acid molecule comprises a sequence encoding at least a portion of the lasso peptide or functional fragment of lasso peptide forming part of the conjugate. In specific embodiments, the sequence information carried by the unique nucleic acid molecule can be obtained by amplifying and sequencing the nucleic acid molecule via methods known in the art.

Conjugation between the lasso peptide or functional fragment of lasso peptide and the unique nucleic acid molecule can be achieved using systems and methods known in the art. For example, in specific embodiments, the core peptides or the lasso peptides produced by cell-free biosynthesis are modified further through chemical steps, for example through chemical steps that allow the attachment of chemical linker units connected to small molecules to the C-terminus of the core peptide or the lasso peptide, or the attachment of chemical linkers connected to small molecules to the side chain of functionalized amino acids (e.g., the OH or serine, threonine, or tyrosine, or the N of lysine). In other embodiments, the lasso core peptides or the lasso peptides produced by cell-free biosynthesis are modified further through chemical steps, for example, by PEGylation or biotinylation, or through the formation of esters, sulfonyl esters, phosphonate esters, or amides by reaction with the side chain of functionalized amino acids (e.g., the OH or serine, threonine, or tyrosine, or the N of lysine). In yet other embodiments, the core peptides or the lasso peptides produced by cell-free biosynthesis may contain non-natural amino acids which are modified further through chemical steps, for example, by the use of click chemistry involving amino acids with azide or alkyne functionality within the side chains (See: Presolski, S. I., et al., Curr Protoc Chem Biol., 2011, 3, 153-162), or through metathesis chemistry involving alkene or alkyne groups within the amino acid side chains (See: Cromm, P. M., et al., Nat. Comm., 2016, 7, 11300; Gleeson, E. C., et al., Tetrahedron Lett., 2016, 57, 4325-4333).

### 5.3.3 Production of Lasso Peptide Libraries

Provided herein are methods and systems for producing lasso peptides. In certain embodiments, the lasso peptides are provided in the form of (i) intact lasso peptides (ii) functional fragments of lasso peptides; (iii) fusion proteins each comprising a lasso peptide or a functional fragment of lasso peptide; (iv) protein complexes each comprising a lasso peptide or a functional fragment of lasso peptide; or (v) conjugates each comprising a lasso peptide or a functional fragment of lasso peptide. Particularly, (ii)-(v) are collectively referred to as related molecules of lasso peptides.

In certain embodiments, the methods provided herein can produce a large number of distinct lasso peptides and/or related molecules thereof in a short period of time. In some embodiments, the methods provided herein can produce a plurality of diversified species of lasso peptides and/or related molecules thereof simultaneously.

Also provided herein are methods and systems for assembling a plurality of diversified species of lasso peptides and/or related molecules thereof into a library. In various embodiments, the lasso peptide library comprises (i) intact lasso peptides, (ii) functional fragments of lasso peptides, (iii) fusion proteins each comprising a lasso peptide or a functional fragment of lasso peptide, (iv) protein complexes each comprising a lasso peptide or a functional fragment of lasso peptide, (v) conjugates each comprising a lasso peptide or a functional fragment of lasso peptide, or (vi) any combinations of (i) to (v). In particular embodiments, the lasso peptide library is a display library as provided herein. In particular embodiments, the lasso peptide library is a molecule display library as provided herein.

### 5.3.3.1 Genomic Mining Tools for Genes Coding Natural Lasso Peptides

Some naturally existing lasso peptides are encoded by a lasso peptide biosynthetic gene cluster, which typically comprises three main genes: one encodes for a lasso precursor peptide (referred to as Gene A), and two encode for processing enzymes including a lasso peptidase (referred to as Gene B) and a lasso cyclase (referred to as Gene C). The lasso precursor peptide comprises a lasso core peptide and additional peptidic fragments known as the “leader sequence” that facilitates recognition and processing by the processing enzymes. The leader sequence may determine substrate specificity of the processing enzymes. The processing enzymes encoded by the lasso peptide gene cluster convert the lasso precursor peptide into a matured lasso peptide having the lariat-like topology. Particularly, the lasso peptidase removes additional sequences from the precursor peptide to generate a lasso core peptide, and the lasso cyclase cyclizes a terminal portion of the core peptide around a terminal tail portion to form the lariat-like topology. Some lasso gene clusters further encodes for additional protein elements that facilitates the post-translational modification, including a facilitator protein known as the post-translationally modified peptide (RiPP) recognition element (RRE). Some lasso gene clusters further encodes for lasso peptide transporters, kinases, or proteins that play a role in immunity, such as isopeptidase. (Burkhart, B. J., et al., Nat. Chem. Biol., 2015, 11, 564-570; Knappe, T. A. et al., J. Am. Chem. Soc., 2008, 130, 11446-11454; Solbiati, J. O. et al. J. Bacteriol., 1999, 181, 2659-2662; Fage, C. D., et al., Angew. Chem. Int. Ed., 2016, 55, 12717-12721; Zhu, S., et al., J. Biol. Chem. 2016, 291, 13662-13678).

Computer-based genome-mining tools can be used to identify lasso biosynthetic gene clusters based on known genomic information. For example, one algorithm known as RODEO can rapidly analyze a large number of biosynthetic gene clusters (BGCs) by predicting the function for genes flanking query proteins. This is accomplished by retrieving sequences from GenBank followed by analysis with HMMER3. The results are compared against the Pfam database with the data being returned to the users in the form of spreadsheet. For analysis of BGCs not encoding proteins not covered by Pfam, RODEO allows usage of additional pHMMs (either curated databases or user-generated). Taking advantage of RODEO's ability to rapidly analyze genes neighboring a query, it is possible to compile a list of all observable lasso peptide biosynthetic gene clusters in GeneBank (Online Methods). A comprehensive evaluation of this data set would provide great insight into the lasso peptide family. Lasso peptide biosynthetic gene clusters can be identified by looking for the local presence of genes encoding proteins matching the Pfams for the lasso cyclase, lasso peptidase, and RRE.

To confidently predict lasso precursors, RODEO next performed a six-frame translation of the intergenic regions within each of the identified potential lasso biosynthetic gene clusters. The resulting peptides can be assessed based on length and essential sequence features and split into predicted leader and core regions. A series of heuristics based on known lasso peptide characteristics can be defined to predict precursors from a pool of false positives. After optimization of heuristic scoring, good prediction accuracy for biosynthetic gene clusters closely related to known lasso peptides can be obtained.

Machine learning, particularly, support vector machine (SVM) classification, would be effective in locating precursor peptides from predicted BGCs more distant to known lasso peptides. SVM is well-suited for RiPP discovery due to availability of SVM libraries that perform well with large data sets with numerous variables and the ability of SVM to minimize unimportant features. The SVM classifier can be optimized using a randomly selected and manually curated training set from the unrefined whole data. Of these, a random subpopulation was withheld as a test set to avoid over-fitting. By combining SVM classification with motif (MEME) analysis, along with our original heuristic scoring, prediction accuracy was greatly enhanced as evaluated by recall and precision metrics. This tripartite procedure can yield a high-scoring, well-separated population of lasso precursor peptide from candidate peptides. The training set was found to display nearly identical scoring distributions upon comparison to the full data set.

Other examples of genomic or biosynthetic gene search engine that can be used in connection with the present disclosure include the WARP DRIVE BIO™ software, anti-SMASH (ANTI-SMASH™) software (See: Blin, K., et al., Nucleic Acids Res., 2017, 45, W36-W41), iSNAP™ algorithm (See: Ibrahim, A., et al., Proc. Nat. Acad. Sci., USA., 2012, 109, 19196-19201), CLUSTSCAN™ (Starcevic, et al., Nucleic Acids Res., 2008, 36, 6882-6892), NP searcher (Li et al. (2009) Automated genome mining for natural products. BMC Bioinformatics, 10, 185), SBSPKS™ (Anand, et al. Nucleic Acids Res., 2010, 38, W487-W496), BAGEL3™ (Van Heel, et al., Nucleic Acids Res., 2013, 41, W448-W453), SMURF™ (Khaldi et al., Fungal Genet. Biol., 2010, 47, 736-741), ClusterFinder (CLUSTERFINDER™) or ClusterBlast (CLUSTERBLAST™) algorithms, and an Integrated Microbial Genomes (IMG)-ABC system (DOE Joint Genome Institute (JGI)). In some embodiments, lasso peptide biosynthetic gene clusters for use in CFB methods and processes as provided herein are identified by mining genome sequences of known bacterial natural product producers using established genome mining tools, such as anti-SMASH, BAGEL3, and RODEO. These genome mining tools can also be used to identify novel biosynthetic genes (for use in CFB systems and processes as provided herein) within metagenomic based DNA sequences. Lasso peptide biosynthetic gene clusters can be used in the methods and systems described herein to produce various lasso peptides and libraries of lasso peptides.

### 5.3.3.2 Nucleic Acids for CFB Systems

In alternative embodiments, CFB methods and systems, provided herein to produce lasso peptides and related molecules thereof from a minimal set of lasso peptide biosynthetic pathway components, including the use of whole cell, cytoplasmic or nuclear extracts, comprise the use of nucleic acids, which can be substantially isolated or synthetic nucleic acids, comprising or encoding: a lasso precursor peptide; a lasso core peptide; a lasso peptide synthesizing enzyme or enzymes; a biosynthetic gene cluster, a lasso peptide biosynthetic pathway operon; optionally a lasso peptide biosynthetic gene cluster comprising coding sequences for all or substantially all or a minimum set of enzymes needed in the synthesis of a lasso peptide or related molecules thereof; a plurality of enzyme-encoding nucleic acids; a plurality of enzyme-encoding nucleic acids for at least two, several or all of the steps in the synthesis of a lasso peptide or related molecules thereof. In alternative embodiments, the substantially isolated or synthetic nucleic acids are in a linear or a circular form, or are contained in a circular or a linearized plasmid, vector or phage DNA. In alternative embodiments, the substantially isolated or synthetic nucleic acids comprise enzyme coding sequences operably linked to a homologous or a heterologous transcriptional regulatory sequence, optionally a transcriptional regulatory sequence is a promoter, an enhancer, or a terminator of transcription. In alternative embodiments, the substantially isolated or synthetic nucleic acids comprise at least about 50, 100, 200, 250, 300, 350, 400, 450, 500, 550, 600 or more base pair ends upstream of the promoter and/or downstream of the terminator.

In alternative embodiments, expression constructs, vehicles or vectors are provided to make, or to include, or contain within, one or more nucleic acids used in the CFB methods and processes, provided herein for the synthesis of lasso peptides and related molecules thereof from a minimal set of lasso peptide biosynthetic pathway components. In alternative embodiments, nucleic acids used in the CFB methods and processes, provided herein for the synthesis of lasso peptides and related molecules thereof from a minimal set of lasso peptide biosynthetic pathway components, are operably linked to an expression (e.g., transcription or translational) control sequence, e.g., a promoter or enhancer, e.g., a control sequence functional in a cell from which an extract has been derived. In alternative embodiments, expression constructs, expression vehicles or vectors, plasmids, phage vectors, viral vectors or recombinant viruses, episomes and artificial chromosomes, including vectors and selection sequences or markers containing nucleic acids are used to make or express the lasso peptide pathway genes as provided herein. In alternative embodiments, the expression vectors also include one or more selectable marker genes and appropriate expression control sequences.

Selectable marker genes also can be included, for example, on plasmids that contain genes for lasso peptide synthesis to provide resistance to antibiotics or toxins, to complement auxotrophic deficiencies, or to supply critical nutrients not in an extract. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vehicle (e.g., a vector or plasmid) or in separate expression vehicles. For single vehicle/vector expression, the encoding nucleic acids can be operationally linked to one common expression control sequence or linked to different expression control sequences, such as one inducible promoter and one constitutive promoter.

In alternative embodiments, nucleic acid analysis such as Northern blots or polymerase chain reaction (PCR) amplification of mRNA, or immunoblotting, are used for analysis of expression of gene products, e.g., enzyme-encoding message; any analytical method can be used to test the expression of an introduced nucleic acid sequence or its corresponding gene product. The exogenous nucleic acid can be expressed in a sufficient amount to produce the desired product, and expression levels can be optimized to obtain sufficient expression.

In alternative embodiments, multiple enzyme-encoding nucleic acids (e.g., two or more genes) are fabricated on one polycistronic nucleic acid. In alternative embodiments, one or more enzyme-coding nucleic acids of a desired lasso peptide synthetic pathway are fabricated on one linear or circular DNA. In alternative embodiments, all or a subset of the enzyme-encoding nucleic acid of an enzyme-encoding lasso peptide synthesizing operon or biosynthetic gene cluster are contained on separate linear nucleic acids (separate nucleic acid strands), optionally in equimolar concentrations in a whole cell, cytoplasmic or nuclear extract, as described above, and optionally, each separate linear nucleic acid comprises one, two, three, 4, 5, 6, 7, 8, 9, or 10 or more genes or enzyme-encoding sequences, and optionally the linear nucleic acid is present in a cell extract at a concentration of about 10 nM (nanomolar), 15 nM, 20 nM, 25 nM, 30 nM, 35 nM, 40 nM, 45 nM or 50 nM or more or between about 1 nM and 100 nM.

Identifying and Modifying Lasso Peptide Biosynthetic Genes, Gene Clusters, Enzymes, and Pathways

Provided herein are methods of identifying and/or modifying an enzyme-encoding lasso peptide synthesizing operon; a lasso peptide biosynthetic gene cluster; a plurality of enzyme-encoding nucleic acids for lasso precursor peptides or lasso core peptides and at least one, several or all of the steps in the synthesis of a lasso peptide or related molecules thereof upon transforming a lasso precursor peptide or lasso core peptide. In alternative embodiments, provided are engineered or modified enzyme-encoding lasso peptide synthesizing operons; lasso peptide biosynthetic gene clusters; and/or enzyme-encoding nucleic acids for lasso precursor peptides or lasso core peptides and at least one, several or all of the steps in the synthesis of a lasso peptide or related molecules thereof upon transforming a lasso precursor peptide or lasso core peptide, or libraries thereof, made by these methods. In alternative embodiments, provided are libraries of lasso peptides or related molecules thereof made by these methods, and compositions as provided herein. In alternative embodiments, these modifications comprise one or more combinatorial modifications that result in generation of desired lasso peptides or related molecules thereof, or libraries of lasso peptides or related molecules thereof.

In alternative embodiments, the one or more combinatorial modifications comprise deletion or inactivation one or more individual genes, in a gene cluster for the biosynthesis, or altered biosynthesis, ultimately leading to a minimal optimum gene set for the biosynthesis of lasso peptides or related molecules thereof.

In alternative embodiments, the one or more combinatorial modifications comprise domain engineering to fused protein (e.g., enzyme) domains, shuffled domains, adding an extra domain, exchange of one or more (multiple) domains, or other modifications to alter substrate activity or specificity of an enzyme involved in the biosynthesis or modification of the lasso peptides or related molecules thereof.

In alternative embodiments, the one or more combinatorial modifications comprise modifying, adding or deleting a “tailoring” enzyme that act after the biosynthesis of a core backbone of the lasso peptide or related molecules thereof is completed, optionally comprising N-methyltransferases, O-methyltransferases, biotin ligases, glycosyltransferases, esterases, acylases, acyltransferases, aminotransferases, amidases, hydroxylases, dehydrogenases, halogenases, kinases, RiPP heterocyclases, RiPP cyclodehydratases, peptidylarginine deiminase (Nat Chem Biol. 2017 May; 13(5):470-478) and prenyltransferases. In this embodiment, lasso peptides or related molecules thereof are generated by the action (e.g., modified action, additional action, or lack of action (as compared to wild type)) of the “tailoring” enzymes.

In alternative embodiments, the one or more combinatorial modifications comprise combining lasso peptide biosynthetic genes from various sources to construct artificial lasso peptide biosynthesis gene clusters, or modified lasso peptide biosynthesis gene clusters.

In alternative embodiments, functional or bioinformatic screening methods are used to discover and identify biocatalysts, genes and gene clusters, e.g., lasso peptide biosynthetic gene clusters, for use the CFB methods and processes as described herein. Environmental habitats of interest for the discovery of lasso peptides includes soil and marine environments, for example, through DNA sequence data generated through either genomic or metagenomic sequencing.

In alternative embodiments, enzyme-encoding lasso peptide synthesizing operons; lasso peptide biosynthetic gene clusters; and/or enzyme-encoding nucleic acids for lasso precursor peptides or lasso core peptides and at least one, several or all of the steps in the synthesis of a lasso peptide or related molecules thereof upon transforming a lasso precursor peptide or lasso core peptide, or libraries thereof, made by the CFB methods and processes provided herein, are identified by methods comprising e.g., use of: a genomic or biosynthetic search engine, optionally WARP DRIVE BIO™ software, anti-SMASH (ANTI-SMASH™) software (See: Blin, K., et al., Nucleic Acids Res., 2017, 45, W36-W41), iSNAP™ algorithm (See: Ibrahim, A., et al., Proc. Nat. Acad. Sci., USA., 2012, 109, 19196-19201), CLUSTSCAN™ (Starcevic, et al., Nucleic Acids Res., 2008, 36, 6882-6892), NP searcher (Li et al. (2009) Automated genome mining for natural products. BMC Bioinformatics, 10, 185), SBSPKS™ (Anand, et al. Nucleic Acids Res., 2010, 38, W487-W496), BAGEL3™ (Van Heel, et al., Nucleic Acids Res., 2013, 41, W448-W453), SMURF™ (Khaldi et al., Fungal Genet. Biol., 2010, 47, 736-741), ClusterFinder (CLUSTERFINDER™) or ClusterBlast (CLUSTERBLAST™) algorithms, the RODEO algorithm (See: Tietz, J. I., et al., Nature Chem Bio, 2017, 13, 470-478), or a combination there of; or, an Integrated Microbial Genomes (IMG)-ABC system (DOE Joint Genome Institute (JGI)).

In alternative embodiments, lasso peptide biosynthetic gene clusters for use in CFB methods and processes as provided herein are identified by mining genome sequences of known bacterial natural product producers using established genome mining tools, such as anti-SMASH, BAGEL3, and RODEO. These genome mining tools can also be used to identify novel biosynthetic genes (for use in CFB systems and processes as provided herein) within metagenomic based DNA sequences.

In alternative embodiments, CFB reaction mixtures and cell extracts as provided herein use (incorporate, or comprise) protein machinery that is responsible for the biosynthesis of secondary metabolites inside prokaryotic and eukaryotic cells; this “machinery” can comprise enzymes encoded by gene clusters or operons. In alternative embodiments, so-called “secondary metabolite biosynthetic gene clusters (SMBGCs) are used; they contain all the genes required for the biosynthesis, regulation and/or export of a product, e.g., a lasso peptide. In vivo genes are encoded (physically located) side-by-side, and they can be used in this “side-by-side” orientation in (e.g., linear or circular) nucleic acids used in the CFB method and processes using cell extracts as provided herein, or they can be rearranged, or segmented into one or more linear or circular nucleic acids.

In alternative embodiments, the identified lasso peptide biosynthetic gene clusters and/or biosynthetic genes are ‘refactored’, e.g., where the native regulatory parts (e.g. promoter, RBS, terminator, codon usage etc.) are replaced e.g., by synthetic, orthogonal regulation with the goal of optimization of enzyme expression in a cell extract as provided herein and/or in a heterologous host (See: Tan, G.-Y., et al., Metabolic Engineering, 2017, 39, 228-236). In alternative embodiments, refactored lasso peptide biosynthetic gene clusters and/or genes are modified and combined for the biosynthesis of other lasso peptide analogs (combinatorial biosynthesis). In alternative embodiments, refactored gene clusters are added to a CFB reaction mixture with a cell extract as provided herein, and they can be added in the form of linear or circular DNA, e.g., plasmid or linear DNA.

In alternative embodiments, refactoring strategies comprise changes in a start codon, for example, for Streptomyces it might be advantageous to change the start codon, e.g., to TTG. For Streptomyces it has been shown that genes starting with TTG are better transcribed than genes starting with ATG or GTG (See: Myronovskyi et al., Applied and Environmental Microbiology, 2011; 77, 5370-5383).

In alternative embodiments, refactoring strategies comprise changes in ribosome binding sites (RBSs), and RBSs and their relationship to a promoter, e.g., promoter and RBS activity can be context dependent. For example, the rate of transcription can be decoupled from the contextual effect by using ribozyme-based insulators between the promoter and the RBS to create uniform 5′-UTR ends of mRNA, (See: Lou, et al., Nat. Biotechnol., 2012, 30, 1137-42.

In alternative embodiment, exemplary processes and protocols for the functional optimization of biosynthetic gene clusters by combinatorial design and assembly comprise methods described herein including next generation sequencing and identification of genes, genes clusters and networks, and gene recombineering or recombination-mediated genetic engineering (See: Smanski et al., Nat. Biotechnol., 2014, 32, 1241-1249).

In parallel, refactored linear DNA fragments can also be cloned into a suitable expression vector for transformation into a heterologous expression host or for use in CFB methods and processes, as provided herein. In alternative embodiments, provided are CFB methods and reactions comprising refactored gene clusters with single organism or mixed cell extracts.

In alternative embodiments, products of the CFB methods and processes, including CFB reaction mixtures, are subjected to a suite of “-omics” based approaches including: metabolomics, transcriptomics and proteomics, towards understanding the resulting proteome and metabolome, as well as the expression of lasso peptide biosynthetic genes and gene clusters. In alternative embodiments, lasso peptides produced within CFB reaction mixtures as provided herein are identified and characterized using a combination of high-throughput mass spectrometry (MS) detection tools as well as chemical and biological based assays. Following the characterization of the CFB produced lasso peptides, the corresponding biosynthetic genes and gene clusters may be cloned into a suitable vector for expression and scale up in a heterologous or native expression host. Production of lasso peptides can be scaled up in an in vitro bioreactor or using a fermenter involving a heterologous or native expression host.

In alternative embodiments, metagenomics, the analysis of DNA from a mixed population of organisms, is used to discover and identify biocatalysts, genes, and biosynthetic gene clusters, e.g., lasso peptide biosynthetic gene clusters. In alternative embodiments, metagenomics is used initially to involve the cloning of either total or enriched DNA directly from the environment (eDNA) into a host that can be easily cultivated (See: Handelsman, J., Microbiol. Mol. Biol. Rev., 2004, 68, 669-685). Next generation sequencing (NGS) technologies also can be used e.g., to allow isolated eDNA to be sequenced and analyzed directly from environmental samples (See: Shokralla, et al., Mol. Ecol. 2012, 21, 1794-1805).

As described herein the CFB methods and reaction mixtures can produce analogs of known compounds, for example lasso peptide analogs. Accordingly, CFB reaction mixture compositions can be used in the processes described herein that generate lasso peptide diversity. Methods provided herein include a cell free (in vitro) method for making, synthesizing or altering the structure of a lasso peptide, or a library thereof, comprising using the CFB reaction mixture compositions and CFB methods described herein. The CFB methods can produce in the CFB reaction mixture at least two or more of the altered lasso peptides to create a library of altered lasso peptides; preferably the library is a lasso peptide analog library, prepared, synthesized or modified by a CFB method comprising use of the cell extracts or extract mixtures described herein or by using the process or method described herein. Also provided is a library of lasso peptides or related molecules thereof, or a combination thereof, prepared, synthesized or modified by a CFB method comprising a CFB reaction mixture that produces lasso peptides or related molecules thereof from a minimal set of lasso peptide biosynthesis components, as described herein or by using the process or method described herein.

### 5.3.3.3 Cell-free Biosynthesis of Lasso Peptides

In one aspect, provided herein are methods for producing one or more lasso peptides or related molecules thereof in a CFB system. Relative to recombinant production of lasso peptides in cells, the use of a CFB system to produce lasso peptides and related molecules thereof not only simplifies the process, lowers the cost, and reduces the time required for lasso peptide production and screening, but also enables the use of liquid handling and robotic automation in order to generate large libraries of lasso peptides and functional fragments of lasso peptides in a high throughput manner.

In some embodiments, the method for producing a lasso peptide comprises (a) providing a CFB system comprising a minimal set of lasso peptide biosynthesis components; and (b) incubating the CFB system under a suitable condition to produce the lasso peptide.

In some embodiments, the minimal set of lasso peptide biosynthesis components comprises one or more components functions to provide a lasso precursor peptide, and one or more components function to process the lasso precursor peptide into the lasso peptide. In some embodiments, the one or more components function to process the lasso precursor peptide into the lasso peptide consist of a lasso peptidase and a lasso cyclase. In some embodiments, the one or more components function to process the lasso precursor peptide into the lasso peptide consists of a lasso peptidase, a lasso cyclase and an RRE.

In some embodiments, the minimal set of lasso peptide biosynthesis components comprises one or more components functions to provide a lasso core peptide, and one or more components function to process the lasso core peptide into the lasso peptide. In some embodiments, the one or more components function to process the lasso core peptide into the lasso peptide comprises one or more selected from a lasso peptidase, a lasso cyclase and an RRE. In some embodiments, the one or more components function to process the lasso core into the lasso peptide consist of a lasso cyclase.

In various embodiments, the one or more components function to provide a peptide or protein (e.g., a lasso precursor peptide, a lasso core peptide, or lasso peptide biosynthetic enzymes and proteins) in a CFB system can be provided in the form of the peptide or protein are provided in the form of the peptide or protein per se.

In some embodiments, at least some of the peptide or protein components in the CFB system can be natural peptides or polypeptides. In some embodiments, at least some of the peptide or protein components in the CFB system are derivatives of natural peptides or polypeptides. In some embodiments, at least some of the peptide or protein components in the CFB system are non-natural peptides. In some embodiments, the one or more peptide or protein components of the CFB system can be isolated from nature, such as isolated from microorganisms producing the lasso precursor peptides. In some embodiments, the one or more peptide or protein components of the CFB system can be synthetically or recombinantly produced, using methods known in the art. In some embodiments, the one or more peptide or protein components of the CFB system can be synthesized using the CFB system as described herein, followed by purifying the biosynthesized peptide or protein components from the CFB system.

Additionally or alternatively, the one or more components function to provide a peptide or protein (e.g., a lasso precursor peptide, a lasso core peptide, or lasso peptide biosynthetic enzymes and proteins) in a CFB system can be provided in the form of a nucleic acid encoding the peptide or protein and in vitro TX-TL machinery capable of producing the peptide or protein vial in vitro TX-TL of the coding sequences. In various embodiments, the coding nucleic acid can be DNA, RNA or cDNA. In various embodiments, one or more coding nucleic acid sequences can be contained in the same nucleic acid molecule, such as a vector.

It is understood that when more than one coding nucleic acid sequences are included in a CFB system, such more than one encoding nucleic acid sequences can be introduced on separate nucleic acid molecules, on polycistronic nucleic acid molecules, or a combination thereof. For example, as disclosed herein, a microbial organism or a cell extract can be engineered to express two or more exogenous nucleic acids encoding lasso precursor peptide, lasso core peptide, lasso peptidase, lasso cyclase or RRE. In the case where two exogenous nucleic acids encoding a desired activity are introduced into a host microbial organism or into a cell extract, it is understood that the two exogenous nucleic acids can be introduced as a single nucleic acid, for example, on a single plasmid or as linear strands of DNA, or on separate plasmids, or can be integrated into the host chromosome at a single site or multiple sites, and still be considered as two exogenous nucleic acids. Similarly, it is understood that more than two exogenous nucleic acids can be introduced into a host organism or into a cell extract in any desired combination, for example, on a single plasmid, or on separate plasmids, or as linear strands of DNA, or can be integrated into the host chromosome at a single site or multiple sites.

In some embodiments, the in vitro TX-TL machinery is purified from a host cell. In some embodiments, the in vitro TX-TL machinery is provided in the form of a cell extract of a host cell. An exemplary procedure for obtaining a cell extract comprises the steps of (i) growing cells, (ii) breaking open or lysing the cells by mechanical, biological or chemical means, (iii) removing cell debris and insoluble materials e.g., by filtration or centrifugation, and (iv) optionally treating to remove residual RNA and DNA, but retaining the active enzymes and biosynthetic machinery for transcription and translation, and optionally the metabolic pathways for co-factor recycle, including but not limited to co-factors such as THF, S-adenosylmethionine, ATP, NADH, NAD and NADP and NADPH. In some embodiments, a cell extract may be further supplemented for improved performance in in vitro TX-TL.

In some embodiments, a cell extract can be further supplemented with some or all of the twenty proteinogenic naturally-occurring amino acids and corresponding transfer ribonucleic acids (tRNAs), and optionally, may be supplemented with additional components, including but not limited to: (1) glucose, xylose, fructose, sucrose, maltose, or starch, (2) adenosine triphosphate (ATP), and/or adenosine diphosphate (ADP), purine and guanidine nucleotides, adenosine triphosphate, guanosine triphosphate, cytosine triphosphate, and/or uridine triphosphate, or combinations thereof, (3) cyclic-adenosine monophosphate (cAMP) and/or 3-phosphoglyceric acid (3-PGA), (4) nicotimamide adenine dinucleotides NADH and/or NAD, or nicotimamide adenine dinucleotide phosphates, NADPH, and/or NADP, or combinations thereof, (5) amino acid salts such as magnesium glutamate and/or potassium glutamate, (6) buffering agents such as HEPES, TRIS, spermidine, or phosphate salts, (7) inorganic salts, including but not limited to, potassium phosphate, sodium chloride, magnesium phosphate, and magnesium sulfate, (8) cofactors such as folinic acid and co-enzyme A (CoA), L(−)-5-formyl-5,6,7,8-tetrahydrofolic acid (THF), and/or biotin, (8) RNA polymerase, (9) 1,4-dithiothreitol (DTT), (10) magnesium acetate, and/or ammonium acetate, and/or (11) crowding agents such as PEG 8000, Ficoll 70, or Ficoll 400, or combinations thereof. In some embodiments, the cell extracts or supplemented cell extracts can be used as a reaction mixture to carry out in vitro TX-TL. In some embodiments, supplementations or adjustments can be made to the cell extract to provide a suitable condition for lasso formation.

In some embodiments, the in vitro TX-TL machinery is provided in the form of a cell extract or supplemented cell extract of a host cell. In some embodiments, the host cell is the cell of the same organism where the coding nucleic acid is derived from. For CFB of lasso peptides and related molecules thereof, the coding nucleic acid sequences can be identified using one or more computer-based genomic mining tools described herein or known in the art. For example, U.S. Provisional Application Nos. 62/652,213 and 62/651,028 disclose thousands of sequences from lasso peptide biosynthesis gene clusters identified from various organisms, and provide GenBank accession numbers for various sequences for lasso precursor peptides, lasso peptidase, lasso cyclase and/or RRE. Host organisms where the lasso peptide biosynthesis gene clusters originate can be identified based on the GenBank accession numbers, including but not limited to Caulobacteraceae species (e.g., Caulobacter sp. K31, Caulobacter henricii), Streptomyces species (e.g. Streptomyces nodosus, Streptomyces caatingaensis), Burkholderiaceae species (e.g., Burkholderia thailandensis E264), Pseudomallei species, Bacillus species, Burkholderia species (e.g., Burkholderia thailandensis MSMB43, Burkholderia oklahomensis, Burkholderia pseudomallei), Sphingomonadaceae species (e.g., Sphingobium sp. YBL2, Sphingobium chlorophenolicum, Sphingobium yanoikuyae). In other embodiments, the host cell is a microbial organism known to be applicable to fermentation processes. Exemplary bacteria include species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Vibrio natriegens, Pseudomonas fluorescens, and Pseudomonas putida. Exemplary yeasts or fungi include species selected from Saccharomyces cerevisiae, Schizosaccharomyces pombe, Kluyveromyces lactis, Kluyveromyces marxianus, Aspergillus terreus, Aspergillus niger and Pichia pastoris. E. coli is a particularly useful host organism since it is a well characterized microbial organism suitable for genetic engineering. Other particularly useful host organisms include yeast such as Saccharomyces cerevisiae.

In some embodiments, the CFB system is configured to produce a lasso peptide. In specific embodiments, the CFB system comprises one or more components configured to provide (i) a lasso precursor peptide, (ii) a lasso peptidase, (iii) a lasso cyclase. In specific embodiments, the CFB system comprises one or more components configured to provide (i) a lasso core peptide, and (ii) a lasso cyclase. In some embodiments, the CFB system further comprises one or more components configured to provide (iv) an RRE. In some embodiments, all of (i) to (iv) above are provided in the CFB system as the corresponding peptide or protein. In alternative embodiments, at least one of (i) to (iv) above is provided in the CFB system as a nucleic acid encoding the corresponding protein, and the CFB system further comprises in vitro TX-TL machinery for producing the corresponding protein from the coding nucleic acid. In these embodiments, the CFB systems can be incubated under a condition suitable for lasso formation to produce the lasso peptide. The incubation condition can be designed and adjusted based on various factors known to skilled artisan in the art, including for example, condition suitable for maintain stability of components of the CFB system, conditions suitable for the lasso processing enzymes to exert enzymatic activities, and/or conditions suitable for the in vitro TX-TL of the coding sequences present in the CFB system. Exemplary suitable conditions are illustrated in Examples 1-7 of the present disclosure.

Without being bound by the theory, it is contemplated that different lasso peptidase can process the same lasso precursor peptide into different lasso core peptide by recognizing and cleaving different leader peptide off the lasso precursor. Additionally, different lasso cyclase can process the same lasso core peptide into distinct lasso peptides by cyclizing the core peptide at different ring-forming amino acid residues. Additionally, different RREs can facilitate different processing by the lasso peptidase and/or lasso cyclase, and thus lead to formation of distinct lasso peptides from the same lasso precursor peptide.

Accordingly, in some embodiments, to produce a natural lasso peptide, the CFB system comprises the lasso precursor peptide, lasso peptidase, and lasso cyclase produced from coding sequences of the same lasso peptide biosynthetic gene cluster (such as Genes A, B, and C of the same lasso peptide biosynthetic gene cluster). In some embodiments, to produce a natural lasso peptide, the CFB system comprises the lasso precursor peptide, lasso peptidase, lasso cyclase, and RRE produced from coding sequences of the same lasso peptide biosynthetic gene cluster.

In some embodiments, to produce a natural lasso peptide, the CFB system comprises the lasso core peptide, and lasso cyclase produced from coding sequences of the same lasso peptide biosynthetic gene cluster (such as Genes A and C of the same lasso peptide biosynthetic gene cluster). In some embodiments, to produce a natural lasso peptide, the CFB system comprises the lasso core peptide, lasso cyclase, and RRE produced from coding sequences of the same lasso peptide biosynthetic gene cluster.

In alternative embodiments, to produce a derivative of a natural lasso peptide, at least two of the lasso precursor peptide, lasso peptidase and lasso cyclase in the CFB system are produced from coding sequences of different lasso peptide biosynthetic gene clusters (such as Gene A from one, and Genes B and C from another, lasso peptide biosynthetic gene cluster). In alternative embodiments, to produce a derivative of a natural lasso peptide, at least two of the lasso precursor peptide, lasso peptidase, lasso cyclase and RRE in the CFB system are produced from coding sequences of different lasso peptide biosynthetic gene clusters.

In alternative embodiments, to produce a derivative of a natural lasso peptide, the lasso core peptide and lasso cyclase in the CFB system are produced from coding sequences of different lasso peptide biosynthetic gene clusters (such as Gene A from one, and Gene C from another, lasso peptide biosynthetic gene cluster). In alternative embodiments, to produce a derivative of a natural lasso peptide, at least two of the lasso core peptide, lasso cyclase and RRE in the CFB system are produced from coding sequences of different lasso peptide biosynthetic gene clusters.

In some embodiments, cell-free biosynthesis of lasso peptides is conducted with isolated peptide and enzyme components in standard buffered media, such as phosphate-buffered saline or tris-buffered saline, in each case containing salts, ATP, and co-factors required for lasso peptidase and lasso cyclase enzymatic activity. In some embodiments, cell-free biosynthesis of lasso peptides is conducted using genes that require transcription (TX) and translation (TL) to afford the lasso precursor peptide and/or lasso peptide biosynthetic enzymes in situ, and such in vitro biosynthesis processes are conducted in cell extracts derived from prokaryotic or eukaryotic cells (See: Gagoski, D., et al., Biotechnol. Bioeng. 2016; 113: 292-300; Culler, S. et al., PCT Appl. No. WO2017/031399).

In some embodiments, CFB reactions are conducted with a minimal set of lasso peptide biosynthesis components combined with genes that encode additional peptides, proteins or enzymes, including genes that encode RiPP recognition elements (RREs) or oligonucleotides that encode RREs that are fused to the 5′ or 3′ end of a lasso precursor peptide gene, a lasso core peptide gene, a lasso peptidase gene or a lasso cyclase gene. In other embodiments, CFB reactions are conducted with a minimal set of lasso peptide biosynthesis components, including lasso precursor peptides, lasso peptidases, or lasso cyclase that are fused to RREs at the N-terminus or C-terminus. In other embodiments, CFB reactions are conducted with a minimal set of lasso peptide biosynthesis components combined and contacted with additional isolated proteins or enzymes, including RiPP recognition elements (RREs).

In some embodiments, CFB reactions are conducted with a minimal set of lasso peptide biosynthesis components combined and contacted with genes that encode additional proteins or enzymes, including genes that encode lasso peptide modifying enzymes such as N-methyltransferases, O-methyltransferases, biotin ligases, glycosyltransferases, esterases, acylases, acyltransferases, aminotransferases, amidases, hydroxylases, dehydrogenases, halogenases, kinases, RiPP heterocyclases, RiPP cyclodehydratases, peptidylarginine deiminase, and prenyltransferases.

In some embodiments, CFB reactions are conducted with a minimal set of lasso peptide biosynthesis components combined and contacted with additional isolated proteins or enzymes, including lasso peptide modifying enzymes such as N-methyltransferases, O-methyltransferases, biotin ligases, glycosyltransferases, esterases, acylases, acyltransferases, aminotransferases, amidases, hydroxylases, dehydrogenases, halogenases, kinases, RiPP heterocyclases, RiPP cyclodehydratases, peptidylarginine deiminase, and prenyltransferases.

CFB methods and systems provided herein for the synthesis of lasso peptides and related molecules thereof from a minimal set of lasso peptide biosynthetic pathway components, are conducted in a CFB reaction mixture, comprising one or more cell extracts that are supplemented with all twenty proteinogenic naturally occurring amino acids and corresponding transfer ribonucleic acids (tRNAs). Cell extracts used in the CFB reaction mixture, provided herein for the synthesis of lasso peptides and related molecules thereof from a minimal set of lasso peptide biosynthetic pathway components also may be supplemented with additional components, including but not limited to, glucose, xylose, fructose, sucrose, maltose, starch, adenosine triphosphate (ATP), and/or adenosine diphosphate (ADP), purine and guanidine nucleotides, adenosine triphosphate, guanosine triphosphate, cytosine triphosphate, and uridine triphosphate, cyclic-adenosine monophosphate (cAMP) and/or 3-phosphoglyceric acid (3-PGA), nicotimamide adenine dinucleotides NADH and/or NAD, or nicotimamide adenine dinucleotide phosphates, NADPH, and/or NADP, or combinations thereof, amino acid salts such as magnesium glutamate and/or potassium glutamate, buffering agents such as HEPES, TRIS, spermidine, or phosphate salts, inorganic salts, including but not limited to, potassium phosphate, sodium chloride, magnesium phosphate, and magnesium sulfate, folinic acid and co-enzyme A (CoA), crowding agents such as PEG 8000, Ficoll 70, or Ficoll 400, L(−)-5-formyl-5,6,7,8-tetrahydrofolic acid, RNA polymerase, biotin, 1,4-dithiothreitol (DTT), magnesium acetate, ammonium acetate, or combinations thereof. For a general description of cell-free extract production and preparation, see: Krinsky, N., et al., PLoS ONE, 2016, 11(10): e0165137.

In alternative embodiments, the preparation CFB reaction mixtures and cell extracts employed for the CFB methods as provided herein, comprises characterization of the CFB reaction mixtures and cell extracts using proteomic approaches to assess and quantify the proteome available for the production of lasso peptides and related molecules thereof. In alternative embodiments, ¹³C metabolic flux analysis (MFA) and/or metabolomics studies are conducted on CFB reaction mixtures and cell extracts to create a flux map and characterize the resulting metabolome of the CFB reaction mixture and cell extract or extracts.

In other embodiments, the CFB method is performed using: one or a combination of two or more cell extracts from various “chassis” organisms, such as E. coli, optionally mixed with one or a combination of two or more cell extracts derived from other species, e.g., a native lasso peptide-producing organism or relative. This can give the advantage of a robust transcription/translation machinery, combined with any unknown components of the native species that might be needed for proper protein folding or activity, or to supply precursors for the lasso peptide pathway. In alternative embodiments, if these factors are known they can be expressed in the chassis organism prior to making the cell extract or these factors can be isolated and purified and added directly to the CFB reaction mixture or cell extract.

In alternative embodiments, CFB methods and systems provided herein to produce lasso peptides and related molecules thereof from a minimal set of lasso peptide biosynthetic pathway components, including the use of cell extracts for in vitro TX-TL systems express lasso peptide biosynthetic gene clusters without the regulatory constraints of the cell. In alternative embodiments, some or all of the lasso peptide pathway biosynthetic genes are refactored to remove native transcriptional and translational regulation. In alternative embodiments, some or all of the lasso peptide pathway biosynthetic genes are refactored and constructed into operons on plasmids.

In alternative embodiments, CFB methods, systems and processes, including in vitro TX-TL systems, provided herein to produce lasso peptides and related molecules thereof from a minimal set of lasso peptide biosynthetic pathway components, are cell-free platforms that can use whole cell, cytoplasmic or nuclear extract from a single organism such as E. coli or Saccharomyces cerevisiae (S. cerevisiae) or from an organism of the Actinomyces genus, e.g., a Streptomyces. In alternative embodiments, CFB methods, systems and processes, including in vitro TX-TL systems, provided herein to produce lasso peptides and related molecules thereof from a minimal set of lasso peptide biosynthetic pathway components, are cell-free platforms that can use mixtures of whole cell, cytoplasmic, and/or nuclear extracts from the same or different organisms. In alternative embodiments, strain engineering approaches as well as modification of the growth conditions are used (on the organism from which at least one extract is derived) towards the creation of cell extracts as provided herein, to generate mixed cell extracts with varying proteomic and metabolic capabilities in the final CFB reaction mixture. In alternative embodiments, both approaches are used to tailor or design a final CFB reaction mixture for the purpose of synthesizing and characterizing lasso peptides, or for the creation of lasso peptide analogs through combinatorial biosynthesis approaches.

In alternative embodiments, cell extracts used in the CFB methods, provided herein to produce lasso peptides and related molecules thereof from a minimal set of lasso peptide biosynthetic pathway components, comprise whole cell, cytoplasmic or nuclear extracts from a bacterial cell or eukaryotic cell, including insect, plant, fungal, yeast, or mammalian cells. In alternative embodiments, cell extracts used in the CFB methods, provided herein to produce lasso peptides and related molecules thereof from a minimal set of lasso peptide biosynthetic pathway components, comprise whole cell, cytoplasmic or nuclear extracts from a bacterial cell or eukaryotic cell, including insect, plant, fungal, yeast, or mammalian cells, and are designed, produced and processed in a way to maximize efficacy and yield in the production of desired lasso peptides or related molecules thereof.

In an alternative embodiment, cell extracts used in the CFB methods, provided herein to produce lasso peptides and related molecules thereof from a minimal set of lasso peptide biosynthetic pathway components, derive from at least two different bacterial cells, two different fungal cells; two different yeast cells, two different insect cells, two different plant cells or two different mammalian cells, or combinations of cell extracts from different species and genera thereof. In alternative embodiments, cell extracts used in the CFB methods, provided herein to produce lasso peptides and related molecules thereof from a minimal set of lasso peptide biosynthetic pathway components, comprises an extract derived from: an Escherichia or a Escherichia coli (E. coli); a Streptomyces or an Actinobacteria; an Ascomycota, Basidiomycota, or a Saccharomycetales; a Penicillium or a Trichocomaceae; a Spodoptera, a Spodoptera frugiperda, a Trichoplusia or a Trichoplusia ni; a Poaceae, a Triticum, or a wheat germ; a rabbit reticulocyte or a HeLa cell.

In alternative embodiments, provided are libraries of: lasso peptide or related molecules thereof, or a combination thereof, prepared, synthesized or modified by a CFB method or system comprising use of a CFB reaction mixture with a cell extract as provided herein, or by using a CFB method or system as provided herein. In alternative embodiments, the method for preparing, synthesizing or modifying the lasso peptide or related molecules thereof, or the combination thereof, comprises using a CFB reaction mixture with a cell extract from an Escherichia or from an Actinomyces, optionally a Streptomyces.

In alternative embodiments, cell extracts used in the CFB methods, provided herein to produce lasso peptides and related molecules thereof from a minimal set of lasso peptide biosynthetic pathway components, comprises a cell extract from or comprises an extract derived from: any prokaryotic and eukaryotic organism including, but not limited to, bacteria, including Archaea, eubacteria, and eukaryotes, including yeast, plant, insect, animal, and mammal, including human cells. In alternative embodiments, at least one of the cell extracts used in the CFB methods provided herein comprises an extract from or comprises an extract derived from: Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Homo sapiens, Oryctolagus cuniculus, Rhodobacter spaeroides, Thermoanaerobacter brockii, Metallosphaera sedula, Leuconostoc mesenteroides, Chloroflexus aurantiacus, Roseiflexus castenholzii, Erythrobacter, Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrificans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium loti, Bos taurus, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4.

In alternative embodiments, at least one cell, cytoplasmic or nuclear extract used in the CFB methods, provided herein to produce lasso peptides and related molecules thereof from a minimal set of lasso peptide biosynthetic pathway components, comprises a cell extract from or comprises an extract derived from: Acinetobacter baumannii Naval-82, Acinetobacter sp. ADP 1, Acinetobacter sp. strain M-1, Actinobacillus succinogenes 130Z, Allochromatium vinosum DSM 180, Amycolatopsis methanolica, Arabidopsis thaliana, Atopobium parvulum DSM 20469, Azotobacter vinelandii DJ, Bacillus alcalophilus ATCC 27647, Bacillus azotoformans LMG 9581, Bacillus coagulans 36D1, Bacillus megaterium, Bacillus methanolicus MGA3, Bacillus methanolicus PB1, Bacillus methanolicus PB-1, Bacillus selenitireducens MLS10, Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi 001, Butyrate producing bacterium L2-50, Campylobacter jejuni, Candida albicans, Candida boidinii, Candida methylica, Carboxydothermus hydrogenoformans, Carboxydothermus hydrogenoformans Z-2901, Caulobacter sp. AP07, Chloroflexus aggregans DSM 9485, Chloroflexus aurantiacus J-10-fl, Citrobacter freundii, Citrobacter koseri ATCC BAA-895, Citrobacter youngae, Clostridium, Clostridium acetobutylicum, Clostridium acetobutylicum ATCC 824, Clostridium acidurici, Clostridium aminobutyricum, Clostridium asparagiforme DSM 15981, Clostridium beijerinckii, Clostridium beijerinckii NCIMB 8052, Clostridium bolteae ATCC BAA-613, Clostridium carboxidivorans P7, Clostridium cellulovorans 743B, Clostridium difficile, Clostridium hiranonis DSM 13275, Clostridium hylemonae DSM 15053, Clostridium kluyveri, Clostridium kluyveri DSM 555, Clostridium ljungdahli, Clostridium ljungdahlii DSM 13528, Clostridium methylpentosum DSM 5476, Clostridium pasteurianum, Clostridium pasteurianum DSM 525, Clostridium perfringens, Clostridium perfringens ATCC 13124, Clostridium perfringens str. 13, Clostridium phytofermentans ISDg, Clostridium saccharobutylicum, Clostridium saccharoperbutylacetonicum, Clostridium saccharoperbutylacetonicum NI-4, Clostridium tetani, Corynebacterium glutamicum ATCC 14067, Corynebacterium glutamicum R, Corynebacterium sp. U-96, Corynebacterium variabile, Cupriavidus necator N-1, Cyanobium PCC7001, Desulfatibacillum alkenivorans AK-01, Desulfitobacterium hafniense, Desulfitobacterium metallireducens DSM 15288, Desulfotomaculum reducens MI-1, Desulfovibrio africanus str. Walvis Bay, Desulfovibrio fructosovorans JJ, Desulfovibrio vulgaris str. Hildenborough, Desulfovibrio vulgaris str. ‘Miyazaki F’, Dictyostelium discoideum AX4, Escherichia coli, Escherichia coli K-12, Escherichia coli K-12 MG1655, Eubacterium hallii DSM 3353, Flavobacterium frigoris, Fusobacterium nucleatum subsp. polymorphum ATCC 10953, Geobacillus sp. Y4.1MC1, Geobacillus themodenitrificans NG80-2, Geobacter bemidjiensis Bem, Geobacter sulfurreducens, Geobacter sulfurreducens PCA, Geobacillus stearothermophilus DSM 2334, Haemophilus influenzae, Helicobacter pylori, Homo sapiens, Hydrogenobacter thermophilus, Hydrogenobacter thermophilus TK-6, Hyphomicrobium denitrificans ATCC 51888, Hyphomicrobium zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobium loti MAFF 303099, Metallosphaera sedula, Methanosarcina acetivorans, Methanosarcina acetivorans C2A, Methanosarcina barkeri, Methanosarcina mazer Tuc01, Methylobacter marinus, Methylobacterium extorquens, Methylobacterium extorquens AM1, Methylococcus capsulatas, Methylomonas aminofaciens, Moorella thermoacetica, Mycobacter sp. strain JC1 DSM 3803, Mycobacterium avium subsp. paratuberculosis K-10, Mycobacterium bovis BCG, Mycobacterium gastri, Mycobacterium marinum M Mycobacterium smegmatis, Mycobacterium smegmatis MC2 155, Mycobacterium tuberculosis, Nitrosopumilus salaria BD31, Nitrososphaera gargensis Ga9.2, Nocardia farcinica IFM 10152, Nocardia lowensis (sp. NRRL 5646), Nostoc sp. PCC 7120, Ogataea angusta, Ogataea parapolymorpha DL-1 (Hansenula polymorpha DL-1), Paenibacillus peoriae KCTC 3763, Paracoccus denitrificans, Penicillium chrysogenum, Photobacterium profundum 3TCK, Phytofermentans ISDg, Pichia pastoris, Picrophilus torridus DSM9790, Porphyromonas gingivalis, Porphyromonas gingivalis W83, Pseudomonas aeruginosa PA01, Pseudomonas denitrificans, Pseudomonas knackmussii, Pseudomonas putida, Pseudomonas sp, Pseudomonas syringae pv. syringae B728a, Pyrobaculum islandicum DSM 4184, Pyrococcus abyssi, Pyrococcus furiosus, Pyrococcus horikoshii OT3, Ralstonia eutropha, Ralstonia eutropha H16, Rhodobacter capsulatus, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. LT2, Salmonella enterica typhimurium, Salmonella typhimurium, Schizosaccharomyces pombe, Sebaldella termitidis ATCC 33386, Shewanella oneidensis MR-1, Sinorhizobium meliloti 1021, Streptomyces coelicolor, Streptomyces griseus subsp. griseus NBRC 13350, Sulfolobus acidocalarius, Sulfolobus solfataricus P-2, Synechocystis str. PCC 6803, Syntrophobacter fumaroxidans, Thauera aromatica, Thermoanaerobacter sp. X514, Thermococcus kodakaraensis, Thermococcus litoralis, Thermoplasma acidophilum, Thermoproteus neutrophilus, Thermotoga maritima, Thiocapsa roseopersicina, Tolumonas auensis DSM 9187, Trichomonas vaginalis G3, Trypanosoma brucei, Tsukamurella paurometabola DSM 20162, Vibrio cholera, Vibrio harveyi ATCC BAA-1116, Xanthobacter autotrophicus Py2, Yersinia intermedia, or Zea mays.

In alternative embodiments, cell extracts used in the CFB methods and processes, provided herein for the synthesis of lasso peptides and related molecules thereof from a minimal set of lasso peptide biosynthetic pathway components, e.g., including at least one of the cell, cytoplasmic or nuclear extracts, have added to them, or further comprise, supplemental ingredients, compositions or compounds, reagents, ions, trace metals, salts, or elements, buffers and/or solutions. In alternative embodiments, the CFB method and system of the present disclosure, provided herein for the synthesis of lasso peptides and related molecules thereof from a minimal set of lasso peptide biosynthetic pathway components, use or fabricate environmental conditions to optimize the rate of formation or yield of a lasso peptide or related molecules thereof.

In alternative embodiments, CFB reaction mixtures and cell extracts used in the CFB methods and systems, provided herein for the synthesis of lasso peptides and related molecules thereof from a minimal set of lasso peptide biosynthetic pathway components, are supplemented with a carbon source and other essential nutrients. The CFB production system, including cell extracts used in the CFB methods and processes, provided herein for the synthesis of lasso peptides and related molecules thereof from a minimal set of lasso peptide biosynthetic pathway components, can include, for example, any carbohydrate source. Such sources of sugars or carbohydrate substrates include glucose, xylose, maltose, arabinose, galactose, mannose, maltodextrin, fructose, sucrose and starch.

In alternative embodiments, CFB methods and systems provided herein for the synthesis of lasso peptides and related molecules thereof from a minimal set of lasso peptide biosynthetic pathway components, are conducted in a CFB reaction mixture, comprising cell extracts that are supplemented with all twenty proteinogenic naturally occurring amino acids and corresponding transfer ribonucleic acids (tRNAs). In alternative embodiments, cell extracts used in the CFB reaction mixture, provided herein for the synthesis of lasso peptides and related molecules thereof from a minimal set of lasso peptide biosynthetic pathway components, are supplemented with adenosine triphosphate (ATP), and/or adenosine diphosphate (ADP). In alternative embodiments, cell extracts used in the CFB reaction mixture, provided herein for the synthesis of lasso peptides and related molecules thereof from a minimal set of lasso peptide biosynthetic pathway components, are supplemented with glucose, xylose, maltose, arabinose, galactose, mannose, maltodextrin, fructose, sucrose and/or starch. In alternative embodiments, cell extracts used in the CFB reaction mixture, provided herein for the synthesis of lasso peptides and related molecules thereof from a minimal set of lasso peptide biosynthetic pathway components, are supplemented with purine and guanidine nucleotides, adenosine triphosphate, guanosine triphosphate, cytosine triphosphate, and uridine triphosphate. In alternative embodiments, cell extracts used in the CFB reaction mixture, provided herein for the synthesis of lasso peptides and related molecules thereof from a minimal set of lasso peptide biosynthetic pathway components, are supplemented with cyclic-adenosine monophosphate (cAMP) and/or 3-phosphoglyceric acid (3-PGA). In alternative embodiments, cell extracts used in the CFB reaction mixture, provided herein for the synthesis of lasso peptides and related molecules thereof from a minimal set of lasso peptide biosynthetic pathway components, are supplemented with nicotimamide adenine dinucleotides NADH and/or NAD, or nicotimamide adenine dinucleotide phosphates, NADPH, and/or NADP, or combinations thereof. In alternative embodiments, cell extracts used in the CFB reaction mixture, provided herein for the synthesis of lasso peptides and related molecules thereof from a minimal set of lasso peptide biosynthetic pathway components, are supplemented with amino acid salts such as magnesium glutamate and/or potassium glutamate. In alternative embodiments, cell extracts used in the CFB reaction mixture, provided herein for the synthesis of lasso peptides and related molecules thereof from a minimal set of lasso peptide biosynthetic pathway components, are supplemented with buffering agents such as HEPES, TRIS, spermidine, or phosphate salts. In alternative embodiments, cell extracts used in the CFB reaction mixture, provided herein for the synthesis of lasso peptides and related molecules thereof from a minimal set of lasso peptide biosynthetic pathway components, are supplemented with salts, including but not limited to, potassium phosphate, sodium chloride, magnesium phosphate, and magnesium sulfate. In alternative embodiments, cell extracts used in the CFB reaction mixture, provided herein for the synthesis of lasso peptides and related molecules thereof from a minimal set of lasso peptide biosynthetic pathway components, are supplemented with folinic acid and co-enzyme A (CoA). In alternative embodiments, cell extracts used in the CFB reaction mixture, provided herein for the synthesis of lasso peptides and related molecules thereof from a minimal set of lasso peptide biosynthetic pathway components, are supplemented with crowding agents such as PEG 8000, Ficoll 70, or Ficoll 400, or combinations thereof. For a general description of cell-free extract production and preparation, see: Krinsky, N., et al., PLoS ONE, 2016, 11(10): e0165137.

In alternative embodiments, the CFB reaction mixture, provided herein for the synthesis of lasso peptides and related molecules thereof from a minimal set of lasso peptide biosynthetic pathway components, is maintained under aerobic or substantially aerobic conditions, where such conditions can be achieved, for example, by sparging with air or oxygen, shaking under an atmosphere of air or oxygen, stirring under an atmosphere of air or oxygen, or combinations thereof.

In alternative embodiments, the CFB reaction mixture, provided herein for the synthesis of lasso peptides and related molecules thereof from a minimal set of lasso peptide biosynthetic pathway components, is maintained under anaerobic or substantially anaerobic conditions, where such conditions can be achieved, for example, by first sparging the medium with nitrogen and then sealing the wells or reaction containers, or by shaking or stirring under a nitrogen atmosphere. Briefly, anaerobic conditions refer to an environment devoid of oxygen. Substantially anaerobic conditions include, for example, CFB processes conducted such that the dissolved oxygen concentration in the medium remains between 0 and 10% of saturation. Substantially anaerobic conditions also include performing the CFB methods and processes inside a sealed chamber maintained with an atmosphere of less than 1% oxygen. The percent of oxygen can be maintained by, for example, sparging the CFB reaction with an N₂/CO₂ mixture or other suitable non-oxygen gas or gases.

If desired, the pH of the CFB reaction mixture, including cell extracts, used in the CFB methods and systems, provided herein for the synthesis of lasso peptides and related molecules thereof from a minimal set of lasso peptide biosynthetic pathway components, can be maintained at a desired pH, in particular neutral pH, such as a pH of around 7 by addition of a buffer, a base, such as NaOH or other bases, or an acid, as needed to maintain the production system at a desirable pH for high rates and yields in the production of lasso peptides and related molecules thereof.

In alternative embodiments, CFB reaction mixture, including cell extracts, used in the CFB methods and systems, provided herein for the synthesis of lasso peptides and related molecules thereof from a minimal set of lasso peptide biosynthetic pathway components, is supplemented with one or more enzymes (or the nucleic acids that encode them) of central metabolism pathways, for example, one or more (or all of the) central metabolism enzymes from the tricarboxylic acid cycle (TCA, or Krebs cycle), the glycolysis pathway or the Citric Acid Cycle, or enzymes that promote the production of amino acids.

Metabolic modeling and simulation algorithms can be utilized to optimize conditions for the CFB process and to optimize lasso peptide production rates and yields in the CFB system. Modeling can also be used to design gene knockouts that additionally optimize utilization of the lasso peptide pathway (see, for example, U.S. patent publications US 2002/0012939, US 2003/0224363, US 2004/0029149, US 2004/0072723, US 2003/0059792, US 2002/0168654 and US 2004/0009466, and U.S. Pat. No. 7,127,379). Modeling analysis allows reliable predictions of the effects on shifting the primary metabolism towards more efficient production of lasso peptides and related molecules thereof.

One computational method for identifying and designing metabolic alterations favoring biosynthesis of a desired product is the OptKnock computational framework (Burgard et al., Biotechnol. Bioeng., 2003, 84, 647-657). OptKnock is a metabolic modeling and simulation program that suggests gene deletion or disruption strategies that result in genetically stable metabolic network which overproduces the target product. Specifically, the framework examines the complete metabolic and/or biochemical network in order to suggest genetic manipulations that lead to maximum production of a lasso peptide or related molecules thereof. Such genetic manipulations can be performed on strains used to produce cell extracts for the CFB methods and processes provided herein. Also, this computational methodology can be used to either identify alternative pathways that lead to biosynthesis of a desired lasso peptide or used in connection with non-naturally occurring systems for further optimization of biosynthesis of a desired lasso peptide.

Briefly, OptKnock is a term used herein to refer to a computational method and system for modeling cellular metabolism. The OptKnock program relates to a framework of models and methods that incorporate particular constraints into flux balance analysis (FBA) models. These constraints include, for example, qualitative kinetic information, qualitative regulatory information, and/or DNA microarray experimental data. OptKnock also computes solutions to various metabolic problems by, for example, tightening the flux boundaries derived through flux balance models and subsequently probing the performance limits of metabolic networks in the presence of gene additions or deletions. OptKnock computational framework allows the construction of model formulations that allow an effective query of the performance limits of metabolic networks and provides methods for solving the resulting mixed-integer linear programming problems. The metabolic modeling and simulation methods referred to herein as OptKnock are described in, for example, U.S. publication 2002/0168654, filed Jan. 10, 2002, in International Patent No. PCT/US02/00660, filed Jan. 10, 2002, and U.S. publication 2009/0047719, filed Aug. 10, 2007.

Another computational method for identifying and designing metabolic alterations favoring biosynthetic production of a product is a metabolic modeling and simulation system termed SimPheny®. This computational method and system is described in, for example, U.S. publication 2003/0233218, filed Jun. 14, 2002, and in International Patent Application No. PCT/US03/18838, filed Jun. 13, 2003. SimPheny® is a computational system that can be used to produce a network model in silico and to simulate the flux of mass, energy or charge through the chemical reactions of a biological system to define a solution space that contains any and all possible functionalities of the chemical reactions in the system, thereby determining a range of allowed activities for the biological system. This approach is referred to as constraints-based modeling because the solution space is defined by constraints such as the known stoichiometry of the included reactions as well as reaction thermodynamic and capacity constraints associated with maximum fluxes through reactions. The space defined by these constraints can be interrogated to determine the phenotypic capabilities and behavior of the biological system or of its biochemical components.

These computational approaches are consistent with biological realities because biological systems are flexible and can reach the same result in different ways. Biological systems are designed through evolutionary mechanisms that have been restricted by fundamental constraints that all living systems must face. Therefore, constraints-based modeling strategy embraces these general realities. Further, the ability to continuously impose further restrictions on a network model via the tightening of constraints results in a reduction in the size of the solution space, thereby enhancing the precision with which biosynthetic performance can be predicted.

Given the teachings and guidance provided herein, those skilled in the art will be able to apply various computational frameworks for metabolic modeling and simulation to design and implement biosynthesis of lasso peptides or related molecules thereof using cell extracts and the CFB methods and processes provided herein for the synthesis of lasso peptides and related molecules thereof from a minimal set of lasso peptide biosynthetic pathway genes. Such metabolic modeling and simulation methods include, for example, the computational systems exemplified above as SimPheny® and OptKnock. Those skilled in the art will know how to apply the identification, design and implementation of the metabolic alterations using OptKnock to any of such other metabolic modeling and simulation computational frameworks and methods well known in the art.

Suitable purification and/or assays to test for the production of lasso peptides or functional fragments of lasso peptides can be performed using well known methods. Suitable replicates such as triplicate CFB reactions, can be conducted and analyzed to verify lasso peptide production and concentrations. The final product of lasso peptides, functional fragments of lasso peptides, intermediates, and other organic compounds, can be analyzed by methods such as HPLC (High Performance Liquid Chromatography), GC-MS (Gas Chromatography-Mass Spectrometry), LC-MS (Liquid Chromatography-Mass Spectrometry), MALDI or other suitable analytical methods using routine procedures well known in the art. Byproducts and residual amino acids or glucose can be quantified by HPLC using, for example, a refractive index detector for glucose and saturated fatty acids, and a UV detector for amino acids and other organic acids (Lin et al., Biotechnol. Bioeng., 2005, 90, 775-779), or other suitable assay and detection methods well known in the art. The individual enzyme or protein activities encoded by exogenous or endogenous DNA sequences can also be assayed using methods well known in the art.

Biosynthesized peptide or polypeptide can be isolated, separated purified from other components in the CFB reaction mixtures using a variety of methods well known in the art. Such separation methods include, for example, extraction procedures, including extraction of CFB reaction mixtures using organic solvents such as methanol, butanol, ethyl acetate, and the like, as well as methods that include continuous liquid-liquid extraction, solid-liquid extraction, solid phase extraction, pervaporation, membrane filtration, membrane separation, reverse osmosis, electrodialysis, dialysis, distillation, crystallization, centrifugation, extractive filtration, ion exchange chromatography, size exclusion chromatography, adsorption chromatography, ultrafiltration, medium pressure liquid chromatograpy (MPLC), and high pressure liquid chromatography (HPLC). All of the above methods are well known in the art and can be implemented in either analytical or preparative modes.

### 5.3.3.4 Diversifying Lasso Peptides

In some embodiments, the CFB system is configured to produce a library comprising a plurality of distinct species of lasso peptides or related molecules thereof. In some embodiments, CFB systems are used to facilitate the creation of mutational variants of lasso peptides using methods involving, for example, the synthesis of codon mutants of the lasso precursor peptide or lasso core peptide gene sequence. Lasso precursor peptide or lasso core peptide gene or oligonucleotide mutants can be used in a CFB process, thus enabling the creation of high density lasso peptide diversity libraries. In some embodiments, cell-free biosynthesis is used to facilitate the creation of large mutational lasso peptide libraries using, for example, site-saturation mutagenesis and recombination methods, or in vitro display technologies such as, for example, phage display, RNA display or DNA display (See: Josephson, K., et al., Drug Discov. Today, 2014, 19, 388-399; Doi, N., et al., PLoS ONE, 2012, 7, e30084, pp 1-8; Josephson, K., et al., J. Am. Chem. Soc., 2005, 127, 11727-11735; Odegrip, R., et al., Proc. Nat. Acad. Sci. U.S.A., 2004, 101, 2806-2810; Gamkrelidze, M., Dabrowska, K., Arch Microbiol, 2014, 196, 473-479; Kretz, K. A., et al, Methods Enzymol., 2004, 388, 3-11; Nannemann, D. P, et al., Future Med Chem., 2011, 3, 809-819). In some embodiments, CFB systems are used to facilitate the creation of mutational variants of lasso peptides by introducing non-natural amino acids into the core peptide sequence, followed by formation of the lasso structure using the CFB methods for lasso peptide production as described herein.

In specific embodiments, the CFB system comprises one or more components configured to provide (i) a lasso precursor peptide, (ii) a plurality of different lasso peptidases, (iii) and a lasso cyclase. In some embodiments, the CFB system further comprises one or more components configured to provide (iv) an RRE. In specific embodiments, the CFB system comprises one or more components configured to provide (i) a lasso precursor peptide, (ii) a lasso peptidase, (iii) and a plurality of different lasso cyclases. In some embodiments, the CFB system further comprises one or more components configured to provide (iv) an RRE. In specific embodiments, the CFB system comprises one or more components configured to provide (i) a lasso precursor peptide, (ii) a lasso peptidase, (iii) and a lasso cyclase, and (iv) a plurality of different RREs. In some embodiments, all of (i) to (iv) above are provided in the CFB system as the corresponding peptide or protein. In alternative embodiments, at least one of (i) to (iv) above is provided in the CFB system as a nucleic acid encoding the corresponding protein, and the CFB system further comprises in vitro TX-TL machinery for producing the corresponding protein from the coding nucleic acid.

In specific embodiments, the CFB system comprises one or more components configured to provide (i) a lasso core peptide and (ii) a plurality of different lasso cyclases. In some embodiments, the CFB system further comprises one or more components configured to provide (iv) an RRE. In specific embodiments, the CFB system comprises one or more components configured to provide (i) a lasso core peptide, (ii) and a lasso cyclase, and (iii) a plurality of different RREs. In some embodiments, all of (i) to (iii) above are provided in the CFB system as the corresponding peptide or protein. In alternative embodiments, at least one of (i) to (iii) above is provided in the CFB system as a nucleic acid encoding the corresponding protein, and the CFB system further comprises in vitro TX-TL machinery for producing the corresponding protein from the coding nucleic acid.

In some embodiments, the CFB system is configured to produce a library comprising a plurality of distinct species of lasso peptides or related molecules thereof. In specific embodiments, the CFB system comprises one or more components configured to provide (i) a plurality of different lasso precursor peptides, (ii) at least one lasso peptidase, (iii) and at least one lasso cyclase. In specific embodiments, the CFB system comprises one or more components configured to provide (i) a plurality of different lasso core peptides, and (ii) at least one lasso cyclase. In some embodiments, the CFB system further comprises one or more components configured to provide (iv) at least one RRE. In some embodiments, all of (i) to (iv) above are provided in the CFB system as the corresponding peptide or protein. In alternative embodiments, at least one of (i) to (iv) above is provided in the CFB system as a nucleic acid encoding the corresponding protein, and the CFB system further comprises in vitro TX-TL machinery for producing the corresponding protein from the coding nucleic acid. In these embodiments, the CFB systems can be incubated under a condition suitable for lasso formation to produce the lasso peptide. The incubation condition can be designed and adjusted based on various factors known to skilled artisan in the art, including for example, condition suitable for maintain stability of components of the CFB system, conditions suitable for the lasso processing enzymes to exert enzymatic activities, and/or conditions suitable for the in vitro TX-TL of the coding sequences present in the CFB system. Exemplary suitable conditions are illustrated in Examples 9, 15, 16, and 21 of the present disclosure.

In some embodiments, the nucleic acid sequences coding for a plurality of distinct lasso precursor peptides are derivatives of natural sequences. In some embodiments, the nucleic acid sequences coding for a plurality of distinct lasso precursor peptides are derived from different natural sequences. In specific embodiments, the nucleic acid sequences coding for a plurality of distinct lasso precursor peptides are derived from different Gene A sequences or open reading frame thereof. In specific embodiments, the nucleic acid sequences coding for a plurality of distinct lasso precursor peptides are derived from the same natural sequence. In specific embodiments, the nucleic acid sequences coding for a plurality of distinct lasso precursor peptides are derived from the same Gene A sequence or open reading frame thereof. In specific embodiments, derivation of a nucleic acid sequence (e.g., a Gene A sequence) is performed by introducing one or more mutation(s) to the nucleic acid sequence. In various embodiments, the one or more mutation(s) are one or more selected from amino acid substitution, deletion, and addition. In various embodiments, the one or more mutation(s) can be introduced using mutation methods described herein and/or known in the art.

Alternatively or additionally, in some embodiments, the one or more components function to provide a lasso precursor peptide in a CFB system comprises one or more lasso precursor peptides. In some embodiments, the one or more components function to provide a lasso precursor peptide comprises a plurality of lasso precursor peptides. In some embodiments, at least some of the plurality of lasso precursor peptides are naturally existing. In some embodiments, at least some of the plurality of lasso precursor peptides are derivatives of natural peptides or polypeptides. In some embodiments, at least some of the plurality of lasso precursor peptides are non-natural peptides. In some embodiments, at least some of the plurality of lasso precursor peptides are derived from the same natural peptide or polypeptide. In some embodiments, the one or more lasso precursor peptides can be isolated from nature, such as isolated from microorganisms producing the lasso precursor peptides. In some embodiments, the one or more lasso precursor peptides can be synthetically or recombinantly produced, using methods known in the art. In some embodiments, the one or more lasso precursor peptides can be synthesized using the CFB system as described herein, followed by purifying the biosynthesized lasso precursor peptides from the CFB system.

Particularly, in specific embodiments, the CFB system comprises a plurality of coding sequences each encoding a different lasso precursor peptide. In some embodiments, the plurality of coding sequences comprise sequences from a plurality of different lasso peptide biosynthetic gene clusters (such as a plurality of different Gene A sequences or open reading frames thereof). In some embodiments, the plurality of coding sequences are derived from one or more Gene A sequences or open reading frames thereof.

In some embodiments, the plurality of coding sequences are derived from the same Gene A sequence or open reading frame thereof. In specific embodiments, to produce a library comprising diversified species of lasso peptides, a coding sequence of lasso precursor peptide of interest is mutated to produce a plurality of coding sequences encoding lasso precursor peptides having different amino acid sequences. In some embodiments, a lasso peptide having one or more desirable target properties is selected, and its corresponding precursor peptide is used as the initial scaffold to generate the diversified species of precursor peptides in a library. In some embodiments, one or more mutation(s) are introduced by methods of directed mutagenesis. In alternative embodiments, one or more mutation(s) are introduced by methods of random mutagenesis.

Without being bound by the theory, it is contemplated that the leader sequence of a lasso precursor peptide is recognized by the lasso processing enzymes and can determine specificity and selectivity of the enzymatic activity of the lasso peptidase or lasso cyclase. Accordingly, in some embodiments, only the core peptide portion of the lasso precursor peptide is mutated, while the leader sequence remains unchanged. In some embodiments, the leader sequence of a lasso precursor peptide is replaced by the leader sequence of a different lasso precursor peptide.

Without being bound by theory, it is contemplated that certain lasso cyclases can cyclize the lasso core peptide by joining the N-terminal amino group with the carboxyl group on side chains of glutamate or aspartate residue located at the 7^{th}, 8^{th} or 9^{th} position (counting from the N-terminus) in the core peptide. Accordingly, in some embodiments, random mutations can be introduced to any amino acid residues in a lasso core peptide, or a core peptide region of a lasso precursor peptide, except that at least one of the 7^{th}, 8^{th} or 9^{th} positions (counting from the N-terminus) in the lasso core peptide or core peptide region of a lasso precursor has a glutamate or aspartate residue. In some embodiments, a glutamate residue is introduced to the 7^{th}, 8^{th} or 9^{th} positions (counting from the N-terminus) in the lasso core peptide or core peptide region of a lasso precursor by amino acid addition or amino acid substitution mutations using the methods described herein and/or known in the art. In some embodiments, an aspartate residue is introduced to the 7^{th}, 8^{th} or 9^{th} positions (counting from the N-terminus) in the lasso core peptide or core peptide region of a lasso precursor by amino acid addition or amino acid substitution mutations using the methods described herein and/or known in the art.

Without being bound by theory, it is contemplated that intra-peptide disulfide bond(s), including one or more disulfide bonds (i) between the loop and the ring portions, (ii) between the ring and tail portions, (iii) between the loop and tail portions, and/or (iv) between different amino acid residues of the tail portion of a lasso peptide can contribute to maintain or improve stability of the lariat-like topology of a lasso peptide. Accordingly, in some embodiments, a lasso core peptide or lasso precursor peptide is engineered to have at least two cysteine residues. In specific embodiments, at least two cysteine residues locate on the loop and ring portions of a lasso peptide, respectively. In specific embodiments, at least two cysteine residues locate on the ring and tail portions of a lasso peptide, respectively. In specific embodiments, the at least two cysteine residues locate on the loop and tail portions of a lasso peptide, respectively. In specific embodiments, at least two cysteine residues locate on tail portion of a lasso peptide, respectively. In various embodiments, one or more cysteine residues as described herein are introduced to the nucleic acid sequence of a lasso peptide by amino acid addition or amino acid substitution mutations using the methods described herein and/or known in the art.

Without being bound by theory, it is contemplated that steric effects (e.g., steric hindrance) can contribute to maintain or improve stability of the lariat-like topology of a lasso peptide. Accordingly, in some embodiments, amino acid residues having sterically bulky side chains are located and/or introduced to the locations in the lasso core peptide or the core peptide region of a lasso precursor peptide that are in close proximity to the plane of the ring. In some embodiments, at least one amino acid residue(s) having sterically bulky side chains are located and/or introduced to the tail portion of the lasso peptide. In particular embodiments, multiple bulky amino acids can be consecutive amino acid residues in the tail portion of the lasso peptide. The bulky amino acid residue(s) prevent the tail from unthreading from the ring. In some embodiments, amino acid residue(s) having sterically side chains are located and/or introduced to both the loop and the tail portions of the lasso peptide. In particular embodiments, a bulky amino acid residue in the loop portion is away from a bulky amino acid residue in the tail portion of the lasso peptide by at least 1 non-bulky amino acid residues. In particular embodiments, a bulky amino acid residue in the loop portion is away from a bulky amino acid residue in the tail portion of the lasso peptide by about 2, 3, 4, 5, or 6 non-bulky amino acid residues. In various embodiments, one or more sterically bulky amino acid residues as described herein are introduced to the nucleic acid sequence of a lasso peptide by amino acid addition or amino acid substitution mutations using the methods described herein and/or known in the art.

Various methods have been developed for mutagenesis of genes. A few examples of such mutagenesis methods are provided below. One or more of these methods can be used in connection with the present disclosure to produced diversified nucleic acids sequences coding for different lasso precursor peptides or lasso core peptides, which can be used to produce libraries of lasso peptides using the CFB methods and systems described herein.

Error-prone PCR, or epPCR (Pritchard, L., D. Come, D. Kell, J. Rowland, and M. Winson, 2005, A general model of error-prone PCR. J Theor. Biol 234:497-509.), introduces random point mutations by reducing the fidelity of DNA polymerase in PCR reactions by the addition of Mn²⁺ ions, by biasing dNTP concentrations, or by other conditional variations. The five step cloning process to confine the mutagenesis to the target gene of interest involves: 1) error-prone PCR amplification of the gene of interest; 2) restriction enzyme digestion; 3) gel purification of the desired DNA fragment; 4) ligation into a vector; 5) expression of the gene variants using a CFB system and screening of the library of expressed lasso peptides for improved performance. This method can generate multiple mutations in a single gene or coding sequence simultaneously, which can be useful. A high number of mutants can be generated by epPCR, so a high-throughput screening assay or a selection method (especially using robotics) is useful to identify those with desirable characteristics.

Error-prone Rolling Circle Amplification (epRCA) (Fujii, R., M. Kitaoka, and K. Hayashi, 2004, One-step random mutagenesis by error-prone rolling circle amplification. Nucleic Acids Res 32:e145; and Fujii, R., M. Kitaoka, and K. Hayashi, 2006, Error-prone rolling circle amplification: the simplest random mutagenesis protocol. Nat. Protoc. 1:2493-2497.) has many of the same elements as epPCR except a whole circular plasmid is used as the template and random 6-mers with exonuclease resistant thiophosphate linkages on the last 2 nucleotides are used to amplify the plasmid followed by expression of the variants in a CFB system, in which the plasmid is re-circularized at tandem repeats. Adjusting the Mn²⁺ concentration can vary the mutation rate somewhat. This technique uses a simple error-prone, single-step method to create a full copy of the plasmid with 3-4 mutations/kbp. No restriction enzyme digestion or specific primers are required. Additionally, this method is typically available as a kit.

DNA or Family Shuffling (Stemmer, W. P. 1994, DNA shuffling by random fragmentation and reassembly: in vitro recombination for molecular evolution. Proc Natl Acad Sci U S.A 91:10747-10751; and Stemmer, W. P. 1994. Rapid evolution of a protein in vitro by DNA shuffling. Nature 370:389-391.) typically involves digestion of 2 or more variant genes or coding sequences with nucleases such as DNase I or EndoV to generate a pool of random fragments that are reassembled by cycles of annealing and extension in the presence of DNA polymerase to create a library of chimeric genes. Fragments prime each other and recombination occurs when one copy primes another copy (template switch). This method can be used with >1 kbp DNA sequences. In addition to mutational recombinants created by fragment reassembly, this method introduces point mutations in the extension steps at a rate similar to error-prone PCR.

Staggered Extension (StEP) (Zhao, H., L. Giver, Z. Shao, J. A. Affholter, and F. H. Arnold, 1998, Molecular evolution by staggered extension process (StEP) in vitro recombination. Nat. Biotechnol., 16:258-261.) entails template priming followed by repeated cycles of 2-step PCR with denaturation and very short duration of annealing/extension (as short as 5 sec). Growing fragments anneal to different templates and extend further, which is repeated until full-length sequences are made. Template switching means most resulting fragments have multiple parents. Combinations of low-fidelity polymerases (Taq and Mutazyme) reduce error-prone biases because of opposite mutational spectra.

In Random Priming Recombination (RPR) random sequence primers are used to generate many short DNA fragments complementary to different segments of the template. (Shao, Z., H. Zhao, L. Giver, and F. H. Arnold, 1998, Random-priming in vitro recombination: an effective tool for directed evolution. Nucleic Acids Res, 26:681-683.) Base misincorporation and mispriming via epPCR give point mutations. Short DNA fragments prime one another based on homology and are recombined and reassembled into full-length by repeated thermocycling. Removal of templates prior to this step assures low parental recombinants. This method, like most others, can be performed over multiple iterations to evolve distinct properties. This technology avoids sequence bias, is independent of gene length, and requires very little parent DNA for the application.

In Heteroduplex Recombination linearized plasmid DNA is used to form heteroduplexes that are repaired by mismatch repair. (Volkov, A. A., Z. Shao, and F. H. Arnold. 1999. Recombination and chimeragenesis by in vitro heteroduplex formation and in vivo repair. Nucleic Acids Res, 27:e18; and Volkov, A. A., Z. Shao, and F. H. Arnold. 2000. Random chimeragenesis by heteroduplex recombination. Methods Enzymol., 328:456-463.) The mismatch repair step is at least somewhat mutagenic. Heteroduplexes transform more efficiently than linear homoduplexes. This method is suitable for large genes and whole operons.

Random Chimeragenesis on Transient Templates (RACHITT) (Coco, W. M., W. E. Levinson, M. J. Crist, H. J. Hektor, A. Darzins, P. T. Pienkos, C. H. Squires, and D. J. Monticello, 2001, DNA shuffling method for generating highly recombined genes and evolved enzymes. Nat. Biotechnol., 19:354-359.) employs DNase I fragmentation and size fractionation of ssDNA. Homologous fragments are hybridized in the absence of polymerase to a complementary ssDNA scaffold. Any overlapping unhybridized fragment ends are trimmed down by an exonuclease. Gaps between fragments are filled in, and then ligated to give a pool of full-length diverse strands hybridized to the scaffold (that contains U to preclude amplification). The scaffold then is destroyed and is replaced by a new strand complementary to the diverse strand by PCR amplification. The method involves one strand (scaffold) that is from only one parent while the priming fragments derive from other genes; the parent scaffold is selected against. Thus, no reannealing with parental fragments occurs. Overlapping fragments are trimmed with an exonuclease. Otherwise, this is conceptually similar to DNA shuffling and StEP. Therefore, there should be no siblings, few inactives, and no unshuffled parentals. This technique has advantages in that few or no parental genes are created and many more crossovers can result relative to standard DNA shuffling.

Recombined Extension on Truncated templates (RETT) entails template switching of unidirectionally growing strands from primers in the presence of unidirectional ssDNA fragments used as a pool of templates. (Lee, S. H., E. J. Ryu, M. J. Kang, E.-S. Wang, Z. C. Y. Piao, K. J. J. Jung, and Y. Shin, 2003, A new approach to directed gene evolution by recombined extension on truncated templates (RETT). J. Molec. Catalysis 26:119-129.) No DNA endonucleases are used. Unidirectional ssDNA is made by DNA polymerase with random primers or serial deletion with exonuclease. Unidirectional ssDNA are only templates and not primers. Random priming and exonucleases don't introduce sequence bias as true of enzymatic cleavage of DNA shuffling/RACHITT. RETT can be easier to optimize than StEP because it uses normal PCR conditions instead of very short extensions. Recombination occurs as a component of the PCR steps—no direct shuffling. This method can also be more random than StEP due to the absence of pauses.

In Degenerate Oligonucleotide Gene Shuffling (DOGS) degenerate primers are used to control recombination between molecules; (Bergquist, P. L. and M. D. Gibbs, 2007, Degenerate oligonucleotide gene shuffling. Methods Mol. Biol., 352:191-204; Bergquist, P. L., R. A. Reeves, and M. D. Gibbs, 2005, Degenerate oligonucleotide gene shuffling (DOGS) and random drift mutagenesis (RNDM): two complementary techniques for enzyme evolution. Biomol. Eng., 22:63-72; Gibbs, M. D., K. M. Nevalainen, and P. L. Bergquist, 2001, Degenerate oligonucleotide gene shuffling (DOGS): a method for enhancing the frequency of recombination with family shuffling. Gene 271:13-20.) this can be used to control the tendency of other methods such as DNA shuffling to regenerate parental genes. This method can be combined with random mutagenesis (epPCR) of selected gene segments. This can be a good method to block the reformation of parental sequences. No endonucleases are needed. By adjusting input concentrations of segments made, one can bias towards a desired backbone. This method allows DNA shuffling from unrelated parents without restriction enzyme digests and allows a choice of random mutagenesis methods.

Incremental Truncation for the Creation of Hybrid Enzymes (ITCHY) creates a combinatorial library with 1 base pair deletions of a gene or gene fragment of interest. (Ostermeier et al., Proc. Natl. Acad. Sci. U S.A. 96:3562-3567 (1999); Ostermeier et al., 1999 Nat. Biotechnol., 17:1205-1209 (1999)) Truncations are introduced in opposite direction on pieces of 2 different genes. These are ligated together and the fusions are cloned. This technique does not require homology between the 2 parental genes. When ITCHY is combined with DNA shuffling, the system is called SCRATCHY (see below). A major advantage of both is no need for homology between parental genes; for example, functional fusions between an E. coli and a human gene were created via ITCHY. When ITCHY libraries are made, all possible crossovers are captured.

Thio-Incremental Truncation for the Creation of Hybrid Enzymes (THIO-ITCHY) is almost the same as ITCHY except that phosphothioate dNTPs are used to generate truncations. (Lutz, S., M. Ostermeier, and S. J. Benkovic, 2001, Rapid generation of incremental truncation libraries for protein engineering using alpha-phosphothioate nucleotides. Nucleic Acids Res 29:E16.) Relative to ITCHY, THIO-ITCHY can be easier to optimize, provide more reproducibility, and adjustability.

SCRATCHY-ITCHY combined with DNA shuffling is a combination of DNA shuffling and ITCHY; therefore, allowing multiple crossovers. (Lutz et al., Proc. Natl. Acad. Sci. U S.A. 98:11248-11253 (2001).) SCRATCHY combines the best features of ITCHY and DNA shuffling. Computational predictions can be used in optimization. SCRATCHY is more effective than DNA shuffling when sequence identity is below 80%.

In Random Drift Mutagenesis (RNDM) mutations made via epPCR followed by screening/selection for those retaining usable activity. (Bergquist et al., Biomol. Eng., 22:63-72 (2005).) Then, these are used in DOGS to generate recombinants with fusions between multiple active mutants or between active mutants and some other desirable parent. Designed to promote isolation of neutral mutations; its purpose is to screen for retained catalytic activity whether or not this activity is higher or lower than in the original gene. RNDM is usable in high throughput assays when screening is capable of detecting activity above background. RNDM has been used as a front end to DOGS in generating diversity. The technique imposes a requirement for activity prior to shuffling or other subsequent steps; neutral drift libraries are indicated to result in higher/quicker improvements in activity from smaller libraries. Though published using epPCR, this could be applied to other large-scale mutagenesis methods.

Sequence Saturation Mutagenesis (SeSaM) is a random mutagenesis method that: 1) generates pool of random length fragments using random incorporation of a phosphothioate nucleotide and cleavage; this pool is used as a template to 2) extend in the presence of “universal” bases such as inosine; 3) replication of a inosine-containing complement gives random base incorporation and, consequently, mutagenesis. (Wong et al., Biotechnol J. 3:74-82 (2008); Wong Nucleic Acids Res 32:e26; Wong et al., Anal. Biochem., 341:187-189 (2005).) Using this technique it can be possible to generate a large library of mutants within 2-3 days using simple methods. This is very non-directed compared to mutational bias of DNA polymerases. Differences in this approach makes this technique complementary (or alternative) to epPCR.

In Synthetic Shuffling, overlapping oligonucleotides are designed to encode “all genetic diversity in targets” and allow a very high diversity for the shuffled progeny. (Ness, et al., Nat. Biotechnol., 20:1251-1255 (2002).) In this technique, one can design the fragments to be shuffled. This aids in increasing the resulting diversity of the progeny. One can design sequence/codon biases to make more distantly related sequences recombine at rates approaching more closely related sequences and it doesn't require possessing the template genes physically.

Nucleotide Exchange and Excision Technology NexT exploits a combination of dUTP incorporation followed by treatment with uracil DNA glycosylase and then piperidine to perform endpoint DNA fragmentation. (Muller et al., Nucleic Acids Res 33:e117 (2005)) The gene is reassembled using internal PCR primer extension with proofreading polymerase. The sizes for shuffling are directly controllable using varying dUTP::dTTP ratios. This is an end point reaction using simple methods for uracil incorporation and cleavage. One can use other nucleotide analogs such as 8-oxo-guanine with this method. Additionally, the technique works well with very short fragments (86 bp) and has a low error rate. Chemical cleavage of DNA means very few unshuffled clones.

In Sequence Homology-Independent Protein Recombination (SHIPREC) a linker is used to facilitate fusion between 2 distantly/unrelated genes; nuclease treatment is used to generate a range of chimeras between the two. Result is a single crossover library of these fusions. (Sieber, V., C. A. Martinez, and F. H. Arnold. 2001. Libraries of hybrid proteins from distantly related sequences. Nat. Biotechnol., 19:456-460.) This produces a limited type of shuffling; mutagenesis is a separate process. This technique can create a library of chimeras with varying fractions of each of 2 unrelated parent genes. No homology is needed. SHIPREC was tested with a heme-binding domain of a bacterial CP450 fused to N-terminal regions of a mammalian CP450; this produced mammalian activity in a more soluble enzyme.

Saturation mutagenesis is a random mutagenesis technique, in which a single codon or set of codons is randomised to produce all possible amino acids at the position. Saturation mutagenesis is commonly achieved by artificial gene synthesis, with a mixture of nucleotides used at the codons to be randomised. Different degenerate codons can be used to encode sets of amino acids. Because some amino acids are encoded by more codons than others, the exact ratio of amino acids cannot be equal. Additionally, it is usual to use degenerate codons that minimise stop codons (which are generally not desired). Consequently, the fully randomised ‘NNN’ is not ideal, and alternative, more restricted degenerate codons are used. ‘NNK’ and ‘NNS’ have the benefit of encoding all 20 amino acids, but still encode a stop codon 3% of the time. Alternative codons such as ‘NDT’, ‘DBK’ avoid stop codons entirely, and encode a minimal set of amino acids that still encompass all the main biophysical types (anionic, cationic, aliphatic hydrophobic, aromatic hydrophobic, hydrophilic, small).

Gene Reassembly is a DNA shuffling method that can be applied to multiple genes at one time or to creating a large library of chimeras (multiple mutations) of a single gene. Typically this technology is used in combination with ultra-high-throughput screening to query the represented sequence space for desired improvements. This technique allows multiple gene recombination independent of homology. The exact number and position of cross-over events can be pre-determined using fragments designed via bioinformatic analysis. This technology leads to a very high level of diversity with virtually no parental gene reformation and a low level of inactive genes. Combined with GSSM, a large range of mutations can be tested for improved activity. The method allows “blending” and “fine tuning” of DNA shuffling, e.g. codon usage can be optimized.

In Gene Site Saturation Mutagenesis (GSSM) the starting materials are a supercoiled dsDNA plasmid with insert and 2 primers degenerate at the desired site for mutations. (Kretz, K. A., T. H. Richardson, K. A. Gray, D. E. Robertson, X. Tan, and J. M. Short, 2004, Gene site saturation mutagenesis: a comprehensive mutagenesis approach. Methods Enzymol., 388:3-11.) Primers carry the mutation of interest and anneal to the same sequence on opposite strands of DNA; mutation in the middle of the primer and ˜20 nucleotides of correct sequence flanking on each side. The sequence in the primer is NNN or NNK (coding) and MNN (noncoding) (N=all 4, K=G, T, M=A, C). After extension, Dpnl is used to digest dam-methylated DNA to eliminate the wild-type template. This technique explores all possible amino acid substitutions at a given locus (i.e., one codon). The technique facilitates the generation of all possible replacements at one site with no nonsense codons and equal or near-equal representation of most possible alleles. It does not require prior knowledge of structure, mechanism, or domains of the target enzyme. If followed by shuffling or Gene Reassembly, this technology creates a diverse library of recombinants containing all possible combinations of single-site up-mutations. The utility of this technology combination has been demonstrated for the successful evolution of over 50 different enzymes, and also for more than one property in a given enzyme.

Combinatorial Cassette Mutagenesis (CCM) involves the use of short oligonucleotide cassettes to replace limited regions with a large number of possible amino acid sequence alterations. (Reidhaar-Olson, J. F., J. U. Bowie, R. M. Breyer, J. C. Hu, K. L. Knight, W. A. Lim, M. C. Mossing, D. A. Parse11, K. R. Shoemaker, and R. T. Sauer, 1991, Random mutagenesis of protein sequences using oligonucleotide cassettes. Methods Enzymol., 208:564-586; and Reidhaar-Olson, J. F. and R. T. Sauer, 1988, Combinatorial cassette mutagenesis as a probe of the informational content of protein sequences. Science 241:53-57.) Simultaneous substitutions at 2 or 3 sites are possible using this technique. Additionally, the method tests a large multiplicity of possible sequence changes at a limited range of sites. It has been used to explore the information content of lambda repressor DNA-binding domain.

Combinatorial Multiple Cassette Mutagenesis (CMCM) is essentially similar to CCM except it is employed as part of a larger program: 1) Use of epPCR at high mutation rate, 2) Identification of hot spots and hot regions and then 3) extension by CMCM to cover a defined region of protein sequence space. (Reetz, M. T., S. Wilensek, D. Zha, and K. E. Jaeger, 2001, Directed Evolution of an Enantioselective Enzyme through Combinatorial Multiple-Cassette Mutagenesis. Angew. Chem. Int. Ed Engl. 40:3589-3591.) As with CCM, this method can test virtually all possible alterations over a target region. If used along with methods to create random mutations and shuffled genes, it provides an excellent means of generating diverse, shuffled proteins. This approach was successful in increasing, by 51-fold, the enantioselectivity of an enzyme.

In the Mutator Strains technique conditional is mutator plasmids allow increases of 20- to 4000-X in random and natural mutation frequency during selection and to block accumulation of deleterious mutations when selection is not required. (Selifonova, O., F. Valle, and V. Schellenberger, 2001, Rapid evolution of novel traits in microorganisms. Appl Environ Microbiol., 67:3645-3649.) This technology is based on a plasmid-derived mutD5 gene, which encodes a mutant subunit of DNA polymerase III. This subunit binds to endogenous DNA polymerase III and compromises the proofreading ability of polymerase III in any of the strain that harbors the plasmid. A broad-spectrum of base substitutions and frameshift mutations occur. In order for effective use, the mutator plasmid should be removed once the desired phenotype is achieved; this is accomplished through a temperature sensitive origin of replication, which allows plasmid curing at 41° C. It should be noted that mutator strains have been explored for quite some time (e.g., see Winter and coworkers, 1996, J. Mol. Biol. 260, 359-3680. In this technique very high spontaneous mutation rates are observed. The conditional property minimizes non-desired background mutations. This technology could be combined with adaptive evolution to enhance mutagenesis rates and more rapidly achieve desired phenotypes.

“Look-Through Mutagenesis (LTM) is a multidimensional mutagenesis method that assesses and optimizes combinatorial mutations of selected amino acids.” (Rajpal, A., N. Beyaz, L. Haber, G. Cappuccilli, H. Yee, R. R. Bhatt, T. Takeuchi, R. A. Lerner, and R. Crea, 2005, A general method for greatly improving the affinity of antibodies by using combinatorial libraries. Proc. Natl. Acad. Sci. USA., 102:8466-8471.) Rather than saturating each site with all possible amino acid changes, a set of 9 is chosen to cover the range of amino acid R-group chemistry. Fewer changes per site allows multiple sites to be subjected to this type of mutagenesis. A >800-fold increase in binding affinity for an antibody from low nanomolar to picomolar has been achieved through this method. This is a rational approach to minimize the number of random combinations and should increase the ability to find improved traits by greatly decreasing the numbers of clones to be screened. This has been applied to antibody engineering, specifically to increase the binding affinity and/or reduce dissociation. The technique can be combined with either screens or selections.

In Silico Protein Design Automation PDA is an optimization algorithm that anchors the structurally defined protein backbone possessing a particular fold, and searches sequence space for amino acid substitutions that can stabilize the fold and overall protein energetics. (Hayes, R. J., J. Bentzien, M. L. Ary, M. Y. Hwang, J. M. Jacinto, J. Vielmetter, A. Kundu, and B. I. Dahiyat, 2002, Combining computational and experimental screening for rapid optimization of protein properties. Proc. Natl. Acad. Sci. USA., 99:15926-15931.) This technology allows in silico structure-based entropy predictions in order to search for structural tolerance toward protein amino acid variations. Statistical mechanics is applied to calculate coupling interactions at each position—structural tolerance toward amino acid substitution is a measure of coupling. Ultimately, this technology is designed to yield desired modifications of protein properties while maintaining the integrity of structural characteristics. The method computationally assesses and allows filtering of a very large number of possible sequence variants (10⁵⁰). Choice of sequence variants to test is related to predictions based on most favorable thermodynamics and ostensibly only stability or properties that are linked to stability can be effectively addressed with this technology. The method has been successfully used in some therapeutic proteins, especially in engineering immunoglobulins. In silico predictions avoid testing extraordinarily large numbers of potential variants. Predictions based on existing three-dimensional structures are more likely to succeed than predictions based on hypothetical structures. This technology can readily predict and allow targeted screening of multiple simultaneous mutations, something not possible with purely experimental technologies due to exponential increases in numbers.

Iterative Saturation Mutagenesis (ISM) involves: (1) use knowledge of structure/function to choose a likely site for enzyme improvement, (2) saturation mutagenesis at the chosen site using Agilent QuickChange™ (or other suitable means), (3) screen/select for desired properties, (4) with improved clone(s), start over at another site and continue repeating. (Reetz, M. T. and J. D. Carballeira, 2007, Iterative saturation mutagenesis (ISM) for rapid directed evolution of functional enzymes. Nat. Protoc. 2:891-903; and Reetz, M. T., J. D. Carballeira, and A. Vogel, 2006, Iterative saturation mutagenesis on the basis of B factors as a strategy for increasing protein thermos stability. Angew. Chem. Int. Ed Engl. 45:7745-7751.) This is a proven methodology assures all possible replacements at a given position are made for screening/selection.

Any of the aforementioned methods for mutagenesis can be used alone or in any combination. Additionally, any one or combination of the directed evolution methods can be used in conjunction with adaptive evolution techniques.

In various embodiments described herein, the one or more components function to provide a lasso precursor peptide in a CFB system comprises at least one nucleic acid sequence coding for a fusion protein comprising the lasso precursor peptide. Alternatively or additionally, the one or more components function to provide a lasso precursor peptide in a CFB system comprises at least one lasso precursor peptide(s) forming part of a fusion protein.

In specific embodiments, the fusion protein comprises the lasso precursor peptide fused at its N-terminus. In specific embodiments, the fusion protein comprises the lasso precursor peptide fused at its C-terminus. In some embodiments, the fusion protein further comprises a non-lasso domain configured for associating with another peptide or polypeptide. In some embodiments, the fusion protein further comprise a non-lasso domain configured for associating with a nucleic acid molecule. In some embodiments, in the fusion protein, the non-lasso domain is connected with the lasso precursor peptide via a cleavable peptidic linker. Exemplary endo- and exo-proteases that can be used for cleaving the peptidic linker and thus the separation of the non-lasso domain from the lasso precursor peptide include but are not limited to Enteropeptidase, Enterokinase, Thrombin, Factor Xa, TEV protease, Rhinovirus 3C protease; a SUMO-specific and a NEDD8-specific protease from Brachypodium distachyon (bdSENP1 and bdNEDP1), the NEDP1 protease from Salmo salar (ssNEDP1), Saccharomyces cerevisiae Atg4p (scAtg4) and Xenopus laevis Usp2 (x1Usp2). Additional examples of proteases and their recognition site (i.e., sequences that can be used to form the peptidic linker) for cleavage can be found in Waugh Protein Expr Purif. 2011 December; 80(2): 283-293. In some embodiments, commercially available proteases and corresponding recognition site sequences can be used in connection with the present disclosure.

In some embodiments, the nucleic acid sequence coding for the lasso precursor peptide encodes a fusion protein comprising the lasso precursor peptide. In specific embodiments, the fusion protein comprises a lasso precursor peptide fused at its C-terminus to a streptavidin domain. In specific embodiments, the fusion protein comprises a lasso precursor peptide fused at its C-terminus to a domain comprising a streptavidin binding protein. In specific embodiments, the nucleic acid sequence coding for the lasso precursor peptide is biotinylated.

In specific embodiments, the nucleic acid sequence coding for the lasso precursor peptide is biotinylated, and encodes a fusion protein comprising the lasso precursor peptide fused at its C-terminus to a streptavidin domain. In specific embodiments, the nucleic acid sequence coding for the lasso precursor peptide is biotinylated, and encodes a fusion protein comprising the lasso precursor peptide fused at its C-terminus to a domain comprising a streptavidin binding protein. In specific embodiments, the nucleic acid sequence coding for the lasso precursor peptide is biotinylated, and encodes a fusion protein comprising the lasso precursor peptide fused at its C-terminus to a domain comprising a streptavidin binding domain, and the CFB system further comprises a solid support coated with streptavidin.

In some embodiments, the nucleic acid sequence coding for the lasso precursor peptide is not biotinylated, and encodes a fusion protein comprising the lasso precursor peptide fused at its C-terminus to a streptavidin domain, and the CFB system further comprises a biotinylated unique nucleic acid Barcode. In some embodiments, the nucleic acid sequence coding for the lasso precursor peptide is not biotinylated, and encodes a fusion protein comprising the lasso precursor peptide fused at its C-terminus to a domain comprising a streptavidin binding protein, and the CFB system further comprises a biotinylated unique nucleic acid Barcode and a solid support coated with streptavidin. In various embodiments described herein, the streptavidin binding protein is the streptavidin-binding peptide (SBP) (See: Wilson et al., PNAS, 2001, 98 (7), 3750-3755), Strep-tag (See: Schmidt and Skerra, Protein Eng. 1993, 6(1):109-22), Strep-tag II (See: Schmidt et al., J Mot Biol. 1996, 255(5):753-66) or Nano-tag (See: Lamla and Erdmann, Protein Expr Purif. 2004, 33(1):39-47).

In some embodiments, the nucleic acid sequence coding for the lasso precursor peptide encodes a fusion protein comprising the lasso precursor peptide and a non-lasso domain. In some embodiments, the non-lasso domain is a peptidic tag configured to purify the lasso precursor peptide. In some embodiments, the non-lasso domain produces a signal detectable from the CFB system. In some embodiments, the non-lasso domain is configured to associate with other proteins to form a protein complex comprising the lasso precursor peptide.

In some embodiments, the plurality of different lasso precursor peptides are combined with a plurality of different lasso peptidase, a plurality of different lasso cyclase, and/or a plurality of different RREs in the CFB system to further diversify the lasso peptides and molecules related thereof, which the CFB system is able to produce.

Additional diversification of a lasso peptide library can be achieved using the combinational biosynthesis approaches. In specific embodiments, combinatorial biosynthesis approaches are executed through the variation and modification of lasso peptide pathway genes, using different refactored lasso peptide gene cluster combinations, using combinations of genes from different lasso peptide gene clusters, using genes that encode enzymes that introduce chemical modifications before or after formation of the lasso peptide, using alternative lasso peptide precursor combinations (e.g., varied amino acids), using different CFB reaction mixtures, supplements or conditions, or by a combination of these alternatives.

Combinatorial CFB methods as provided herein can be used to produce libraries of new compounds, including lasso peptide libraries. For example, an exemplary refactored lasso peptide pathway can vary enzyme specificity at any step or add enzymes to introduce new functional groups and analogs at any one or more sites in a lasso peptide. Exemplary processes can vary enzyme specificity to allow only one functional group in a mixture to pass to the next step, thus allowing each reaction mixture to generate a specific lasso peptide analog. Exemplary processes can vary the availability of functional groups at any step to control which group or groups are added at that step. Exemplary processes can vary a domain of an enzyme to modify its specificity and lasso peptide analog created. Exemplary processes can add a domain of an enzyme or an entire enzyme module to add novel chemical reaction steps to the lasso peptide pathway.

Additional diversification of a lasso peptide library can be achieved via chemical or enzymatic modifications. In specific embodiments of the libraries: the lasso peptide analogs, or the diversity of lasso peptide analogs, is generated by a CFB method or system comprising the capability of modifying the lasso peptide chemically or by enzyme modification, wherein optionally the enzyme modification comprises modification of the lasso peptide by: halogenation, lipidation, pegylation, glycosylation, adding hydrophobic groups, myristoylation, palmitoylation, isoprenylation, prenylation, lipoylation, adding a flavin moiety (optionally comprising addition of: a flavin adenine dinucleotide (FAD) an FADH2, a flavin mononucleotide (FMN), an FMNH2), phospho-pantetheinylation, heme C addition, phosphorylation, acylation, alkylation, butyrylation, carboxylation, malonylation, hydroxylation, adding a halide group, iodination, propionylation, S-glutathionylation, succinylation, glycation, adenylation, thiolation, condensation (optionally the “condensation” comprising addition of: an amino acid to an amino acid, an amino acid to a fatty acid, an amino acid to a sugar), or a combination thereof, and optionally the enzyme modification comprises modification of the lasso peptide by one or more enzymes comprising: a CoA ligase, a phosphorylase, a kinase, a glycosyl-transferase, a halogenase, a methyltransferase, a hydroxylase, a lambda phage GamS enzyme (optionally used with a bacterial or an E. coli extract, optionally at a concentration of about 3.5 mM), a Dsb (disulfide bond) family enzyme (optionally DsbA), or a combination thereof; or optionally the enzymes comprise one or more central metabolism enzyme (optionally tricarboxylic acid cycle (TCA, or Krebs cycle) enzymes, glycolysis enzymes or Pentose Phosphate Pathway enzymes), and optionally the chemical or enzyme modification comprises addition, deletion or replacement of a substituent or functional groups, optionally a hydroxyl group, an amino group, a halogen, an alkyl or a cycloalkyl group, optionally by hydration, biotinylation, hydrogenation, an aldol condensation reaction, condensation polymerization, halogenation, oxidation, dehydrogenation, or creating one or more double bonds.

In some embodiments, the diversified species of lasso peptides are screened for one or more desirable target properties, and one or more lasso peptides are further selected to serve as the new scaffold for at least one additional round of mutagenesis and screening.

### 5.3.3.5 Generating Lasso Peptide Libraries Using the Cell-Free Biosynthesis System

Provided herein are methods for providing diversified species of lasso peptides or related molecules thereof in a library, including display libraries and more specifically, molecular display libraries. The libraries provided herein can be generated using the CFB system of the present disclosure. Particularly, individual members of the library can be generated sequentially or simultaneously using the CFB system of the present disclosure.

For example, in one embodiment, the CFB system comprises a minimal set of lasso peptide biosynthesis components in a CFB reaction mixture. To generate a plurality of diversified members of a lasso peptide library, in some embodiments, the CFB system can comprise multiple units, each unit configured for cell-free biosynthesis of a unique member of the library. In some embodiments, to generate a display library, the biosynthesized library members are each associated with a mechanism for identifying and/or distinguishing such member before the members are combined to form the display library. In specific embodiments, to generate a molecular display library, the biosynthesized library members are each associated with a unique nucleic acid molecule for identifying and/or distinguishing such member before the members are combined to form the molecular display library. For the purpose of illustration only, FIGS. 5A, 5B, 6A, 6B, and 6C. provide various exemplary procedures for producing lasso peptide libraries, including display libraries and molecular display libraries.

As shown in FIG. 5A, a first nucleic acid molecule comprising a sequence encoding a lasso precursor peptide is provided. In some embodiments, the coding sequence can comprise a wild-type or mutated Gene A sequence. A second nucleic acid molecule comprising sequences coding for a lasso peptidase, a lasso cyclase and an RRE is provided. Cell-free TX-TL of the first and second nucleic acid molecules are performed to produce the lasso precursor peptide, lasso peptidase, lasso cyclase and RRE proteins, respectively. As shown in this figure, both the first and second nucleic acid molecules are plasmids.

In some embodiments, an aliquot (e.g. in a tube, a plate, or water-in-oil emulsion) of a CFB reaction mixture comprising the in vitro TX-TL machinery is added with both the first and the second nucleic acid molecules. The aliquot is then incubated under a condition suitable for in vitro TX-TL of the encoded proteins (e.g., the lasso precursor peptide, lasso peptidase, lasso cycles and RRE), and for the lasso peptide biosynthetic enzymes and proteins (e.g., the lasso peptidase, lasso cycles and RRE) to convert the lasso precursor peptide into a matured lasso peptide. In alternative embodiments, the first and the second nucleic acid molecules are added into separate aliquots of the CFB reaction mixture comprising the in vitro TX-TL machinery, and the aliquots are incubated under a suitable condition for in vitro TX-TL of the lasso precursor peptide and the lasso peptide biosynthetic enzymes and proteins (e.g., the lasso peptidase, lasso cycles and RRE) separately. Then, the biosynthesized lasso precursor peptide and lasso peptide biosynthetic enzymes and proteins are contacted with each other under a suitable condition for the lasso peptide biosynthetic enzymes and proteins to convert the lasso precursor peptide into a matured lasso peptide.

In some embodiments, the aliquot containing the first nucleic acid molecule is supplemented with the second nucleic acid molecule and/or one or more of a lasso peptidase, lasso cyclase, and RRE. One or more of the lasso peptidase, lasso cyclase, and RRE can be chemically synthetized or recombinantly produced. In the exemplary embodiments as shown in FIG. 5A, the lasso peptidase, lasso cyclase and RRE are biosynthesized using the CFB system and methods described herein. In particular embodiments as shown in FIG. 5A, the peptidase, lasso cyclase and RRE are each fused to a purification tag, such as the maltose binding protein (MBP-tag), for purifying the proteins from the CFB system.

In some embodiments, to produce a library of lasso peptides using the CFB system, a plurality of versions of the first nucleic acid molecule comprising coding sequences for different lasso precursor peptides (e.g., Gene A coding sequences obtained from different lasso peptide biosynthetic gene clusters, or coding sequences derived from the same Gene A sequence) are provided (e.g., by cloning sequences from different lasso peptides biosynthetic gene clusters as identified by the RODEO algorithm, or mutated versions of a Gene A sequences of interest). The plurality of different versions of the first nucleic acid molecule are added to one aliquot of the CFB reaction mixture. Accordingly, in these embodiments, a plurality of distinct species of lasso peptides are produced in a mixture. In these embodiments, each of the plurality of distinct species of lasso peptides is a member of the library.

In some embodiments, a CFB reaction mixture comprising the in vitro TX-TL machinery is divided into multiple aliquots (e.g. in multiple separate tubes, plates, or water-in-oil droplets). In some embodiments, a plurality of versions of the first nucleic acid molecule comprising coding sequences for different lasso precursor peptides (e.g., Gene A coding sequences obtained from different lasso peptide biosynthetic gene clusters, or coding sequences derived from the same Gene A sequence) are provided (e.g., by cloning sequences from different lasso peptides biosynthetic gene clusters as identified by the RODEO algorithm, or mutated versions of a Gene A sequences of interest). In some embodiments, the plurality of the different versions of the first nucleic acid molecule are each added to a separate aliquot of the CFB reaction mixture. Accordingly, in these embodiments, a plurality of distinct species of lasso peptides are produced in separate aliquots.

Specifically, in some embodiments, to produce a display library for lasso peptides, the first nucleic acid molecule further comprises a sequence encoding one or more peptidic linker (e.g. a cleavable linker), and a sequence encoding a streptavidin binding peptide (SBP-tag), both fused in frame with the sequence encoding the lasso precursor peptide. Accordingly, in these embodiments, a lasso peptide fused to a SBP-tag is produced.

In some embodiments, the fusion protein comprising the lasso peptide and the SBP-tag is contacted with a solid support coated with streptavidin, under a suitable condition for the fusion protein to associate with the solid support. In specific embodiments, the solid support is located at a unique location, whereby the spatial information of the unique location can identify and/or distinguish the lasso peptide forming part of the fusion protein. For example, in some embodiments as shown in FIG. 5A, the lasso peptide display library comprises a multi-well plate coated with streptavidin, and each well houses a unique member of the library. In alternative embodiments, the solid support is associated with a unique nucleic acid molecule, whereby the sequential information of the unique amino acid can identify and/or distinguish the lasso peptide forming part of the fusion protein. For example, in some embodiments as shown in FIG. 5A, each fusion protein comprising a lasso peptide and the SBP-tag is associated with biotinylated DNA barcode through a streptavidin-coated bead. In some embodiments, multiple members of the molecular display library can be combined together to form the molecular display library.

FIG. 5B shows alternative exemplary embodiments for producing lasso peptide libraries, where instead of the circular plasmids shown in FIG. 5A, both the first and second nucleic acid molecules are provided as linear nucleic acid molecules, such as linear double-stranded DNA (dsDNA) molecules.

FIG. 6A shows alternative exemplary embodiments for producing a molecular display library of lasso peptides. As shown, the first nucleic acid molecule encoding the lasso precursor peptide is provided as a linear nucleic acid molecule. The first nucleic acid molecule encodes for a fusion protein comprising a lasso precursor peptide fused at C terminus to a SBP-tag via a cleavable linker. In some embodiments, the first nucleic acid molecule comprises a wild-type or mutated Gene A sequence. The first nucleic acid molecule is amplified using biotinylated 5′ DNA primer to produce biotinylated first nucleic acid molecule. In some embodiments, a second nucleic acid molecule comprising sequences coding for a lasso peptidase, a lasso cyclase and an RRE is provided. In the exemplary embodiments as shown in FIG. 6A, the second nucleic acid molecule is a plasmid.

As shown in FIG. 6A, in some embodiments, the biotinylated first nucleic acid molecule is immobilized on streptavidin-coated solid support through the binding of streptavidin on the solid support to the biotin moiety of the first nucleic acid molecule. The immobilized biotinylated first nucleic acid molecule is then added to an aliquot of the CFB reaction mixture comprising the in vitro TX-TL machinery. In various embodiments as shown in FIG. 6A, the streptavidin-coated solid support can be a streptavidin-coated surface in a tube or a well that houses an aliquot of the CFB reaction mixture comprising the in vitro TX-TL machinery. In alternative embodiments, streptavidin-coated solid support can be streptavidin-coated beads that is free-floating in an aliquot of the CFB reaction mixture comprising the in vitro TX-TL machinery (e.g., in tube, or well, or water-in-oil emulsion).

In some embodiments, the aliquot comprising the immobilized biotinylated first nucleic acid is further supplemented with the second nucleic acid, and/or with one or more of lasso peptidase, lasso cyclase and RRE, and the aliquot is incubated under a suitable condition to produce a fusion protein comprising a lasso peptide fused at the end of its tail portion to the SBP-tag. The fusion protein then becomes immobilized on the solid support through the binding of the SBP-tag to the streptavidin-coated solid support as shown in FIG. 6A, and as such, the fusion protein is associated with the first nucleic acid molecule encoding the fusion protein. Particularly, one or more of lasso peptidase, lasso cyclase and RRE can be recombinantly produced or synthesized.

FIG. 6B shows alternative exemplary embodiments for producing a molecular display library of lasso peptides. As shown, the first nucleic acid molecule encoding the lasso precursor peptide is provided as a linear nucleic acid molecule. The first nucleic acid molecule encodes for a fusion protein comprising a lasso precursor peptide fused at the C-terminus to streptavidin (STA-tag) via a cleavable linker. In some embodiments, the first nucleic acid molecule comprises a wild-type or mutated Gene A sequence. The first nucleic acid molecule is amplified using a biotinylated 5′ DNA primer to produce a biotinylated first nucleic acid molecule. In some embodiments, a second nucleic acid molecule comprising sequences coding for a lasso peptidase, a lasso cyclase and an RRE is provided. In the exemplary embodiments as shown in FIG. 6B, the second nucleic acid molecule is a plasmid.

As shown in FIG. 6B, in some embodiments, the biotinylated first nucleic acid molecule is added to an aliquot of the CFB reaction mixture comprising the in vitro TX-TL machinery. In some embodiments, the aliquot containing the biotinylated first nucleic acid molecule is further supplemented with the second nucleic acid molecule, and/or with one or more of purified lasso peptidase, lasso cyclase and RRE, and the aliquot is incubated under a suitable condition to produce a fusion protein comprising a lasso peptide fused at the end of its tail portion to the STA-tag. The fusion protein then becomes associated with the biotinylated first nucleic acid molecule through binding of the STA-tag with the biotinylated moiety of the first nucleic acid molecule.

FIG. 6C shows alternative exemplary embodiments for producing a molecular display library of lasso peptides. As shown, the first nucleic acid molecule encoding the lasso precursor peptide is provided as a linear nucleic acid molecule. The first nucleic acid molecule encodes for a fusion protein comprising a lasso precursor peptide fused at the C-terminus to replication protein RepA (RepA-tag) via a cleavable linker. The first nucleic acid molecule further comprises the replication origin R (oriR) sequence and the cis-acting element (CIS) of RepA. In some embodiments, the first nucleic acid molecule comprises a wild-type or mutated Gene A sequence. In some embodiments, a second nucleic acid molecule comprising sequences coding for a lasso peptidase, a lasso cyclase and an RRE is provided. In the exemplary embodiments as shown in FIG. 6C, the second nucleic acid molecule is a plasmid.

As shown in FIG. 6C, in some embodiments, the first nucleic acid molecule is added to an aliquot of the CFB reaction mixture comprising the in vitro TX-TL machinery. In some embodiments, the aliquot containing the first nucleic acid molecule is further supplemented with the second nucleic acid molecule, and/or with one or more of purified lasso peptidase, lasso cyclase and RRE, and the aliquot is incubated under a suitable condition to produce a fusion protein comprising a lasso peptide fused at the end of its tail portion to the RepA-tag. The fusion protein then becomes associated with the first nucleic acid molecule through binding of the RepA-tag with the oriR sequence in the first nucleic acid molecule.

In some embodiments, to produce a molecular display library of lasso peptides using the CFB system, a plurality of versions of the first nucleic acid molecule comprising coding sequences for fusion proteins comprising distinct species of lasso peptides are provided. In vitro TX-TL of the different versions of the first nucleic acid molecules separately using, for example the procedures illustrated in FIGS. 6A, 6B and 6C, can generate a plurality of library members each comprising a unique species of lasso peptide and associated with its encoding first nuclei acid molecule. The plurality of members can be combined into a molecular display library of lasso peptides.

To be clear, the exemplary embodiments as shown in FIGS. 5A, 5B, 6A, 6B, and 6C of the present disclosure are solely for the purpose of illustration. Various modifications to these exemplary embodiments can be envisioned by a skilled artisan in the art, based on the present disclosure or knowledge in the art. For example, in various embodiments, one or more of the various protein components as illustrated in these figures, including the lasso precursor peptides, lasso core peptides, lasso peptidase, lasso cyclase, RREs, can be produced via chemical synthesis, or recombinantly produced, or biosynthesized using the CFB systems and methods disclosed herein. In some embodiments, one or more purification steps can be added to the exemplary procedures. In some embodiments, a fusion protein comprising a lasso peptide may not comprise a linker fragment between the lasso peptide fragment and non-lasso fragment of the fusion protein. In some embodiments, the lasso peptidase, lasso cyclase, and RRE can be encoded by multiple plasmids. In some embodiments, the first nucleic acid encodes a lasso core peptide or a fusion protein comprising a lasso core peptide, and the second nucleic acid does not encode at least one of the lasso peptidase and RRE.

In some embodiments, the molecular display library comprises a plurality of unique nucleic acid molecules as an identification mechanism for identifying a library member.

### 5.4 Screen and Evolution

According to the present disclosure, the lasso peptide libraries provided herein can be screened for candidate library members having one or more target properties. Furthermore, the lasso peptide libraries can be used in directed evolution of candidate lasso peptides for the generation of improved lasso peptides having those target properties.

Characteristics of lasso peptides that can be target properties include, for example, binding selectivity or specificity—for target-specific effects and avoiding off-target side effects or toxicity; binding affinity—for target-modulating potency and duration; temperature stability—for robust high temperature processing; pH stability—for bioprocessing under lower or higher pH conditions; expression level—increased protein yields. Other desirable target properties include, for example, solubility, metabolic stability, and pharmacokinetics. The present methods thus enable the discovery and optimization of lasso peptides and related molecules thereof for use in pharmaceutical, agricultural, and consumer applications.

Screening of the libraries can be accomplished by various techniques known in the art. For example, a target molecule (e.g., a GPCR polypeptide or fragment) can be used to coat the wells of adsorption plates, expressed on host cells affixed to adsorption plates or used in cell sorting, conjugated to biotin for capture with streptavidin-coated beads, or used in any other method for panning display libraries. The selection of lasso peptides with slow dissociation kinetics (e.g., good binding affinities) can be promoted by use of long washes and stringent panning conditions as described in Bass et al., 1990, Proteins 8:309-14 and WO 92/09690, and by use of a low coating density of target molecules as described in Marks et al., 1992, Biotechnol. 10:779-83.

Lasso peptides having one or more desirable target property(ies) can be obtained by designing a suitable screening procedure to select for one or more candidate members from the lasso peptide display library as scaffold(s), followed by evolving the scaffolds towards improved target property.

### 5.4.1 Screening Lasso Peptides for Desirable Target Properties

Provided herein is a lasso peptide display library comprising a plurality of library members. As described herein, in various embodiments, the lasso peptide library comprises (i) intact lasso peptides, (ii) functional fragments of lasso peptides, (iii) fusion proteins each comprising a lasso peptide or a functional fragment of lasso peptide, (iv) protein complexes each comprising a lasso peptide or a functional fragment of lasso peptide, (v) conjugates each comprising a lasso peptide or a functional fragment of lasso peptide, or (vi) any combinations of (i) to (v).

The lasso peptide display library can be screened for one or more target properties. In some embodiments, the lasso peptide display library is screened for library member(s) that shows affinity to a target molecule. In some embodiments, the lasso peptide display library is screened for library member(s) that specifically binds to a target molecule. In some embodiments, the lasso peptide display library is screened for library member(s) that specifically binds to a target site within a target molecule that has multiple sites capable of being bound by a ligand. In some embodiments, the lasso peptide display library is screened for library member(s) that compete for binding with a known ligand to a target molecule. In specific embodiments, such known ligand can also be a lasso peptide. In other embodiments, such known molecule can be a non-lasso ligand of the target molecule, such as a drug compound or a non-lasso protein. Various binding assays have been developed for testing the binding activity of members of a lasso peptide display library to a target molecule.

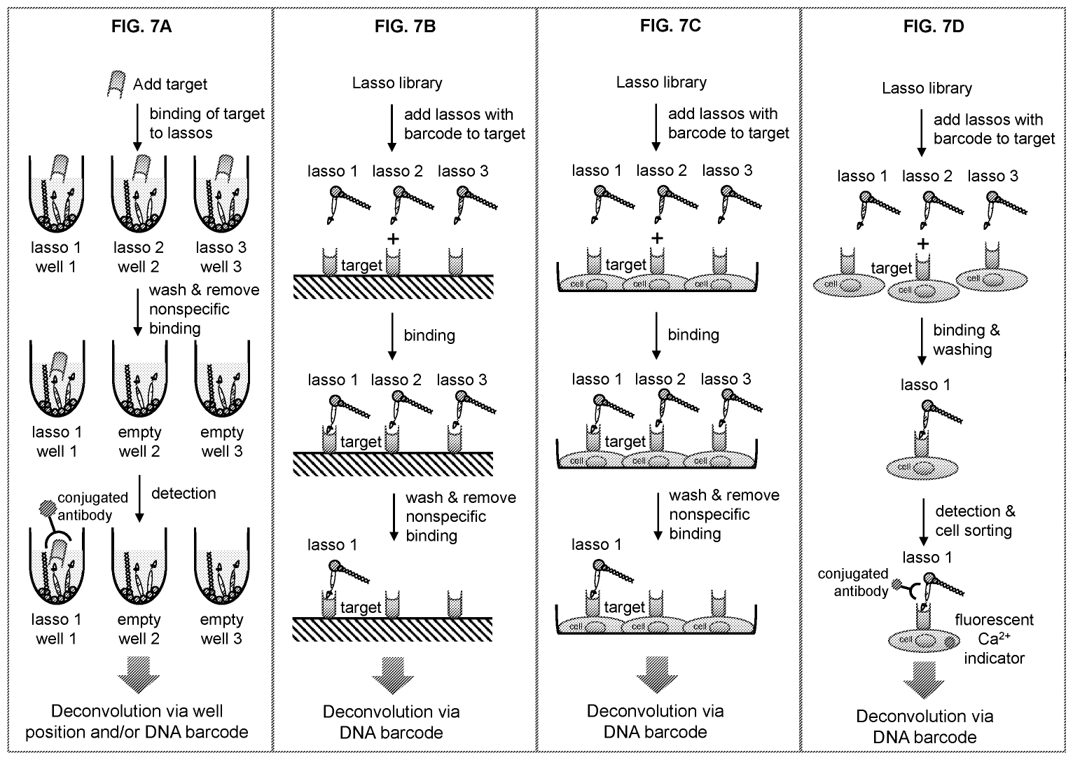

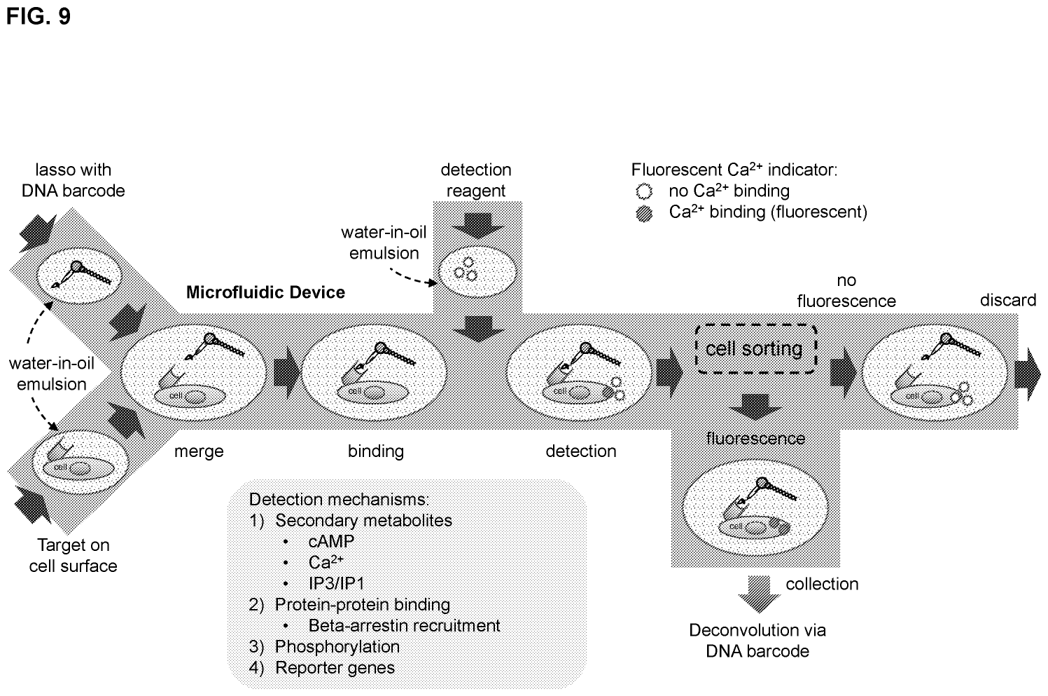

Various binding assays can be used in connection with the present disclosure include immunoassays (e.g., ELISA, fluorescent immunosorbent assay, chemiluminescence immune assay, radioimmunoassay (RIA), enzyme multiplied immunoassay, solid phase radioimmunoassay (SPRIA)), a surface plasmon resonance (SPR) assay (e.g., Biacore®), a fluorescence polarization assay, a fluorescent resonance energy transfer (FRET) assay, Dot-blot assay, fluorescence activated cell sorting (FACS) assay. FIGS. 7A through 7D, and FIG. 9 illustrate exemplary embodiments for performing the binding assay. Example 20 provides an exemplary compete assay for screening lasso peptide library for candidates that specifically targets different binding pockets of the same target molecule.

In some embodiments, the target molecule is a cell surface protein. In some embodiments, the lasso peptide display library is screened for library members(s) that is capable of modulating one or more cellular activities mediated by the cell surface protein. In some embodiments, a lasso peptide display library is subjected to a biological assay that monitors the level of a cellular activity of interest, after the library is contacted with a cell expressing the target molecule. In some embodiments, a lasso peptide display library is subjected to a biological assay that monitors a phenotype of interest of a cell after the library is contacted with a cell expressing the target molecule. In some embodiments, the target molecule is an unidentified cell surface protein expressed by a cell of interest. In some embodiments, a lasso peptide display library is subjected to a biological assay that monitors the level of a cellular activity of interest, after the library is contacted with a population of the cells of interest. Additionally or alternatively, in some embodiments, a lasso peptide display library is subjected to a biological assay that monitors a phenotype of the cell of interest, after the library is contacted with the cell.

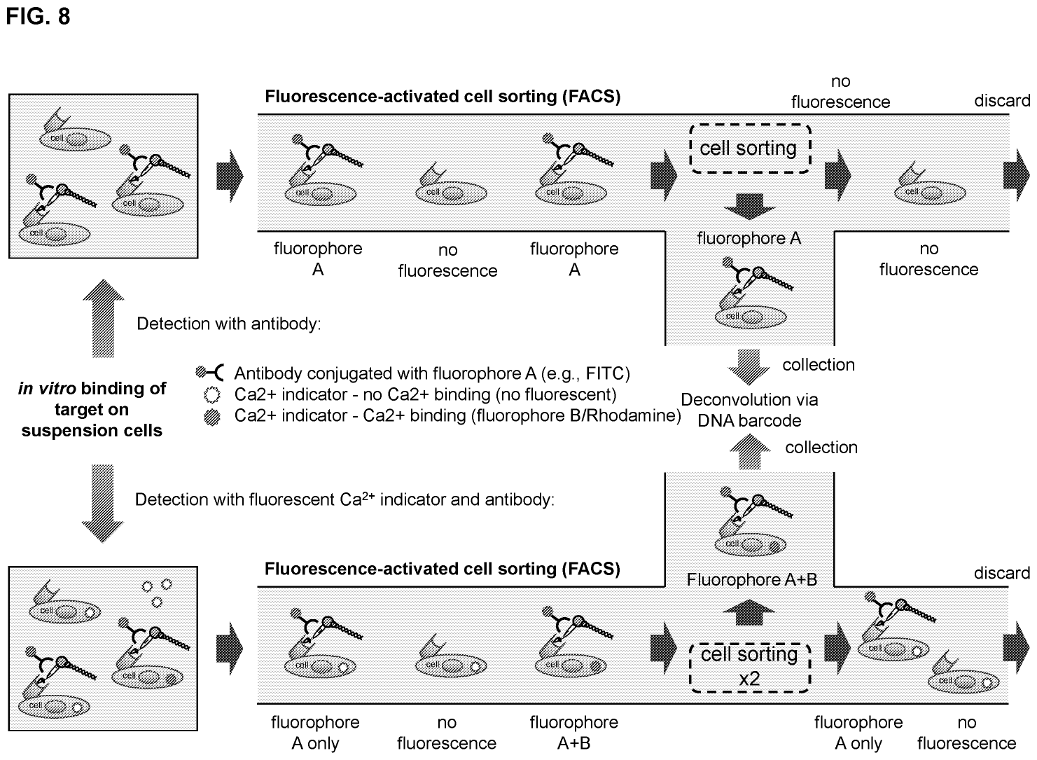

Various biological assays have been developed and can be used in connection with the present disclosure. Depending on the target cellular activity of interest, selection of a suitable biological assay can be made using knowledge in the art. For example, as shown in FIG. 7D and FIG. 8, to screen for lasso peptides that are capable of modulating cell surface G protein-coupled receptors (GPCRs), a first assay that detects binding between the lasso peptide and the target molecule and a second assay that measures Ca²⁺ mobility (i.e., release of calcium from the endoplasmic reticulum to the cytoplasm) or intracellular Ca²⁺ concentration are used. As shown in FIG. 7D, after contacting the lasso peptide display library with a population of cells, the cells are further contacted with detecting reagents including an antibody conjugated with fluorophore A (e.g., FITC) and a Ca²⁺ indicator conjugated with fluorophore B (e.g., Rhodamine). The antibody specifically binds to the lasso peptide fusion protein and produces a fluorophore A signal (i.e., a fluorescent signal within the corresponding emission spectra after an initial excitation of fluorophore A). The Ca²⁺ indicator, upon binding with intracellular Ca²⁺, produces a fluorophore B signal (i.e., a fluorescent signal within the corresponding emission spectra after an initial excitation of fluorophore B). As shown in FIG. 8, fluorescence-activated cell sorting (FACS) is used to identify a first population of cells that produce only the fluorophore A signal, and a second population of cells that produce both fluorophore A and B signals. Lasso peptides bound to the two cell populations are identified by analyzing their respective DNA barcodes. The lasso peptide(s) that bind to the first cell population are identified as binder(s) for the GPCR, and the lasso peptide(s) that bind to the second cell population are identified as binder(s) of the GPCR and modulator(s) of the GPCR and its associated cellular activities. Further, as shown in FIG. 9, in exemplary embodiments, contacting the lasso peptide display library with a population of cells, further contacting the cell population with assay reagents, cell sorting, cell isolation, and cell collection, can be performed using a microfluidic device. Various detection mechanisms known in the art, such as measuring levels of secondary metabolites (e.g., cAMP, Ca²⁺, IP3/IP1 etc.), protein-protein binding interaction (e.g., Beta-arrestin recruitment), phosphorylation, or via reporter genes, can be used.

Additionally, Examples 17, 18 and 19 provide exemplary procedures and parameters for screening lasso peptide library for antagonists of GCGR, by measuring calcium mobility using a commercially available calcium assay. In some embodiments, library member(s) of a lasso peptide display library that causes and/or enhances a cellular activity and/or cell phenotype of interest is selected. In other embodiments, library member(s) of a lasso peptide display library that reduces and/or prevents a cellular activity and/or cell phenotype of interest is selected.

In some embodiments, a lasso peptide display library is subjected to biological assays that monitor multiple related cellular activities. For example, in some embodiments, each of the multiple related cellular activities induces or inhibits the same cellular signaling pathway. In some embodiments, the multiple related cellular activities are implicated in the same pathological process. In some embodiments, the multiple related cellular activities are implicated in regulating the cell cycle. In some embodiments, each of the multiple related cellular activities induces or inhibits cell proliferation. In some embodiments, each of the multiple related cellular activities induces or inhibits cell differentiation. In some embodiments, each of the multiple related cellular activities induces or inhibits cell apoptosis. In some embodiments, each of the multiple related cellular activities induces or inhibits cell migration.

In some embodiments, library member(s) identified as responsible for a detected change in at least one monitored cellular activity is selected. In some embodiments, library member(s) identified as responsible for a detected change in at least two monitored cellular activities is selected. In some embodiments, library member(s) identified as responsible for a detected change in at least three monitored cellular activities is selected. In some embodiments, library member(s) identified as responsible for a detected change in at least 10% monitored cellular activities is selected. In some embodiments, library member(s) identified as responsible for a detected change in at least 20% monitored cellular activities is selected. In some embodiments, library member(s) identified as responsible for a detected change in at least 30% monitored cellular activities is selected. In some embodiments, library member(s) identified as responsible for a detected change in at least 40% monitored cellular activities is selected. In some embodiments, library member(s) identified as responsible for a detected change in at least 50% monitored cellular activities is selected. In some embodiments, library member(s) identified as responsible for a detected change in at least 60% monitored cellular activities is selected. In some embodiments, library member(s) identified as responsible for a detected change in at least 70% monitored cellular activities is selected. In some embodiments, library member(s) identified as responsible for a detected change in at least 80% monitored cellular activities is selected. In some embodiments, library member(s) identified as responsible for a detected change in at least 90% monitored cellular activities is selected.

In some embodiments, members of a first lasso peptide display library selected during a first round of screening for a first desirable property are assembled to into a second lasso peptide display library, the second lasso peptide display library having an enriched population of members having the first desirable property. In some embodiments, the second lasso peptide display library is further subjected to a second round of screening for a second desirable property, and the selected library members are assembled into a third lasso peptide display library. The screening and selection processes can be repeated multiple times to produce one or more final selected member. In various embodiments, the first desirable property is the same as the second desirable property, and/or desirable property(ies) screened for in further round(s) of screens. In alternative embodiments, the first desirable property is different from the second desirable property, and/or desirable property(ies) screened for in further round(s) of screens. In some embodiments, the same desirable property is screened for under different conditions during the first and the second, or further round(s) of screens. For example, in specific embodiments, the desirable property is binding specificity of candidate library members to a target molecule, and during the sequential rounds of screens, the lasso peptide library is subjected to more and more stringent conditions for the library members to bind to the target molecule. For example, in specific embodiments, the first desirable property is a high binding affinity (e.g., binding affinity above a certain threshold value) of the candidate library members to a cell surface molecule, and the second desirable property is the ability of the candidate library members to enhance cell apoptosis mediated by the cell surface molecule.

In some embodiments, the lasso peptide display library comprises a plurality of separate units (e.g., a solid support having a plurality of reaction wells) each housing a unique member of the library, and the library members selected during the screening is identified based on its unique location. In certain embodiments, each member of the lasso peptide display library is associated with a detectable probe purported to produce a unique detectable signal, and the detectable signal is sufficiently unique to identify the associated member and/or distinguish the associated member from another member of the library, exemplary detectable signals that can be used in connection with the present disclosure include but are not limited to a chemiluminescent signal, a radiological signal, a fluorescent signal, a digital signal, a color signal, etc. In some embodiments, the lasso peptide display library is a molecular display library, and the unique nucleic acid molecule associated with library members selected during the screen is amplified and sequenced to identify the lasso peptide contained in the selected library member.

In alternative embodiments, any method for screening for a desired enzyme activity, e.g., production of a desired product, e.g., such as a lasso peptide or related molecule thereof, can be used. Any method for isolating enzyme products or final products, e.g., lasso peptides or related molecules thereof, can be used. In alternative embodiments, methods and compositions of the present disclosure comprise use of any method or apparatus to detect a purposefully biosynthesized organic product, e.g., lasso peptide or related molecule thereof, or supplemented or microbially-produced organic products (e.g., amino acids, CoA, ATP, carbon dioxide), by e.g., employing invasive sampling of either cell extract or headspace followed by subjecting the sample to gas chromatography or liquid chromatography often coupled with mass spectrometry.

In alternative embodiments, the apparatus and instruments are designed or configured for High Throughput Screening (HTS) and analysis of products, e.g., lasso peptides or related molecules thereof, produced by CFB methods and processes as provided herein, by detecting and/or measuring the products, e.g., lasso peptides, either directly or indirectly, in soluble form by sampling a CFB cell-free extract or medium. For example, either the FastQuan™ High-Throughput LCMS System from Thermo Fisher Scientific (Waltham, Mass., USA) or the StreamSelect™ LCMS System from Agilent Technologies (Santa Clara, Calif., USA) can be used to rapidly assay and identify production of lasso peptides or related molecules thereof in a CFB process implemented using 96-well, 384-well, or 1536-well plates.

In alternative embodiments, CFB methods and processes are automatable and suitable for use with laboratory robotic systems, eliminating or reducing operator involvement, while providing for high-throughput biosynthesis and screening.

Also provided are methods for screening a lasso peptide or related molecules thereof or a library of lasso peptides or related molecules thereof, produced by a CFB method or process, including the use of a TX-TL system, for an activity of interest. For example, the activity can be for a pharmaceutical, agricultural, nutraceutical, nutritional or animal veterinary or health and wellness function.

Also provided are methods for screening the CFB reaction mixture for: (i) a modulator of protein activity or metabolic function; (ii) a toxic metabolite, peptide or protein; (iii) an inhibitor of transcription or translation, comprising: (a) providing a CFB reaction mixture as described or provided herein, wherein the CFB reaction mixture comprises at least one protein-encoding nucleic acid which leads to the formation of a lasso peptide or related molecules thereof; (b) providing a test compound; (c) combining or mixing the test compound with the CFB reaction mixture under conditions wherein the CFB reaction mixture initiates or completes transcription and/or translation, or modifies a molecule, optionally a protein, a small molecule, a natural product, a lasso peptide, or a related molecule thereof, and, (d) determining or measuring any change in the functioning of the CFB reaction mixture, or the transcription and/or translation machinery, or in the formation of lasso peptide products, wherein determining or measuring a change in the protein activity, transcription or translation or metabolic function identifies the test compound as a modulator of that protein activity, transcription or translation or metabolic function.

Also provided are methods screening for: a modulator of protein activity, transcription, or translation or cell function; a toxic metabolite or a protein; a cellular toxin; an inhibitor of transcription or translation, comprising: (a) providing a CFB method and a cell extract or TX-TL composition described herein, wherein the composition comprises at least one protein-encoding nucleic acid; (b) providing a test compound; (c) combining or mixing the test compound with the cell extract under conditions wherein the TX-TL extract initiates or completes transcription and/or translation, or modifies a molecule (optionally a protein, a small molecule, a natural product, natural product analog, a lasso peptide, or a lasso peptide analog) and (d) determining or measuring any change in the functioning or products of the extract, or the transcription and/or translation, wherein determining or measuring a change in the protein activity, transcription or translation or cell function identifies the test compound as a modulator of that protein activity, transcription or translation or cell function.

Also provided are methods for screening of lasso peptides or related molecules thereof produced in a CFB system, whereby the CFB reaction mixture is directly assayed for biological activity, or optionally lasso peptides and related molecules thereof are substantially isolated and purified, comprising: (a) providing a CFB reaction mixture with a cell extract as described herein, wherein the composition comprises at least one protein-encoding nucleic acid; (b) providing a lasso precursor peptide, lasso precursor peptide gene, lasso core peptide, or lasso core peptide gene; (c) combining or mixing the lasso precursor peptide, lasso precursor gene, lasso core peptide, or lasso core peptide gene with the cell extract under conditions wherein the lasso precursor peptide, lasso peptide gene, lasso core peptide, or lasso core peptide gene is converted to form a lasso peptide or related molecules thereof, and (d) directly contacting the CFB reaction mixture, containing the products of transcription and/or translation, including lasso peptides or related molecules thereof, with a protein, enzyme, receptor, or cell, wherein a change in protein activity, transcription or translation, or cell function is measured and detected and identifies the lasso peptide or related molecules thereof as a modulator of biological activity, such as protein binding, enzyme activity, cell surface receptor activity, or cell growth; or (e) optionally substantially isolating and purifying the lasso peptides or related molecules thereof and contacting the lasso peptides or related molecules thereof, with a protein, enzyme, receptor, or cell, wherein the biological activity or cell function is measured and detected and identifies the lasso peptide or related molecules thereof as a modulator of biological activity, such as protein binding, enzyme activity, cell surface receptor activity, or cell growth.

### 5.4.2 Directed Evolution of Lasso Peptides

As disclosed herein, a set of nucleic acids encoding the desired activities of a lasso peptide biosynthesis pathway can be introduced into a host organism to produce a lasso peptide, or can be introduced into a CFB reaction mixture containing a cell extract or other suitable medium to produce a lasso peptide. In some cases, it can be desirable to modify the properties or biological activities of a lasso peptide to improve its therapeutic potential. In other cases, it can be desirable to modify the activity or specificity of lasso peptide biosynthesis pathway enzymes or proteins to improve the production of lasso peptides. For example, mutations can be introduced into an encoding nucleic acid molecule (e.g., a gene), which ultimately leads to a change in the amino acid sequence of a protein, enzyme, or peptide, and such mutated proteins, enzymes, or peptides can be screened for improved properties. Such optimization methods can be applied, for example, to increase or improve the activity or substrate scope of an enzyme, protein, or peptide and/or to decrease an inhibitory activity. Lasso peptides are derived from precursor peptides that are ribosomally produces by transcription and translation of a gene. Ribosomally produced peptides, such as lasso precursor peptides, are known to be readily evolved and optimized through variation of nucleotide sequences within genes that encode for the amino acid residues that comprise the peptide. Large libraries of peptide mutational variants have been produced by methods well known in the art, and some of these methods are referred to as directed evolution.

Directed evolution is a powerful approach that involves the introduction of mutations targeted to a specific gene or an oligonucleotide sequence containing a gene in order to improve and/or alter the properties or production of an enzyme, protein or peptide (e.g., a lasso peptide). Improved and/or altered enzymes, proteins or peptides can be identified through the development and implementation of sensitive high-throughput assays that allow automated screening of many enzyme or peptide variants (for example, >10⁴). Iterative rounds of mutagenesis and screening typically are performed to afford an enzyme or peptide with optimized properties.

Computational algorithms that can help to identify areas of the gene for mutagenesis also have been developed and can significantly reduce the number of enzyme or peptide variants that need to be generated and screened (See: Fox, R. J., et al., Trends Biotechnol., 2008, 26, 132-138; Fox, R. J., et al., Nature Biotechnol., 2007, 25, 338-344). Numerous directed evolution technologies have been developed and shown to be effective at creating diverse variant libraries, and these methods have been successfully applied to the improvement of a wide range of properties across many enzyme and protein classes (for reviews, see: Hibbert et al., Biomol. Eng., 2005, 22,11-19; Huisman and Lalonde, In Biocatalysis in the pharmaceutical and biotechnology industries, pgs. 717-742 (2007), Patel (ed.), CRC Press; Otten and Quax, Biomol. Eng., 2005, 22, 1-9; and Sen et al., Appl. Biochem. Biotechnol., 2007, 143, 212-223). Enzyme and protein characteristics that have been improved and/or altered by directed evolution technologies include, for example: selectivity/specificity, for conversion of non-natural substrates; temperature stability, for robust high temperature processing; pH stability, for bioprocessing under lower or higher pH conditions; substrate or product tolerance, so that high product titers can be achieved; binding (Kₘ), including broadening of ligand or substrate binding to include non-natural substrates; inhibition (Kᵢ), to remove inhibition by products, substrates, or key intermediates; activity (k_{cat}), to increase enzymatic reaction rates to achieve desired flux; isoelectric point (pI) to improve protein or peptide solubility; acid dissociation (pKα) to vary the ionization state of the protein or peptide with respect to pH; expression levels, to increase protein or peptide yields and overall pathway flux; oxygen stability, for operation of air-sensitive enzymes or peptides under aerobic conditions; and anaerobic activity, for operation of an aerobic enzyme or peptide in the absence of oxygen.

In one embodiment, a lasso peptide of interest is selected as the initial scaffold for directed evolution. Random mutations are introduced to a nucleic acid sequence encoding the initial scaffold, thereby producing a plurality of different mutated versions of the coding nucleic acid sequence. In some embodiments, a coding sequence of lasso precursor or lasso core peptide is mutated using the methods described herein or known in the art to produce a plurality of mutated versions of the coding sequence. The plurality of mutated versions of the coding sequence are then used to produce a first lasso peptide display library comprising a plurality of distinct lasso peptides or functional fragments of lasso peptides using, for example, the CFB system and methods disclosed herein. The library is then screened for candidate members having a desirable target property. Sequences of library members selected during the screen are analyze to identify beneficial mutations that lead to or improves the target property of the lasso peptides. One or more beneficial mutations are then introduced to the nucleic acid molecule encoding the initial scaffold to produce an improved version of the lasso peptide.

Optionally, in some embodiments, the coding sequence of the improved version of the lasso peptide is further mutated to introduce one or more additional mutations, while maintain the beneficial mutations, in the coding sequence. In some embodiments, a plurality of mutated versions of the coding sequences, each comprising at least one beneficial mutation identified in the first round of screen and at least one additional mutation is provided. These plurality of mutated versions of the coding sequences are then used to produce a second lasso peptide display library using, for example, the CFB system and methods disclosed herein. As such, the second lasso peptide display library is enriched with lasso peptides having at least one beneficial mutations. In some embodiments, the second lasso peptide display library is subjected to at least one more round of screening to identify improved members having the desirable target property. In some embodiments, additional beneficial mutations can be identified during the second round of the screening, and these additional beneficial mutations can also be used to design improved versions of the lasso peptide.

In some embodiments, additional beneficial mutations are also incorporated into members of a third or further lasso peptide display library(ies), which library(ies) can be subjected to a third or further round of screening and selection to identify candidate member(s) having the desirable target property. Additional beneficial mutations can be further identified for the evolution of the initial scaffold toward variants having improved target property. Examples 19 and 20 provide detailed exemplary procedures for directed evolution of lasso peptides.

In some embodiments, a later round of screening is performed at a more stringent condition as compared to an earlier round of screening, such that in the later round of screening, library members exhibiting the target property to a great extent (i.e. a better candidate) can be identified. Various adjustments for obtaining a more stringent screening condition are within the knowledge and skill in the art. For example, in specific embodiments, to identify lasso peptides that specifically binds to a target molecule, a more stringent screening condition can be achieved by performing the screening in the presence of a higher concentration of a molecule known to compete for binding to the target molecule. For example, in specific embodiments, to identify lasso peptides of improved thermal stability, a more stringent screening condition can be achieved by performing the screening at a higher temperature. For example, in specific embodiments, to identify lasso peptides capable of modulating a cellular activity or cell phenotype of interest, a more stringent screening condition can be achieved by performing the screening using less (or at a lower concentration of) candidate lasso peptides. In other embodiments, a more stringent screening condition can be achieved by setting forth a higher threshold for selection (e.g., a lower EC₅₀ or IC₅₀ in an assay measuring modulation of a cellular activity of interest, or a lower CC₅₀ in an assay measuring induced cell death, or a lower K_{d} in a binding assay, etc.).

Furthermore, a number of exemplary methods have been developed for the mutagenesis and diversification of genes and oligonucleotides to introduce into, and/or improve desirable target properties of, specific enzymes, proteins and peptides. Such methods are well known to those skilled in the art. Any of these can be used to alter and/or optimize the activity of a lasso peptide biosynthetic pathway enzyme, protein, or peptide, including a lasso precursor peptide, a lasso core peptide, or a lasso peptide. Such methods include, but are not limited to error-prone polymerase chain reaction (epPCR), which introduces random point mutations by reducing the fidelity of DNA polymerase in PCR reactions (See: Pritchard et al., J. Theor. Biol., 2005, 234:497-509); Error-prone Rolling Circle Amplification (epRCA), which is similar to epPCR except a whole circular plasmid is used as the template and random 6-mers with exonuclease resistant thiophosphate linkages on the last 2 nucleotides are used to amplify the plasmid followed by transformation into cells in which the plasmid is re-circularized at tandem repeats (Fujii et al., Nucleic Acids Res., 2004, 32:e145; and Fujii et al., Nat. Protoc., 2006, 1, 2493-2497); DNA, Gene, or Family Shuffling, which typically involves digestion of two or more variant genes with nucleases such as DNase I or EndoV to generate a pool of random fragments that are reassembled by cycles of annealing and extension in the presence of DNA polymerase to create a library of chimeric genes (Stemmer, Proc. Natl. Acad. Sci. U.S.A., 1994, 91, 10747-10751; and Stemmer, Nature, 1994, 370, 389-391); Staggered Extension (StEP), which entails template priming followed by repeated cycles of 2-step PCR with denaturation and very short duration of annealing/extension (as short as 5 sec) (Zhao et al., Nat. Biotechnol., 1998,16, 258-261); Random Priming Recombination (RPR), in which random sequence primers are used to generate many short DNA fragments complementary to different segments of the template (Shao et al., Nucleic Acids Res., 1998, 26, 681-683).

Additional methods include Heteroduplex Recombination, in which linearized plasmid DNA is used to form heteroduplexes that are repaired by mismatch repair (See: Volkov et al, Nucleic Acids Res., 1999, 27:e18; Volkov et al., Methods Enzymol., 2000, 328, 456-463); Random Chimeragenesis on Transient Templates (RACHITT), which employs DNase I fragmentation and size fractionation of single-stranded DNA (ssDNA) (See: Coco et al., Nat. Biotechnol., 2001, 19, 354-359); Recombined Extension on Truncated Templates (RETT), which entails template switching of unidirectionally growing strands from primers in the presence of unidirectional ssDNA fragments used as a pool of templates (See: Lee et al., J. Mol. Cat., 2003, 26, 119-129); Degenerate Oligonucleotide Gene Shuffling (DOGS), in which degenerate primers are used to control recombination between molecules; (Bergquist and Gibbs, Methods Mol. Biol., 2007, 352, 191-204; Bergquist et al., Biomol. Eng., 2005, 22, 63-72; Gibbs et al., Gene, 2001, 271, 13-20); Incremental Truncation for the Creation of Hybrid Enzymes (ITCHY), which creates a combinatorial library with 1 base pair deletions of a gene or gene fragment of interest (See: Ostermeier et al., Proc. Natl. Acad. Sci. U.S.A., 1999, 96, 3562-3567; and Ostermeier et al., Nat. Biotechnol., 1999, 17, 1205-1209); Thio-Incremental Truncation for the Creation of Hybrid Enzymes (THIO-ITCHY), which is similar to ITCHY except that phosphothioate dNTPs are used to generate truncations (See: Lutz et al., Nucleic Acids Res., 2001, 29, E16); SCRATCHY, which combines two methods for recombining genes, ITCHY and DNA Shuffling (See: Lutz et al., Proc. Natl. Acad. Sci. U.S.A., 2001, 98, 11248-11253); Random Drift Mutagenesis (RNDM), in which mutations made via epPCR are followed by screening/selection for those retaining usable activity (See: Bergquist et al., Biomol. Eng., 2005, 22, 63-72); Sequence Saturation Mutagenesis (SeSaM), a random mutagenesis method that generates a pool of random length fragments using random incorporation of a phosphothioate nucleotide and cleavage, which is used as a template to extend in the presence of “universal” bases such as inosine, and replication of an inosine-containing complement gives random base incorporation and, consequently, mutagenesis (See: Wong et al., Biotechnol. J. 2008, 3, 74-82; Wong et al., Nucleic Acids Res., 2004, 32, e26; Wong et al., Anal. Biochem., 2005, 341, 187-189); Synthetic Shuffling, which uses overlapping oligonucleotides designed to encode “all genetic diversity in targets” and allows a very high diversity for the shuffled progeny (See: Ness et al., Nat. Biotechnol., 2002, 20, 1251-1255); Nucleotide Exchange and Excision Technology NexT, which exploits a combination of dUTP incorporation followed by treatment with uracil DNA glycosylase and then piperidine to perform endpoint DNA fragmentation (See: Muller et al., Nucleic Acids Res., 33:e117).

Further methods include Sequence Homology-Independent Protein Recombination (SHIPREC), in which a linker is used to facilitate fusion between two distantly related or unrelated genes, and a range of chimeras is generated between the two genes, resulting in libraries of single-crossover hybrids (See: Sieber et al., Nat. Biotechnol., 2001, 19, 456-460); Gene Site Saturation Mutagenesis™ (GSSM™), in which the starting materials include a supercoiled double stranded DNA (dsDNA) plasmid containing an insert and two primers which are degenerate at the desired site of mutations, enabling all amino acid variations to be introduced individually at each position of a protein or peptide (See: Kretz et al., Methods Enzymol., 2004, 388, 3-11); Combinatorial Cassette Mutagenesis (CCM), which involves the use of short oligonucleotide cassettes to replace limited regions with a large number of possible amino acid sequence alterations (See: Reidhaar-Olson et al. Methods Enzymol., 1991, 208, 564-586; Reidhaar-Olson et al. Science, 1988, 241, 53-57); Combinatorial Multiple Cassette Mutagenesis (CMCM), which is essentially similar to CCM and uses epPCR at high mutation rate to identify hot spots and hot regions and then extension by CMCM to cover a defined region of protein sequence space (See: Reetz et al., Angew. Chem. Int. Ed Engl., 2001, 40, 3589-3591); the Mutator Strains technique, in which conditional is mutator plasmids, utilizing the mutD5 gene, which encodes a mutant subunit of DNA polymerase III, to allow a 20 to 4000-fold increase in random and natural mutation frequency during selection and block accumulation of deleterious mutations when selection is not required (See: Selifonova et al., Appl. Environ. Microbiol., 2001, 67, 3645-3649); Low et al., J. Mol. Biol., 1996, 260, 3659-3680).

Additional exemplary methods include Look-Through Mutagenesis (LTM), which is a multidimensional mutagenesis method that assesses and optimizes combinatorial mutations of a selected set of amino acids (See: Rajpal et al., Proc. Natl. Acad. Sci. U.S.A., 2005, 102, 8466-8471); Gene Reassembly, which is a homology-independent DNA shuffling method that can be applied to multiple genes at one time or to create a large library of chimeras (multiple mutations) of a single gene (See: Short, J. M., U.S. Pat. No. 5,965,408, Tunable GeneReassembly™); in Silico Protein Design Automation (PDA), which is an optimization algorithm that anchors the structurally defined protein backbone possessing a particular fold, and searches sequence space for amino acid substitutions that can stabilize the fold and overall protein energetics, and generally works most effectively on proteins with known three-dimensional structures (See: Hayes et al., Proc. Natl. Acad. Sci. U.S.A., 2002, 99, 15926-15931); and Iterative Saturation Mutagenesis (ISM), which involves using knowledge of structure/function to choose a likely site for enzyme improvement, performing saturation mutagenesis at chosen site using a mutagenesis method such as Agilent QuikChange Lightning Site-Directed Mutagenesis (Agilent Technologies; Santa Clara Calif.), screening/selecting for desired properties, and, using improved clone(s), starting over at another site and continue repeating until a desired activity is achieved (See: Reetz et al., Nat. Protoc., 2007, 2, 891-903; Reetz et al., Angew. Chem. Int. Ed Engl., 2006, 45, 7745-7751).

In some embodiments, the systems and libraries disclosed herein may be used in connection with a display technology, such that the components in the present systems and/or libraries may be conveniently screened for a property of interest. Various display technologies are known in the art, for example, involving the use of microbial organism to present a substance of interest (e.g., a lasso peptide or lasso peptide analog) on their cell surface. Such display technology may be used in connection with the present disclosure.

Furthermore, a rapid way to create large libraries of diverse peptides involves the use of display technologies (For a review, see: Ullman, C. G., et al., Briefings Functional Genomics, 2011, 10, 125-134). Peptide display technologies offer the benefit that specific peptide encoding information (e.g., RNA or DNA sequence information) is linked to, or otherwise associated with, each corresponding peptide in a library, and this information is accessible and readable (e.g., by amplifying and sequencing the attached DNA oligonucleotide) after a screening event, thus enabling identification of the individual peptides within a large library that exhibit desirable properties (e.g., high binding affinity). The cell-free biosynthesis methods provided herein can facilitate and enable the creation of large lasso peptide libraries containing lasso peptide analogs that can be screened for favorable properties. Lasso peptide mutants that exhibit the desired improved properties (hits) may be subjected to additional rounds of mutagenesis to allow creation of highly optimized lasso peptide variants. The CFB methods and systems described herein for the production of lasso peptides and related molecules thereof, used in combination with peptide display technologies, establishes a platform to rapidly produce high density libraries of lasso peptide variants and to identify promising lasso peptides with desirable properties.

In addition to biological methods for the evolution of lasso peptides, also can be conducted using chemical synthesis methods. For example, large combinatorial peptide libraries (e.g., >10⁶ members) containing mutational variants can be synthesized by using known solution phase or solid phase peptide synthesis technologies (See review: Shin, D.-S., et al., J. Biochem. Mol. Bio., 2005, 38, 517-525). Chemical peptide synthesis methods can be used to produce lasso precursor peptide variants, or alternatively, lasso core peptide variants, containing a wide range of alpha-amino acids, including the natural proteinogenic amino acids, as well as non-natural and/or non-proteinogenic amino acids, such as amino acids with non-proteinogenic side chains, or alternatively D-amino acids, or alternatively beta-amino acids. Cyclization of these chemically synthesized lasso precursor peptides or lasso core peptides can provide vast lasso peptide diversity that incorporates stereochemical and functional properties not seen in natural lasso peptides.

Any of the aforementioned methods for lasso peptide mutagenesis and/or display can be used alone or in any combination to improve the performance of lasso peptide biosynthesis pathway enzymes, proteins, and peptides. Similarly, any of the aforementioned methods for mutagenesis and/or display can be used alone or in any combination to enable the creation of lasso peptide variants which may be selected for improved properties.

In one embodiment of the present disclosure, a mutational library of lasso peptide precursor peptides is created and converted by a lasso peptidase and a lasso cyclase into a library of lasso peptide variants that are screened for improved properties. In another embodiment, a mutational library of lasso core peptides is created and converted by a lasso cyclase into a library of lasso peptide variants that are screened for improved properties.

In other embodiments of the present disclosure, a mutational library of lasso peptidases is created and screened for improved properties, such as increased temperature stability, tolerance to a broader pH range, improved activity, improved activity without requiring an RRE, broader lasso precursor peptide substrate scope, improved tolerance and rate of conversion of lasso precursor peptide mutational variants, improved tolerance and rate of conversion of lasso precursor peptide N-terminal or C-terminal fusions, improved yield of lasso peptides and related molecules thereof, and/or lower product inhibition. In other embodiments of the present disclosure, a mutational library of lasso cyclases is created and screened for improved properties, such as increased temperature stability, tolerance to a broader pH range, improved activity when used in combination with a lasso peptidase to convert a lasso precursor peptide, improved activity on a core peptide lacking a leader peptide, broader lasso precursor peptide substrate scope, broader lasso core peptide substrate scope, improved tolerance and rate of conversion of lasso core peptide mutational variants, improved tolerance and rate of conversion of lasso core peptide C-terminal fusions, improved yield of lasso peptides and related molecules thereof, and/or lower product inhibition.

In alternative embodiments, the present disclosure provides a method or composition according to any embodiment of the present disclosure, substantially as herein before described, or described herein, with reference to any one of the examples. In alternative embodiments, practicing the present disclosure comprises use of any conventional technique commonly used in molecular biology, microbiology, and recombinant DNA, which are within the skill of the art. Such techniques are known to those of skill in the art and are described in numerous texts and reference works (See e.g., Green and Sambrook, “Molecular Cloning: A Laboratory Manual,” 4th Edition, Cold Spring Harbor, 2012; and Ausubel et al., “Current Protocols in Molecular Biology,” 1987). Unless defined otherwise herein, all technical and scientific terms used herein have the same meaning as commonly understood by one of ordinary skill in the art to which the present disclosure pertains. For example, Singleton and Sainsbury, Dictionary of Microbiology and Molecular Biology, 2d Ed., John Wiley and Sons, N Y (1994); and Hale and Marham, The Harper Collins Dictionary of Biology, Harper Perennial, N.Y. (1991) provides those of skill in the art with general dictionaries of many of the terms used in the present disclosure. Although any methods and materials similar or equivalent to those described herein find use in the practice of the present disclosure, the preferred methods and materials are described herein. Accordingly, the terms defined below are more fully described by reference to the Specification as a whole.

### 6. EXAMPLES

### General Methods for Examples 1 to 10

Molecular biology and CFB reactions were conducted using standard plates, vial, and flasks typically employed when working with biological molecules such as DNA, RNA and proteins. LC-MS/MS analyses (including Hi-Res analysis) were performed on an Agilent 6530 Accurate-Mass Q-TOF MS equipped with a dual electrospray ionization source and an Agilent 1260 LC system with diode array detector. MS and UV data were analyzed with Agilent MassHunter Qualitative Analysis version B.05.00. Preparative HPLC was carried out using an Agilent 218 purification system (ChemStation software, Agilent) equipped with a ProStar 410 automatic injector, Agilent ProStar UV-Vis Dual Wavelength Detector, a 440-LC fraction collector and preparative HPLC column indicated below. Semi-preparative HPLC purifications were performed on an Agilent 1260 Series Instrument with a multiple wavelength detector and Phenomenex Luna 5 μm C8(2) 250×100 mm semi preparative column. Unless otherwise specified, all HPLC purifications utilized 10 mM aq. NH4HCO3/MeCN and all analytical LCMS methods included a 0.1% formic acid buffer. NMR data are acquired using a 600 MHz Bruker Avance III spectrometer with a 1.7 mm cryoprobe. All signals are reported in ppm with the internal DMSO-d6 signal at 2.50 ppm (¹H-NMR) or 39.52 ppm (¹³C-NMR). 1D data is reported as s=singlet, d=doublet, t=triplet, q=quadruplet, m=multiplet or unresolved, br=broad signal, coupling constant(s) in Hz.

To prepare cell extracts, E. coli BL21 Star(DE3) cells were grown in the minimum medium containing MM9 salts (13 g/L), calcium chloride (0.1 mM), magnesium sulfate (2 mM), trace elements (2 mM) and glucose (10 g/L), in a 10 L bioreactor (Satorius) to the mid-log growth phase. The grown cells were then harvested and pelleted. The crude cell extracts were prepared as described in Kay, J., et al., Met. Eng., 2015, 32, 133-142 and Sun, Z. Z., J. Vis. Exp. 2013, 79, e50762, doi:10.3791/50762. For calibration of additional magnesium, potassium and DTT levels, a green fluorescence protein (GFP) reporter was used to determine the additional amount of Mg-glutamate, K-glutamate, and DTT that were subsequently added to each batch of the crude cell extracts to prepare the optimized cell extracts for optimal transcription-translation activities. Prior to cell-free biosynthesis of lasso peptide, the optimized cell extracts were pre-mixed with buffer that contains ATP, GTP, TTP, CTP, amino acids, t-RNA, magnesium glutamate, potassium glutamate, potassium phosphate, and other salts, NAD+, NADPH, glucose, 0.5 mM IPTG and 3 mM DTT to achieve a desirable reaction volume. An exemplary cell extract comprises the ingredients, and optionally with the amounts, as set forth in the following Table 1.

TABLE 1

| Ingredients | Concentration | |
|---|---|---|
| E. coli BL21 Star(DE3) | 33% v/v (10 mg/ml of | |
| extracts | protein or higher) | |
| Amino Acids | 1.5 mM each | |
| | (Leucine, 1.25 mM) | |
| HEPES | 50 | mM |
| ATP | 1.5 | mM |
| GTP | 1.5 | mM |
| CTP & UTP | 0.9 | mM |
| tRNA | 0.2 | mg/mL |
| CoA | 0.26 | mM |
| NAD+ | 0.33 | mM |
| cAMP | 0.75 | mM |
| Folinic acid | 0.068 | mM |
| spermidine | 1 | mM |
| pEG-8000 | 2% | |
| magnesium glutamate | 4-12 | mM |
| potassium glutamate | 8-160 | mM |
| potassium phosphate | 1-10 | mM |
| DTT | 0-5 | mM |
| NADPH | 1 | mM |
| maltodextrin | 35 | mM |
| IPTG (optional) | 0.5 | mM |
| pyruvate | 30 | mM |
| NADH | 1 | mM |

Affinity chromatography procedures are carried out according to the manufacturers' recommendations to isolate lasso peptides fused to an affinity tag; for examples, Strep-tag® II based affinity purification (Strep-Tactin® resin, IBA Lifesciences), His-tag-based affinity purification (Ni-NTA resin, Thermo Fisher Scientific), maltose-binding protein based affinity purification (amylose resin, New England BioLabs). The sample of lasso peptides fused to an affinity tag is lyophilized and resuspended in a binding buffer with respect to its affinity tag according to the manufacturer's recommendation. The resuspended lasso peptide sample is directly applied to an immobilized matrix corresponding to its fused affinity tag (Tactin for Strep-tag® II, Ni-NTA for His-tag, or amylose resin for maltose binding protein) and incubated at 4° C. for an hour. The matrix is then washed with at least 40× volume of washing buffer and eluted with three successive 1× volume of elution buffer containing 2.5 mM desthiobiotin for Strep-Tactin® resin, 250 mM imidizole for Ni-NTA resin or 10 mM maltose for amylose resin. The eluted fractions are analyzed on a gradient (10-20%) Tris-Tricine SDS-PAGE gel (Mini-PROTEAN, BioRad) and then stained with Coomassie brilliant blue.

The purity of eluted lasso peptide was examined by LC-MSMS on an Agilent 6530 Accurate-Mass Q-TOF mass spectrometer. Where possible, MSMS fragmentation is used to further characterize lasso peptides based on the rule described in Fouque, K. J. D, et al., Analyst, 2018,143, 1157-1170. If impurities are observed in chromatographic spectra, preparative chromatography is performed to further enrich the purity of lasso peptides.

### Analytical LCMS Analytical Method:

Column: Phenomenex Kinetex 2.6μ XB-C18 100 A, 150×4.6 mm column.
Flow rate: 0.7 mL/min
Temperature: RTMobile Phase A: 0.1% formic acid in water
Mobile Phase B: 0.1% formic acid in acetonitrile
Injection amount: 2 μL
HPLC Gradient: 10% B for 3.0 min, then 10 to 100% B over 20 minutes follow by 100% B for 3 min. 4 minute post run equilibration time

Preparative HPLC was carried out using an Agilent 218 purification system (ChemStation software, Agilent) equipped with a ProStar 410 automatic injector, Agilent ProStar UV-Vis Dual Wavelength Detector, a 440-LC fraction collector. Fractions containing lasso peptides were identified using the LCMS method described above, or by direct injection (bypassing the LC column in the above method) prior to combining and freeze-drying. Analytical LC/MS (see method above) was then performed on the combined and concentrated lasso peptides.

### Preparative HPLC Method:

Column: Phenomenex Luna® preparative column 5 μM C18(2) 100 Å 100×21.2 mm
Flow rate: 15 mL/min

### Temperature: RT

Mobile Phase A: 10 mM aq. NH4HCO3
Mobile Phase B: acetonitrile
Injection amount: varies
HPLC Gradient: 20-40% MeCN for 20 min, then 40-95% MeCN for 5 min

If necessary, semi-preparative HPLC purifications were performed on an Agilent 1260 Series Instrument with a multiple wavelength detector

### Semipreparative HPLC Method:

### Column: Phenomenex Luna® 5 μm C18(2) 250×100 mm

Flow rate: 4 mL/min

### Temperature: RT

Mobile Phase A: 10 mM aq. NH4HCO3
Mobile Phase B: acetonitrile
Injection amount: varies
HPLC Gradient: 20-40% MeCN for 20 min, then 40-95% MeCN for 5 min

Monoisotopic masses were extrapolated from the lasso peptide charge envelop [(M+H)¹⁺, (M+2H)²⁺, (M+3H)³⁺ in the m/z 500-3,200 range using a Agilent 6530 Accurate-Mass Q-TOF MS equipped with a dual electrospray ionization source and an Agilent 1260 LC system using an internal reference (see analytical procedure described above). Both MS and MS/MS analyses were performed in positive-ion mode.

NMR samples are dissolved in DMSO-d6 (Cambridge Isotope Lab-oratories). All NMR experiments are run on a 600 MHz Bruker Avance III spectrometer with a 1.7 mm cryoprobe. All signals are reported in ppm with the internal DMSO-d6 signal at 2.50 ppm (¹H-NMR) or 39.52 ppm (¹³C-NMR). Where applicable, structural characterization of lasso peptide follow the methods described in the literatures listed below:

1. Knappe et al., J. Am. Chem. Soc., 2008, 130 (34), 11446-11454

2. Maksimov et al., PNAS, 2012, 109 (38), 15223-15228

3. Tietz et al., Nature Chem. Bio., 2017,13, 470-478

4. Zheng and Price, Prog Nucl Magn Reson Spectrosc, 2010, 56 (3), 267-288

5. Marion et al., J Magn Reson, 1989, 85 (2), 393-399

6. Davis et al., J Magn Reson, 1991, 94 (3), 637-644

7. Rucker and Shaka, Mol Phys, 1989, 68 (2), 509-517

8. Hwang and Shaka, J Magn Reson A, 1995, 112 (2), 275-27

Table 2 below lists examples of lasso peptides produced with cell-free biosynthesis using a minimum set of genes.

TABLE 2
Minimum set of genes for cell-free biosynthesis of lasso peptides.

| | | | | | Cyclase- | | RRE- |
|---|---|---|---|---|---|---|---|
| | | Precursor | Peptidase | Cyclase | RRE | RRE | peptidase |
| Lasso | Molecular | SEQ ID | SEQ ID | SEQ ID | SEQ ID | SEQ ID | SEQ ID |
| peptide | mass | NO: | NO: | NO: | NO: | NO: | NO: |
| Microcin J25 | 2107.02 | 25 | 26 | 27 | | | |
| ukn22 | 2269.18 | 28 | 29 | 30 | | 31 | |
| capistruin | 2048.01 | 32 | 33 | 34 | — | — | — |
| lariatin | 2204.12 | 35 | 36 | 37 | — | 38 | — |
| ukn16 | 2306.07 | 39 | 40 | — | 41 | — | — |
| adanomysin | 1675.66 | 42 | — | 43 | — | — | 44 |

TABLE 3
The list of protein sequences described in the following Examples 1-10.

| | | | GenBank |
|---|---|---|---|
| SEQ ID | | | Accession #; |
| NO: | Name | A.A. sequence (leader/core junction | GI # |
| 25 | microcin j25 | MIKHFHFNKLSSGKKNNVPSPAKGVIQIK | N/A |
| | precursor | KSASQLTKGGAGHVPEYFVGIGTPISFYG | |
| | | (37/38) | |
| 26 | microcin j25 | MIRYCLTSYREDLVILDIINDSFSIVPDAGS | WP_001513515; |
| | peptidase | LLKERDKLLKEFPQLSYFFDSEYHIGSVSR | 486129256 |
| | | NSDTSFLEERWFLPEPDKTLYKCSLFKRFI | |
| | | LLLKVFYYSWNIEKKGMAWIFISNKKEN | |
| | | RLYSLNEEHLIRKEISNLSIIFHLNIFKSDC | |
| | | LTYSYALKRILNSRNIDAHLVIGVRTQPF | |
| | | YSHSWVEVGGQVINDAPNMRDKLSVIAEI | |
| 27 | microcin j25 | MEIFNVKLNDTSIRIIFCKTLSAFRTENTIV | WP_001513514; |
| | cyclase | MLKGKAVSNGKPVSTEEIARVVEEKGVS | 486129253 |
| | | EVIENLDGVFCILIYHFNDLLIGKSIQSGPA | |
| | | LFYCKKNMDIFVSDKISDIKFLNPDMTFS | |
| | | LNIKMAEHYLSGNRIATQESLITGIYKVN | |
| | | NGEFIKFNNQLKPVLLRDEFSITKKNNSTI | |
| | | DSIIDNIEMMRDNRKIALLFSGGLDSALIF | |
| | | HTLKES | |
| 28 | ukn22 precursor | MEKKKYTAPQLAKVGEFKEATGWYTAE | |
| | | WGLELIFVFPRFI (22/23) | |
| 29 | ukn22 peptidase | MSENVVLQRSNVRLSWRTKWAARCAVG | WP_011291590; |
| | | AARLLARKPPERIRATLLRLRGEVRPATY | 499610856 |
| | | EEAKAARDAVLAVSLRCAGLRACLQRSL | |
| | | AIALLCRMRGTWATWCVGVPRRPPFIGH | |
| | | AWVEAEGRLVEEGVGYDYFSRLITVD | |
| 30 | ukn22 cyclase | MVGCISPYFAVFPDKDVLGQATDRLPAA | WP_011291592; |
| | | QTLASHPSGRPWLVGALPADQLLLVEAG | 499610858 |
| | | ERRLAVIGHCSAEPERLRAELAQIDDVAQ | |
| | | FDRIARTLDGSFHLVVVVGDQMRIQGSV | |
| | | SGLRRVFHAHVGTARIAADRSDVLAAVL | |
| | | GVSPDPDVLALRMFNGLPYPLSELPPWPG | |
| | | VEHVPAWHYLSLGLHDGRHRVVQWWH | |
| | | PPEAELAVTAAAPLLRTALAGAVDTRTR | |
| | | GGGVVSADLSGGLDSTPLCALAARGPAK | |
| | | VVALTFSSGLDTDDDLRWAKIAHQSFPS | |
| | | VEHVVLSPEDIPGFYAGLDGEFPLLDEPS | |
| | | VAMLSTPRILSRLHTARAHGSRLHMDGL | |
| | | GGDQLLTGSLSLYHDLLWQRPWTALPLI | |
| | | RGHRLLAGLSLSETFASLADRRDLRAWL | |
| | | ADIRHSIATGEPPRRSLFGWDVLPKCGPW | |
| | | LTAEARERVLARFDAVLESLEPLAPTRGR | |
| | | HADLAAIRAAGRDLRLLHQLGSSDLPRM | |
| | | ESPFLDDRVVEACLQVRHEGRMNPFEFK | |
| | | SLMKTAMASLLPAEFLTRQSKTDGTPLA | |
| | | AEGFTEQRDRIIQIWRESRLAELGLIHPDV | |
| | | LVERVKQPYSFRGPDWGMELTLTVELWL | |
| | | RSRERVLQGANGGDNRS | |
| 31 | ukn22 RRE | METTGAEFRLRPEISVAQTDYGMVLLDG | WP_011291591; |
| | | RSGEYWQLNDTAALIVQRLLDGHSPADV | 499610857 |
| | | AQFLTSEYEVERTDAERDIAALVTSLKEN | |
| | | GMALP | |
| 32 | capistruin | MVRLLAKLLRSTIHGSNGVSLDAVSSTH | |
| | precursor | GTPGFQTPDARVISRFGFN (28/29) | |
| 33 | capistruin | MTPASHCHIAVFDQAIVALDMQRSRYFL | WP_009905509; |
| | peptidase | YDEACAKAFADHYLDFKPIDAPHALKPLI | 497591325 |
| | | SDRIVVAASPASVPKRIADYRGWAFDAF | |
| | | DSGIWASRTLGERSAAGFEWLPFWRIVR | |
| | | GAVSLKMRGFRALSALDRLARLDAGAE | |
| | | QRARTDGGPSRTAERYLRASIWSPFRITC | |
| | | LQMSFALATHLRRENVPAQLVIGVRPMP | |
| | | FVAHAWVEIDGRVCGDEPELKKSYGEIY | |
| | | RTPRHDERAGPFGLAA | |
| 34 | capistruin cyclase | MTLLEAGARARAYLRDAHSRIERSLARA | WP_045600732; |
| | | RTLQEARDTVTRSVWGAYLLVLDEAASG | 782674010 |
| | | RRLFMPDPLHSVRLYYRTDERGRVDVDP | |
| | | RAANLLDRASIDWNLDYLIEFACTQFGPL | |
| | | DETPFASVRVVPPGCALVVGPDGRCAIER | |
| | | AWLPRAQAAGDVRASCAAALDDVYSRI | |
| | | AHSHPSVCAALSGGVDSSAGAIFLRKALG | |
| | | ANAP | |
| 35 | lariatin precursor | MTSQPSKKTYNAPSLVQRGKFARTTAGS | |
| | | QLVYREWVGHSNVIKPGP (26/27) | |
| 36 | lariatin peptidase | MPVVGAMAIPSKTRISATERLRLASALSL | BAL72549; |
| | | GKALSHLPPGLLRRSMTAFAAKARPASY | 380356107 |
| | | REAEAAVVSITQYSKASAGPGSCLQRSIS | |
| | | VCILMRLDGRWPTWCVGVPSKPPFRAHA | |
| | | WIEAGGQIVAELGDMNSYSRLMTISTHA | |
| | | ERTES | |
| 37 | lariatin cyclase- | MNGIDIAVVTDDPAILKSVHERYPDGSKH | BAL72547; |
| | | SIELEHGHNVHIFVRTATLVLSSYIRDNEA | 380356105 |
| | | IAVLGYSNVHESSMRAILESSPGVAHMNS | |
| | | ALGQLIGAQWVVAIRKGAVRIQGTVSGL | |
| | | SRVYWSKRGSRFVASNRSRELARLLGSE | |
| | | LDPTQVAFRTIHPMQHPFTSSSCWKDLEG | |
| | | VLPGEYLEVTGRSSPRTERWWTPATSYR | |
| | | SLEDGANETAAALFSVVRNQLSDHSAAS | |
| | | CDISGGLDSSSIAAIAANAAKSGETHTVL | |
| | | HGTTSVSRDEFNSDADWAIELSKSLKLDS | |
| | | HSFLSWNDMPKEYDDLDALASYDLDEPS | |
| | | IASISHSRFTHLINVARSKGSQVHLTGFGG | |
| | | DELFIGSPTFCVDLFKTQPLLSARLLLTYR | |
| | | AMYRWTFRSLVRPLTTPMSYQQWMRTK | |
| | | SLSTDRSTLRIPPLSWGFHGVIPPWITRDA | |
| | | RHSMYDHVRSASSATFPLAPTPGRHFELE | |
| | | NLYQCARLFRTMSDIVSQTSGVLLVAPM | |
| | | LEQAVVEAAISVRTPERLTPHKYKPVLTH | |
| | | ATRGLLPAVVAERQTKGGEDTDAAIGFS | |
| | | ENISAIRELWDESRLASLGIVDGDYLSNA | |
| | | LRRPDSAEFDDCAIAKTLATELWLRSLEK | |
| 38 | lariatin RRE | MVLRLRKNVIITPTEYGAVALDERSGDY | BAL72548; |
| | | YQLNSTAALILDQLTKKIPVESIAARIALD | 380356106 |
| | | FEVSKAQASADLDEYLRMLREQGLLR | |
| 39 | ukn16 precursor | MKDYVPPVVEVIASFKEATNGVWFGNY | |
| | | VDVGGAKAPFPWGSN (20/21) | |
| 40 | ukn16 peptidase | MSIPLQPEATTRVNFHDRLVALIAIIIGRR | KFI86627; |
| | | MERQRIGKFCRRLETWSERYPPADADMA | 672991436 |
| | | KRYRNAVCSVSRRCRSQQGCLLRSLSTA | |
| | | AACRISRRSVTWCTGFTDRPFRAHAWVE | |
| | | ANGIPIGEPDAVRRYTITRTSSERKDTQ | |
| 41 | ukn16 cyclase- | MMPFTHKNPNRTVIVRGGKPTDRSKLPA | KFI86628; |
| | RRE fusion | SISINDEGTCVQVPLLSGDRMFWTVSKDE | 672991437 |
| | | VLLSDSAFRLASITNADLDLERIIMDLLPS | |
| | | LPDSLRGDKSPWRNIHSVPAATTLHLDKS | |
| | | NRPRYTRAEPSPIKHVSNDVILASLRSRFL | |
| | | TIADEWRNEAPLSADVSGGVDSAAIAYIF | |
| | | ASEGVRMPLYHETPDDPMNQDSRWAERI | |
| | | SEDVRMPLIKIGRVVDGNRSFESTAEYPN | |
| | | REIPEEPVFWSDIEGYLSRISEMEADTSRI | |
| | | HVTGFGGDELFASMPSSSWSCLREHPLRL | |
| | | RDIRRQYSADYRVPPYQAILDLTDSTDLH | |
| | | EELRSSLLDAEQDHGHRSSPCGWHDAIRI | |
| | | PEFLTAKARDTLYGAIESQLKHADIRPLSP | |
| | | DRSRHQALYSLSMQARMLNQVNRTFAS | |
| | | DDITFRSPYLDRGIVGYALCAPISARTEGN | |
| | | LHKAVLYRALKGIVPETIFRRPVKGDHSY | |
| | | SLYLAWQRSKDTLLDSIAGGVLDEEGLID | |
| | | IPAVRRRASMPMPDITFLFEMQRVAAVE | |
| | | GGDMQTDNTMANTSLKDQISLYPKEGG | |
| | | AIVFVNDTGEYLQVNEIGRIILDGLMHGK | |
| | | TVEDCMNAIAEEYQTDRQIIVRDTERFLA | |
| | | DIGKHVRL | |
| 42 | adanomysin | MFYEPPVVVDLGSVRDVTLGSSTSGTAD | |
| | precursor | ANSQYYW (19/20) | |
| 43 | adanomysin | MRNGLLGVFPPASSGEVVRVQGPWREGE | WP_031228349; |
| | cyclase | LRRVDGPEGTVAVLGQCLSDDDRLRRTA | 665861142 |
| | | LRALASGGPGELTRLPGSYLCLVIRHEEL | |
| | | TAYVDAAGQYPLFFRDTGTRLVFGTRPV | |
| | | SVADAAGARRRPDTAVLAAGIFCPGAPS | |
| | | LTGERSVVAGVSKVGGGQALRRTARGK | |
| | | VERWVHEPLETDPGVSLARSAEALRDAL | |
| | | ETAVRLRVAGTERVSADFSGGLDSTSLAF | |
| | | LTLRHRPGPLPVTTYRGAASACDDLVHA | |
| | | ERFARLDPRLRMEVVTGTRETLTYQGLG | |
| | | DRSGGAGHDSDEPDPAVVALARSRLRLD | |
| | | QVARLGAGVHLGGEGGDALLVAPPGYL | |
| | | AALARPERLRQLAKESRVLARARQEAPS | |
| | | AVAARAVGLARTPLATALRRLADGFERH | |
| | | ATGGTGRAGAGDVGWLDAIAWWPGPGS | |
| | | ETEWLTRAASAELAGLAREAAGSAGRTA | |
| | | GSRAGDLTALDNVRTSGAVQRQLSEMA | |
| | | RPFGVWPQAPFLDSAVIRACAALPAHLR | |
| | | AAPPAFKPLLGAALADLVPAPVLARRTK | |
| | | GDYGDEDYQGARACARELRGLLVDSRL | |
| | | AELGVVEPSAVVAALDRAVMGLRVPFPA | |
| | | LNRLLAAEIWLRNTTWH | |
| 44 | adanomysin RRE- | MAAFHIPEHVHESSGPHGGTVLLDARTG | WP_023536418; |
| | peptidase fusion | QWYAMNGTARALWSEWRESGDFDAGV | 558881359 |
| | | RTVAARFPPALGERVRTDAGQLAETLLQ | |
| | | RGLVSAEPSSDGSGRCLRPVRRAGRRFSA | |
| | | APRRNRSGATAALVVALCLLRLPFGVTV | |
| | | RVVAALTSRCPHPATHAQAEQALAAVRR | |
| | | VSRRYPGRVACLELSLAATVRLALAGLG | |
| | | AQWCLGSADDPYRFHAWIEAGGRPVTSP | |
| | | SEGELSGFRKVLTV | |
| 45 | microcin j25 | GGAGHVPEYFVGIGTPISFYG | N/A |
| 46 | ukn22 | WYTAEWGLELIFVFPRFI | N/A |
| 47 | capistruin | GTPGFQTPDARVISRFGFN | N/A |
| 48 | lariatin | GSQLVYREWVGHSNVIKPGP | N/A |
| 49 | ukn16 | GVWFGNYVDVGGAKAPFPWGSN | N/A |
| 50 | adanomysin | GSSTSGTADANSQYYW | N/A |

### 6.1 Example 1: Cell-free Synthesis of Microcin J25

Synthesis of microcin J25 (MccJ25) lasso peptide GGAGHVPEYFVGIGTPISFYG (SEQ ID NO: 45) where the N-terminal amine group of a glycine (G) residue at the first position was cyclized with the side-chain carboxylic acid group of a glutamic acid (E) residue at the eighth position

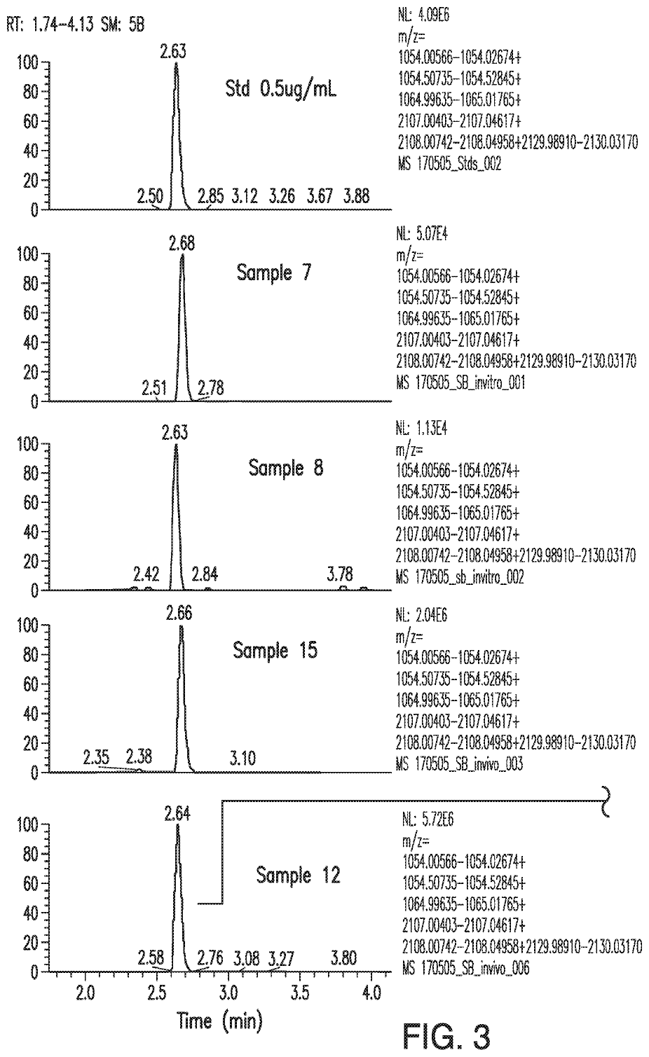

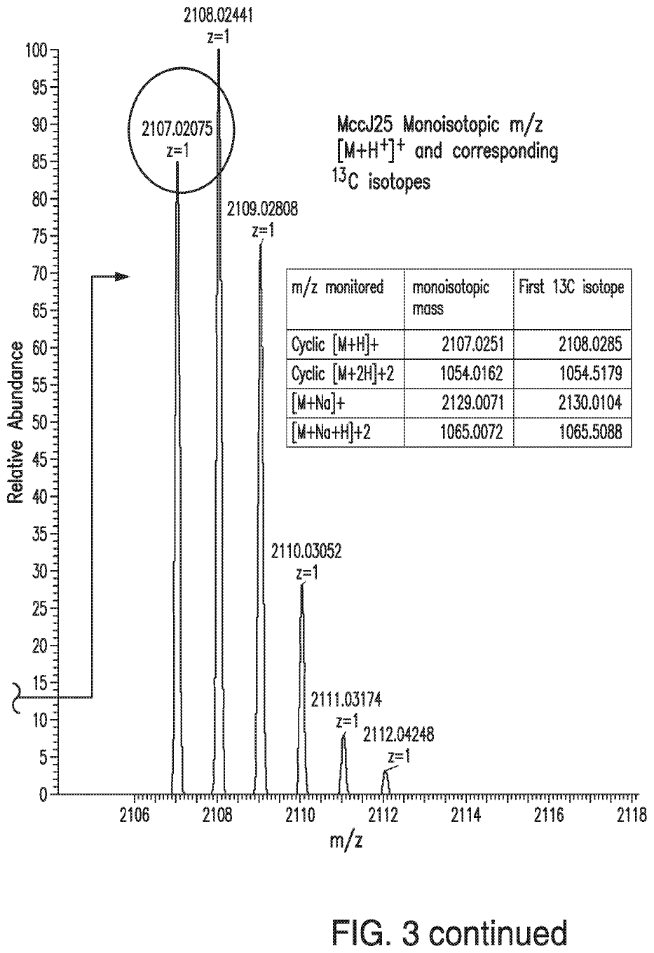

DNA encoding the sequences for the MccJ25 precursor peptide (SEQ ID NO: 25), peptidase (SEQ ID NO: 26), and cyclase (SEQ ID NO: 27) from Escherichia coli were synthesized (Thermo Fisher Scientific, Carlsbad, Calif.) and individually cloned into a pZE expression vector behind a T7 promoter (Expressys). The resulting plasmids encoding genes for the MccJ25 precursor peptide (SEQ ID NO: 25) without a C-terminal affinity tag, peptidase (SEQ ID NO: 26) with a C-terminal Strep-tag®, and cyclase (SEQ ID NO: 27) also with a C-terminal Strep-tag® were used for subsequent cell-free biosynthesis. The MccJ25 precursor peptide (SEQ ID NO: 25) was produced using the PURE system (New England BioLabs) according to the manufacturer's recommended protocol. The peptidase (SEQ ID NO: 26) and cyclase (SEQ ID NO: 27) were expressed in Escherichia coli as described by Yan et al., Chembiochem. 2012, 13(7):1046-52 and purified using Tactin resin (IBA Lifesciences) according to the manufacturer's recommendation. Production of MccJ25 lasso peptide was initiated by adding 5 μL of the PURE reaction containing the MccJ25 precursor peptide (SEQ ID NO: 25), and 10 μL of purified peptidase (SEQ ID NO: 26), and 20 ΞL of purified cyclase (SEQ ID NO: 27) in buffer that contains 50 mM Tris (pH8), 5 mM MgCl₂, 2 mM DTT and 1 mM ATP to achieve a total volume of 50 μL. The cell-free biosynthesis of MccJ25 lasso peptide was accomplished by incubating the reaction for 3 hours at 30° C. The reaction sample was subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid faction was subjected to LC/MS analysis on an Applied Biosystems 3200 APCI triple quadrupole mass spectrometer for lasso peptide detection. The molecular mass of 2107.02 m/z corresponding to MccJ25 lasso peptide (GGAGHVPEYFVGIGTPISFYG minus H₂O) was observed (FIG. 3). The collected lasso peptide sample is further purified by affinity chromatography and/or preparative HPLC, followed by high resolution mass spectrometry and NMR for structural characterization.

### 6.2 Example 2: Synthesis of Ukn22 Lasso Peptide

Synthesis of ukn22 lasso peptide WYTAEWGLELIFVFPRFI (SEQ ID NO: 46) where the N-terminal amine group of a tryptophan (W) residue at the first position was cyclized with the side-chain carboxylic acid group of a glutamic acid (E) residue at the ninth position

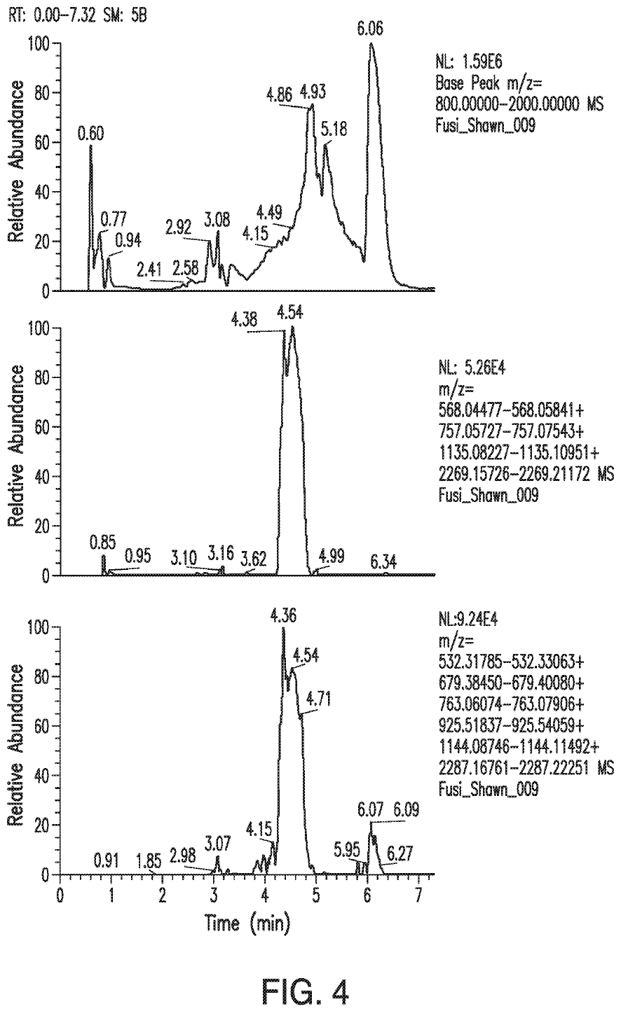

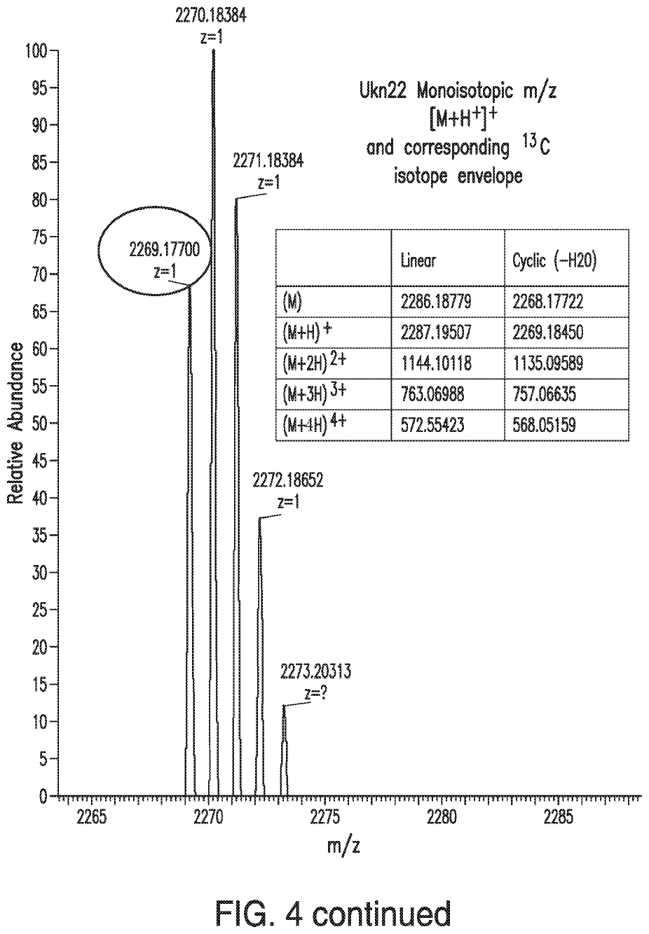

DNA encoding the sequences for the ukn22 precursor peptide (SEQ ID NO: 28), peptidase (SEQ ID NO: 29), cyclase (SEQ ID NO: 30) and RRE (SEQ ID NO: 31) from Thermobifida fusca were used. Each of the DNA sequences was cloned into a pET28 plasmid vector behind a maltose binding protein (MBP) sequence to create an N-terminal MBP fusion protein. The resulting plasmids encoding fusion genes for the MBP-ukn22 precursor peptide (SEQ ID NO: 28), MBP-peptidase (SEQ ID NO: 29), MBP-cyclase (SEQ ID NO: 30) and MBP-RRE (SEQ ID NO: 31) were driven by an IPTG-inducible T7 promoter. Production of ukn22 lasso peptide was initiated by adding the MBP-ukn22 precursor peptide (SEQ ID NO: 28), MBP-peptidase (SEQ ID NO: 29), MBP-cyclase (SEQ ID NO: 30) and MBP-RRE (SEQ ID NO: 31) (20 nM each) to the optimized E. coli BL21 Star(DE3) cell extracts, which was pre-mixed with buffer as described earlier to achieve a total volume of 50 μL. The cell-free biosynthesis of ukn22 lasso peptide was accomplished by incubating the reaction for 16 hours at 22° C. The reaction sample was subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid faction was subjected to LC/MS analysis on an Applied Biosystems 3200 APCI triple quadrupole mass spectrometer for lasso peptide detection. The molecular mass of 2269.18 m/z corresponding to ukn22 lasso peptide (WYTAEWGLELIFVFPRFI minus H₂O) was observed (FIG. 4). The collected lasso peptide sample is further purified by affinity chromatography and/or preparative HPLC, followed by high resolution mass spectrometry and NMR for structural characterization.

### 6.3 Example 3: Synthesis of Capistruin Lasso Peptide

Synthesis of capistruin lasso peptide GTPGFQTPDARVISRFGFN (SEQ ID NO: 47) where the N-terminal amine group of a glycine (G) residue at the first position is cyclized with the side-chain carboxylic acid group of an aspartic acid (D) residue at the ninth position

Codon-optimized DNA encoding the sequences for the capistruin precursor peptide (SEQ ID NO: 32), peptidase (SEQ ID NO: 33) and cyclase (SEQ ID NO: 34) from Burkholderia thailandensis are synthesized (Thermo Fisher Scientific, Carlsbad, Calif.) and individually cloned into a pZE expression vector behind a T7 promoter (Expressys). The resulting plasmids encoding genes for the capistruin precursor peptide (SEQ ID NO: 32), peptidase (SEQ ID NO: 33) and cyclase (SEQ ID NO: 34) are used with or without a C-terminal affinity tag. Production of capistruin lasso peptide is initiated by adding the capistruin precursor peptide (SEQ ID NO: 32), peptidase (SEQ ID NO: 33) and cyclase (SEQ ID NO: 34) (15 nM each) to the optimized E. coli BL21 Star(DE3) cell extracts, which is pre-mixed with buffer that contains ATP, GTP, TTP, CTP, amino acids, t-RNA, magnesium glutamate, potassium glutamate, potassium phosphate, and other salts, NAD+, NADPH, and glucose to achieve a total volume of 400 μL. The cell-free biosynthesis of capistruin lasso peptide is accomplished by incubating the reaction for 18 hours at 22° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid faction is subjected to LC/MS analysis on an Agilent 6530 Accurate-Mass Q-TOF MS equipped with a dual electrospray ionization source and an Agilent 1260 LC system with diode array detector for lasso peptide detection. The molecular mass of 2048.01 m/z corresponding to capistruin lasso peptide (GTPGFQTPDARVISRFGFN (SEQ ID NO: 47) minus H₂O) is observed. The collected lasso peptide sample is further purified by affinity chromatography and/or preparative HPLC, followed by high resolution mass spectrometry and NMR for structural characterization.

### 6.4 Example 4: Synthesis of Lariatin Lasso Peptide

Synthesis of lariatin lasso peptide GSQLVYREWVGHSNVIKPGP (SEQ ID NO: 48) where the N-terminal amine group of a glycine (G) residue at the first position is cyclized with the side-chain carboxylic acid group of a glutamic acid (E) residue at the eighth position

Codon-optimized DNA encoding the sequences for the lariatin precursor peptide (SEQ ID NO: 35), peptidase (SEQ ID NO: 36), cyclase (SEQ ID NO: 37) and RRE (SEQ ID NO: 38) from Rhodococcus jostii are synthesized (Thermo Fisher Scientific, Carlsbad, Calif.) and individually cloned into a pZE expression vector behind a T7 promoter (Expressys). The resulting plasmids encoding genes for the lariatin precursor peptide (SEQ ID NO: 35), peptidase (SEQ ID NO: 36), cyclase (SEQ ID NO: 37) and RRE (SEQ ID NO: 38) are used with or without a C-terminal affinity tag. Production of lariatin lasso peptide is initiated by adding the lariatin precursor peptide (SEQ ID NO: 35), peptidase (SEQ ID NO: 36), cyclase (SEQ ID NO: 37) and RRE (SEQ ID NO: 38) (15 nM each) to the optimized E. coli BL21 Star(DE3) cell extracts, which is pre-mixed with buffer that contains ATP, GTP, TTP, CTP, amino acids, t-RNA, magnesium glutamate, potassium glutamate, potassium phosphate, and other salts, NAD+, NADPH, and glucose to achieve a total volume of 400 μL. The cell-free biosynthesis of lariatin lasso peptide is accomplished by incubating the reaction for 18 hours at 22° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid faction is subjected to LC/MS analysis on an Agilent 6530 Accurate-Mass Q-TOF MS equipped with a dual electrospray ionization source and an Agilent 1260 LC system with diode array detector for lasso peptide detection. The molecular mass of 2204.12 m/z corresponding to lariatin lasso peptide (GSQLVYREWVGHSNVIKPGP (SEQ ID NO: 48) minus H₂O) is observed. The collected lasso peptide sample is further purified by affinity chromatography and/or preparative HPLC, followed by high resolution mass spectrometry and NMR for structural characterization.

### 6.5 Example 5: Synthesis of Ukn16 Lasso Peptide

Synthesis of ukn16 lasso peptide GVWFGNYVDVGGAKAPFPWGSN (SEQ ID NO: 49) where the N-terminal amine group of a glycine (G) residue at the first position is cyclized with the side-chain carboxylic acid group of an aspartic acid (D) residue at the ninth position

Codon-optimized DNA encoding the sequences for the ukn16 precursor peptide (SEQ ID NO: 39), peptidase (SEQ ID NO: 40), and cyclase-RRE fusion protein (SEQ ID NO: 41) from Bifidobacterium reuteri DSM 23975 are synthesized (Thermo Fisher Scientific, Carlsbad, Calif.) and individually cloned into a pZE expression vector behind a T7 promoter (Expressys). The resulting plasmids encoding genes for the ukn16 precursor peptide (SEQ ID NO: 39), peptidase (SEQ ID NO: 40), and cyclase-RRE fusion protein (SEQ ID NO: 41) are used with or without a C-terminal affinity tag. Production of ukn16 lasso peptide is initiated by adding the ukn16 precursor peptide (SEQ ID NO: 39), peptidase (SEQ ID NO: 40), and cyclase-RRE fusion protein (SEQ ID NO: 41) (15 nM each) to the optimized E. coli BL21 Star(DE3) cell extracts, which is pre-mixed with buffer that contains ATP, GTP, TTP, CTP, amino acids, t-RNA, magnesium glutamate, potassium glutamate, potassium phosphate, and other salts, NAD+, NADPH, and glucose to achieve a total volume of 400 μL. The cell-free biosynthesis of ukn16 lasso peptide is accomplished by incubating the reaction for 18 hours at 22° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid faction is subjected to LC/MS analysis on an Agilent 6530 Accurate-Mass Q-TOF MS equipped with a dual electrospray ionization source and an Agilent 1260 LC system with diode array detector for lasso peptide detection. The molecular mass of 2306.07 m/z corresponding to ukn16 lasso peptide (GVWFGNYVDVGGAKAPFPWGSN (SEQ ID NO: 49) minus H₂O) is observed. The collected lasso peptide sample is further purified by affinity chromatography and/or preparative HPLC, followed by high resolution mass spectrometry and NMR for structural characterization.

### 6.6 Example 6: Synthesis of Adanomysin Lasso Peptide

Synthesis of adanomysin lasso peptide GSSTSGTADANSQYYW (SEQ ID NO: 50) where the N-terminal amine group of a glycine (G) residue at the first position is cyclized with the side-chain carboxylic acid group of an aspartic acid (D) residue at the ninth position

Codon-optimized DNA encoding the sequences for the adanomysin precursor peptide (SEQ ID NO: 42), cyclase (SEQ ID NO: 43), and RRE-peptidase fusion protein (SEQ ID NO: 44) from Streptomyces niveus are synthesized (Thermo Fisher Scientific, Carlsbad, Calif.) and individually cloned into a pZE expression vector behind a T7 promoter (Expressys). The resulting plasmids encoding genes for the adanomysin precursor peptide (SEQ ID NO: 42), cyclase (SEQ ID NO: 43), and RRE-peptidase fusion protein (SEQ ID NO: 44) are used with or without a C-terminal affinity tag. Production of adanomysin lasso peptide is initiated by adding the adanomysin precursor peptide (SEQ ID NO: 42), cyclase (SEQ ID NO: 43), and RRE-peptidase fusion protein (SEQ ID NO: 44) (15 nM each) to the optimized E. coli BL21 Star(DE3) cell extracts, which is pre-mixed with buffer that contains ATP, GTP, TTP, CTP, amino acids, t-RNA, magnesium glutamate, potassium glutamate, potassium phosphate, and other salts, NAD+, NADPH, and glucose to achieve a total volume of 400 μL. The cell-free biosynthesis of adanomysin lasso peptide is accomplished by incubating the reaction for 18 hours at 22° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid faction is subjected to LC/MS analysis on an Agilent 6530 Accurate-Mass Q-TOF MS equipped with a dual electrospray ionization source and an Agilent 1260 LC system with diode array detector for lasso peptide detection. The molecular mass of 1675.66 m/z corresponding to adanomysin lasso peptide (GSSTSGTADANSQYYW (SEQ ID NO: 50) minus H₂O) is observed. The collected lasso peptide sample is further purified by affinity chromatography and/or preparative HPLC, followed by high resolution mass spectrometry and NMR for structural characterization.

### 6.7 Example 7: Synthesis of Ukn22 Lasso Peptide

Synthesis of ukn22 lasso peptide WYTAEWGLELIFVFPRFI (SEQ ID NO: 46) where the N-terminal amine group of a tryptophan (W) residue at the first position is cyclized with the side-chain carboxylic acid group of a glutamic acid (E) residue at the ninth position

Codon-optimized DNA encoding the sequences for the ukn22 precursor peptide (SEQ ID NO: 28), peptidase (SEQ ID NO: 29), cyclase (SEQ ID NO: 30) and RRE (SEQ ID NO: 31) from Thermobifida fusca are synthesized (Thermo Fisher Scientific, Carlsbad, Calif.) and individually cloned into a pZE expression vector (Expressys) behind a maltose binding protein (MBP) sequence to create an N-terminal MBP fusion protein. The resulting plasmids encoding fusion genes for the MBP-ukn22 precursor peptide (SEQ ID NO: 28), MBP-peptidase (SEQ ID NO: 29), MBP-cyclase (SEQ ID NO: 30) and MBP-RRE (SEQ ID NO: 31) are driven by a constitutive T7 promoter. The MBP fusion proteins are produced either separately in individual vessels or in combination in one single vessel by introducing DNA plasmid vectors into the vessel containing E. coli BL21 Star(DE3) cell extracts (15 mg/mL total protein) which is pre-mixed with the buffer described above to achieve a total volume of 50 μL. The MBP fusion proteins are then purified using amylose resin (New England BioLabs) according to the manufacturer's recommendation. The cell-free biosynthesis of ukn22 lasso peptide is accomplished by incubating the isolated MBP fusion proteins for 16 hours at 22° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid faction is subjected to LC/MS analysis on an Agilent 6530 Accurate-Mass Q-TOF MS equipped with a dual electrospray ionization source and an Agilent 1260 LC system with diode array detector for lasso peptide detection. The molecular mass of 2269.18 m/z corresponding to ukn22 lasso peptide (WYTAEWGLELIFVFPRFI (SEQ ID NO: 46) minus H₂O) is observed. The collected lasso peptide sample is further purified by affinity chromatography and/or preparative HPLC, followed by high resolution mass spectrometry and NMR for structural characterization.

### 6.8 Example 8: Screening of Lariatin Lasso Peptide Against G Protein-Couple Receptors (GPCRs)

Isolated lariatin lasso peptide is lyophilized and reconstituted in 100% DMSO to achieve 10 mM stock. Screening of lariatin lasso peptide against a panel of G protein-couple receptors (GPCRs) follows the manufacturer's recommendation (PathHunter® β-Arrestin eXpress GPCR Assay, Eurofins DiscoverX). The screen is performed at both “agonist” and “antagonist” modes if a known nature ligand is available, and only at “agonist” mode if no known ligand is available. The effect of lariatin lasso peptide on the selected GPCRs is measured by β-Arrestin recruitment using a technology developed by Eurofins DiscoverX called Enzyme Fragment Complementation (EFC) with β-galactosidase (β-Gal) as the functional reporter. PathHunter GPCR cells are expanded from freezer stocks according to the manufacture's procedures. Cells are seeded in a total volume of 20 μL into white walled, 384-well microplates and incubated at 37° C. for the appropriate time prior to testing. For agonist determination, cells are incubated with sample to induce response. Intermediate dilution of sample stocks is performed to generate 5× sample in assay buffer. Five microliters of 5× sample is added to cells and incubated at 37° C. or room temperature for 90 to 180 minutes. Vehicle (DMSO) concentration is 1%. For inverse agonist determination, cells are incubated with sample to induce response. Intermediate dilution of sample stocks is performed to generate 5× sample in assay buffer. Five microliters of 5× sample is added to cells and incubated at 37° C. or room temperature for 3 to 4 hours. Vehicle (DMSO) concentration is 1%. Extended incubation is typically required to observe an inverse agonist response in the PathHunter arrestin assay. For antagonist determination, cells are preincubated with antagonist followed by agonist challenge at the EC80 concentration. Intermediate dilution of sample stocks is performed to generate 5× sample in assay buffer. Five microliters of 5× sample is added to cells and incubated at 37° C. or room temperature for 30 minutes. Vehicle (DMSO) concentration is 1%. Five microliters of 6× EC80 agonist in assay buffer is added to the cells and incubated at 37° C. or room temperature for 90 or 180 minutes. After appropriate compound incubation, assay signal is generated through a single addition of 12.5 μL (50% v/v) of PathHunter Detection reagent cocktail for agonist and inverse agonist assays, followed by a one hour incubation at room temperature. For some GPCRs that exhibit low basal signal, activity is detected using a high sensitivity detection reagent (PathHunter Flash Kit) to improve assay performance. For these assays an equal volume (25 μL) of detection reagent is added to the wells and incubated for 1 hour at room temperature. Microplates are read following signal generation with a PerkinElmer Envision® instrument for chemiluminescent signal detection.

### 6.9 Example 9: Creation of a Lasso Peptide Library

To create a library of lasso peptides, codon-optimized DNA encoding the sequences described above for capistruin precursor peptide (SEQ ID NO: 32), capistruin peptidase (SEQ ID NO: 33), capistruin cyclase (SEQ ID NO: 34), lariatin precursor peptide (SEQ ID NO: 35), lariatin peptidase (SEQ ID NO: 36), lariatin cyclase (SEQ ID NO: 37), lariatin RRE (SEQ ID NO: 38), ukn16 precursor peptide (SEQ ID NO: 39), ukn16 peptidase (SEQ ID NO: 40), ukn16 cyclase-RRE fusion protein (SEQ ID NO: 41), adanomysin precursor peptide (SEQ ID NO: 42), adanomysin cyclase (SEQ ID NO: 43), and adanomysin RRE-peptidase fusion protein (SEQ ID NO: 44) are synthesized (Thermo Fisher Scientific, Carlsbad, Calif.) and individually cloned into a pZE expression vector behind a T7 promoter (Expressys). The resulting plasmids encode genes for biosynthesis of capistruin, lariatin, ukn16 and adanomysin with or without a C-terminal affinity tag. Production of the fours lasso peptides in one single vessel are initiated by adding all the plasmids (15 nM each) to the optimized E. coli BL21 Star(DE3) cell extracts, which is pre-mixed with buffer that contains ATP, GTP, TTP, CTP, amino acids, t-RNA, magnesium glutamate, potassium glutamate, potassium phosphate, and other salts, NAD+, NADPH, and glucose to achieve a total volume of 400 μL. The cell-free biosynthesis of the four lasso peptides are accomplished by incubating the reaction for 18 hours at 22° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid fraction is subjected to LC/MS analysis on an Agilent 6530 Accurate-Mass Q-TOF MS equipped with a dual electrospray ionization source and an Agilent 1260 LC system with diode array detector for lasso peptide detection. The molecular mass of 2048.01 m/z corresponding to capistruin lasso peptide (GTPGFQTPDARVISRFGFN minus H₂O), the molecular mass of 2204.12 m/z corresponding to lariatin lasso peptide (GSQLVYREWVGHSNVIKPGP minus H₂O), the molecular mass of 2306.07 m/z corresponding to ukn16 lasso peptide (GVWFGNYVDVGGAKAPFPWGSN minus H₂O), and the molecular mass of 1675.66 m/z corresponding to adanomysin lasso peptide (GSSTSGTADANSQYYW minus H₂O) are observed. The collected lasso peptide sample is further purified by affinity chromatography and/or preparative HPLC, followed by analysis using high resolution mass spectrometry and NMR for structural characterization.

### 6.10 Example 10: Evolution of Lariatin Lasso Peptide Via Site-Saturation Mutagenesis

Codon-optimized DNA encoding the sequences for the lariatin precursor peptide (SEQ ID NO: 35), peptidase (SEQ ID NO: 36), cyclase (SEQ ID NO: 37) and RRE (SEQ ID NO: 38) from Rhodococcus jostii are synthesized (Thermo Fisher Scientific, Carlsbad, Calif.) and individually cloned into a pZE expression vector behind a T7 promoter (Expressys). The resulting plasmids encoding genes for the lariatin precursor peptide (SEQ ID NO: 35), peptidase (SEQ ID NO: 36), cyclase (SEQ ID NO: 37) and RRE (SEQ ID NO: 38) are used with or without a C-terminal affinity tag. To generation a site-saturation library of lariatin lasso peptide variants, each amino acid codon of lariatin core peptide GSQLVYREWVGHSNVIKPGP (SEQ ID NO: 48) is mutagenized to non-parental amino acid codons with the exception of the glutamic acid (E) at the eighth position. The site-saturation mutagenesis is performed using QuikChange Lightning Site-Directed Mutagenesis kit (Agilent Technologies, CA) following the manufacturer's recommended protocol. The mutagenic oligonucleotide primers are synthesized (Integrated DNA Technologies, IL) and used either individually to incorporate a non-parental codon into the lariatin core peptide in a single vessel or in combination to incorporate more than one non-parental codons (e.g., NNK) into the lariatin core peptide in a single vessel. To create combinatorial mutation variants of lariatin lasso peptide during a lasso peptide evolution cycle, the mutagenic oligonucleotide primers are synthesized (Integrated DNA Technologies, IL) to simultaneously incorporate more than one codon change.

Production of a lariatin lasso peptide variant is initiated by adding a mutated lariatin precursor peptide (variant of SEQ ID NO: 35), lariatin peptidase (SEQ ID NO: 36), lariatin cyclase (SEQ ID NO: 37) and lariatin RRE (SEQ ID NO: 38) (15 nM each) in a single vessel containing the optimized E. coli BL21 Star(DE3) cell extracts, which is pre-mixed with buffer that contains ATP, GTP, TTP, CTP, amino acids, t-RNA, magnesium glutamate, potassium glutamate, potassium phosphate, and other salts, NAD+, NADPH, and glucose to achieve a total volume of 400 μL. The cell-free biosynthesis of a lariatin lasso peptide variant is accomplished by incubating the reaction for 18 hours at 22° C. The reaction sample is subsequently diluted in MeOH at 1:1 ratio (v/v) and thoroughly mixed at room temperature for 30 minutes, followed by centrifugation at 14,000 rpm in an Eppendorf benchtop centrifuge to remove precipitated protein. The resulting liquid faction is subjected to LC/MS analysis on an Agilent 6530 Accurate-Mass Q-TOF MS equipped with a dual electrospray ionization source and an Agilent 1260 LC system with diode array detector for lasso peptide detection. The molecular mass corresponding to the lariatin lasso peptide variant (linear peptide sequence minus H₂O) is observed. The collected lasso peptide sample is further purified by affinity chromatography and/or preparative HPLC, followed by high resolution mass spectrometry and NMR for structural characterization.

TABLE 4
The list of protein sequences described in the following Examples 11-17.

| SEQ ID | | | GenBank |
|---|---|---|---|
| NO: | Name | A.A. sequence | Accession # |
| 1 | Ukn22 | WYTAEWGLELIFVFPRFI (W1-E9 cyclized) | N/A |
| | (Thermobifida | | |
| | fusca) | | |
| 2 | Ukn22 precursor A | MEKKKYTAPQLAKVGEFKEATGWYTAE | N/A |
| | (Thermobifida | WGLELIFVFPRFI | |
| | fusca) | | |
| 3 | Ukn22 peptidase B | MSENVVLQRSNVRLSWRTKWAARCAVG | WP_011291590 |
| | (Thermobifida | AARLLARKPPERIRATLLRLRGEVRPATY | |
| | fusca) | EEAKAARDAVLAVSLRCAGLRACLQRSL | |
| | | AIALLCRMRGTWATWCVGVPRRPPFIGH | |
| | | AWVEAEGRLVEEGVGYDYFSRLITVD | |
| 4 | Ukn22 cyclase C | MVGCISPYFAVFPDKDVLGQATDRLPAA | WP_011291592 |
| | (Thermobifida | QTLASHPSGRPWLVGALPADQLLLVEAG | |
| | fusca) | ERRLAVIGHCSAEPERLRAELAQIDDVAQ | |
| | | FDRIARTLDGSFHLVVVVGDQMRIQGSV | |
| | | SGLRRVFHAHVGTARIAADRSDVLAAVL | |
| | | GVSPDPDVLALRMFNGLPYPLSELPPWPG | |
| | | VEHVPAWHYLSLGLHDGRHRVVQWWH | |
| | | PPEAELAVTAAAPLLRTALAGAVDTRTR | |
| | | GGGVVSADLSGGLDSTPLCALAARGPAK | |
| | | VVALTFSSGLDTDDDLRWAKIAHQSFPS | |
| | | VEHVVLSPEDIPGFYAGLDGEFPLLDEPS | |
| | | VAMLSTPRILSRLHTARAHGSRLHMDGL | |
| | | GGDQLLTGSLSLYHDLLWQRPWTALPLI | |
| | | RGHRLLAGLSLSETFASLADRRDLRAWL | |
| | | ADIRHSIATGEPPRRSLFGWDVLPKCGPW | |
| | | LTAEARERVLARFDAVLESLEPLAPTRGR | |
| | | HADLAAIRAAGRDLRLLHQLGSSDLPRM | |
| | | ESPFLDDRVVEACLQVRHEGRMNPFEFK | |
| | | SLMKTAMASLLPAEFLTRQSKTDGTPLA | |
| | | AEGFTEQRDRIIQIWRESRLAELGLIHPDV | |
| | | LVERVKQPYSFRGPDWGMELTLTVELWL | |
| | | RSRERVLQGANGGDNRS | |
| 5 | Ukn22 RRE | METTGAEFRLRPEISVAQTDYGMVLLDG | WP_011291591 |
| | (Thermobifida | RSGEYWQLNDTAALIVQRLLDGHSPADV | |
| | fusca) | AQFLTSEYEVERTDAERDIAALVTSLKEN | |
| | | GMALP | |
| 6 | BI-32169 | GLPWGCPSDIPGWNTPWAC (G1-D9 | N/A |
| | (Streptomyces sp. | cyclized) | |
| | DSM 14996) | | |
| 7 | BI-32169 analog | GLPWGCPNDLFFVNTPFAC (G1-D9 | N/A |
| | (Kibdelosporangium | cyclized) | |
| | sp. MJ126-NF4) | | |
| 8 | BI-32169 analog | MIKDDEIYEVPTLVEVGDFAELTLGLPWG | N/A |
| | precursor A | CPNDLFFVNTPFAC | |
| | (Kibdelosporangium | | |
| | sp. MJ126-NF4) | | |
| 9 | Hybrid BI-32169 | MIKDDEIYEVPTLVEVGDFAELTLGLPWG | N/A |
| | precursor A | CPSDIPGWNTPWAC | |
| 10 | BI-32169 analog | MTMPVAAETTVPLPWHRHITARLATGSA | WP_042177890 |
| | peptidase B | RVLIRLRPRRLRVVLRMVSRGARPATAA | |
| | (Kibdelosporangium | QALSARQAVVSVSVRCAGQGCLQRAVA | |
| | sp. MJ126-NF4) | TALLCRLAGDWPDWCTGFRTRPFRAHA | |
| | | WVEAEGGAVGEPGDMPLFHTVISVRHPA | |
| | | REAR | |
| 11 | BI-32169 analog | MRDRRWRAGVRPSTADAGTKGKGLLVG | WP_083466052 |
| | cyclase C | GNEFLVFPDCPVALDAPGGRTVPHASGR | |
| | (Kibdelosporangium | PWLVGDWSDDDIVVISAGTRRLAIVGQA | |
| | sp. MJ126-NF4) | RVNVHAVERSLEAAGSVRDLDAVVGTIP | |
| | | GNFHLIASIDGRTRVQGTVSTVRQVFTAT | |
| | | IVGTTVAASGPGLLAAATGSRVDGDALA | |
| | | LRLVPVVPWPLCLRPVWSGVEQVAAGH | |
| | | WL | |
| 12 | BI-32169 analog | MTIALTPNVTATDSEDGLVLLNESTGRY | WP_042177888 |
| | RRE | WTLNGTGAATLRLLLAGNSPAQTASRLA | |
| | (Kibdelosporangium | ERYPDAVDRTQRDVVALLAALRNARLV | |
| | sp. MJ126-NF4) | TSS | |
| 13 | PelB secretion | MKYLLPTAAAGLLLLAAQPAMA↓ | N/A |
| | sequence (ssPelB) | | |
| 14 | TorA secretion | MNNNDLFQASRRRFLAQLGGLTVAGML | N/A |
| | sequence (ssTorA) | GPSLLTPRRATA↓AQA | |
| 15 | TEV cleavage site | ENLYFQ↓G | N/A |
| 16 | Linker 1 | GAAAKGAAAKGAAAKGAAAK | N/A |
| 17 | Linker 2 | SGGGGSGGGGSGGGGSGGGGSGGGG | N/A |
| 18 | Truncated | MKIEEGKLVIWINGDKGYNGLAEVGKKF | WP_052916395 |
| | maltose-binding | EKDTGIKVTVEHPDKLEEKFPQVAATGD | |
| | protein (MBP) | GPDIIFWAHDRFGGYAQSGLLAEITPDKA | |
| | (deletion 2-29) | FQDKLYPFTWDAVRYNGKLIAYPIAVEA | |
| | | LSLIYNKDLLPNPPKTWEEIPALDKELKA | |
| | | KGKSALMFNLQEPYFTWPLIAADGGYAF | |
| | | KYENGKYDIKDVGVDNAGAKAGLTFLV | |
| | | DLIKNKHMNADTDYSIAEAAFNKGETAM | |
| | | TINGPWAWSNIDTSKVNYGVTVLPTFKG | |
| | | QPSKPFVGVLSAGINAASPNKELAKEFLE | |
| | | NYLLTDEGLEAVNKDKPLGAVALKSYEE | |
| | | ELAKDPRIAATMENAQKGEIMPNIPQMS | |
| | | AFWYAVRTAVINAASGRQTVDEALKDA | |
| | | QTRITK | |
| 19 | Streptavidin- | MDEKTTGWRGGHVVEGLAGELEQLRAR | N/A |
| | binding Peptide | LEHHPQGQREP | |
| | (SBP) | | |
| 20 | Replication protein | MTDLHQTYYRQVKNPNPVFTPREGAGTP | AAA99917 |
| | RepA (RepA) | KFREKPMEKAVGLTSRFDFAIHVAHARS | |
| | | RGLRRRMPPVLRRRAIDALLQGLCFHYD | |
| | | PLANRVQCSITTLAIECGLATESGAGKLSI | |
| | | TRATRALTFLSELGLITYQTEYDPLIGCYI | |
| | | PTDITFTLALFAALDVSEDAVAAARRSRV | |
| | | EWENKQRKKQGLDTLGMDELIAKAWRF | |
| | | VRERFRSYQTELQSRGIKRARARRDANR | |
| | | ERQDIVTLVKRQLTREISEGRFTANGEAV | |
| | | KREVERRVKERMILSRNRNYSRLATASP | |
| 21 | Capistruin | GTPGFQTPDARVISRFGFN(G1-D9 | |
| | (Burkholderia | cyclized) | |
| | thailandensis) | | |
| 22 | Capistruin | MVRLLAKLLRSTIHGSNGVSLDAVSSTH | WP_009905508 |
| | precursor A | GTPGFQTPDARVISRFGFN | |
| | (Burkholderia | | |
| | thailandensis) | | |
| 23 | Capistruin | MTPASHCHIAVFDQAIVALDMQRSRYFL | WP_009905509 |
| | peptidase B | YDEACAKAFADHYLDFKPIDAPHALKPLI | |
| | (Burkholderia | SDRIVVAASPASVPKRIADYRGWAFDAF | |
| | thailandensis) | DSGIWASRTLGERSAAGFEWLPFWRIVR | |
| | | GAVSLKMRGFRALSALDRLARLDAGAE | |
| | | QRARTDGGPSRTAERYLRASIWSPFRITC | |
| | | LQMSFALATHLRRENVPAQLVIGVRPMP | |
| | | FVAHAWVEIDGRVCGDEPELKKSYGEIY | |
| | | RTPRHDERAGPFGLAA | |
| 24 | Capistruin cyclase | MTLLEAGARARAYLRDAHSRIERSLARA | WP_045600732 |
| | C | RTLQEARDTVTRSVWGAYLLVLDEAASG | |
| | (Burkholderia | RRLFMPDPLHSVRLYYRTDERGRVDVDP | |
| | thailandensis) | RAANLLDRASIDWNLDYLIEFACTQFGPL | |
| | | DETPFASVRVVPPGCALVVGPDGRCAIER | |
| | | AWLPRAQAAGDVRASCAAALDDVYSRI | |
| | | AHSHPSVCAALSGGVDSSAGAIFLRKALG | |
| | | ANAPLAAVHLYSTSSPDCYERDMAARVA | |
| | | DSIGAQLICIDIDRHLPFSERIVRTPPAALN | |
| | | QDMLFLGIDRAVSNALGPSSVLLEGQGG | |
| | | DLLFRAVPDANAVLDALRSNGWSFALRT | |
| | | AEKLAMLHNDSIPRILLMAAKIALRRRLF | |
| | | GQDAPASQQTMSRLFASSAPRAAAGRSR | |
| | | RHAPRADAPLDESISMLDRFVSIMTPVTD | |
| | | AAYTSRLNPYLAQPVVEAAFGLRSYDSF | |
| | | DHRNDRIVLREIASAHTPVDVLWRRTKG | |
| | | SFGIGFVKGIVSHYDALRELIRDGVLMRS | |
| | | GRLDEAELEHALKAVRVGQNAAAISVAL | |
| | | VGCVEVFCASWQNFVTNRHAAVC | |
| 51 | Fusilassin | WYTAEWGLELIFVFPRFI (W1-E9 cyclized) | |
| 52 | FusA | MEKKKYTAPQLAKVGEFKEATGWYTAE | NC_07333.1 |
| | (Thermobifida | WGLELIFVFPRFI | |
| | fusca) | | |
| 53 | Fusilassin cyclase | MVGCISPYFAVFPDKDVLGQATDRLPAA | WP_011291592.1 |
| | FusC | QTLASHPSGRPWLVGALPADQLLLVEAG | |
| | (Thermobifida | ERRLAVIGHCSAEPERLRAELAQIDDVAQ | |
| | fusca) | FDRIARTLDGSFHLVVVVGDQMRIQGSV | |
| | | SGLRRVFHAHVGTARIAADRSDVLAAVL | |
| | | GVSPDPDVLALRMFNGLPYPLSELPPWPG | |
| | | VEHVPAWHYLSLGLHDGRHRVVQWWH | |
| | | PPEAELAVTAAAPLLRTALAGAVDTRTR | |
| | | GGGVVSADLSGGLDSTPLCALAARGPAK | |
| | | VVALTFSSGLDTDDDLRWAKIAHQSFPS | |
| | | VEHVVLSPEDIPGFYAGLDGEFPLLDEPS | |
| | | VAMLSTPRILSRLHTARAHGSRLHMDGL | |
| | | GGDQLLTGSLSLYHDLLWQRPWTALPLI | |
| | | RGHRLLAGLSLSETFASLADRRDLRAWL | |
| | | ADIRHSIATGEPPRRSLFGWDVLPKCGPW | |
| | | LTAEARERVLARFDAVLESLEPLAPTRGR | |
| | | HADLAAIRAAGRDLRLLHQLGSSDLPRM | |
| | | ESPFLDDRVVEACLQVRHEGRMNPFEFK | |
| | | SLMKTAMASLLPAEFLTRQSKTDGTPLA | |
| | | AEGFTEQRDRIIQIWRESRLAELGLIHPDV | |
| | | LVERVKQPYSFRGPDWGMELTLTVELWL | |
| | | RSRERVLQGANGGDNRS | |
| 54 | Fusilassin | MSENVVLQRSNVRLSWRTKWAARCAVG | WP_011291590.1 |
| | peptidase FusB | AARLLARKPPERIRATLLRLRGEVRPATY | |
| | (Thermobifida | EEAKAARDAVLAVSLRCAGLRACLQRSL | |
| | fusca) | AIALLCRMRGTWATWCVGVPRRPPFIGH | |
| | | AWVEAEGRLVEEGVGYDYFSRLITVD | |
| 55 | Fusilassin RRE | METTGAEFRLRPEISVAQTDYGMVLLDG | WP_011291591.1 |
| | FusE | RSGEYWQLNDTAALIVQRLLDGHSPADV | |
| | (Thermobifida | AQFLTSEYEVERTDAERDIAALVTSLKEN | |
| | fusca) | GMALP | |
| 56 | Fusilassin ABC | MPLSPPRSLRLLVAHLWPHRRAVAFGAL | WP_011291589.1 |
| | transporter FusD | LGLLGGIGTLAEPLVAMAVVDALGEGSP | |
| | (Thermobifida | LGWLLALLTVLVVGGAALAGLSSYVLHR | |
| | fusca) | TAESMVAAARRRLVSHILLLRVPELDRLK | |
| | | PGDLLSRVTSDTTYIRSAAGQALVDSGSA | |
| | | LLVAIGSIVLMAWIDLPLLLVCLAVIGVIG | |
| | | VGSAVMMPPIRRANERSQRAVGEVGALV | |
| | | ERALGAFRTLKASSAERREISAAKAAVRT | |
| | | AWREGVRSAAWTAATNVAVVVTSQAAF | |
| | | LVVLGAGGARVAMGAIDVSELIAFLLYL | |
| | | MRLTGFVAQLAQAVSSLQSGLAAMRRIA | |
| | | EVEQLPVEHIGVPPRRTPAATSAASVSFT | |
| | | GVSFRYREDGPWTLRNVTLDVPAGGLTA | |
| | | LVGPSGAGKTTLFSLVERFYDPHEGVVAI | |
| | | DGVDVRDIPLVRLRSMIGYVEQDAPILAG | |
| | | TLRDNLCFAAPHADEEEIRRVVELTRLTS | |
| | | LVERLPDGLDTQVGHRGTTLSGGERQRV | |
| | | AIARALLRRPRLLLLDEATSQLDATNETA | |
| | | LRDVVVAIAKTTTVIIIAHRLSTVVDADRI | |
| | | AVVEGGRIRAVGRHTDLLLIDDLYRELIE | |
| | | AQLLAS | |
| 57 | MBP-FusA-TEV- | MKIEEGKLVIWINGDKGYNGLAEVGKKF | |
| | SBP | EKDTGIKVTVEHPDKLEEKFPQVAATGD | |
| | | GPDIIFWAHDRFGGYAQSGLLAEITPDKA | |
| | | FQDKLYPFTWDAVRYNGKLIAYPIAVEA | |
| | | LSLIYNKDLLPNPPKTWEEIPALDKELKA | |
| | | KGKSALMFNLQEPYFTWPLIAADGGYAF | |
| | | KYENGKYDIKDVGVDNAGAKAGLTFLV | |
| | | DLIKNKHMNADTDYSIAEAAFNKGETAM | |
| | | TINGPWAWSNIDTSKVNYGVTVLPTFKG | |
| | | QPSKPFVGVLSAGINAASPNKELAKEFLE | |
| | | NYLLTDEGLEAVNKDKPLGAVALKSYEE | |
| | | ELAKDPRIAATMENAQKGEIMPNIPQMS | |
| | | AFWYAVRTAVINAASGRQTVDEALKDA | |
| | | QTNSSSHRHHHHANSVPLVPRGSENLYF | |
| | | QSGSMEKKKYTAPQLAKVGEFKEATGW | |
| | | YTAEWGLELIFVFPRFIGGGGSGGGGSGG | |
| | | GGSYPYDVPDYAENLYFQGMDEKTTGW | |
| | | RGGHVVEGLAGELEQLRARLEHHPQGQR | |
| | | EP | |
| 58 | Fusilassin-TEV- | WYTAEWGLELIFVFPRFIGGGGSGGGGS | |
| | SBP | GGGGSYPYDVPDYAENLYFQGMDEKTT | |
| | | GWRGGHVVEGLAGELEQLRARLEHHPQ | |
| | | GQREP (W1-E9 cyclized) | |
| 59 | TEV protease- | WYTAEWGLELIFVFPRFIGGGGSGGGGS | |
| | cleaved Fusilassin | GGGGSYPYDVPDYAENLYFQ (W1-E9 | |
| | | cyclized) | |
| 60 | Streptavidin core | GSGAAEAGITGTWYNQLGSTFIVTAGAD | |
| | region (SAV) | GALTGTYESAVGNAESRYVLTGRYDSAP | |
| | | ATDGSGTALGWTVAWKNNYRNAHSATT | |
| | | WSGQYVGGAEARINTQWLLTSGTTEAN | |
| | | AWKSTLVGHDTFTKVKPSAASIDAAKKA | |
| | | GVNNGNPLDAVQQ | |
| 61 | FusA-TEV-SAV | MEKKKYTAPQLAKVGEFKEATGWYTAE | |
| | | WGLELIFVFPRFIGGGSVSYTHLRAHETE | |
| | | NLYFQGSGAAEAGITGTWYNQLGSTFIVT | |
| | | AGADGALTGTYESAVGNAESRYVLTGR | |
| | | YDSAPATDGSGTALGWTVAWKNNYRNA | |
| | | HSATTWSGQYVGGAEARINTQWLLTSGT | |
| | | TEANAWKSTLVGHDTFTKVKPSAASIDA | |
| | | AKKAGVNNGNPLDAVQQ | |
| 62 | MBP-FusA-TEV- | MKIEEGKLVIWINGDKGYNGLAEVGKKF | |
| | SAV | EKDTGIKVTVEHPDKLEEKFPQVAATGD | |
| | | GPDIIFWAHDRFGGYAQSGLLAEITPDKA | |
| | | FQDKLYPFTWDAVRYNGKLIAYPIAVEA | |
| | | LSLIYNKDLLPNPPKTWEEIPALDKELKA | |
| | | KGKSALMFNLQEPYFTWPLIAADGGYAF | |
| | | KYENGKYDIKDVGVDNAGAKAGLTFLV | |
| | | DLIKNKHMNADTDYSIAEAAFNKGETAM | |
| | | TINGPWAWSNIDTSKVNYGVTVLPTFKG | |
| | | QPSKPFVGVLSAGINAASPNKELAKEFLE | |
| | | NYLLTDEGLEAVNKDKPLGAVALKSYEE | |
| | | ELAKDPRIAATMENAQKGEIMPNIPQMS | |
| | | AFWYAVRTAVINAASGRQTVDEALKDA | |
| | | QTNSSSHRHHHHANSVPLVPRGSENLYF | |
| | | QSGSMEKKKYTAPQLAKVGEFKEATGW | |
| | | YTAEWGLELIFVFPRFIGGGGSGGGGSGG | |
| | | GGSYPYDVPDYAENLYFQGSGAAEAGIT | |
| | | GTWYNQLGSTFIVTAGADGALTGTYESA | |
| | | VGNAESRYVLTGRYDSAPATDGSTALG | |
| | | WTVAWKNNYRNAHSATTWSGQYVGGA | |
| | | EARINTQWLLTSGTTEANAWKSTLVGHD | |
| | | TFTKVKPSAASIDAAKKAGVNNGNPLDA | |
| | | VQQ | |
| 63 | Fusilassin-TEV- | WYTAEWGLELIFVFPRFIGGGGSGGGGS | |
| | SAV | GGGGSYPYDVPDYAENLYFQGSGAAEA | |
| | | GITGTWYNQLGSTFIVTAGADGALTGTY | |
| | | ESAVGNAESRYVLTGRYDSAPATDGSTA | |
| | | LGWTVAWKNNYRNAHSATTWSGQYVG | |
| | | GAEARINTQWLLTSGTTEANAWKSTLVG | |
| | | HDTFTKVKPSAASIDAAKKAGVNNGNPL | |
| | | DAVQQ (W1-E9 cyclized) | |
| 64 | TEV cleavage site | ENLYFQ | |
| | partial sequence | | |

### 6.11 Example 11: Linking Lasso Peptide and DNA Barcode on Beads in Individual Wells

To display a lasso peptide on the surface of a bead as shown in FIG. 5A, two recombinant DNA plasmid vectors are generated: (1) the ukn22 A-TEV-SBP plasmid vector for production of a ukn22 precursor peptide A fused at the C-terminus to the TEV protease recognition sequence and the streptavidin binding peptide (SBP) and (2) the MBP-B/MBP-C/MBP-RRE plasmid vector for production of ukn22 peptidase (B), cyclase (C) and RiPP Recognition Element (RRE), each of which is fused to the C-terminus of maltose binding protein (MBP). The expression of these four fusion proteins is carried out using Cell-Free Biosynthesis (CFB) technology in an in vitro transcription-translation (TX-TL) reaction. During the incubation of the TX-TL reaction, the ukn22 precursor peptide A is expressed, cleaved and cyclized by the ukn22 synthetase enzymes B, C and RRE to produce ukn22 lasso peptide fusion protein—“ukn22-TEV-SBP.” The generated ukn22-TEV-SBP is then mixed with streptavidin-coated magnetic beads, which are pre-bound with biotinylated dsDNA molecules that serve as a DNA barcode. The presence of the TEV protease recognition sequence in the ukn22-TEV-SBP fusion protein allows TEV protease-mediated cleavage to release ukn22 for validation of lasso conformation by mass spectrometry.

To generate the ukn22 A-TEV-SBP plasmid vector, the coding sequence for ukn22 precursor peptide A is cloned in front of the SBP coding sequence and behind a constitutive T7 promoter. The coding sequence for the TEV protease recognition site (Glu-Asn-Leu-Tyr-Phe-Gln⬇Gly) (SEQ ID NO:15) flanked by two linker sequences, Linker 1 and Linker 2, is then inserted in-frame between the ukn22 precursor peptide A and the SBP. The constructed ukn22 A-TEV-SBP coding sequence is then cloned into a plasmid vector containing a pUC E. coli replication origin and the ampicillin resistance gene.

To generate the MBP-B/MBP-C/MBP-RRE plasmid vector, the coding sequences for ukn22 peptidase (B), cyclase (C) and RiPP recognition element (RRE) are cloned in-frame behind the maltose binding protein (MBP) to create three fusion proteins, MBP-B, MBP-C and MBP-RRE, each of which is expressed from an independent, constitutive T7 promoter on a plasmid containing the chloramphenicol resistance gene.

To link lasso peptide and DNA barcode on the same bead, the ukn22 A-TEV-SBP plasmid vector (10 ng) and the MBP-B/MBP-C/MBP-RRE plasmid vector (10 ng) are added into a total of 40 μL CFB reaction in a well of the 384-well PCR plate. The reaction is incubated at 37° C. for 16 hours to produce the ukn22 A-TEV-SBP, MBP-B, MBP-C and MBP-RRE fusion proteins. During the 16-hour incubation, the ukn22 leader sequence at the N-terminus of precursor peptide A is cleaved and the core peptide of A is cyclized by MBP-B, MBP-C and MBP-RRE to form ukn22 lasso peptide with a threaded tail fused to TEV and SBP—“ukn22-TEV-SBP.” Following the 16 hour incubation, the streptavidin-coated magnetic beads (Dynabeads™ MyOne™ Streptavidin T1, Thermo Fisher Scientific, Cat. #65601) pre-bound with biotinylated dsDNA molecules (Integrated DNA Technologies) are added to the well containing the produced ukn22-TEV-SBP fusion protein. The quantity of the bound biotinylated dsDNA is adjusted so that at least more than 95% of streptavidin-coated bead surface remains available for SBP-streptavidin binding. The conjugation reaction takes place at 4° C. for an hour with gentle shaking. Following the one-hour incubation, the 384-well PCR plate is placed on a 384 magnet plate (Alpaqua) to immobilize the magnetic beads and the TX-TL reaction mixture within the well is aspirated. The immobilized magnetic beads in the well are washed three times with 50 μL ice-cold TNTB Wash Buffer (0.1 M Tris pH 7.5, 0.15 M NaCl, 0.05% Tween-20, 1% bovine serum albumin). Upon the aspiration of the last Wash Buffer, the immobilized magnetic beads are resuspended in 20 μL of TNTB buffer and used for affinity selection.

To verify successful display of ukn22 lasso peptide on the beads, 5 μL of the resuspended magnetic beads is treated with TEV protease (Sigma Cat. #T4455) to release ukn22 lasso peptide following the manufacturer's instructions. An equal volume of methanol is then added to the digestion reaction and thoroughly mixed. The ukn22 lasso peptide released into the supernatant post-digestion is aspirated and transferred to a new 384-well PCR plate while the TEV-SBP fusion protein bound to the magnetic beads remain immobilized on the original 384-well PCR plate by a 384 magnet plate. The collected supernatant is subsequently concentrated and subjected to MALDT-TOF MS analysis to verify the presence of ukn22 lasso peptide fused to Linker 1 and part of TEV protease recognition site (Ukn22-Linker 1-Glu-Asn-Leu-Tyr-Phe-Gln (SEQ ID NO:64)). To confirm the simultaneous presence of the DNA barcode on the beads, 1 μL of the resuspended magnetic beads is used for DNA amplification with polymerase chain reaction (PCR). The amplified dsDNA is subjected to DNA sequencing to verify the presence of the DNA barcode.

The following example demonstrates that a Fusilassin-TEV-SBP (SEQ ID NO:58) fusion protein produced by cell free biosynthesis bound a magnetic streptavidin bead and was correctly formed. A linear DNA template for MBP-FusA-TEV-SBP (SEQ ID NO:57) was generated by PCR containing a T7 promoter and ribosomal binding site upstream of the coding region. The linear DNA template thus obtained was incubated with the PURExpress System (New England Biolabs, Ipswich, Mass.) per the manufacturer's recommendation to obtain the MBP-FusA-TEV-SBP. In a similar fashion, a linear DNA template for MBP-FusA-TEV-SBP was incubated with E. coli BL21 DE3 lysate supplemented by GamS (both provided by Genomatica, Inc., San Diego, Calif.) to produce MBP-FusA-TEV-SBP.

To the cell-free reactions above containing MBP-FusA-TEV-SBP was added the purified enzymes (20 μM each) FusB (SEQ ID NO:54), FusC (SEQ ID NO:53), and FusE (SEQ ID NO:55). Incubation for 12 h led to full conversion to the folded lasso peptide product Fusilassin-TEV-SBP.

Fusilassin-TEV-SBP formed in the cell free biosynthesis reactions above was incubated with magnetic streptavidin beads (Dynabeads, Thermo Fisher, Waltham, Mass.) to demonstrate binding and purification. Fifty microliters of magnetic streptavidin beads (0.5 mg) in PBS buffer (10 mM Na₂HPO₄, 1.8 mM KH₂PO₄ pH=7.4, 137 mM NaCl, 2.7 mM KCl) were added and the reactions were incubated for 60 min. The beads with Fusilassin-TEV-SBP bound were separated from the solution with a magnet and washed three times with PBS buffer. The beads were incubated with TEV protease to release the cleaved lasso peptide product (SEQ ID NO:59). After separating the beads with a magnet, the eluate was purified and concentrated with a ZipTip (EMD Millipore, Burlington, Mass.) and analyzed using MALDI MS. A clear m/z peak of 5095 was demonstrated as expected for the correctly formed TEV protease-cleaved Fusilassin product which was liberated from the bead.

Enzymes used in this Example were produced as maltose binding protein (MBP) fusions in E. coli. Chemically competent E. coli BL21 (DE3) cells were co-transformed with pET28-MBP-FusB or pET28-MBP-FusE and plated on LB agar plates supplemented with 50 μg/mL kanamycin and grown at 37° C. overnight. For FusC, cells were co-transformed with pET28-MBP-FusC and pGro7 chaperone plasmid (Takara Bio USA, Inc., Mountain View, Calif.) and plated on LB agar plates supplemented with 50 μg/mL kanamycin and 37 μg/mL chloramphenicol and grown at 37° C. overnight. A single colony was used to inoculate 10 mL of LB supplemented with kanamycin and chloramphenicol (as needed), grown for 12 h at 37° C. Cultures were used to inoculate 1L of LB containing 25 μg/mL kanamycin, 17 μg/mL chloramphenicol, and 0.5-4 mg/mL L-arabinose, which were grown at 37° C. to an OD600 of 0.7-0.8. Protein expression was induced by the addition of IPTG to a final concentration of 0.5 mM and cultures were grown at 18° C. for 16 h. Protein purification by amylose resin affinity chromatography was performed by applying the sonicated pellet lysate to a pre-equilibrated amylose resin (5 mL of resin per L of culture, New England Biolabs, Ipswich, Mass.). The column was washed with 10 column volumes (CV) of lysis buffer followed by 10 CV of wash buffer (lysis buffer without Triton X-100) per the manufacturer's recommended protocol. The MBP-tagged proteins were eluted with 15 mL elution buffer (lysis buffer with 300 mM NaCl, 10 mM maltose, and lacking Triton X-100) and collected into an appropriate molecular weight cutoff (MWCO) Amicon Ultra centrifugal filter (EMD Millipore, Burlington, Mass.). Protein eluent was concentrated to ˜1.5 mL and exchanged with 10× volume of protein storage buffer [50 mM HEPES pH 7.5, 300 mM NaCl, 0.5 mM tris-(2-carboxyethyl)-phosphine (TCEP), 2.5% glycerol (v/v)]. Protein concentrations were assayed using 280 nm absorbance (theoretical extinction coefficients were calculated using the ExPASy ProtParam tool; http://web.expasy.org/protparam/protpar-ref.html). Final protein purity was assessed visually using a Coomassie-stained SDS-PAGE gel.

The following Example demonstrates production of Fusilassin-TEV-SBP (SEQ ID NO: 58) directly from a biotinylated linear DNA template bound to a magnetic streptavidin bead and the resulting product Fusilassin-TEV-SBP thus formed also bound to the same bead. A linear DNA template for MBP-FusA-TEV-SBP (SEQ ID NO: 57) was generated by PCR containing a T7 promoter and ribosomal binding site upstream of the coding region. Furthermore, the 3′ primer containing a biotin tag introduced a 3′ biotin into the DNA amplicon. The linear DNA template (500 ng) was incubated with 50 μl magnetic streptavidin beads (0.5 mg, Thermo Fisher, Waltham, Mass.) in Bind&Wash buffer (50 mM Tris pH=7.5, 0.5 mM EDTA, 1M NaCl) for 30 min. The linear DNA template plus beads were separated from the solution with a magnet and washed three times with Bind&Wash buffer. The DNA-bound beads were combined with E. coli BL21 DE3 cell lysate (provided by Genomatica, Inc., San Diego, Calif.), and the purified enzymes FusB, FusC and FusE, and the cell free reaction was incubated overnight at room temperature in the presence of GamS enzyme (5 μM). Beads containing both the linear biotinylated DNA template and newly produced Fusilassin-TEV-SBP were separated from the solution with a magnet and were washed three times with PBS buffer (10 mM Na₂HPO₄, 1.8 mM KH₂PO₄ pH=7.4, 137 mM NaCl, 2.7 mM KCl) and then incubated with TEV protease for 3 h to release the formed lasso peptide product (SEQ ID NO: 59). The beads were separated from the solution with a magnet and the eluate was purified and concentrated with an EMD Millipore ZipTip and analyzed using MALDI MS. A clear m/z peak of 5095 was observed as expected for folded mature Fusilassin product cleaved from the beads by TEV protease. Similar studies with a biotin tag linked to the 5′ DNA template demonstrated identical results.

In a similar fashion, the biotinylated linear DNA template for MBP-FusA-TEV-SBP was incubated for 30 min with the PURExpress System (New England Biolabs, Ipswich, Mass.) and magnetic streptavidin beads. Subsequently, purified enzymes FusB, FusC, and FusE were added to the reaction to form Fusilassin-TEV-SBP bound to the bead. Beads containing the linear biotinylated DNA template and Fusilassin-TEV-SBP were separated from the solution with a magnet and were washed three times with PBS buffer (10 mM Na₂HPO₄, 1.8 mM KH₂PO₄ pH=7.4, 137 mM NaCl, 2.7 mM KCl) and incubated with TEV protease for 3 h to release the formed lasso peptide product. The beads were separated from the solution with a magnet and the eluate was purified and concentrated with an EMD Millipore ZipTip and analyzed by MALDI MS. A clear m/z peak of 5095 was observed, as expected for the folded mature Fusilassin product cleaved from the bead by TEV protease. Similar studies with a biotin tag linked to the 5′ DNA template demonstrated consistent results.

### 6.12 Example 12: Linking Lasso Peptide and DNA Barcode on Beads in a Water-in-Oil Emulsion

To display a lasso peptide on the surface of a bead as shown in FIG. 6A, two recombinant DNA molecules are generated: (1) a linear, biotinylated dsDNA sequence encoding ukn22 A-TEV-SBP fusion protein and (2) the MBP-B/MBP-C/MBP-RRE plasmid vector for production of ukn22 peptidase (B), cyclase (C) and RiPP Recognition Element (RRE), each of which is fused to the C-terminus of maltose binding protein (MBP). The biotinylated dsDNA sequence is designed to simultaneously serve as a unique DNA barcode for identification (genotype) and the DNA template for expression of the ukn22 A-TEV-SBP fusion protein (phenotype). To link genotype and phenotype on the same solid support, the biotinylated dsDNA molecule is pre-bound to streptavidin-coated beads at the 1:1 ratio of dsDNA molecules to beads, followed by the addition of the MBP-B/MBP-C/MBP-RRE plasmid vector and the CFB cell extracts containing all necessary components for in vitro transcription-translation (TX-TL) reaction. The combined TX-TL reaction is used as the aqueous phase to generate a water-in-oil emulsion as described by Tawfik and Griffiths (See: Nat. Biotech. 1998, 652-656). The emulsion is then incubated at 37° C. for two hours to express the four fusion proteins, ukn22 A-TEV-SBP, MBP-B, MBP-C and MBP-RRE, in a single aqueous droplet. Upon expression of the ukn22 A-TEV-SBP fusion protein, the streptavidin binding peptide (SBP) at the C-terminus binds to the streptavidin-coated beads in the same aqueous droplet. To catalyze the lasso formation, the emulsion is further incubated at 37° C. for 14 hours. During the 14-hour incubation, the leader sequence at the N-terminus of ukn22 precursor peptide A is cleaved and cyclized by MBP-B, MBP-C and MBP-RRE to form ukn22 lasso peptide with a threaded tail fused to TEV and SBP—“ukn22-TEV-SBP.” The presence of the TEV protease recognition sequence in the ukn22-TEV-SBP fusion protein allows TEV protease-mediated cleavage to release ukn22 from the rest of the fusion protein for validation of lasso conformation by mass spectrometry.

To generate the biotinylated dsDNA molecule, the dsDNA sequence, including a T7 promoter and the coding sequence for ukn22 A-TEV-SBP fusion protein, is synthesized by a DNA manufacturer (Integrated DNA Technologies). Biotinylation is achieved by incorporating a biotinylated 5′ DNA primer into the amplified dsDNA molecules with polymerase chain reaction (PCR). The biotinylated dsDNA molecule is then mixed with streptavidin-coated magnetic beads (Dynabeads™ MyOne™ Streptavidin T1, Thermo Fisher Scientific, Cat. #65601) in 50 μL of TNTB buffer (0.1 M Tris pH 7.5, 0.15 M NaCl, 0.05% Tween-20, 1% bovine serum albumin). The dsDNA/bead mixture is then incubated overnight at 4° C. to achieve the 1:1 ratio of dsDNA molecules to beads. After the overnight incubation, the beads are washed twice with TNTB buffer and then resuspended in 50 μl of ice-cooled CFB cell extracts containing the MBP-B/MBP-C/MBP-RRE plasmid vector.

To create an water-in-oil emulsion, the oil phase is freshly prepared by dissolving 4.5% (vol/vol) Span 80 (Sigma, CAT. #85548) in mineral oil (Sigma, CAT. #M5904) followed by 0.5% (vol/vol) Tween 80 (Sigma, CAT. #P1754). The ice-cooled beads/CFB mixtures (50 μL) are added gradually to 950 μL of ice-cooled oil phase. The aqueous phase and the oil phase are then stirred and mixed with a magnetic stirring bar at 1,150 rpm for 1 minute on ice to generate a water-in-oil emulsion.

To link lasso peptide and DNA barcode on the same bead, the emulsion is incubated at 37° C. for a total of 16 hours. During the incubation, the ukn22 A-TEV-SBP fusion protein is expressed and processed by MBP-B, MBP-C and MBP-RRE to form ukn22 lasso peptide with a threaded tail fused to TEV and SBP—“ukn22-TEV-SBP.” Following the 16-hour incubation, the aqueous reaction mixtures are recovered by centrifugation of the emulsion at 3,000 g for 5 minutes. The oil phase is removed while the concentrated emulsion remains at the bottom of the tube. Quenching buffer, containing TNTB, 1 mg/ml salmon sperm DNA and 1 μM biotin, and 2 ml of water-saturated diisopropyl ether (Sigma, CAT. #38270) are added. The mixture is vortexed and centrifuged to separate the aqueous phase from the ether phase which is subsequently removed from the tube. The aqueous phase is exposed to a vacuum to remove residual diisopropyl either. The resulting beads are resuspended in 20 μL of TNTB and used for affinity selection.

To verify successful display of ukn22 lasso peptide on the beads, 5 μL of the resuspended magnetic beads is treated with TEV protease (Sigma Cat. #T4455) to release ukn22 lasso peptide following the manufacturer's instructions. An equal volume of methanol is then added to the digestion reaction and thoroughly mixed. The ukn22 lasso peptide released into the supernatant post-digestion is aspirated and transferred to a new tube while the TEV-SBP fusion protein bound to the magnetic beads remain immobilized at the bottom of the original tube by a magnet tube holder. The collected supernatant is subsequently concentrated and subjected to MALDT-TOF MS analysis to verify the presence of ukn22 lasso peptide fused to Linker 1 and part of TEV protease recognition site (ukn22-Linker 1-Glu-Asn-Leu-Tyr-Phe-Gln (SEQ ID NO:64)). To confirm the simultaneous presence of the corresponding DNA barcode on the beads, 1 μL of the resuspended magnetic beads is used for DNA amplification with polymerase chain reaction (PCR). The amplified dsDNA is subjected to DNA sequencing to verify the presence of the DNA barcode.

### 6.13 Example 13: Linking Lasso Peptide and DNA Barcode Via Streptavidin (STA)-Biotin Binding

To link genotype and phenotype without a solid support as shown in FIG. 6B, we generate two recombinant DNA molecules: (1) a linear, biotinylated dsDNA sequence encoding ukn22 A-TEV-STA-His fusion protein and (2) the MBP-B/MBP-C/MBP-RRE plasmid vector for production of ukn22 peptidase (B), cyclase (C) and RiPP Recognition Element (RRE), each of which is fused to the C-terminus of maltose binding protein (MBP). The biotinylated dsDNA sequence is designed to simultaneously serve as a unique DNA barcode for identification (genotype) and the DNA template for expression of the ukn22 A-TEV-STA-His fusion protein (phenotype). Moreover, the biotin moiety of the dsDNA molecule enables the high affinity binding of the ukn22 A-TEV-STA-His fusion protein to the dsDNA molecule (See: Doi et al. PLos ONE, 2012, 7:e30084), thus linking genotype to phenotype. Following this design principle, the biotinylated dsDNA molecule and the MBP-B/MBP-C/MBP-RRE plasmid vector are added into the CFB cell extracts containing all necessary components for in vitro transcription-translation (TX-TL) reaction. The combined TX-TL reaction is used as the aqueous phase to generate a water-in-oil emulsion as described by Tawfik and Griffiths (See: Nat. Biotech., 1998, 652-656). The emulsion is then incubated at 37° C. for two hours to express the four fusion proteins, ukn22 A-TEV-STA-His, MBP-B, MBP-C and MBP-RRE, in a single aqueous droplet. Upon expression of the ukn22 A-TEV-STA-His fusion protein, the streptavidin (STA) at the C-terminus binds to the biotin moiety of the dsDNA molecule in the same aqueous droplet. To catalyze the lasso formation, the emulsion is further incubated at 37° C. for 14 hours. During the 14-hour incubation, the ukn22 precursor peptide A at the N-terminus is cleaved and cyclized by MBP-B, MBP-C and MBP-RRE to form ukn22 lasso peptide with a threaded tail fused to TEV, STA and His tags—“ukn22-TEV-STA-His.” The presence of the TEV protease recognition sequence in the ukn22-TEV-STA-His fusion protein allows TEV protease-mediated cleavage to release ukn22 from the rest of the fusion protein for validation of lasso conformation by mass spectrometry. The six histidine (His) tag allows isolation and further purification of the ukn22-TEV-STA-His fusion protein.

To generate the biotinylated dsDNA molecule, the DNA sequence, including a T7 promoter and the coding sequence for ukn22 A-TEV-STA-His fusion protein, is synthesized by a DNA manufacturer (Integrated DNA Technologies). Biotinylation is achieved by incorporating a biotinylated 5′ DNA primer into the amplified dsDNA molecules with polymerase chain reaction (PCR). The biotinylated dsDNA molecule is then added into 50 μl of ice-cooled CFB cell extracts containing the MBP-B/MBP-C/MBP-RRE plasmid vector.

To create an water-in-oil emulsion, the oil phase is freshly prepared by dissolving 4.5% (vol/vol) Span 80 (Sigma, CAT. #85548) in mineral oil (Sigma, CAT. #M5904) followed by 0.5% (vol/vol) Tween 80 (Sigma, CAT. #P1754). The ice-cooled beads/CFB mixtures (50 μL) are added gradually to 950 μL of ice-cooled oil phase while stirring with a magnetic bar. Stirring is continued at 1,150 rpm for another 1 minute on ice to generate an water-in-oil emulsion.

To link lasso peptide and DNA barcode, the emulsion is incubated at 37° C. for a total of 16 hours. During the incubation, the ukn22 A-TEV-STA-His fusion protein is expressed and processed by MBP-B, MBP-C and MBP-RRE to form ukn22 lasso peptide with a threaded tail fused to TEV, STA and His tags—“ukn22-TEV-STA-His.” Following the 16 hour incubation, the aqueous reaction mixtures are recovered by centrifugation of the emulsion at 3,000 g for 5 minutes. The oil phase is removed while the concentrated emulsion remains at the bottom of the tube. Quenching buffer, containing TNTB, 1 mg/ml salmon sperm DNA and 1 μM biotin, and 2 ml of water-saturated diisopropyl ether (Sigma, CAT. #38270) are added. The mixture is vortexed and centrifuged to separate the aqueous phase from the ether phase which is subsequently removed from the tube. The aqueous phase is exposed to a vacuum to remove residual diisopropyl either. The resulting materials are re-suspended in 20 μL of TNTB and used for affinity selection.

To verify successful linking of ukn22 lasso peptide to the dsDNA molecule, nickel resins (Pierce™ Ni-NTA Magnetic Agarose Beads, Thermo Fisher Scientific, Cat. #78606) are added into the resuspended materials to pull down the complex of the ukn22-TEV-STA-His fusion and dsDNA. The unbound components in the supernatant are removed. The “ukn22-TEV-STA-His fusion/dsDNA” complex bound to the nickel resins are treated with TEV protease (Sigma Cat. #T4455) to release ukn22 lasso peptide from the TEV-STA-His fusion protein. The ukn22 lasso peptide released into the supernatant post-digestion is aspirated and transferred to a new tube. An equal volume of methanol is then added to the collected supernatant in the new tube and thoroughly mixed. The resulting sample is subsequently concentrated and subjected to MALDT-TOF MS analysis to verify the presence of ukn22 lasso peptide fused to Linker 1 and part of TEV protease recognition site (ukn22-Linker 1-Glu-Asn-Leu-Tyr-Phe-Gln (SEQ ID NO:64)). To confirm the simultaneous presence of the corresponding DNA barcode, 1 μL of the “ukn22-TEV-STA-His fusion/dsDNA” complex from the pull-down sample is used for DNA amplification with polymerase chain reaction (PCR). The amplified dsDNA is subjected to DNA sequencing to verify the presence of the DNA barcode.

The following Example demonstrates that MBP-fused FusA-TEV-SAV (SEQ ID NO:62) was converted to Fusilassin-TEV-SAV (Seq ID No: 63) and that MBP-fused FusA-TEV-SAV bound its corresponding biotinylated DNA. E. coli BL21 (DE3) cells were transformed with pET28-MBP-FusA-TEV-SAV. Cells were grown overnight on LB agar plates containing 50 μg/mL kanamycin and 34 μg/mL chloramphenicol at 37° C. A single colony was used to inoculate 10 mL of LB containing 50 μg/mL kanamycin and 34 μg/mL chloramphenicol and grown at 30° C. for 12 h. This culture was used to inoculate 250 mL of LB containing 25 μg/mL kanamycin and 17 μg/mL chloramphenicol which was grown at 37° C. to an optical density at 600 nm (OD600) of 0.7-0.8. Expression was then induced by the addition of 0.5 mM (final concentration) isopropyl β-D-1-thiogalactopyranoside (IPTG). Expression was allowed to proceed for 3 h at 37° C. Cells were harvested by centrifugation at 4,500×g for 10 min. MBP-FusA-TEV-SAV purification by amylose resin affinity chromatography was performed by applying the sonicated pellet lysate to a pre-equilibrated amylose resin (5 mL of resin per 1L of culture, New England Biolabs, Ipswich, Mass.). The column was washed with 10 column volumes (CV) of lysis buffer followed by 10 CV of wash buffer (lysis buffer without Triton X-100) per the manufacturer's recommended protocol. The MBP-tagged FusA-TEV-SAV was eluted with 15 mL elution buffer (lysis buffer with 300 mM NaCl, 10 mM maltose, and lacking Triton X-100) and collected into an appropriate molecular weight cutoff (MWCO) Amicon Ultra centrifugal filter (EMD Millipore, Burlington, Mass.). Protein eluent was concentrated to −1.5 mL and exchanged with 10× volume of protein storage buffer [50 mM HEPES pH 7.5, 300 mM NaCl, 0.5 mM tris-(2-carboxyethyl)-phosphine (TCEP), 2.5% glycerol (v/v)]. Protein concentrations were assayed using 280 nm absorbance (theoretical extinction coefficients were calculated using the ExPASy ProtParam tool; http://web.expasy.org/protparam/protpar-ref.html). Final protein purity was assessed visually using a Coomassie-stained SDS-PAGE gel.

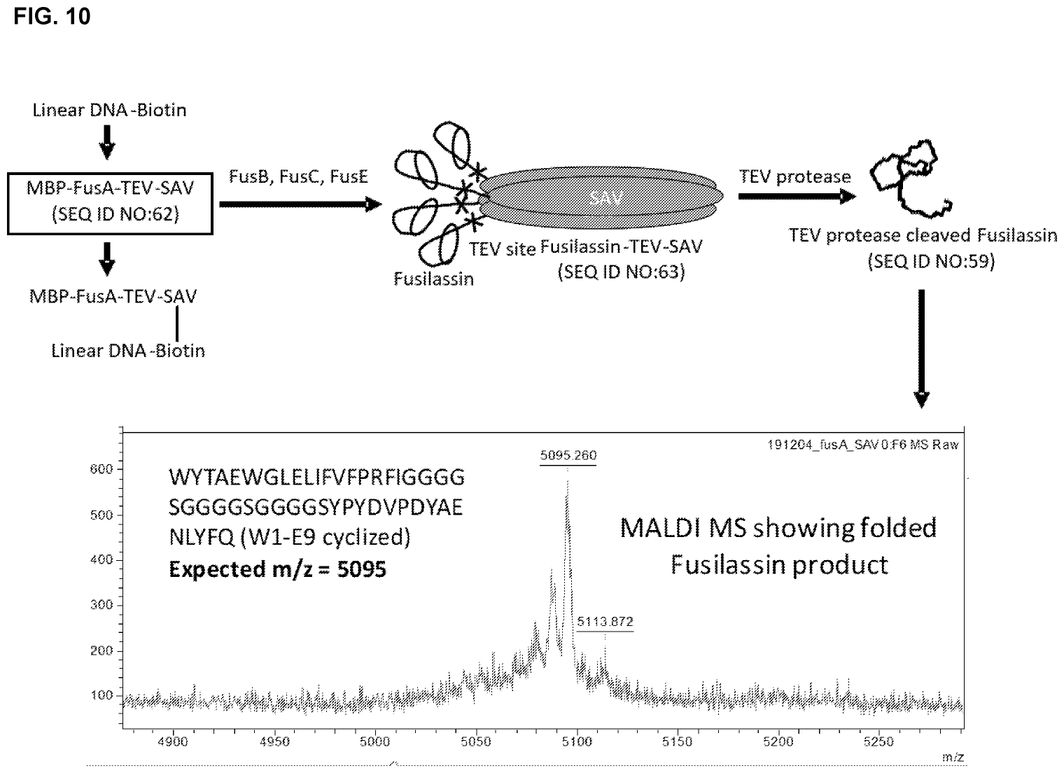

Formation of Fusilassin-TEV-SAV. MBP-FusA-TEV-SAV (20 μM) produced above was combined with 10 μM each of the purified enzymes FusB, FusC, and FusE in buffer and the cell-free reactions were incubated at 37° C. for 12 h. Following treatment with TEV protease for 3 h to release the folded mature lasso peptide product, the reaction mixture was purified and concentrated with an EMD Millipore ZipTip and analyzed by MALDI MS. A clear m/z peak of 5095 was observed, as expected for the folded Fusilassin product (SEQ ID NO:59) cleaved with TEC protease (See FIG. 10).

Binding of biotinylated DNA to translated product MBP-FusA-TEV-SAV. Linear DNA template for MBP-FusA-TEV-SAV was amplified by PCR where the reverse primer was modified with a 5′ biotin such that the amplicon had a biotin attached to the 3′ end. The biotinylated DNA (100 ng/μl) thus produced was incubated with MBP-FusA-TEV-SAV (10 μM) or FusA (10 μM, negative control) for 2 hrs at room temperature with shaking. The samples were further incubated with free streptavidin beads (Thermo Fisher, Waltham, Mass.) for 1 hr at room temperature with shaking to remove unbound DNA. Three to five times more biotinylated DNA was retained in the supernatant by the MBP-FusA-TEV-SAV relative to the FusA control, demonstrating the SAV in FusA-TEV-SAV bound its cognate biotinylated DNA.

### 6.14 Example 14: Linking Lasso Peptide and DNA Barcode Via Binding of RepA to the Plasmid Origin of Replication (oriR) Sequence

To link genotype and phenotype without a solid support as shown in FIG. 6C, we generate two recombinant DNA molecules: (1) a linear dsDNA sequence encoding ukn22 A-TEV-RepA-His fusion protein and (2) the MBP-B/MBP-C/MBP-RRE plasmid vector for production of ukn22 peptidase (B), cyclase (C) and RiPP Recognition Element (RRE), each of which is fused to the C-terminus of maltose binding protein (MBP). The dsDNA sequence is designed to simultaneously serve as a unique DNA barcode for identification (genotype) and the DNA template for expression of the ukn22 A-TEV-RepA-His fusion protein (phenotype). In addition, the presence of the CIS and oriR DNA sequences at the 3′ untranslated region (3′ UTR) of the dsDNA template enables the high-affinity binding of RepA in cis to the oriR sequence of the same dsDNA template from which the fusion protein is expressed (See: Masai and Arai. Nucleic Acid Research, 1988, 16:6493-6514; Odegrip et al. PNAS, 2004, 101:2806-2810). Such in cis high-affinity binding is mediated by the CIS sequence that serves as a rho-dependent transcriptional terminator for repA messenger RNA (mRNA). During transcription, a rho-dependent terminator causes stalling or pausing of RNA polymerase. Owing to this transcriptional pause, the newly transcribed repA mRNA molecule is anchored to its parent dsDNA template via the stalled RNA polymerase; thus, the nascent RepA protein translated from the anchored repA mRNA molecule is brought in close proximity to the oriR sequence downstream of the CIS sequence. As a result, the close proximity of RepA and the oriR sequence catalyzes the in cis high-affinity binding of RepA to the oriR sequence of the parent dsDNA template, thus linking genotype to phenotype. Following this design principle, the dsDNA molecule and the MBP-B/MBP-C/MBP-RRE plasmid vector are added into the CFB cell extracts containing all necessary components for in vitro transcription-translation (TX-TL) reaction. The combined TX-TL reaction is used as the aqueous phase to generate a water-in-oil emulsion as described by Tawfik and Griffiths (See: Nat. Biotech., 1998, 652-656). The emulsion is then incubated at 37° C. for two hours to expressed the four fusion proteins, ukn22 A-TEV-RepA-His, MBP-B, MBP-C and MBP-RRE, in a single aqueous droplet. Upon expression of the ukn22 A-TEV-RepA-His fusion protein, the RepA domain of the fusion protein acts in cis and binds to the oriR sequence of the dsDNA template from which the fusion protein is expressed. To catalyze the lasso formation, the emulsion is further incubated at 37° C. for 14 hours. During the 14 hour incubation, the ukn22 leader sequence at the N-terminus of precursor peptide A is cleaved and cyclized by MBP-B, MBP-C and MBP-RRE to form ukn22 lasso peptide with a threaded tail fused to TEV, RepA and His tags—“ukn22-TEV-RepA-His.” The presence of the TEV protease recognition sequence in the ukn22-TEV-RepA-His fusion protein allows TEV protease-mediated cleavage to release ukn22 from the rest of the fusion protein for validation of lasso conformation by mass spectrometry. The six histidine (His) tag allows isolation of the ukn22-TEV-RepA-His fusion protein.

To generate the dsDNA molecule, the DNA sequence, including a T7 promoter and the coding sequence for ukn22 A-TEV-RepA-His fusion protein, is synthesized by a DNA manufacturer (Integrated DNA Technologies). The synthesized dsDNA molecule is further amplified with polymerase chain reaction (PCR). The amplified dsDNA molecule is then added into 50 μl of ice-cooled CFB cell extracts containing the MBP-B/MBP-C/MBP-RRE plasmid vector.

To create an water-in-oil emulsion, the oil phase is freshly prepared by dissolving 4.5% (vol/vol) Span 80 (Sigma, CAT. #85548) in mineral oil (Sigma, CAT. #M5904) followed by 0.5% (vol/vol) Tween 80 (Sigma, CAT. #P1754). The ice-cooled beads/CFB mixtures (50 μL) are added gradually to 950 μL of ice-cooled oil phase while stirring with a magnetic bar. Stirring is continued at 1,150 rpm for another 1 minute on ice to generate a water-in-oil emulsion.

To link lasso peptide and DNA barcode, the emulsion is incubated at 37° C. for a total of 16 hours. During the incubation, the ukn22 A-TEV-RepA-His fusion protein is expressed and processed by MBP-B, MBP-C and MBP-RRE to form ukn22 lasso peptide with a threaded tail fused to TEV, RepA and His tags—“ukn22-TEV-RepA-His.” Following the 16 hour incubation, the aqueous reaction mixtures are recovered by centrifugation of the emulsion at 3,000 g for 5 minutes. The oil phase is removed while the concentrated emulsion remains at the bottom of the tube. Quenching buffer, containing TNTB, 1 mg/ml salmon sperm DNA and 1 μM biotin, and 2 ml of water-saturated diisopropyl ether (Sigma, CAT. #38270) are added. The mixture is vortexed and centrifuged to separate the aqueous phase from the ether phase which is subsequently removed from the tube. The aqueous phase is exposed to a vacuum to remove residual diisopropyl either. The resulting materials are resuspended in 20 μL of TNTB and used for affinity selection.

To verify successful linking of ukn22 lasso peptide to the dsDNA molecule, nickel resins (Pierce™ Ni-NTA Magnetic Agarose Beads, Thermo Fisher Scientific, Cat. #78606) are added into the resuspended materials to pull down the complex of the ukn22-TEV-RepA-His fusion and dsDNA. The unbound components in the supernatant are removed. The “ukn22-TEV-RepA-His fusion/dsDNA” complex bound to the nickel resins are treated with TEV protease (Sigma Cat. #T4455) to release ukn22 lasso peptide from the TEV-RepA-His fusion protein. The ukn22 lasso peptide released into the supernatant post-digestion is aspirated and transferred to a new tube. An equal volume of methanol is then added to the collected supernatant in the new tube and thoroughly mixed. The resulting sample is subsequently concentrated and subjected to MALDT-TOF MS analysis to verify the presence of ukn22 lasso peptide fused to Linker 1 and part of TEV protease recognition site (ukn22-Linker 1-Glu-Asn-Leu-Tyr-Phe-Gln). To confirm the simultaneous presence of the corresponding DNA barcode, 1 μL of the “ukn22-TEV-RepA-His fusion/dsDNA” complex from the pull-down sample is used for DNA amplification with polymerase chain reaction (PCR). The amplified dsDNA is subjected to DNA sequencing to verify the presence of the DNA barcode.

### 6.15 Example 15: Production of a DNA Displayed Lasso Peptide Library in Individual Wells

To produce a DNA displayed lasso peptide library in individual wells (FIG. 5A), the coding sequence for ukn22 precursor peptide A is replaced with a library of ukn22 precursor peptide A variants (ukn22 A*) to generate a library of the ukn22 A*-TEV-SBP plasmid vectors. The MBP-B/MBP-C/MBP-RRE plasmid vector is also generated for production of ukn22 peptidase (B), cyclase (C) and RiPP Recognition Element (RRE), each of which is fused to the C-terminus of maltose binding protein (MBP). The ukn22 A*-TEV-SBP plasmid vectors are individually added into single wells of the 384-well PCR plate, followed by the addition of the MBP-B/MBP-C/MBP-RRE plasmid vector to all wells. The in vitro transcription-translation (TX-TL) of these four fusion proteins is carried out by adding Cell-Free Biosynthesis (CFB) cell extracts into individual wells, followed by the incubation at 37° C. for 16 hours. During the incubation of the TX-TL reactions, the ukn22 precursor peptide A variants are individually expressed, cleaved and cyclized by the ukn22 synthetase enzymes B, C and RRE to produce the variants of ukn22 lasso peptide fusion protein—“ukn22*-TEV-SBP.” Each of the generated ukn22*-TEV-SBP variants is then mixed with streptavidin-coated magnetic beads, which are pre-bound with biotinylated dsDNA molecules that serves as a DNA barcode. The resulting DNA displayed lasso peptide library has each ukn22*-TEV-SBP variant linked to a unique DNA barcode on beads in a single well. The presence of the TEV protease recognition sequence in the ukn22-TEV-SBP fusion protein allows TEV protease-mediated cleavage to release ukn22 for validation of lasso conformation by mass spectrometry.

To generate a library of the ukn22 A*-TEV-SBP plasmid vectors, the coding sequences for ukn22 precursor peptide A variants are cloned in front of the SBP coding sequence and behind a constitutive T7 promoter. The coding sequence for the TEV protease recognition site (Glu-Asn-Leu-Tyr-Phe-Gln⬇Gly) (SEQ ID NO:15) flanked by two linker sequences, Linker 1 and Linker 2, is then inserted in-frame in between the ukn22 precursor peptide A variants and the SBP. The constructed ukn22 A*-TEV-SBP coding sequences are then cloned into a plasmid vector containing a pUC E. coli replication origin and the ampicillin resistance gene. To generate the MBP-B/MBP-C/MBP-RRE plasmid vector, the coding sequences for ukn22 peptidase (B), cyclase (C) and RiPP recognition element (RRE) are cloned in-frame behind the maltose binding protein (MBP) to create three fusion proteins, MBP-B, MBP-C and MBP-RRE, each of which is expressed from an independent, constitutive T7 promoter on a plasmid containing the chloramphenicol resistance gene.

To link lasso peptide and DNA barcode on the same bead, each of the ukn22 A*-TEV-SBP plasmid vector (10 ng) and the MBP-B/MBP-C/MBP-RRE plasmid vector (10 ng) are added into a total of 40 μL CFB reaction in a well of the 384-well PCR plate. The reactions are incubated at 37° C. for 16 hours to produce the ukn22 A*-TEV-SBP, MBP-B, MBP-C and MBP-RRE fusion proteins. During the 16 hour incubation, the leader sequence at the N-terminus of ukn22 precursor peptide A variants are cleaved and cyclized by MBP-B, MBP-C and MBP-RRE to form ukn22 lasso peptide variants with a threaded tail fused to TEV and SBP—“ukn22*-TEV-SBP.” Following the 16 hour incubation, the streptavidin-coated magnetic beads (Dynabeads™ MyOne™ Streptavidin T1, Thermo Fisher Scientific, Cat. #65601) pre-bound with biotinylated dsDNA molecules (Integrated DNA Technologies), unique to each well, are added to the individual wells containing the produced ukn22*-TEV-SBP fusion proteins. The quantity of the bound biotinylated dsDNA is adjusted so that at least more than 95% of streptavidin-coated bead surface remains available for SBP-streptavidin binding. The conjugation reactions take place at 4 C for an hour with gentle shaking. Following the one hour incubation, the 384-well PCR plate is placed on a 384 magnet plate (Alpaqua) to immobilize the magnetic beads and the TX-TL reaction mixtures within the wells are aspirated. The immobilized magnetic beads are washed three times with 50 μL ice-cold TNTB Wash Buffer (0.1 M Tris pH 7.5, 0.15 M NaCl, 0.05% Tween-20, 1% bovine serum albumin). Upon the aspiration of the last Wash Buffer, the immobilized magnetic beads in each well are resuspended in 20 μL of TNTB buffer and used for affinity selection.

To verify successful display of ukn22 lasso peptide variants on the beads, ten wells are randomly chosen and 5 μL of the resuspended magnetic beads from each well is treated with TEV protease (Sigma Cat. #T4455) to release ukn22 lasso peptide variants following the manufacturer's instructions. An equal volume of methanol is then added to each digestion reaction and thoroughly mixed. The ukn22 lasso peptide variants released into the supernatant post-digestion are aspirated and transferred to individual wells of a new 384-well PCR plate while the TEV-SBP fusion protein bound to the magnetic beads remain immobilized on the original 384-well PCR plate by a 384 magnet plate. The collected samples are subsequently concentrated and subjected to MALDT-TOF MS analysis to verify the presence of ukn22 lasso peptide variants, each of which fused to Linker 1 and part of TEV protease recognition site (ukn22-Linker 1-Glu-Asn-Leu-Tyr-Phe-Gln (SEQ ID NO:64)). To confirm the simultaneous presence of the corresponding DNA barcode on the beads, 1 μL of the resuspended magnetic beads from each of the chose wells is used for DNA amplification with polymerase chain reaction (PCR). The amplified dsDNA molecules are subjected to DNA sequencing to verify the presence of the expected DNA barcodes.

### 6.16 Example 16: Production of a DNA Displayed Lasso Peptide Library in a Water-in-Oil Emulsion

To produce a DNA displayed lasso peptide library in a water-in-oil emulsion (FIG. 6A), the coding sequence for ukn22 precursor peptide A is replaced with a library of ukn22 precursor peptide A variants (ukn22 A*) to generate a library of linear, biotinylated dsDNA sequences coding for ukn22 A*-TEV-SBP fusion proteins. The MBP-B/MBP-C/MBP-RRE plasmid vector is also generated for production of ukn22 peptidase (B), cyclase (C) and RiPP Recognition Element (RRE), each of which is fused to the C-terminus of maltose binding protein (MBP). The biotinylated dsDNA molecules are designed to simultaneously serve as a unique DNA barcode for identification (genotype) and the DNA templates for expression of each ukn22 A*-TEV-SBP fusion variant (phenotype). To link genotype and phenotype on the same solid support, the biotinylated dsDNA molecules are pre-bound to streptavidin-coated beads at the 1:1 ratio of dsDNA molecules to beads, followed by the addition of the MBP-B/MBP-C/MBP-RRE plasmid vector and the CFB cell extracts containing all necessary components for in vitro transcription-translation (TX-TL) reaction. The combined TX-TL reactions are used as the aqueous phase to generate a water-in-oil emulsion as described by Tawfik and Griffiths (See: Nat. Biotech., 1998, 652-656). The emulsion is then incubated at 37° C. for two hours to express the four fusion proteins, ukn22 A*-TEV-SBP, MBP-B, MBP-C and MBP-RRE, in a single aqueous droplet. Upon expression of the ukn22 A*-TEV-SBP fusion proteins, the streptavidin binding peptide (SBP) at the C-terminus binds to the streptavidin-coated beads in the same aqueous droplet. To catalyze the lasso formation, the emulsion is further incubated at 37° C. for 14 hours. During the 14 hour incubation, the leader sequence at the N-terminus of ukn22 precursor peptide A variants are cleaved and the resulting core sequences are cyclized by MBP-B, MBP-C and MBP-RRE to form ukn22 lasso peptide variants, each of which with a threaded tail fused to TEV and SBP—“ukn22*-TEV-SBP.” The presence of the TEV protease recognition sequence in each ukn22*-TEV-SBP fusion protein allows TEV protease-mediated cleavage to release each ukn22 variant from the rest of the fusion protein for validation of lasso conformation by mass spectrometry.

To generate the biotinylated dsDNA molecules, the dsDNA sequences, including a T7 promoter and the coding sequences for ukn22 A*-TEV-SBP fusion proteins, are synthesized by a DNA manufacturer (Twist Bioscience). Biotinylation is achieved by incorporating a biotinylated 5′ DNA primer into the amplified dsDNA molecules with polymerase chain reaction (PCR). The biotinylated dsDNA sequences are then mixed with streptavidin-coated magnetic beads (Dynabeads™ MyOne™ Streptavidin T1, Thermo Fisher Scientific, Cat. #65601) in 50 μL) of TNTB buffer (0.1 M Tris pH 7.5, 0.15 M NaCl, 0.05% Tween-20, 1% bovine serum albumin). The dsDNA/bead mixture is then incubated overnight at 4° C. to achieve the 1:1 ratio of dsDNA molecules to beads. After the overnight incubation, the beads are washed twice with TNTB buffer and then resuspended in 50 μl of ice-cooled CFB cell extracts containing the MBP-B/MBP-C/MBP-RRE plasmid vector.

To create a water-in-oil emulsion, the oil phase is freshly prepared by dissolving 4.5% (vol/vol) Span 80 (Sigma, CAT. #85548) in mineral oil (Sigma, CAT. #M5904) followed by 0.5% (vol/vol) Tween 80 (Sigma, CAT. #P1754). The ice-cooled beads/CFB mixtures (50 μL) are added gradually to 950 μL of ice-cooled oil phase while stirring with a magnetic bar. Stirring is continued at 1,150 rpm for another 1 minute on ice to generate an water-in-oil emulsion.

To link lasso peptide and DNA barcode on the same bead, the emulsion is incubated at 37° C. for a total of 16 hours. During the incubation, the ukn22 A*-TEV-SBP fusion proteins are expressed and processed by MBP-B, MBP-C and MBP-RRE to form ukn22 lasso peptide variants, each of which with a threaded tail fused to TEV and SBP—“ukn22*-TEV-SBP.” Following the 16 hour incubation, the aqueous reaction mixtures are recovered by centrifugation of the emulsion at 3,000 g for 5 minutes. The oil phase is removed while the concentrated emulsion remains at the bottom of the tube. Quenching buffer, containing TNTB, 1 mg/ml salmon sperm DNA and 1 μM biotin, and 2 ml of water-saturated diisopropyl ether (Sigma, CAT. #38270) are added. The mixture is vortexed and centrifuged to separate the aqueous phase from the ether phase which is subsequently removed from the tube. The aqueous phase is exposed to a vacuum to remove residual diisopropyl either. The resulting beads are resuspended in 20 μL of TNTB and used for affinity selection.

To verify successful display of ukn22 lasso peptide on the beads, 5 μL of the resuspended magnetic beads is treated with TEV protease (Sigma Cat. #T4455) to release ukn22 lasso peptide following the manufacturer's instructions. An equal volume of methanol is then added to the digestion reaction and thoroughly mixed. The ukn22 lasso peptide variants released into the supernatant post-digestion are aspirated and transferred to a new tube while the TEV-SBP fusion protein bound to the magnetic beads remain immobilized at the bottom of the original tubes by a magnet tube holder. The collected supernatant is subsequently concentrated and subjected to MALDT-TOF MS analysis to verify the presence of ukn22 lasso peptide variants fused to Linker 1 and part of TEV protease recognition site (Ukn22-Linker 1-Glu-Asn-Leu-Tyr-Phe-Gln (SEQ ID NO:64)). To confirm the simultaneous presence of the corresponding DNA barcodes on the beads, 1 μL of the resuspended magnetic beads is used for DNA amplification with polymerase chain reaction (PCR). The amplified dsDNA molecules are subjected to Next-Gen DNA sequencing (Illumina) to verify the expected DNA barcode sequences.

### 6.17 Example 17: Directed Evolution of a Single Lasso Peptide to Produce High-Affinity Ligands Via Whole Cell Panning Using DNA Display

To evolve a lasso peptide to become a high-affinity antagonist of glucagon receptor (GCGR), BI-32169 (Gly-Leu-Pro-Trp-Gly-Cys-Pro-Ser-Asp-Ile-Pro-Gly-Trp-Asn-Thr-Pro-Trp-Ala-Cys (SEQ ID NO:6)) discovered in Streptomyces sp. (See: Streicher et al., J. Nat. Prod., 2004, 67, 1528-1531) is chosen as a starting lasso scaffold for evolution. Since the sequence of peptidase (B), cyclase (C) and RRE of BI-32169 have not been identified, the peptidase (B), cyclase (C) and RRE of a BI-32169 analog (Gly-Leu-Pro-Trp-Gly-Cys-Pro-Asn-Asp-Leu-Phe-Phe-Val-Asn-Thr-Pro-Phe-Ala-Cys (SEQ ID NO:7)) identified in Kibdelosporangium sp. MJ126-NF4 are chosen to construct the MBP-B/MBP-C/MBP-RRE plasmid. Lasso peptide synthetase enzymes B, C and RRE recognize the leader peptide of a lasso precursor peptide and exhibit plasticity toward the core peptide. Moreover, the amino acid sequence of the core peptide can be altered to include mutations, deletions and C-terminal extension (See: Pan and Link. J. Am. Chem. Soc., 2011, 133:5016-23; Zong et al. ACS Chem. Biol., 2016, 11:61-8). Therefore, the leader peptide sequence of BI-32169 is replaced with the leader peptide sequence of the BI-32169 analog to construct the hybrid BI-32169 precursor peptide A (Met-Ile-Lys-Asp-Asp-Glu-Ile-Tyr-Glu-Val-Pro-Thr-Leu-Val-Glu-Val-Gly-Asp-Phe-Ala-Glu-Leu-Thr-Leu-Gly-Leu-Pro-Trp-Gly-Cys-Pro-Ser-Asp-Ile-Pro-Gly-Trp-Asn- Thr-Pro-Trp-Ala-Cys (SEQ ID NO:9)) so that this hybrid precursor peptide A can be processed by the BI-32169 analog synthetase enzymes B, C and RRE from Kibdelosporangium sp. MJ126-NF4 for formation of BI-32169 lasso peptide. Leveraging the plasticity of lasso peptide synthetase enzymes, a DNA displayed lasso peptide library is generated in a water-in-oil emulsion following the procedures described in Example 16.

To generate BI-32169 variants, the DNA coding sequence for the hybrid BI-32169 precursor peptide A is synthesized with each amino acid codon of the core peptide sequentially replaced with a degenerate codon, such as NNK, except for the aspartic acid residue at the 9th position of the core peptide that is required for the ring formation. These synthesized DNA sequences, including a T7 promoter, the coding sequence for hybrid BI-32169 NNK variants, TEV and SBP, are biotinylated via polymerase reaction (PCR), as described in Example 16. The biotinylated dsDNA molecules are subsequently used to create a DNA displayed lasso peptide library in a water-in-oil emulsion.

To select for antagonists of glucagon receptor (GCGR), the DNA displayed lasso peptide library is screened for its ability to bind to GCGR expressed on the surface of CHO-S cells (Life Technologies) in the presence of glucagon (GCG), a native GCGR ligand. Following a similar protocol to the whole cell panning procedure (FIG. 7C) reported by Jones et al. (See: Sci Rep., 2016, 18; 6:26240), the CHO-S cells expressing GCGR are first washed in PBS, then blocked in 5 mL 2% (w/v) milk-PBS (MPBS) with rotation for 30 minutes at 4° C. The DNA display library is then added to the blocked cells and incubated with rotation for 1 hour at 4° C. in the presence of glucagon. The cells are then washed three times using Wash Buffer (PBS, 0.1% (v/v) Tween-20, pH 5.0), followed by 3 washes with PBS (pH 7.4) to remove unbound beads. The cells bound with beads are harvested, transferred to a 15 mL conical tube and pelleted by brief centrifugation at 4° C. for 5 minutes. After centrifugation, the supernatant is removed and 5 mL of Lysis Buffer (150 mM NaCl, 50 mM Tris-HCl pH 7.4, 1 mM EDTA, and 1% Triton-X 100) is added to the cells, followed by further incubation at 95° C. for 5 minutes to release biotinylated dsDNA molecules from the denatured streptavidin on beads (See: Jenne and Famulok. Biotechniques, 1999, 26:249-52). The biotinylated dsDNA molecules in the supernatant are amplified with polymerase chain reaction (PCR) with the biotinylated 5′ primer and the 3′ primer for Next-Gen DNA sequencing analysis (Illumina) to reveal the amino acids mutations and positions that are beneficial in antagonizing GCG-GCGR binding. These beneficial mutations and positions are then incorporated into the design of a subsequent combinatorial DNA display library for next round of sequence selection. Such sequence selection via whole cell panning can be continued for several rounds with the sequence diversity monitored by DNA sequencing after each round of selection. To evolve for high-affinity antagonists of GCGR, the screening parameters and the composition of binding and washing media, such as incubation time, temperature, pH, salts and detergents, are adjusted to select for antagonists with increased binding affinity. The resulting high-affinity BI32169 mutants are further examined individually for their ability to inhibit calcium influx induced by GCG-GCGR binding using FLIPR® Calcium Assay (Molecular Devices, Cat. #FLIPR Calcium 6) with Ready-to-Assay™ Glucagon Receptor Frozen Cells (EMD Millipore, Cat. #HTS112RTA).

### 6.18 Example 18: In Vitro Selection of a DNA Displayed Lasso Peptide Library to Enrich High-Affinity Ligands Via Whole Cell Panning and Flow Cytometry

To screen for high-affinity antagonists of glucagon receptor (GCGR) using DNA display, a DNA displayed lasso peptide library is designed with the size of the ring ranging from 7, 8 to 9 amino acid residues and each of the core peptide residues mutated, except for the residue(s) required for the ring formation. To produce this DNA display library, ukn22 precursor peptide A (Met-Glu-Lys-Lys-Lys-Tyr-Thr-Ala-Pro-Gln-Leu-Ala-Lys-Val-Gly-Glu-Phe-Lys-Glu-Ala-Thr-Gly⬇Trp-Tyr-Thr-Ala-Glu-Trp-Gly-Leu-Glu-Leu-Ile-Phe-Val-Phe-Pro-Arg-Phe-Ile (SEQ ID NO:28)) is chosen as a starting sequence and follow the procedures described in Examples 10 and 17 to replace the ukn22 core peptide sequence (Trp-Tyr-Thr-Ala-Glu-Trp-Gly-Leu-Glu-Leu-Ile-Phe-Val-Phe-Pro-Arg-Phe-Ile (SEQ ID NO:1)) with one of the following coding sequences NNK-NNK-NNK-NNK-NNK-NNK-Glu-NNK-NNK-NNK-NNK-NNK-NNK-NNK-NNK-NNK-NNK-NNK-NNK (7-member ring), NNK-NNK-NNK-NNK-NNK-NNK-NNK-Glu-NNK-NNK-NNK-NNK-NNK-NNK-NNK-NNK-NNK-NNK-NNK (8-member ring), or NNK-NNK-NNK-NNK-NNK-NNK-NNK-NNK-Glu-NNK-NNK-NNK-NNK-NNK-NNK-NNK-NNK-NNK-NNK (9-member ring). Each of these coding sequences are synthesized as a pool of oligonucleotides by Twist Bioscience, Corp., amplified and biotinylated following the procedures described in Example 16 to produce a large DNA displayed lasso peptide library in a water-in-oil emulsion.

To select for antagonists of glucagon receptor (GCGR) using fluorescence-activated cell sorting (FACS) as shown in FIG. 8 (top), the DNA displayed lasso peptide library is screened for its ability to bind GCGR expressed on the surface of CHO-S cells (Life Technologies) in the presence of glucagon (GCG), a native GCGR ligand. Following a similar procedure (FIG. 7D) to the whole cell panning method reported by Jones et al. (See: Sci Rep., 2016, 18; 6:26240), a cell suspension of the CHO-S cells expressing GCGR are first washed in PBS, then blocked in 5 mL 2% (w/v) milk-PBS (MPBS) with rotation for 30 minutes at 4° C. The DNA display library is then added to the blocked cells and incubated with rotation for 1 hour at 4° C. in the presence of glucagon. The cells are then washed three times using Wash Buffer (PBS, 0.1% (v/v) Tween-20, pH 5.0), followed by 3 washes with PBS (pH 7.4) to remove unbound beads and re-suspended in 5 mL of Suspension Buffer (Hank's Balanced Salt Solution, 25 mM HEPES, and 3% fetal calf serum).

To sort the cells bound to the complex of lasso peptides and beads (FIG. 8 top), the FITC-conjugated anti-SBP monoclonal antibody (Santa Cruz Biotechnology) is added to the re-suspended cells. The cells are incubated for 60 minutes at 4° C. in the dark, followed by two washes with Suspension Buffer without serum. The cells are re-suspended again in Suspension Buffer and the concentration of cells is adjusted to 15-20×10⁶ cells/mL prior to fluorescence-activated cell sorting (FACS) by a flow cytometer. The collected fluorescent cells bound with beads are pelleted by brief centrifugation at 4° C. for 5 minutes. After centrifugation, the supernatant is removed and 5 mL of Lysis Buffer (150 mM NaCl, 50 mM Tris-HCl pH 7.4, 1 mM EDTA, and 1% Triton-X 100) is added to the cells, followed by further incubation at 95° C. for 5 minutes to release biotinylated dsDNA molecules from the denatured streptavidin on beads (Jenne and Famulok. Biotechniques, 1999, 26:249-52). The biotinylated dsDNA molecules in the supernatant are amplified with polymerase chain reaction (PCR) with the biotinylated 5′ primer and the 3′ primer for the generation and screening of the subsequent DNA displayed lasso peptide library. During each round of whole cell panning, a subpopulation of the library is enriched, and the sequence diversity of lasso peptides is monitored by Illumina Next-Gen DNA sequencing.

To evolve for high-affinity antagonists of GCGR, the screening parameters and the composition of binding and washing media, such as incubation time, temperature, pH, salts and detergents, are adjusted to select for antagonists with increased binding affinity. The resulting high-affinity lasso peptides are further examined individually for their ability to inhibit calcium influx induced by GCG-GCGR binding using FLIPR® Calcium Assay (Molecular Devices, Cat. #FLIPR Calcium 6) with Ready-to-Assay™ Glucagon Receptor Frozen Cells (EMD Millipore, Cat. #HTS112RTA).

### 6.19 Example 19: In Vitro Selection and Evolution of a DNA Displayed Lasso Peptide Library to Enrich High-Affinity Ligands Via Whole Cell Panning and Sequential Flow Cytometry

To screen for high-affinity agonists of glucagon-like peptide-1 receptor (GLP-1R) using DNA display, DNA displayed lasso peptide library is designed with the size of the ring ranging from 7, 8 to 9 amino acid residues and each of the core peptide residues mutated, except for the residue(s) required for the ring formation. To produce this library, ukn22 precursor peptide A (Met-Glu-Lys-Lys-Lys-Tyr-Thr-Ala-Pro-Gln-Leu-Ala-Lys-Val-Gly-Glu-Phe-Lys-Glu-Ala-Thr-Gly⬇Trp-Tyr-Thr-Ala-Glu-Trp-Gly-Leu-Glu-Leu-Ile-Phe-Val-Phe-Pro-Arg-Phe-Ile (SEQ ID NO:2)) is chosen as a starting sequence and follow the procedures described in Examples 10 and 17 to replace the ukn22 core peptide sequence (Trp-Tyr-Thr-Ala-Glu-Trp-Gly-Leu-Glu-Leu-Ile-Phe-Val-Phe-Pro-Arg-Phe-Ile (SEQ ID NO:1)) with one of the following coding sequences NNK-NNK-NNK-NNK-NNK-NNK-Glu-NNK-NNK-NNK-NNK-NNK—NNK-NNK-NNK-NNK-NNK-NNK-NNK (7-member ring), NNK-NNK—NNK-NNK—NNK—NNK-NNK-Glu-NNK-NNK-NNK-NNK-NNK-NNK-NNK-NNK-NNK-NNK-NNK (8-member ring), or NNK-NNK-NNK-NNK-NNK-NNK-NNK-NNK-Glu-NNK-NNK-NNK-NNK-NNK—NNK-NNK-NNK-NNK-NNK (9-member ring). Each of these coding sequences are synthesized as a pool of oligonucleotides by Twist Bioscience, Corp., amplified and biotinylated following the procedures described in Example 16 to produce a large DNA displayed lasso peptide library in a water-in-oil emulsion.

To select for agonists of glucagon-like peptide-1 receptor (GLP-1R) using fluorescence-activated cell sorting (FACS) as shown in FIG. 8 (bottom), the DNA displayed lasso peptide library is screened for its ability to bind GLP-1R expressed on the surface of CHO-S cells (Life Technologies). Following a similar procedure (FIG. 7D) to the whole cell panning method reported by Jones et al. (See: Sci Rep., 2016, 18; 6:26240), a cell suspension of the CHO-S cells expressing GLP-1R are first washed in PBS, then blocked in 5 mL 2% (w/v) milk-PBS (MPBS) with rotation for 30 minutes at 4° C. The DNA display library is then added to the blocked cells and incubated with rotation for 1 hour at 4° C. The cells are then washed three times using Wash Buffer (PBS, 0.1% (v/v) Tween-20, pH 5.0), followed by 3 washes with PBS (pH 7.4) to remove unbound beads and re-suspended in 5 mL of Suspension Buffer (Hank's Balanced Salt Solution, 25 mM HEPES, and 3% fetal calf serum).

To sort the cells that are bound to the lasso peptides/beads complex and exhibit intracellular calcium immobilization triggered by lasso peptide-GLP-1R binding (FIG. 8 bottom), the FITC-conjugated anti-SBP monoclonal antibody (Santa Cruz Biotechnology) and FLIPR Calcium 6 (Molecular Devices) are added to the re-suspended cells. The cells are incubated for 60 minutes at 4° C. in the dark, followed by two washes with Suspension Buffer without serum. The cells are re-suspended again in Suspension Buffer and the concentration of cells is adjusted to 15-20×10⁶ cells/mL prior to sequential fluorescence-activated cell sorting (FACS) by a flow cytometer. The double-sorted cells are pelleted by brief centrifugation at 4° C. for 5 minutes. After centrifugation, the supernatant is removed and 5 mL of Lysis Buffer (150 mM NaCl, 50 mM Tris-HCl pH 7.4, 1 mM EDTA, and 1% Triton-X 100) is added to the cells, followed by further incubation at 95° C. for 5 minutes to release biotinylated dsDNA molecules from the denatured streptavidin on beads (Jenne and Famulok. Biotechniques, 1999, 26:249-52). The biotinylated dsDNA molecules in the supernatant are amplified with polymerase chain reaction (PCR) with the biotinylated 5′ primer and the 3′ primer for the generation and screening of the subsequent DNA displayed lasso peptide library. During each round of whole cell panning, a subpopulation of the library is enriched, and the sequence diversity of lasso peptides is monitored by Illumina Next-Gen DNA sequencing.

To evolve for high-affinity agonists of GLP-1R, the screening parameters and the composition of binding and washing media, such as incubation time, temperature, pH, salts and detergents, are adjusted to select for antagonists with increased binding affinity.

### 6.20 Example 20: In Vitro Selection and Evolution of a DNA Displayed Lasso Peptide Library to Enrich High-Affinity Ligands Targeting Different Binding Pockets of PD-1

Inhibition of T-cell immune checkpoints is one of the survival mechanisms that cancer cells elicit to evade the surveillance of the immune system. Among currently known immune checkpoint molecules, programmed cell death protein 1 (PD-1) has attracted much attention from researchers in the immune oncology field in the recent years. The successful development of monoclonal antibodies against PD-1 for treating cancers is typified by nivolumab (Opdivo) and pembrolizumab (Keytruda). At the molecular level, nivolumab and pembrolizumab recognize different epitopes, also known as “binding pockets,” of PD-1; while nivolumab binds the N-loop of PD-1 (Kd=3.06 pM), pembrolizumab targets the CD loop of PD-1 (Kd=29 pM) (See: Fessas et al., Seminars in Oncology, 2017, 44:136-140).

To screen and evolve lasso peptides for high affinity ligands targeting different binding pockets of PD-1, a DNA displayed lasso peptide library is generated following the procedure described in Example 18. The generated lasso peptide library is then used to target immobilized recombinant PD-1 protein in the presence of recombinant PD-L1 (programmed death ligand 1, a native PD-1 ligand), nivolumab or pembrolizumab. Such selection strategies apply directed evolution forces to yield ligands targeting three distinct binding pockets of PD-1 that are separately occupied by PD-L1, nivolumab and pembrolizumab.

To carry out an in vitro bio-panning as shown in FIG. 7B, the recombinant human PD-1/Fc chimera protein is purchased from R&D Systems (Cat. #1086-PD) and immobilized on a Protein A coated plate (Thermo Fisher Scientific, Cat. #15155) following the manufacturer's instruction. The uncoated surface of the plate is blocked with SuperBlock (PBS) blocking buffer (Thermo Fisher Scientific, Cat. #37515) in the presence of 5% bovine serum albumin (BSA). The SuperBlock blocking buffer is removed and replaced with PBS buffer (10 mM bicarbonate phosphate buffer pH 7.4 and 150 mM NaCl). The DNA display lasso library is then applied to the immobilized PD-1 protein on the plate in the presence of PD-L1, nivolumab or pembrolizumab. The plate is incubated for 1 hour at 4° C. and then washed three times to remove the unbound lasso peptides with PBS-T buffer (10 mM bicarbonate phosphate buffer pH 7.4, 150 mM NaCl and 0.05% Tween 20). The bound lasso peptides are eluted off the immobilized PD-1 with a low pH elution buffer (75 mM Citrate, pH 2.3) for 6 min at room temperature, followed by neutralization with 1M Tris (pH 7.5). The dsDNA molecules in the neutralized sample are amplified with polymerase chain reaction (PCR) for the generation and screening of the subsequent DNA displayed lasso peptide library. During each round of in vitro bio-panning, a subpopulation of the library is enriched, and the sequence diversity of lasso peptides is monitored by Illumina Next-Gen DNA sequencing.

To evolve for high-affinity ligands of PD-1, the screening parameters and the composition of binding and washing media, such as incubation time, temperature, pH, salts and detergents, are adjusted to select for ligands with increased binding affinity. The resulting high-affinity lasso peptides are further examined individually for their ability to specifically block the binding of PD-L1, nivolumab or pembrolizumab to PD-1. The Kd values are obtained from a dose-response curve with ELISA using anti-SBP-tag mouse monoclonal antibody (EMD Millipore, Cat. #MAB10764) and goat anti-mouse IgG antibody labeled with Alexa Fluor 488 (Abcam, Cat. #ab150077).

### 6.21 Example 21: Production of a DNA Displayed Lasso Peptide Library from Multiple Lasso Peptide BGCs in Individual Wells

To produce a DNA displayed lasso peptide library from multiple lasso peptide biosynthetic gene clusters (BGCs) in individual wells, the DNA coding sequences of each BGC are codon-optimized and synthesized prior to the construction of the corresponding monocistronic DNA templates as shown in FIG. 5B. The resulting DNA templates encode multiple sets of lasso peptide precursor (A), peptidase (B), cyclase (C) and RiPP Recognition Element (RRE) that are derived from the same lasso peptide BGC. This monocistronic design principle enables rapid biosynthesis of native lasso peptides in individual wells with a minimal set of three (without RRE) or four (with RRE) codon-optimized DNA templates and devoid of the polycistronic configuration of the parental BGCs. Upon in vitro transcription and translation (TX-TL), lasso peptide precursor (A) is expressed as a “lasso precursor A-TEV-SBP” fusion protein while peptidase (B), cyclase (C) and RiPP Recognition Element (RRE) are expressed as MBP fusion proteins. The in vitro TX-TL of these four fusion proteins is carried out by adding Cell-Free Biosynthesis (CFB) cell extracts into individual wells, followed by the incubation at 37° C. for 16 hours. During the 16 hour incubation, lasso precursor peptides are separately expressed in individual wells, cleaved and cyclized by the corresponding native synthetase enzymes B, C and RRE to produce the lasso peptide fusion proteins—“lasso peptide-TEV-SBP.” Each of the generated “lasso peptide-TEV-SBP” fusion proteins is then mixed with streptavidin-coated magnetic beads, which are pre-bound with biotinylated dsDNA molecules that serve as a DNA barcode. The resulting DNA displayed lasso peptide library has each “lasso peptide-TEV-SBP” fusion protein linked to a unique DNA barcode on beads in a single well. The presence of the TEV protease recognition sequence in each “lasso peptide-TEV-SBP” fusion protein allows TEV protease-mediated cleavage to release lasso peptide for validation of lasso conformation by mass spectrometry.

To generate a library of 96 lasso peptides encoded by multiple lasso peptide BGCs, the DNA coding sequences of these lasso peptide BGCs are obtained from the research report published by Tietz et al. (See: Nat. Chem. Biol., 2017, 13(5):470-478). These DNA coding sequences are codon-optimized and synthesized by Twist Bioscience Corp. For simplicity, three exemplary lasso peptides, ukn22, BI-32169 and capistruin, are used for illustration purpose in the following paragraphs.

The coding sequences for ukn22, BI-32169 and Capistruin precursor peptides are cloned in front of the SBP coding sequence and behind a constitutive T7 promoter. The coding sequence for the TEV protease recognition site (Glu-Asn-Leu-Tyr-Phe-Gln⬇Gly) flanked by two linker sequences, Linker 1 and Linker 2, is then inserted in-frame in between each precursor peptide and the SBP to yield three DNA templates encoding ukn22-TEV—SBP, BI-32169-TEV-SBP and Capistruin-TEV-SBP.

The coding sequences of peptidase (B), cyclase (C) and RiPP recognition element (RRE) for ukn22, BI-32169 and capistruin synthetase enzymes are individually cloned in-frame behind the maltose binding protein (MBP) to create fusion proteins, MBP-B, MBP-C and MBP-RRE, each of which is expressed from a constitutive T7 promoter.

To create a lasso peptide library, the four dsDNA templates encoding “ukn22 A-TEV-SBP,” MBP-ukn22 B, MBP-ukn22 C and MBP-ukn22 RRE are added 10 ng each into the well at the A1 position of a 96-well PCR plate. This is followed by addition of the four dsDNA templates for biosynthesis of BI-32169 into the well at the A2 position and those for biosynthesis of capistruin into the well position at the A3 position. For in vitro TX-TL, 40 μL CFB cell extracts is pipetted into each well and the TX-TL reactions are incubated at 37° C. for 16 hours. During the 16 hour incubation, each lasso peptide precursor is cleaved and cyclized by corresponding native lasso peptide synthetase enzymes to form a lasso peptide with a threaded tail fused to TEV and SBP, thus resulting the production of “ukn22-TEV-SBP” in the well at the A1 position, “BI-32169-TEV-SBP” at the A2 position, and “Capistruin-TEV-SBP” at the A3 position. Following the 16 hour incubation, the streptavidin-coated magnetic beads (Dynabeads™ MyOne™ Streptavidin T1, Thermo Fisher Scientific, Cat. #65601) pre-bound with biotinylated dsDNA molecules (Integrated DNA Technologies), unique to each well, are added to the individual wells containing the produced lasso fusion proteins. The quantity of the bound biotinylated dsDNA is adjusted so that at least more than 95% of streptavidin-coated bead surface remains available for SBP-streptavidin binding. The conjugation reactions take place at 4° C. for an hour with gentle shaking. Following the one hour incubation, the 96-well PCR plate is placed on a 96 magnet plate (Alpaqua) to immobilize the magnetic beads and the TX-TL reaction mixtures within the wells are aspirated. The immobilized magnetic beads are washed three times with 50 μL ice-cold TNTB Wash Buffer (0.1 M Tris pH 7.5, 0.15 M NaCl, 0.05% Tween-20, 1% bovine serum albumin). Upon the aspiration of the last Wash Buffer, the immobilized magnetic beads in each well are re-suspended in 20 μL of TNTB buffer and used for affinity selection.

To verify successful display of lasso peptides on the beads, 5 μL of the re-suspended magnetic beads from each well is treated with TEV protease (Sigma Cat. #T4455) to release the lasso peptides following the manufacturer's instructions. An equal volume of methanol is then added to each digestion reaction and thoroughly mixed. The lasso peptides released into the supernatant post-digestion are aspirated and transferred to individual wells of a new 96-well PCR plate while the TEV-SBP fusion protein bound to the magnetic beads remain immobilized on the original 96-well PCR plate by a 96 magnet plate. The collected samples are subsequently concentrated and subjected to MALDT-TOF MS analysis to verify the presence of ukn22, BI-32169 and capistruin, each of which fused to Linker 1 and part of TEV protease recognition site (lasso peptide-Linker 1-Glu-Asn-Leu-Tyr-Phe-Gln). To confirm the simultaneous presence of the corresponding DNA barcode on the beads, 1 μL of the re-suspended magnetic beads from each of the chosen wells is used for DNA amplification with polymerase chain reaction (PCR). The amplified dsDNA molecules are subjected to DNA sequencing to verify the presence of the expected DNA barcode sequences.

What is claimed:

1. A lasso peptide display library comprising a plurality of members, wherein each member comprises a lasso peptide or a functional fragment of lasso peptide; and wherein each member is associated with a unique identification mechanism for distinguishing the plurality of members from one another, wherein the unique identification mechanism is a unique nucleic acid molecule or a unique location.

2. The lasso peptide display library of claim 1, wherein the library further comprises a solid support.

3. The lasso peptide display library of claim 2, wherein each member is associated with the unique identification mechanism through the solid support.

4. The lasso peptide display library of claim 2, wherein the solid support comprises a plurality of unique locations, and each member is associated with one of the plurality of unique locations.

5. The lasso peptide display library of any one of claims 1-4, wherein at least one of the lasso peptide and/or functional fragment of lasso peptide forms part of a fusion protein.

6. The lasso peptide display library of any one of claims 1-5, wherein at least one of the lasso peptide and/or functional fragment of lasso peptide forms part of a protein complex.

7. The lasso peptide display library of any one of claims 1-6, wherein at least one of the lasso peptide and/or functional fragment of lasso peptide forms part of a conjugate.

8. The lasso peptide display library of any one of claims 1-7, wherein the unique identification mechanism is a unique nucleic acid molecule.

9. The lasso peptide display library of claim 8, wherein the lasso peptide or functional fragment of lasso peptide is fused to a first binding partner; and wherein the unique nucleic acid molecule is conjugated with a second binding partner.

10. The lasso peptide display library of claim 9, wherein the first binding partner and the second binding partner are capable of directly or indirectly associating with one another.

11. The lasso peptide display library of claim 9 or 10, wherein the first binding partner and the second binding partner are both configured to associate with the solid support.

12. The lasso peptide display library of claim 11, wherein the solid support is coated with or comprises a third binding partner capable of associating with the first binding partner and the second binding partner.

13. The lasso peptide display library of any one of claims 9-12, wherein the first binding partner is streptavidin; and wherein the second binding partner is biotin moiety conjugated with the unique nucleic acid molecule.

14. The lasso peptide display library of any one of claims 9-12, wherein the first binding partner is a nucleic acid binding protein and the second binding partner is target nucleic acid sequence that is a fragment of the unique nucleic acid molecule.

15. The lasso peptide display library of claim 14, wherein the nucleic acid binding protein is replication protein RepA and the unique nucleic acid molecule comprises replication origin R (oriR) and cis-acting element (CIS) of RepA.

16. The lasso peptide display library of claim 12, wherein the first binding partner is a streptavidin binding protein; wherein the second binding partner is biotin moiety conjugated with the unique nucleic acid molecule; and wherein the third binding partner is streptavidin.

17. The lasso peptide display library of any one of claims 9-16, wherein the solid support is a magnetic bead.

18. The lasso peptide display library of any one of claims 9-17, wherein the lasso peptide or functional fragment thereof is associated with the unique nucleic acid molecule through a cleavable linker.

19. The lasso peptide display library of any one of claims 8-18, wherein the unique nucleic acid molecule is a nucleic acid barcode.

20. The lasso peptide display library of any one of claims 8-18, wherein the unique nucleic acid molecule encodes at least a portion of the lasso peptide or functional fragment thereof associated with the unique nucleic acid.

21. The lasso peptide display library of any one of claims 1-20, further comprising a cell-free biosynthesis system configured for providing the plurality of members.

22. The lasso peptide display library of claim 21, wherein the cell-free biosynthesis system comprises a minimal set of lasso peptide biosynthesis components.

23. The lasso peptide display library of claim 21 or 22, wherein the minimal set of lasso peptide biosynthesis components comprises (i) at least one lasso precursor peptide or (ii) a first nucleic acid sequence encoding the at least one lasso precursor peptide and cell-free transcription-translation machinery.

24. The lasso peptide display library of any one of claims 21-23, wherein the minimal set of lasso peptide biosynthesis components comprises (i) at least one lasso core peptide or (ii) a second nucleic acid sequence encoding the at least one lasso core peptide and cell-free transcription-translation machinery.

25. The lasso peptide display library of any one of claims 21-24, wherein the minimal set of lasso peptide biosynthesis components comprises (i) at least one lasso peptidase or (ii) a third nucleic acid sequence encoding the at least one lasso peptidase and cell-free transcription-translation machinery.

26. The lasso peptide display library of any one of claims 21-25, wherein the minimal set of lasso peptide biosynthesis components comprises (i) at least one lasso cyclase or (ii) a fourth nucleic acid sequence encoding the at least one lasso cyclase and cell-free transcription-translation machinery.

27. The lasso peptide display library of any one of claims 21-26, wherein the minimal set of lasso peptide biosynthesis components comprises (i) at least one RiPP recognition element (RRE) or (ii) a fifth nucleic acid sequence encoding the at least one RRE and cell-free transcription-translation machinery.

28. The lasso peptide display library of any one of claims 21-27, wherein the minimal set of lasso peptide biosynthesis components comprises
(i) a plurality of a first nucleic acid sequences each encoding a unique lasso precursor peptide;
(ii) at least one lasso peptidase or a third nucleic acid sequence encoding the lasso peptidase;
(iii) at least one lasso cyclase or a fourth nucleic acid sequence encoding the lasso cyclase; and
(iv) cell-free transcription-translation machinery.

29. The lasso peptide display library of claim 28, wherein the plurality of the first nucleic acid sequences are derived from a same lasso peptide biosynthesis gene cluster.

30. The lasso peptide display library of claim 29, wherein the plurality of the first nucleic acid sequences are obtained by randomly mutating Gene A of the same lasso peptide biosynthesis gene cluster.

31. The lasso peptide display library of claim 29, wherein the random mutation is introduced to all codons of Gene A except for the ring-forming residue.

32. The lasso peptide display library of claim 31, wherein the ring-forming residue is Glu at position 6, 7, 8, 9, 10, 11, 12, 13, 14, 15, 16, 17, 18, 19 or 20, or Asp at position 6, 7, 8, 9, 10, 11, 12, 13, 14, 15, 16, 17, 18, 19 or 20.

33. The lasso peptide display library of claim 29, wherein the plurality of the first nucleic acid sequences are obtained by changing the position of the codon coding for the ring-forming residue in Gene A of the same lasso peptide biosynthesis gene cluster.

34. The lasso peptide display library of claim 28, wherein the plurality of the first nucleic acid sequences are derived from a plurality of lasso peptide biosynthesis gene cluster.

35. The lasso peptide display library of any one of claims 28-34, wherein the minimal set of lasso peptide biosynthesis components further comprises at least one RiPP recognition element (RRE) or a fifth nucleic acid sequence encoding the RRE.

36. The lasso peptide display library of any one of claims 23-35, wherein at least one of the first, second, third, fourth and fifth nucleic acid sequences are operably linked to an expression control fragment.

37. The lasso peptide display library of any one of claims 23-36, wherein at least two of the first, second, third, fourth and fifth nucleic acid sequences form part of a same nucleic acid molecule.

38. The lasso peptide display library of claim 37, wherein at least two of the third, fourth and fifth nucleic acid sequences are fused in frame with each other in the same nucleic acid molecule.

39. The lasso peptide display library of any one of claims 23-38, wherein at least two of the first, second, third, fourth and fifth nucleic acids sequences comprise sequences derived from the same lasso peptide biosynthesis gene cluster.

40. The lasso peptide display library of any one of claims 23-39, wherein at least two of the first, second, third, fourth and fifth nucleic acid sequences comprise sequences derived from different lasso peptide biosynthesis gene clusters.

41. The lasso peptide display library of claim 40, wherein the third, fourth and fifth nucleic acid sequences comprise sequences derived from the same lasso peptide biosynthesis gene cluster of a host organism; and wherein the transcription-translation machinery is a cell lysate of the same host organism.

42. The lasso peptide display library of any one of claims 23-35, wherein at least one of the first, second, third, fourth and fifth nucleic acid sequences is DNA, mRNA or cDNA sequence.

43. The lasso peptide display library of any one of claims 23-42, wherein at least one of the first, second, third, fourth and fifth nucleic acid sequences further comprises a sequence encoding for a peptidic tag.

44. The lasso peptide display library of claim 43, wherein the peptidic tag is a purification tag.

45. The lasso peptide display library of claim 43, wherein the peptidic tag comprises a cleavable linker.

46. The lasso peptide display library of claim 43, wherein the peptidic tag forms part of a binding partner.

47. The lasso peptide display library of claim 43, wherein the peptidic tag produces a detectable signal.

48. The lasso peptide display library of any one of claims 21-47, wherein the cell-free biosynthesis system comprises cell lysate or supplemented cell lysate.

49. The lasso peptide display library of any one of claims 21-48, wherein the cell-free biosynthesis system comprises components of cellular transcription-translation machinery purified from a cell.

50. The lasso peptide display library of any one of claims 21-49, wherein the cell-free biosynthesis system comprises synthetic or recombinantly produced components of cellular transcription-translation machinery.

51. The lasso peptide display library of any one of claims 1 to 50, wherein the lasso peptide or a functional fragment of lasso peptide comprises at least one unnatural or unusual amino acid.

52. A fusion protein comprising a lasso peptide component fused to a binding partner.

53. The fusion protein according to claim 52, wherein the lasso peptide component is (i) a lasso peptide, (ii) a functional fragment of lasso peptide; (iii) a lasso precursor peptide; or (iv) a lasso core peptide.

54. The fusion protein according to claim 52 or 53, wherein the lasso peptide component is fused to the binding partner via a cleavable linker.

55. The fusion protein according to any one of claims 52 to 54, wherein the binding partner is a streptavidin binding peptide (SBP), a streptavidin protein, or a nucleic acid binding protein.

56. The fusion protein according to claim 55, wherein the nucleic acid binding protein is replication protein RepA.

57. The fusion protein according to any one of claims 52 to 56, further comprising a purification tag.

58. The fusion protein according to claim 57, wherein the purification tag is a His Tag.

59. A nucleic acid molecule encoding the fusion protein according to any one of claim 52 to

60. The nucleic acid molecule of claim 59, wherein the nucleic acid molecule is biotinylated.

61. The nucleic acid molecule of claim 59, wherein the nucleic acid molecule further comprises the replication origin R (oriR) and cis-acting element (CIS) of RepA.

62. A molecular complex comprising the fusion protein of any one of claims 52 to 58 and a nucleic acid molecule.

63. The molecular complex according to claim 62, wherein the nucleic acid molecule encodes at least a portion of the lasso peptide fragment.

64. The molecular complex according to claim 62, wherein the nucleic acid molecule is a unique member of a set of nucleic acid barcodes.

65. The molecular complex according to any one of claims 62 to 64, wherein the nucleic acid molecule is biotinylated.

66. The molecular complex according to claim 65, wherein the binding partner is the streptavidin protein.

67. The molecular complex according to claim 65, wherein the binding partner is the streptavidin binding peptide (SBP), and wherein the molecular complex further comprises a streptavidin protein.

68. The molecular complex according to any one of claims 62 to 64, wherein the nucleic acid molecule comprises the replication origin R (oriR) and cis-acting element (CIS) of RepA, and wherein the binding partner is RepA.

69. The molecular complex according to any one of claims 62 to 68, wherein the nucleic acid molecule is the nucleic acid molecule of any one of claims 59-61.

70. A composition comprising a plurality of the molecular complexes according to any one of claims 62-66, wherein each of the plurality of the molecular complexes comprises a unique lasso peptide or functional fragment of lasso peptide.

71. A method for evolving a lasso peptide of interest for a target property, the method comprising
a. providing a first lasso peptide display library comprising members derived from the lasso peptide of interest, wherein each member of the first lasso peptide display library comprises at least one mutation to the lasso peptide of interest;
b. subjecting the library to a first assay under a first condition to identify members having the target property;
c. identifying the mutations of the identified members as beneficial mutations; and
d. introducing the beneficial mutations into the lasso peptide of interest to provide an evolved lasso peptide.

72. The method of claim 71, wherein the method further comprises:
f. providing an evolved lasso peptide display library comprising members derived from the evolved lasso peptide, wherein the members of the second library retain at least one beneficial mutation; and
g. repeating steps b through d.

73. The method of claim 72, wherein the method further comprises repeating steps f and g for at least one more round.

74. The method of any one of claims 71-73, wherein the evolved lasso peptide display library is subjected to the first assay under a second condition more stringent for the target property than the first condition.

75. The method of any one of claims 72-74, wherein the evolved lasso peptide display library is subjected to a second assay to identify members having the target property.

76. The method of any one of claims 71-75, wherein the method further comprises validating the evolved lasso peptide using at least one additional assay different from the first or second assay.

77. The method of any one of claims 71-76, wherein the target property is binding affinity for a target molecule.

78. The method of any one of claims 71-76, wherein the target property is binding specificity for a target molecule.

79. The method of any one of claims 71-76, wherein the target property is capability of modulating a cellular activity or cell phenotype.

80. The method of claim 78, wherein the modulation is antagonist modulation or agonist modulation.

81. The method of any one of claims 71-80, wherein the mutation comprises substituting at least one amino acid with an unusual or unnatural amino acid.

82. The method of any one of claims 71 to 81, wherein the target property is at least two target properties screened simultaneously.

83. A method for identifying a lasso peptide that specifically binds to a target molecule, the method comprising:
providing a lasso peptide display library comprising a plurality of members, each member comprising a lasso peptide or a functional fragment of lasso peptide;
contacting the library with the target molecule under a suitable condition that allows at least one member of the library to form a complex with the target molecule; and
identifying the member of in the complex.

84. The method of claim 82,
wherein the contacting is performed by contacting the library with the target molecule in the presence of a reference binding partner of the target molecule under a suitable condition that allows at least one member of the library to compete with the reference binding partner for binding to the target molecule; and
wherein the identifying step is performed by detecting reduced binding of the reference binding partner to the target molecule; and identifying the member responsible for the reduced binding.

85. The method of claim 84, wherein the reference binding partner is a ligand for the target molecule.

86. The method of claim 84 or 85, wherein the target molecule comprises one or more target sites, and the reference binding partner specifically binds to a target site of the target molecule.

87. The method of claim 85, wherein the reference binding partner is a natural ligand or synthetic ligand for the target molecule.

88. The method of any one of claims 83 to 87, wherein the target molecule is at least two target molecules.

89. A method for identifying a lasso peptide that modulates a cellular activity, the method comprising
a. providing a lasso peptide display library comprising a plurality of members, each member comprising a lasso peptide or a functional fragment of lasso peptide;
b. subjecting the library to a suitable biological assay configured for measuring the cellular activity;
c. detecting a change in the cellular activity; and
d. identifying the members responsible for the detected change.

90. The method of claim 89, wherein the step b is performed by subjecting the library to multiple biological assays configured for measuring the cellular activity; and the method further comprises selecting the members that have a high probability of being identified as responsible for the detected change in the cellular activity.

91. A method for identifying an agonist or antagonist lasso peptide for a target molecule, the method comprising:
providing a lasso peptide display library comprising a plurality of members, each member comprising a lasso peptide or a functional fragment of lasso peptide;
contacting the library with a cell expressing the target molecule under a suitable condition that allows at least one member of the library to bind to the target molecule;
measuring a cellular activity mediated by the target molecule; and
identifying the member as an agonist ligand for the target molecule if said cellular activity is increased; or identifying the member as an antagonist ligand if said cellular activity is decreased.


SEQUENCE LISTING (64 sequences)

SEQ ID NO: 1 - protein, 18 aa, Thermobifida fusca
  Ukn22
WYTAEWGLELIFVFPRFI

SEQ ID NO: 2 - protein, 40 aa, Thermobifida fusca
  Ukn22 precursor A
MEKKKYTAPQLAKVGEFKEATGWYTAEWGLELIFVFPRFI

SEQ ID NO: 3 - protein, 138 aa, Thermobifida fusca
  Ukn22 peptidase B
MSENVVLQRSNVRLSWRTKWAARCAVGAARLLARKPPERIRATLLRLRGEVRPATYEEAKAARDAVLAVSLRCAGLRACLQRSLAIALLCRMRGTWATWCVGVPRRPPFIGHAWVEAEGRLVEEGVGYDYFSRLITVD

SEQ ID NO: 4 - protein, 610 aa, Thermobifida fusca
  Ukn22 cyclase C
MVGCISPYFAVFPDKDVLGQATDRLPAAQTLASHPSGRPWLVGALPADQLLLVEAGERRLAVIGHCSAEPERLRAELAQIDDVAQFDRIARTLDGSFHLVVVVGDQMRIQGSVSGLRRVFHAHVGTARIAADRSDVLAAVLGVSPDPDVLALRMFNGLPYPLSELPPWPGVEHVPAWHYLSLGLHDGRHRVVQWWHPPEAELAVTAAAPLLRTALAGAVDTRTRGGGVVSADLSGGLDSTPLCALAARGPAKVVALTFSSGLDTDDDLRWAKIAHQSFPSVEHVVLSPEDIPGFYAGLDGEFPLLDEPSVAMLSTPRILSRLHTARAHGSRLHMDGLGGDQLLTGSLSLYHDLLWQRPWTALPLIRGHRLLAGLSLSETFASLADRRDLRAWLADIRHSIATGEPPRRSLFGWDVLPKCGPWLTAEARERVLARFDAVLESLEPLAPTRGRHADLAAIRAAGRDLRLLHQLGSSDLPRMESPFLDDRVVEACLQVRHEGRMNPFEFKSLMKTAMASLLPAEFLTRQSKTDGTPLAAEGFTEQRDRIIQIWRESRLAELGLIHPDVLVERVKQPYSFRGPDWGMELTLTVELWLRSRERVLQGANGGDNRS

SEQ ID NO: 5 - protein, 90 aa, Thermobifida fusca
  Ukn22 RRE
METTGAEFRLRPEISVAQTDYGMVLLDGRSGEYWQLNDTAALIVQRLLDGHSPADVAQFLTSEYEVERTDAERDIAALVTSLKENGMALP

SEQ ID NO: 6 - protein, 19 aa, Streptomyces sp.
  BI-32169
GLPWGCPSDIPGWNTPWAC

SEQ ID NO: 7 - protein, 19 aa, Kibdelosporangium sp.
  BI-32169 analog
GLPWGCPNDLFFVNTPFAC

SEQ ID NO: 8 - protein, 43 aa, Kibdelosporangium sp.
  BI-32169 analog precursor A
MIKDDEIYEVPTLVEVGDFAELTLGLPWGCPNDLFFVNTPFAC

SEQ ID NO: 9 - protein, 43 aa, Artificial Sequence
  Description of Artificial Sequence Synthetic polypeptide
  Hybrid BI-32169 precursor A
MIKDDEIYEVPTLVEVGDFAELTLGLPWGCPSDIPGWNTPWAC

SEQ ID NO: 10 - protein, 142 aa, Kibdelosporangium sp.
  BI-32169 analog peptidase B
MTMPVAAETTVPLPWHRHITARLATGSARVLIRLRPRRLRVVLRMVSRGARPATAAQALSARQAVVSVSVRCAGQGCLQRAVATALLCRLAGDWPDWCTGFRTRPFRAHAWVEAEGGAVGEPGDMPLFHTVISVRHPAREAR

SEQ ID NO: 11 - protein, 197 aa, Kibdelosporangium sp.
  BI-32169 analog cyclase C
MRDRRWRAGVRPSTADAGTKGKGLLVGGNEFLVFPDCPVALDAPGGRTVPHASGRPWLVGDWSDDDIVVISAGTRRLAIVGQARVNVHAVERSLEAAGSVRDLDAVVGTIPGNFHLIASIDGRTRVQGTVSTVRQVFTATIVGTTVAASGPGLLAAATGSRVDGDALALRLVPVVPWPLCLRPVWSGVEQVAAGHWL

SEQ ID NO: 12 - protein, 86 aa, Kibdelosporangium sp.
  BI-32169 analog RRE
MTIALTPNVTATDSEDGLVLLNESTGRYWTLNGTGAATLRLLLAGNSPAQTASRLAERYPDAVDRTQRDVVALLAALRNARLVTSS

SEQ ID NO: 13 - protein, 22 aa, Artificial Sequence
  Description of Artificial Sequence Synthetic peptide
  PelB secretion sequence (ssPelB)
MKYLLPTAAAGLLLLAAQPAMA

SEQ ID NO: 14 - protein, 42 aa, Artificial Sequence
  Description of Artificial Sequence Synthetic polypeptide
  TorA secretion sequence (ssTorA)
MNNNDLFQASRRRFLAQLGGLTVAGMLGPSLLTPRRATAAQA

SEQ ID NO: 15 - protein, 7 aa, Artificial Sequence
  Description of Artificial Sequence Synthetic peptide
  TEV cleavage site
ENLYFQG

SEQ ID NO: 16 - protein, 20 aa, Artificial Sequence
  Description of Artificial Sequence Synthetic peptide
  Linker 1
GAAAKGAAAKGAAAKGAAAK

SEQ ID NO: 17 - protein, 25 aa, Artificial Sequence
  Description of Artificial Sequence Synthetic peptide
  Linker 2
SGGGGSGGGGSGGGGSGGGGSGGGG

SEQ ID NO: 18 - protein, 371 aa, Artificial Sequence
  Description of Artificial Sequence Synthetic polypeptide
  Truncated maltose-binding protein (MBP) (deletion 2-29)
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 19 - protein, 38 aa, Artificial Sequence
  Description of Artificial Sequence Synthetic polypeptide
  Streptavidin-binding Peptide (SBP)
MDEKTTGWRGGHVVEGLAGELEQLRARLEHHPQGQREP

SEQ ID NO: 20 - protein, 285 aa, Artificial Sequence
  Description of Artificial Sequence Synthetic polypeptide
  Replication protein RepA (RepA)
MTDLHQTYYRQVKNPNPVFTPREGAGTPKFREKPMEKAVGLTSRFDFAIHVAHARSRGLRRRMPPVLRRRAIDALLQGLCFHYDPLANRVQCSITTLAIECGLATESGAGKLSITRATRALTFLSELGLITYQTEYDPLIGCYIPTDITFTLALFAALDVSEDAVAAARRSRVEWENKQRKKQGLDTLGMDELIAKAWRFVRERFRSYQTELQSRGIKRARARRDANRERQDIVTLVKRQLTREISEGRFTANGEAVKREVERRVKERMILSRNRNYSRLATASP

SEQ ID NO: 21 - protein, 19 aa, Burkholderia thailandensis
  Capistruin
GTPGFQTPDARVISRFGFN

SEQ ID NO: 22 - protein, 47 aa, Burkholderia thailandensis
  Capistruin precursor A
MVRLLAKLLRSTIHGSNGVSLDAVSSTHGTPGFQTPDARVISRFGFN

SEQ ID NO: 23 - protein, 241 aa, Burkholderia thailandensis
  Capistruin peptidase B
MTPASHCHIAVFDQAIVALDMQRSRYFLYDEACAKAFADHYLDFKPIDAPHALKPLISDRIVVAASPASVPKRIADYRGWAFDAFDSGIWASRTLGERSAAGFEWLPFWRIVRGAVSLKMRGFRALSALDRLARLDAGAEQRARTDGGPSRTAERYLRASIWSPFRITCLQMSFALATHLRRENVPAQLVIGVRPMPFVAHAWVEIDGRVCGDEPELKKSYGEIYRTPRHDERAGPFGLAA

SEQ ID NO: 24 - protein, 535 aa, Burkholderia thailandensis
  Capistruin cyclase C
MTLLEAGARARAYLRDAHSRIERSLARARTLQEARDTVTRSVWGAYLLVLDEAASGRRLFMPDPLHSVRLYYRTDERGRVDVDPRAANLLDRASIDWNLDYLIEFACTQFGPLDETPFASVRVVPPGCALVVGPDGRCAIERAWLPRAQAAGDVRASCAAALDDVYSRIAHSHPSVCAALSGGVDSSAGAIFLRKALGANAPLAAVHLYSTSSPDCYERDMAARVADSIGAQLICIDIDRHLPFSERIVRTPPAALNQDMLFLGIDRAVSNALGPSSVLLEGQGGDLLFRAVPDANAVLDALRSNGWSFALRTAEKLAMLHNDSIPRILLMAAKIALRRRLFGQDAPASQQTMSRLFASSAPRAAAGRSRRHAPRADAPLDESISMLDRFVSIMTPVTDAAYTSRLNPYLAQPVVEAAFGLRSYDSFDHRNDRIVLREIASAHTPVDVLWRRTKGSFGIGFVKGIVSHYDALRELIRDGVLMRSGRLDEAELEHALKAVRVGQNAAAISVALVGCVEVFCASWQNFVTNRHAAVC

SEQ ID NO: 25 - protein, 58 aa, Artificial Sequence
  Description of Artificial Sequence Synthetic polypeptide
  microcin j25 precursor
MIKHFHFNKLSSGKKNNVPSPAKGVIQIKKSASQLTKGGAGHVPEYFVGIGTPISFYG

SEQ ID NO: 26 - protein, 208 aa, Artificial Sequence
  Description of Artificial Sequence Synthetic polypeptide
  microcin j25 peptidase
MIRYCLTSYREDLVILDIINDSFSIVPDAGSLLKERDKLLKEFPQLSYFFDSEYHIGSVSRNSDTSFLEERWFLPEPDKTLYKCSLFKRFILLLKVFYYSWNIEKKGMAWIFISNKKENRLYSLNEEHLIRKEISNLSIIFHLNIFKSDCLTYSYALKRILNSRNIDAHLVIGVRTQPFYSHSWVEVGGQVINDAPNMRDKLSVIAEI

SEQ ID NO: 27 - protein, 214 aa, Artificial Sequence
  Description of Artificial Sequence Synthetic polypeptide
  microcin j25 cyclase
MEIFNVKLNDTSIRIIFCKTLSAFRTENTIVMLKGKAVSNGKPVSTEEIARVVEEKGVSEVIENLDGVFCILIYHFNDLLIGKSIQSGPALFYCKKNMDIFVSDKISDIKFLNPDMTFSLNIKMAEHYLSGNRIATQESLITGIYKVNNGEFIKFNNQLKPVLLRDEFSITKKNNSTIDSIIDNIEMMRDNRKIALLFSGGLDSALIFHTLKES

SEQ ID NO: 28 - protein, 40 aa, Artificial Sequence; identical to SEQ ID NO: 2
  Description of Artificial Sequence Synthetic polypeptide
  ukn22 precursor

SEQ ID NO: 29 - protein, 138 aa, Artificial Sequence; identical to SEQ ID NO: 3
  Description of Artificial Sequence Synthetic polypeptide
  ukn22 peptidase

SEQ ID NO: 30 - protein, 610 aa, Artificial Sequence; identical to SEQ ID NO: 4
  Description of Artificial Sequence Synthetic polypeptide
  ukn22 cyclase

SEQ ID NO: 31 - protein, 90 aa, Artificial Sequence; identical to SEQ ID NO: 5
  Description of Artificial Sequence Synthetic polypeptide
  ukn22 RRE

SEQ ID NO: 32 - protein, 47 aa, Artificial Sequence; identical to SEQ ID NO: 22
  Description of Artificial Sequence Synthetic polypeptide
  capistruin precursor

SEQ ID NO: 33 - protein, 241 aa, Artificial Sequence; identical to SEQ ID NO: 23
  Description of Artificial Sequence Synthetic polypeptide
  capistruin peptidase

SEQ ID NO: 34 - protein, 202 aa, Artificial Sequence
  Description of Artificial Sequence Synthetic polypeptide
  capistruin cyclase
MTLLEAGARARAYLRDAHSRIERSLARARTLQEARDTVTRSVWGAYLLVLDEAASGRRLFMPDPLHSVRLYYRTDERGRVDVDPRAANLLDRASIDWNLDYLIEFACTQFGPLDETPFASVRVVPPGCALVVGPDGRCAIERAWLPRAQAAGDVRASCAAALDDVYSRIAHSHPSVCAALSGGVDSSAGAIFLRKALGANAP

SEQ ID NO: 35 - protein, 46 aa, Artificial Sequence
  Description of Artificial Sequence Synthetic polypeptide
  lariatin precursor
MTSQPSKKTYNAPSLVQRGKFARTTAGSQLVYREWVGHSNVIKPGP

SEQ ID NO: 36 - protein, 147 aa, Artificial Sequence
  Description of Artificial Sequence Synthetic polypeptide
  lariatin peptidase
MPVVGAMAIPSKTRISATERLRLASALSLGKALSHLPPGLLRRSMTAFAAKARPASYREAEAAVVSITQYSKASAGPGSCLQRSISVCILMRLDGRWPTWCVGVPSKPPFRAHAWIEAGGQIVAELGDMNSYSRLMTISTHAERTES

SEQ ID NO: 37 - protein, 604 aa, Artificial Sequence
  Description of Artificial Sequence Synthetic polypeptide
  lariatin cyclase-
MNGIDIAVVTDDPAILKSVHERYPDGSKHSIELEHGHNVHIFVRTATLVLSSYIRDNEAIAVLGYSNVHESSMRAILESSPGVAHMNSALGQLIGAQWVVAIRKGAVRIQGTVSGLSRVYWSKRGSRFVASNRSRELARLLGSELDPTQVAFRTIHPMQHPFTSSSCWKDLEGVLPGEYLEVTGRSSPRTERWWTPATSYRSLEDGANETAAALFSVVRNQLSDHSAASCDISGGLDSSSIAAIAANAAKSGETHTVLHGTTSVSRDEFNSDADWAIELSKSLKLDSHSFLSWNDMPKEYDDLDALASYDLDEPSIASISHSRFTHLINVARSKGSQVHLTGFGGDELFIGSPTFCVDLFKTQPLLSARLLLTYRAMYRWTFRSLVRPLTTPMSYQQWMRTKSLSTDRSTLRIPPLSWGFHGVIPPWITRDARHSMYDHVRSASSATFPLAPTPGRHFELENLYQCARLFRTMSDIVSQTSGVLLVAPMLEQAVVEAAISVRTPERLTPHKYKPVLTHATRGLLPAVVAERQTKGGEDTDAAIGFSENISAIRELWDESRLASLGIVDGDYLSNALRRPDSAEFDDCAIAKTLATELWLRSLEK

SEQ ID NO: 38 - protein, 84 aa, Artificial Sequence
  Description of Artificial Sequence Synthetic polypeptide
  lariatin RRE
MVLRLRKNVIITPTEYGAVALDERSGDYYQLNSTAALILDQLTKKIPVESIAARIALDFEVSKAQASADLDEYLRMLREQGLLR

SEQ ID NO: 39 - protein, 42 aa, Artificial Sequence
  Description of Artificial Sequence Synthetic polypeptide
  ukn16 precursor
MKDYVPPVVEVIASFKEATNGVWFGNYVDVGGAKAPFPWGSN

SEQ ID NO: 40 - protein, 142 aa, Artificial Sequence
  Description of Artificial Sequence Synthetic polypeptide
  ukn16 peptidase
MSIPLQPEATTRVNFHDRLVALIAIIIGRRMERQRIGKFCRRLETWSERYPPADADMAKRYRNAVCSVSRRCRSQQGCLLRSLSTAAACRISRRSVTWCTGFTDRPFRAHAWVEANGIPIGEPDAVRRYTITRTSSERKDTQ

SEQ ID NO: 41 - protein, 615 aa, Artificial Sequence
  Description of Artificial Sequence Synthetic polypeptide
  ukn16 cyclase-RRE fusion
MMPFTHKNPNRTVIVRGGKPTDRSKLPASISINDEGTCVQVPLLSGDRMFWTVSKDEVLLSDSAFRLASITNADLDLERIIMDLLPSLPDSLRGDKSPWRNIHSVPAATTLHLDKSNRPRYTRAEPSPIKHVSNDVILASLRSRFLTIADEWRNEAPLSADVSGGVDSAAIAYIFASEGVRMPLYHETPDDPMNQDSRWAERISEDVRMPLIKIGRVVDGNRSFESTAEYPNREIPEEPVFWSDIEGYLSRISEMEADTSRIHVTGFGGDELFASMPSSSWSCLREHPLRLRDIRRQYSADYRVPPYQAILDLTDSTDLHEELRSSLLDAEQDHGHRSSPCGWHDAIRIPEFLTAKARDTLYGAIESQLKHADIRPLSPDRSRHQALYSLSMQARMLNQVNRTFASDDITFRSPYLDRGIVGYALCAPISARTEGNLHKAVLYRALKGIVPETIFRRPVKGDHSYSLYLAWQRSKDTLLDSIAGGVLDEEGLIDIPAVRRRASMPMPDITFLFEMQRVAAVEGGDMQTDNTMANTSLKDQISLYPKEGGAIVFVNDTGEYLQVNEIGRIILDGLMHGKTVEDCMNAIAEEYQTDRQIIVRDTERFLADIGKHVRL

SEQ ID NO: 42 - protein, 35 aa, Artificial Sequence
  Description of Artificial Sequence Synthetic polypeptide
  adanomysin precursor
MFYEPPVVVDLGSVRDVTLGSSTSGTADANSQYYW

SEQ ID NO: 43 - protein, 602 aa, Artificial Sequence
  Description of Artificial Sequence Synthetic polypeptide
  adanomysin cyclase
MRNGLLGVFPPASSGEVVRVQGPWREGELRRVDGPEGTVAVLGQCLSDDDRLRRTALRALASGGPGELTRLPGSYLCLVIRHEELTAYVDAAGQYPLFFRDTGTRLVFGTRPVSVADAAGARRRPDTAVLAAGIFCPGAPSLTGERSVVAGVSKVGGGQALRRTARGKVERWVHEPLETDPGVSLARSAEALRDALETAVRLRVAGTERVSADFSGGLDSTSLAFLTLRHRPGPLPVTTYRGAASACDDLVHAERFARLDPRLRMEVVTGTRETLTYQGLGDRSGGAGHDSDEPDPAVVALARSRLRLDQVARLGAGVHLGGEGGDALLVAPPGYLAALARPERLRQLAKESRVLARARQEAPSAVAARAVGLARTPLATALRRLADGFERHATGGTGRAGAGDVGWLDAIAWWPGPGSETEWLTRAASAELAGLAREAAGSAGRTAGSRAGDLTALDNVRTSGAVQRQLSEMARPFGVWPQAPFLDSAVIRACAALPAHLRAAPPAFKPLLGAALADLVPAPVLARRTKGDYGDEDYQGARACARELRGLLVDSRLAELGVVEPSAVVAALDRAVMGLRVPFPALNRLLAAEIWLRNTTWH

SEQ ID NO: 44 - protein, 237 aa, Artificial Sequence
  Description of Artificial Sequence Synthetic polypeptide
  adanomysin RRE-peptidase fusion
MAAFHIPEHVHESSGPHGGTVLLDARTGQWYAMNGTARALWSEWRESGDFDAGVRTVAARFPPALGERVRTDAGQLAETLLQRGLVSAEPSSDGSGRCLRPVRRAGRRFSAAPRRNRSGATAALVVALCLLRLPFGVTVRVVAALTSRCPHPATHAQAEQALAAVRRVSRRYPGRVACLELSLAATVRLALAGLGAQWCLGSADDPYRFHAWIEAGGRPVTSPSEGELSGFRKVLTV

SEQ ID NO: 45 - protein, 21 aa, Artificial Sequence
  Description of Artificial Sequence Synthetic peptide
  microcin j25
GGAGHVPEYFVGIGTPISFYG

SEQ ID NO: 46 - protein, 18 aa, Artificial Sequence
  Description of Artificial Sequence Synthetic peptide
  ukn22
WYTAEWGLELIFVFPRFI

SEQ ID NO: 47 - protein, 19 aa, Artificial Sequence
  Description of Artificial Sequence Synthetic peptide
  capistruin
GTPGFQTPDARVISRFGFN

SEQ ID NO: 48 - protein, 20 aa, Artificial Sequence
  Description of Artificial Sequence Synthetic peptide
  lariatin
GSQLVYREWVGHSNVIKPGP

SEQ ID NO: 49 - protein, 22 aa, Artificial Sequence
  Description of Artificial Sequence Synthetic peptide
  ukn16
GVWFGNYVDVGGAKAPFPWGSN

SEQ ID NO: 50 - protein, 16 aa, Artificial Sequence
  Description of Artificial Sequence Synthetic peptide
  adanomysin
GSSTSGTADANSQYYW

SEQ ID NO: 51 - protein, 18 aa, Artificial Sequence
  Description of Artificial Sequence Synthetic peptide
  Fusilassin
WYTAEWGLELIFVFPRFI

SEQ ID NO: 52 - protein, 40 aa, Thermobifida fusca; identical to SEQ ID NO: 2
  FusA

SEQ ID NO: 53 - protein, 610 aa, Thermobifida fusca; identical to SEQ ID NO: 4
  Fusilassin cyclase FusC

SEQ ID NO: 54 - protein, 138 aa, Thermobifida fusca; identical to SEQ ID NO: 3
  Fusilassin peptidase FusB

SEQ ID NO: 55 - protein, 90 aa, Thermobifida fusca; identical to SEQ ID NO: 5
  Fusilassin RRE FusE

SEQ ID NO: 56 - protein, 578 aa, Thermobifida fusca
  Fusilassin ABC transporter FusD
MPLSPPRSLRLLVAHLWPHRRAVAFGALLGLLGGIGTLAEPLVAMAVVDALGEGSPLGWLLALLTVLVVGGAALAGLSSYVLHRTAESMVAAARRRLVSHILLLRVPELDRLKPGDLLSRVTSDTTYIRSAAGQALVDSGSALLVAIGSIVLMAWIDLPLLLVCLAVIGVIGVGSAVMMPPIRRANERSQRAVGEVGALVERALGAFRTLKASSAERREISAAKAAVRTAWREGVRSAAWTAATNVAVVVTSQAAFLVVLGAGGARVAMGAIDVSELIAFLLYLMRLTGFVAQLAQAVSSLQSGLAAMRRIAEVEQLPVEHIGVPPRRTPAATSAASVSFTGVSFRYREDGPWTLRNVTLDVPAGGLTALVGPSGAGKTTLFSLVERFYDPHEGVVAIDGVDVRDIPLVRLRSMIGYVEQDAPILAGTLRDNLCFAAPHADEEEIRRVVELTRLTSLVERLPDGLDTQVGHRGTTLSGGERQRVAIARALLRRPRLLLLDEATSQLDATNETALRDVVVAIAKTTTVIIIAHRLSTVVDADRIAVVEGGRIRAVGRHTDLLLIDDLYRELIEAQLLAS

SEQ ID NO: 57 - protein, 506 aa, Artificial Sequence
  Description of Artificial Sequence Synthetic polypeptide
  MBP-FusA-TEV-SBP
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSHHHHHHANSVPLVPRGSENLYFQSGSMEKKKYTAPQLAKVGEFKEATGWYTAEWGLELIFVFPRFIGGGGSGGGGSGGGGSYPYDVPDYAENLYFQGMDEKTTGWRGGHVVEGLAGELEQLRARLEHHPQGQREP

SEQ ID NO: 58 - protein, 87 aa, Artificial Sequence
  Description of Artificial Sequence Synthetic polypeptide
  Fusilassin-TEV-SBP
WYTAEWGLELIFVFPRFIGGGGSGGGGSGGGGSYPYDVPDYAENLYFQGMDEKTTGWRGGHVVEGLAGELEQLRARLEHHPQGQREP

SEQ ID NO: 59 - protein, 48 aa, Artificial Sequence
  Description of Artificial Sequence Synthetic polypeptide
  TEV protease-cleaved Fusilassin
WYTAEWGLELIFVFPRFIGGGGSGGGGSGGGGSYPYDVPDYAENLYFQ

SEQ ID NO: 60 - protein, 151 aa, Artificial Sequence
  Description of Artificial Sequence Synthetic polypeptide
  Streptavidin core region (SAV)
GSGAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 61 - protein, 212 aa, Artificial Sequence
  Description of Artificial Sequence Synthetic polypeptide
  FusA-TEV-SAV
MEKKKYTAPQLAKVGEFKEATGWYTAEWGLELIFVFPRFIGGGSVSYTHLRAHETENLYFQGSGAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 62 - protein, 617 aa, Artificial Sequence
  Description of Artificial Sequence Synthetic polypeptide
  MBP-FusA-TEV-SAV
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSHHHHHHANSVPLVPRGSENLYFQSGSMEKKKYTAPQLAKVGEFKEATGWYTAEWGLELIFVFPRFIGGGGSGGGGSGGGGSYPYDVPDYAENLYFQGSGAAEAGITGTWYNQLGSTFIVTAGADGALTGTYESAVGNAESRYVLTGRYDSAPATDGSTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 63 - protein, 198 aa, Artificial Sequence
  Description of Artificial Sequence Synthetic polypeptide
  Fusilassin-TEV-SAV
WYTAEWGLELIFVFPRFIGGGGSGGGGSGGGGSYPYDVPDYAENLYFQGSGAAEAGITGTWYNQLGSTFIVTAGADGALTGTYESAVGNAESRYVLTGRYDSAPATDGSTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 64 - protein, 6 aa, Artificial Sequence
  Description of Artificial Sequence Synthetic peptide
  TEV cleavage site partial sequence
ENLYFQ